(19) United States
(12) Patent Application Publication
(10) Pub. No.: US 2024/0166759 A1
(43) Pub. Date: May 23, 2024

(54) COMBINATIONS COMPRISING ANTI-TM4SF1 ANTIBODIES AND IMMUNOTHERAPEUTIC AGENTS AND METHODS OF USING THE SAME

(71) Applicant: Angiex, Inc., Cambridge, MA (US)

(72) Inventors: Paul A. Jaminet, Sudbury, MA (US); Shou-Ching S. Jaminet, Sudbury, MA (US); Gabriel Gonzalez, Needham, MA (US)

(21) Appl. No.: 18/473,511

(22) Filed: Sep. 25, 2023

Related U.S. Application Data
(63) Continuation of application No. PCT/US2022/022035, filed on Mar. 25, 2022.
(60) Provisional application No. 63/166,805, filed on Mar. 26, 2021.

(51) Int. Cl. C07K 16/28; A61K 47/68; A61P 35/00; A61K 39/00

(52) U.S. Cl. CPC C07K 16/2896; A61K 47/68031; A61K 47/68033; A61P 35/00; C07K 16/2818; C07K 16/2827; A61K 2039/507; C07K 2317/565

(57) ABSTRACT

Anti-TM4SF1 antibodies, and antigen-binding fragments thereof, are described and antibody drug conjugates containing the same, and combinations of such antibodies and ADCs with an immunotherapy, such as an immunomodulatory agent.

### CROSS-REFERENCE

This application is a continuation application of International Application No. PCT/US2022/022035, filed Mar. 25, 2022, which claims the benefit of U.S. Provisional Patent Application No. 63/166,805, filed Mar. 26, 2021, each of which is incorporated herein by reference in its entirety.

### SEQUENCE LISTING

This instant application contains a Sequence Listing which has been submitted electronically in XML format and is hereby incorporated by reference in its entirety. Said XML copy, created on Sep. 25, 2023, is named 52628-718_301_SL.xml and is 245,645 bytes in size.

### BACKGROUND

There remains a need in the art for cancer therapeutics, and in particular therapeutics with improved therapeutic margins that can regress primary tumors as well as invasive tumor cells and metastases.

Cancer therapies designed to destroy tumor blood vessels have in the past failed in clinical trials due to toxicity. Examples include the vascular disrupting agents such as Combretastatin (CA4P). See, e.g., Grisham et al. Clinical trial experience with CA4P anticancer therapy: focus on efficacy, cardiovascular adverse events, and hypertension management. Gynecol Oncol Res Pract. 2018; 5:1. CA4P reduced overall survival from 16.2 to 13.6 months in the Phase II FALCON study, and seven patients have experienced heart attacks while being treated with CA4P. Id. As coronary heart disease and stroke are leading causes of death, any vascular targeted toxic therapy may lead to a risk of lethal toxicity.

TM4SF1 is an endothelial marker with a functional role in angiogenesis. See, e.g., Shih et al. The L6 protein TM4SF1 is critical for endothelial cell function and tumor angiogenesis. Cancer Res. 2009; 69(8):3272-7.

### SUMMARY OF THE INVENTION

One embodiment provides a combination comprising: a. an anti-TM4SF1 binding protein; and b. an immunotherapeutic agent. In some embodiments, the immunotherapeutic agent is a cytokine, an adjuvant, or an immune checkpoint inhibitor. In some embodiments, the immunotherapeutic agent is an antibody or an antigen binding fragment. In some embodiments, the immunotherapeutic agent is an antibody or an antigen binding fragment thereof directed against a target present in a cell of a myeloid lineage, a tumor cell, a cell of a lymphoid lineage, or a protein present in a tumor microenvironment. In some embodiments, the immunotherapeutic agent is an antibody or an antigen binding fragment thereof directed against at least one of: PD-1, CTLA-4, CD40, CSF1/CSF1R, SIRPα, CLEC-1. In some embodiments, the immunotherapeutic agent is an antibody or an antigen binding fragment thereof directed against at least one of CCR4, CTLA-4, AIR, A2AR, A3R, TIM-3, BTLA, VISTA, TIGIT, LAG-3, ILRa/CD25, ITGB1/CD29, Ly 24/CD44, CD48, CEACAM1/CD66a, Nt5e/CD73, CD94/NKG2A, FAS/CD95, SLAF1/CD150, NRP1/CD304, GITR/CD357, ICOS, Tnfrs4/OX40, Folr4/JUNO, P2X7, ANXA2, IDO, B7-H6, KIR, GARP (LRRC32), TNFR2. In some embodiments, the immunotherapeutic agent is an antibody or an antigen binding fragment thereof directed against at least one of: PD-L1, PD-L2, B7-H3, B7-H4, CD47, TDO, DcR3. In some embodiments, the immunotherapeutic agent is an antibody or an antigen binding fragment thereof directed against at least one of: an exosome, a cytokine, a interleukin, or a chemokines. In some embodiments, the immunotherapeutic agent is an antibody or an antigen binding fragment thereof directed against at least one of: PS, PD-L1, STING.

One embodiment provides a combination comprising: a. an anti-TM4SF1 binding protein; and b. an immunotherapeutic agent selected from the group consisting of: ipilimumab, nivolumab, pembrolizumab, atezolizumab, durvalumab, tremelimumab, spartalizumab, avelumab, sintilimab, toripalimab, MGA012, MGD013, MGD019, enoblituzumab, MGD009, MGC018, MEDI0680, PDR001, FAZ053, TSR022, MBG453, relatlinab (BMS986016), LAG525, IMP321, REGN2810(cemiplimab), REGN3767, pexidartinib, LY3022855, FPA008, BLZ945, GDC0919, epacadostat, indoximid, BMS986205, CPI-444, MEDI9447, PBF509, lirilumab, IMC-001, Monalizumab, and combinations thereof. In some embodiments, the anti-TM4SF1 binding protein comprises: a heavy chain variable domain comprising a CDR3 domain comprising an amino acid sequence that has at least 75% identity to SEQ ID NO: 8, 20, 32, 44, 56, 68, 80, 96, 118, 119, 120, 121, or 162; a CDR2 domain comprising an amino acid sequence that has at least 75% identity to SEQ ID NO: 7, 19, 31, 43, 55, 67, 79, 95, 116, 117, or 161; and a CDR1 domain comprising an amino acid sequence that has at least 75% identity to SEQ ID NO: 6, 18, 30, 42, 54, 66, 78, 94, 115, or 160; and a light chain variable domain comprising a CDR3 domain comprising an amino acid sequence that has at least 75% identity to SEQ ID NO: 14, 26, 38, 50, 62, 74, 86, 110, 129, or 159; a CDR2 domain comprising an amino acid sequence that has at least 75% identity to SEQ ID NO: 13, 25, 37, 49, 61, 73, 85, or 109, 128, or 158; and a CDR1 comprising an amino acid sequence that has at least 75% identity to SEQ ID NO: 12, 24, 36, 48, 60, 72, 84, 107, 108, 124, 125, 126, 127, or 157.

In some embodiments, the anti-TM4SF1 binding protein comprises an IgG Fc region, and wherein said IgG Fc region comprises a mutation in at least one of the following positions E233, L234, L235, G237, M252, S254, T250Q, T256E, D265, N297, K322, P331, M428, and N434; as numbered by the EU index as set forth in Kabat. In some embodiments, said IgG Fc region comprises at least one of the following mutations: E233P, L234A, L235A, G237A, M252Y, S254T, T250Q, T256E, D265A, N297C, K322A, P331G, M428L, N434A, and N434S; as numbered by the EU index as set forth in Kabat.

In some embodiments, the anti-TM4SF1 binding protein is conjugated to a therapeutic molecule, forming an antibody-drug conjugate. In some embodiments, the therapeutic molecule is selected from the group consisting of: a proteasome inhibitor, a calicheamicin, a pyrrolobenzodiazepine, an auristatin, a duocarmycin, a maytansinoid, and any combination thereof. In some embodiments, the therapeutic molecule comprises the proteasome inhibitor, and wherein the proteasome inhibitor is selected from the group consisting of: bortezomib (Velcade, PS-341), PR-171 (carfilzomib), ixazomib (Ninlaro®), delanzomib, marizomib, oprozomib, VR23, PI-1840, (benzyloxycarbonyl)-Leu-Leu-phenylalaninal, 2,3,5a,6-tetrahydro-6-hydroxy-3-(hydroxymthyl)-2-methyl-10H-3a,10a-epidithio-pyrazinol[1,2a]indole-1,4-dione, 4-hydroxy-3-nitrophenylacetyl-Leu-Leu-Leu-vinyl sulphone, sapojargon, Ac-hFLFL-epoxide, aclacinomycin A, aclarubicin, ACM, AdaK(Bio)Ahx3L3VS, AdaLys(Bio)Ahx3L3VS, Adamantane-acetyl-(6-aminohexanoyl)-3-(leucunyl)-3-vinyl-(methyl)-sulphone, ALLM, ALLN, Calpain Inhibitor I, Calpain Inhibitor II, Carbobenzoxy-L-leucyl-L-leucyl-L-leucinal, Carbobenzoxy-L-leucyl-L-leucyl-L-norvalinal, gliotoxin, isovalery-L-tyrosyl-L-valyl-DL-tyrosinal, clasto-lactacystin-p-lactone, Z-LL-Nva-CHO, Ubiquitin Aldehyde, YU101, MP-LLL-VS, LDN-57444, Z-GPFL-CHO, Z-LLL-CHO, lovastatin, α-methyl-clasto-lactacystin-p-lactone,mevinolin, MK-803, NIP-L3VS, NP-LLL-VS, NPI-0052 (salinosporamide A), MLN519 (PS-519),NLVS (trileucine vinyl-sulfone), ritonavir, Ro106-9920, Z-LLF-CHO, Z-LL-B(OH)2, RRRPRPPYLPR, Tyropeptin A, ZL3VS, PR-11, PR-39, 0106-9920, Proteasome Inhibitor I, Proteasome Inhibitor II, Proteasome Inhibitor III, Proteasome Inhibitor IV, AdaAhx3L3VS, efrapeptin, MG-132, MG-262, MG-115, α-methylomuralide, MG-101, epoxomicin, omuralide, lactacystin, and NEOSH101.

One embodiment provides a kit, comprising the combination of any of the above embodiments and instructions for administering, to a subject, the anti-TM4SF1 binding protein and the immunotherapeutic agent. In some embodiments, the anti-TM4SF1 binding protein and the immunotherapeutic agent are in a single composition. In some embodiments, the anti-TM4SF1 binding protein and the immunotherapeutic agent are in separate compositions.

One embodiment provides a kit, comprising the combination of any one of the above embodiments, and instructions for administering, to a subject, the anti-TM4SF1 binding protein and agent the immunotherapeutic agent. In some embodiments, the binding protein and the immunotherapeutic agent are in a single composition. In some embodiments, the anti-TM4SF1 binding protein and the immunotherapeutic agent are in separate compositions. One embodiment provides a kit comprising any one of a. a composition comprising a therapeutically effective amount of an anti-TM4SF1 binding protein; and b composition comprising a therapeutically effective amount of an agent immunotherapeutic agent, and instructions for administering a. and b., sequentially or concurrently, to a subject.

One embodiment provides a kit comprising any one of: a. a composition comprising a therapeutically effective amount of an anti-TM4SF1 binding protein; and b. a composition comprising a therapeutically effective amount of an immunotherapeutic agent selected from the group consisting of ipilimumab, nivolumab, pembrolizumab, atezolizumab, durvalumab, tremelimumab, spartalizumab, avelumab, sintilimab, toripalimab, MGA012, MGD013, MGD019, enoblituzumab, MGD009, MGC018, MEDI0680, PDR001, FAZ053, TSR022, MBG453, relatlinab (BMS986016), LAG525, IMP321, REGN2810(cemiplimab), REGN3767, pexidartinib, LY3022855, FPA008, BLZ945, GDC0919, epacadostat, indoximid, BMS986205, CPI-444, MEDI9447, PBF509, lirilumab, IMC-001, Monalizumab and combinations thereof and instructions for administering a. and b., sequentially or concurrently, to a subject.

One embodiment provides a pharmaceutical composition comprising a combination according to any one of the above embodiment, or a kit according to any one of the above embodiment, and at least one of a pharmaceutically acceptable carrier, an excipient, a diluent, or any combination thereof. One embodiment provides a method of treating a subject, the method comprising administering a combination comprising: a. an anti-TM4SF1 binding protein; and b. an immune checkpoint inhibitor. In some embodiments, the anti-TM4SF1 binding protein and the immunotherapeutic agent are administered to the subject concurrently. In some embodiments, the anti-TM4SF1 binding protein and the immunotherapeutic agent are administered to the subject sequentially. In some embodiments, the anti-TM4SF1 binding protein is administered first and the immunotherapeutic agent is administered second. In some embodiments, the immunotherapeutic agent is administered first and the anti-TM4SF1 binding protein is administered second. In some embodiments, the first and second administration are separated by 1, 2, 3, 6, 12, 24, 48, or more hours. In some embodiments, the administration results in improved T cell function. In some embodiments, the improved T cell function comprises increased T cell infiltration in the tumor microenvironment (TME). In some embodiments, the improved T cell function comprises increased expression of ICAM-1 and VCAM-1 in tumor vessels.

One embodiment provides a method of treating a subject, the method comprising administering a combination comprising: a. an anti-TM4SF1 binding protein; and b. an immunotherapeutic agent selected from the group consisting of: ipilimumab, nivolumab, pembrolizumab, atezolizumab, durvalumab, tremelimumab, spartalizumab, avelumab, sintilimab, toripalimab, MGA012, MGD013, MGD019, enoblituzumab, MGD009, MGC018, MEDI0680, PDR001, FAZ053, TSR022, MBG453, relatlinab (BMS986016), LAG525, IMP321, REGN2810(cemiplimab), REGN3767, pexidartinib, LY3022855, FPA008, BLZ945, GDC0919, epacadostat, indoximid, BMS986205, CPI-444, MEDI9447, PBF509, lirilumab, IMC-001, Monalizumab, and combinations thereof. In some embodiments, the anti-TM4SF1 binding protein and the immunotherapeutic agent are administered to the subject concurrently. In some embodiments, the anti-TM4SF1 binding protein and the immunotherapeutic agent are administered to the subject sequentially. In some embodiments, the anti-TM4SF1 binding protein is administered first and the immunotherapeutic agent is administered second. In some embodiments, the immunotherapeutic agent is administered first and the anti-TM4SF1 binding protein is administered second. In some embodiments, the first and second administration are separated by 1, 2, 3, 6, 12, 24, 48, or more hours. In some embodiments, the administration results in improved T cell function. In some embodiments, the improved T cell function comprises increased T cell infiltration in the tumor microenvironment (TME). In some embodiments, the improved T cell function comprises increased expression of ICAM-1 and VCAM-1 in tumor vessels.

One embodiment provides a method of improving T cell response in a subject, comprising administering a combination according to any one of the above embodiments, wherein the improved response is associated with improved T cell function. In some embodiments, the improved T cell function comprises increased T cell infiltration in the tumor microenvironment (TME). In some embodiments, the improved T cell function comprises increased expression of ICAM-1 and VCAM-1 in tumor vessels. In some embodiments, the administering the combination therapy comprises a first treatment and a second treatment. In some embodiments, the first treatment comprises the anti-TM4SF1 binding protein and wherein the second treatment comprises the immune checkpoint inhibitor agent. In some embodiments, the first treatment comprises the anti-TM4SF1 binding protein and wherein the second treatment comprises the therapeutic agent. In some embodiments, the first treatment and the second treatment are performed within 1, 2, 3, 4, 5, or 7 days. In some embodiments, the combination is administered, weekly, bi-weekly, monthly, or bi-annually.

In some embodiments, the subject is undergoing a treatment which may induce metastasis. In some embodiments, the treatment comprises surgery, radiation treatment and chemotherapy. In some embodiments, the subject has a cancer. In some embodiments, the cancer is prostate cancer, liver cancer, colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung, non-small cell lung), melanoma, non melanoma skin cancer (squamous and basal cell carcinoma), glioma, renal cancer, lymphoma (NHL or HL), Acute myeloid leukemia (AML), T cell Acute Lymphoblastic Leukemia (T-ALL), Diffuse Large B cell lymphoma, testicular germ cell tumors, mesothelioma, esophageal cancer, Merkel Cells cancer, MSI-high cancer, KRAS mutant tumors, adult T-cell leukemia/lymphoma, and Myelodysplastic syndromes (MDS). In some embodiments, the method of any one of claim 26-55, wherein the subject is a human.

### BRIEF DESCRIPTION OF DRAWINGS

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates binding affinities with the capped anti-TM4SF1 antibodies of the Exemplary Antibody 1 group in mouse tumor and endothelial cells. Typically, the binding affinities for antibodies in the Exemplary Antibody 1 group is at 3-7 nM EC50 in cell-based assays.

FIGS. 2A-2B illustrate the results of in vivo B16F10 (syngeneic to C57Bl/6 mice; FIG. 2A) and CT26 (syngeneic to BALB/c mice; FIG. 2B) tumor regression activities using Exemplary Antibody 1-L1 anti-TM4SF1 antibody drug conjugates, alone or in combination with immune checkpoint inhibitor anti-PD-1 antibody (Clone RMP1-14; BioXCell). In both syngeneic tumor models, anti-PD-1 antibody alone did not show tumor regression activities, whereas the antibody enhanced Exemplary Antibody 1-L1 tumor regression activities when they were co-injected.

FIG. 3 illustrates binding affinities with the capped anti-TM4SF1 antibodies of the Exemplary Antibody 1 group in mouse tumor and endothelial cells.

FIGS. 4A-4H illustrate the results of in vivo CT26 (syngeneic to BALB/c mice) tumor growth activities through one single injection of vehicle or isotype matched control (IS-Ctl), or using Exemplary Antibody 1-L1 anti-TM4SF1 antibody drug conjugates, alone or in combination with immune checkpoint anti-mouse CTLA-4 antibody (Clone 9H10). FIG. 4A represents tumor growth in vehicle control and isotype matched control antibody drug conjugates (IS-Ctl), whereas FIG. 4E indicates tumor growth in the presence of Exemplary Antibody 1-L1 anti-TM4SF1 antibody drug conjugates. FIGS. 4B-4D illustrate results from three different doses (2.5, 5, or 10 mpk) of anti-mouse CTLA-4 antibody alone, whereas FIG. 4F-4H show results from using a combination of the exemplary anti-TM4SF1 antibody 1-L1 with the three different doses of anti-mouse CTLA-4 antibody.

FIGS. 5A-5D illustrate the results of a rechallenge of BALB/c mice that exhibited tumor free (TF) at day 60-70 after the initial dosing (as shown in FIGS. 4A-4H) with freshly prepared CT26 tumor cells. Both 8-week-old naïve BALB/c mice and 6-8 months old retired BALB/c breeders were used as tumor growth control and received the same number of CT26 tumor cells as in rechallenging mice.

FIGS. 6A-6F illustrate the results of in vivo Renca (syngeneic to BALB/c mice) tumor regression activities using Exemplary Antibody 1-L1 anti-TM4SF1 antibody drug conjugates, alone or in combination with immune checkpoint anti-mouse CTLA-4 antibody (Clone 9H10).

FIG. 6A represents tumor growth in vehicle control and isotype matched control antibody drug conjugates (IS-Ctl), whereas FIG. 6D indicates tumor growth in the presence of Exemplary Antibody 1-L1 anti-TM4SF1 antibody drug conjugates. FIGS. 6B-6C illustrate results from two different doses (5, or 10 mpk) of anti-mouse CTLA-4 antibody alone, whereas FIG. 6E-6F show results from using a combination of the exemplary anti-TM4SF1 antibody 1-L1 with the two different doses of anti-mouse CTLA-4 antibody.

FIGS. 7A-7D illustrate the results of a rechallenge of BALB/c mice that exhibited tumor free (TF) (as shown in FIGS. 6A-6H) and retired BALB/c breeders with Renca.

FIGS. 8A-8H illustrate the results of in vivo B16F10 (syngeneic to C57Bl/6 mice) tumor regression activities using Exemplary Antibody 1-L1 anti-TM4SF1 antibody drug conjugates, alone or in combination with immune checkpoint CTLA-4 antibody (Clone 9H10). FIG. 8A represents tumor growth in vehicle control and isotype matched control antibody drug conjugates (IS-Ctl), whereas FIG. 8E indicates tumor growth in the presence of Exemplary Antibody 1-L1 anti-TM4SF1 antibody drug conjugates. FIGS. 8B-8D illustrate results from three different doses (2.5, 5, or 10 mpk) of anti-mouse CTLA-4 antibody alone, whereas FIG. 8F-8H show results from using a combination of the exemplary anti-TM4SF1 antibody 1-L1 with the three different doses of anti-mouse CTLA-4 antibody.

FIGS. 9A-9H illustrate the results of in vivo LLC (syngeneic to C57Bl/6 mice) tumor regression activities using Exemplary Antibody 1-L1 anti-TM4SF1 antibody drug conjugates, alone or in combination with immune checkpoint CTLA-4 antibody (Clone 9H10). FIG. 9A represents tumor growth in vehicle control and isotype matched control antibody drug conjugates (IS-Ctl), whereas FIG. 9E indicates tumor growth in the presence of Exemplary Antibody 1-L1 anti-TM4SF1 antibody drug conjugates. FIGS. 9B-9D illustrate results from three different doses (2.5, 5, or 10 mpk) of anti-mouse CTLA-4 antibody alone, whereas FIG. 9F-9H show results from using a combination of the exemplary anti-TM4SF1 antibody 1-L1 with the three different doses of anti-mouse CTLA-4 antibody.

### DETAILED DESCRIPTION OF THE INVENTION

Transmembrane-4 L six family member-1 (TM4SF1) is a small membrane glycoprotein with tetraspanin topology that is highly expressed on most human tumors of solid tumor origin and in endothelial cells, especially activated endothelial cells in angiogenic vessels.

Provided herein in one embodiment, is a combination that can improve tumor regression through the use of a vascular targeted therapy (e.g., an antibody that interacts endothelial cell biomarker, such as an anti-angiogenic antibody, such as an anti-TM4SF1 antibody or an antigen binding fragment thereof, and conjugated with a payload, e.g., a cytotoxic payload) and an immunotherapeutic agent (e.g., immune check point inhibitor, a cytokine, an adjuvant). This combination may include various attractive features. For example, the anti-angiogenic antibody can provide some advantages at least because: (1) angiogenesis is a hallmark of cancer and a therapy that destroys angiogenic vessels can be a universal treatment for solid tumors; (2) the vascular endothelium is an unmutated host system and unable to evolve resistance to therapy; thus, a vascular-targeted therapy may be able to overcome a common problem with tumor cell targeted therapies, wherein a target tissue evolves and becomes resistant to therapy; (3) the vascular endothelium of tumors is directly exposed to intravenously (IV)-infused drugs and therefore can be accessible to drugs that cannot reach tumor cells; and (4) tumor vessel destruction facilitates immune cell infiltration for wound healing. Immune checkpoint receptors act as T cell receptor (TCR) co-signaling partners that deliver negative signals to T lymphocytes. For example, programmed death protein-1 (PD-1) is a negative co-stimulatory receptor and delivers negative signals to cytotoxic T lymphocytes. Although PD-1 is critical for self-tolerance under normal conditions, however, it can allow tumors to evade the antitumor immune response when interacts its ligand PD-L1 and PD-L2 expressed on tumor cells and lead to the increased T-cell exhaustion and a diminished antitumor response. Blockade of PD-1 via anti-PD-1 antibodies or PD-L1/L2 via anti-PD-L1/L2 antibodies can block the negative signals and help cytotoxic T cells to restore its immune function. The inaccessibility of blockade antibodies such as PD-L1 to reach tumor cells can be a major challenge to target solid tumors, in particular for those tumors resemble the phenotype of pancreatic cancer which have a dense fibrotic stroma that limits access of drugs to tumor cells. Thus, a combination therapy, using an anti-angiogenic antibody to cause tumor vessel injury and to enhance leukocyte infiltration or to deliver immune blockade antibodies to assist T-cells to target tumor cells, is an advantageous approach to bring cold tumors hot to improve immunotherapy. In another embodiment, the disclosure provides a combination comprising an antibody drug conjugate (ADC) comprising an anti-angiogenic ADC (e.g., an anti-TM4SF1 antibody or an antigen-binding fragment thereof conjugated with a payload, e.g., a cytotoxic payload; and an immunotherapeutic agent (e.g., immune checkpoint inhibitor). This disclosure includes, in some examples, methods of using the above mentioned combinations for treating or preventing cancer.

### I. Definitions

Unless otherwise defined herein, scientific and technical terms used in connection with this disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The terms “transmembrane-4 L six family member-1” or “TM4SF1”, as used herein refer to a polypeptide of the transmembrane 4 superfamily/tetraspanin family, which is expressed on vasculature endothelial cells (ECs), ECs of developing retinal vasculature, with higher expression in tumor ECs, tumor cells (TCs), and angiogenic blood vessels. TM4SF1 has two extracellular loops (ECL1 and ECL2) that are separated by four transmembrane domains (M1, M2, M3, and M4), the N- and C-termini, and the intracellular loop (ICL). ECL2 contains two N-glycosylation sites. Exemplary amino acid sequence of human TM4SF1 (hTM4SF1) is described in SEQ ID NO: 166 (see also NCBI Ref Seq No. NP_055035.1).

The term “antibody”, as used herein, means any antigen-binding molecule comprising at least one complementarity determining region (CDR) that specifically binds to or interacts with a particular antigen (e.g., TM4SF1). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-TMS4F1 antibody (or antigen-binding portion thereof) may be identical to the human germline sequences, or may be naturally or artificially modified. An amino acid consensus sequence may be defined based on a side-by-side analysis of two or more CDRs.

The term “intact antibody” refers to an antibody comprising four polypeptide chains, two heavy (H) chains and two light (L) chains inter-connected by disulfide bonds. In one embodiment, the anti-TM4SF1 antibody is an intact antibody. In one embodiment, the intact antibody is an intact human IgG1, IgG2 or IgG4 isotype. In certain embodiments, the anti-TM4SF1 antibody, or antigen-binding fragment thereof, is a human IgG1, IgG2, or IgG4 isotype.

The terms “antigen-binding portion” of an antibody, “antigen-binding fragment,” or “antibody-fragment,” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from intact antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide.

The term “variable region” or “variable domain” of an antibody, or fragment thereof, as used herein refers to the portions of the light and heavy chains of antibody molecules that include amino acid sequences of complementarity determining regions (CDRs; i.e., CDR-1, CDR-2, and CDR-3), and framework regions (FRs). VH refers to the variable domain of the heavy chain. VL refers to the variable domain of the light chain. According to the methods used in this disclosure, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

The term “complementarity determining regions” or “CDRs” as used herein refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia et al., J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

The term “framework regions” (hereinafter FR) as used herein refers to those variable domain residues other than the CDR residues. Each variable domain typically has four FRs identified as FR1, FR2, FR3 and FR4. Common structural features among the variable regions of antibodies, or functional fragments thereof, are well known in the art. The DNA sequence encoding a particular antibody can generally be found following well known methods such as those described in Kabat, et al. 1987 Sequence of Proteins of Immunological Interest, U.S. Department of Health and Human Services, Bethesda MD, which is incorporated herein as a reference. In addition, a general method for cloning functional variable regions from antibodies can be found in Chaudhary, V. K., et al., 1990 Proc. Natl. Acad. Sci. USA 87:1066, which is incorporated herein as a reference.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known. The Fc fragment comprises the carboxy-terminal portions of both H chains held together by di sulfides. The effector functions of antibodies are determined by sequences in the Fc region, which region is also the part recognized by Fc receptors (FcR) found on certain types of cells. The term “Fc region” herein is used to define a C-terminal region of an antibody heavy chain, including, for example, native sequence Fc regions, recombinant Fc regions, and variant Fc regions. Although the boundaries of the Fc region of an antibody heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system as in Kabat et al.) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Further, a composition of intact antibodies in this disclosure may comprise antibody populations with extension of residues after the C-terminal lysine, K447.

A “variant Fc region” comprises an amino acid sequence which differs from that of a native sequence Fc region by virtue of at least one “amino acid modification” as herein defined. A variant Fc region has at least one amino acid substitution compared to a native sequence Fc region or to the Fc region of a parent polypeptide, e.g., from about one to about ten amino acid substitutions, from about one to about five amino acid substitutions in a native sequence Fc region or in the Fc region of the parent polypeptide. In one embodiment, a variant Fc region herein can have a sequence that has at least about 80% homology, at least about 85% homology, at least about 90% homology, at least about 95% homology or at least about 99% homology with a native sequence Fc region. According to another embodiment, the variant Fc region herein can have a sequence that has at least about 80% homology, at least about 85% homology, at least about 90% homology, at least about 95% homology or at least about 99% homology with an Fc region of a parent polypeptide.

The term “humanized antibody” as used herein refers to an antibody or a variant, derivative, analog or fragment thereof, which immunospecifically binds to an antigen of interest (e.g., human TM4SF1), and which comprises a framework (FR) region having substantially the amino acid sequence of a human antibody and a complementary determining region (CDR) having substantially the amino acid sequence of a non-human antibody. Humanized forms of non-human (e.g., murine) antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., 1988, Nature 332:323-7; U.S. Pat. Nos. 5,530,101; 5,585,089; 5,693,761; 5,693,762; and U.S. Pat. No. 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332, all of which are hereby incorporated by reference in their entireties.

The term “monoclonal antibody” as used herein refers to an antibody obtained from a population of substantially homogeneous antibodies, i.e., the individual antibodies comprising the population are identical except for possible mutations, e.g., naturally occurring mutations that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. In contrast to polyclonal antibody preparations, which typically include different antibodies directed against different determinants (epitopes), each monoclonal antibody of a monoclonal-antibody preparation is directed against a single epitope on an antigen.

The term “chimeric antibody” as used herein refers to antibodies (immunoglobulins) that have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81: 6851-6855 (1984)).

The term “epitope” as used herein refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be defined as structural or functional. Functional epitopes are generally a subset of the structural epitopes and have those residues that directly contribute to the affinity of the interaction. Epitopes may also be conformational, that is, composed of non-linear (non-contiguous) amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes can be formed both from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from (e.g., from TREM-1) are tested for reactivity with a given antibody (e.g., anti-TREM-1 antibody). Methods of determining spatial conformation of epitopes include techniques in the art and those described herein, for example, x-ray crystallography, antigen mutational analysis, 2-dimensional nuclear magnetic resonance and HDX-MS (see, e.g., Epitope Mapping Protocols in Methods in Molecular Biology, Vol. 66, G. E. Morris, Ed. (1996)).

The terms “payload,” “drug payload,” “therapeutic molecule, “therapeutic payload”, “therapeutic agents,” “therapeutic moieties,” as used interchangeably herein, refers to a chemical or biological moiety that is conjugated to an anti-TMSF1 antibody or antigen binding fragment (e.g., an anti-TM4SF1 antibody or antigen binding fragment disclosed herein), and can include any therapeutic or diagnostic agent, for example, but not limited to, small molecules, both for cancer and for non-cancer angiogenic indications; a V-ATPase inhibitor; a pro-apoptotic agent; a Bcl2 inhibitor; an MCL1 inhibitor; a HSP90 inhibitor; an IAP inhibitor; an mTor inhibitor; a microtubule stabilizer; a microtubule destabilizer; an auristatin; a dolastatin; a maytansinoid; a MetAP (methionine aminopeptidase); an inhibitor of nuclear export of proteins CRM1; a DPPIV inhibitor; proteasome inhibitors; inhibitors of phosphoryl transfer reactions in mitochondria; a protein synthesis inhibitor; a kinase inhibitor (such as, a CDK2 inhibitor, a CDK9 inhibitor); a kinesin inhibitor, an HDAC inhibitor, a DNA damaging agent, a DNA alkylating agent, a DNA intercalator, a DNA minor groove binder, a DHFR inhibitor, a nucleic acid, a CRISPR enzyme; degraders (such as agents that induce protein degradation, (e.g., HSP90 inhibitor, selective estrogen receptor degraders (SERDs), selective androgen receptor degraders (SARDs); hydrophobic tags that can be used to recruit chaperones to a protein of interest, e.g., Adamantane, Arg-Boc3; E3 ligase recruiting ligands, e.g., Nutlin-3a (MDM2 ligand), Bestatin (cIAP ligand), VHL ligand, Pomalidomide (CRBN ligand); proteolysis-targeting chimeras (PROTACs) that may utilize different D3 ligases to target a protein of interest for degradation)) (see, e.g., Lai A C, Crews C M. Induced protein degradation: an emerging drug discovery paradigm. Nat Rev Drug Discov. 2016; 16(2):101-114); antisense oligonucleotides; RNAi agents (such as siRNA), CRISPR-Cas9 gene editing systems; RNA molecules; DNA e.g., plasmids; an anti-cancer agent, an anti-inflammatory agent, an anti-infective agent (e.g., anti-fungal, antibacterial, anti-parasitic, anti-viral), an anesthetic agent; RNA polymerase II inhibitor; a DNA intercalating agent, a DNA cross-linking agent; an anti-tubulin agent; a cytotoxic drug, a tumor vaccine, an antibody, a peptide, pepti-bodies, a chemotherapeutic agent, a cytotoxic agent; a cytostatic agent; an immunological modifiers, an interferon, an interleukin, an immunostimulatory growth hormone, a cytokine, a vitamin, a mineral, an aromatase inhibitor, a Histone Deacetylase (HDAC), an HDAC inhibitor; a lipid nanoparticle to encapsulate one or more therapeutic molecules.

The term “drug-to-antibody ratio” or “DAR” can refer to the number of drugs (also referred to herein as therapeutic molecules, therapeutic agents, or therapeutic moieties), attached to an anti-TM4SF1 antibody or antigen binding fragments thereof, of the ADCs disclosed herein. The DAR of an ADC typically ranges from 1 to 12, although higher loads, e.g., 16, are also possible depending on the number of linkage sites on an antibody or the use of multivalent linkages in which multiple drug payloads are attached to one linkage site. The term DAR may be used in reference to the number of drug molecules loaded onto an individual antibody, or, alternatively, may be used in reference to the average or mean DAR of a group of ADCs to reflect average drug loading. Compositions, batches, and/or formulations of a plurality of ADCs may be characterized by an average DAR. DAR and average DAR can be determined by various conventional means such as UV spectroscopy, mass spectroscopy, ELISA assay, radiometric methods, hydrophobic interaction chromatography (HIC), electrophoresis and HPLC.

The term “binding affinity” generally refers to the strength of the sum total of noncovalent interactions between a single binding site of a molecule (e.g., a binding protein such as an antibody) and its binding partner (e.g., an antigen). The affinity of a binding molecule X (e.g., anti-TM4SF1 antibody) for its binding partner Y (e.g., human TM4SF1) can generally be represented by the dissociation constant (KD). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “KD” or “KD value” may be measured by assays known in the art, for example by a binding assay. The KD may be measured in a RIA, for example, performed with the Fab version of an antibody of interest and its antigen (Chen et al., 1999, J. Mol Biol 293:865-81). The KD may also be measured by using FACS or surface plasmon resonance assays by BIACORE, using, for example, a BIACORE 2000 or a BIACORE 3000, or by biolayer interferometry using, for example, the OCTET QK384 system. In certain embodiments, the KD of an anti-TM4SF1 antibody is determined using a standard flow cytometry assay with HUVEC cells. An “on-rate” or “rate of association” or “association rate” or “kon” and an “off-rate” or “rate of dissociation” or “dissociation rate” or “koff” may also be determined with the same surface plasmon resonance or biolayer interferometry techniques described above using, for example, a BIACORE 2000 or a BIACORE 3000, or the OCTET QK384 system.

The term “kₒₙ”, as used herein, is intended to refer to the on rate constant for association of an antibody to the antigen to form the antibody/antigen complex, as is known in the art.

The term “koff”, as used herein, is intended to refer to the off rate constant for dissociation of an antibody from the antibody/antigen complex, as is known in the art.

The term “inhibition” or “inhibit,” when used herein, refers to partial (such as, 1%, 2%, 5%, 10%, 20%, 25%, 50%, 75%, 90%, 95%, 99%) or complete (i.e., 100%) inhibition.

The term “cancer” as used herein, refers to or describes the physiological condition in mammals that is typically characterized by unregulated cell growth.

The term “cancer which is associated with a high risk of metastasis”, as used herein, refers to a cancer that is associated with at least one factor known to increase the risk that a subject having the cancer will develop metastatic cancer. Examples of factors associated with increased risk for metastasis include, but are not limited to, the number of cancerous lymph nodes a subject has at the initial diagnosis of cancer, the size of the tumor, histological grading, and the stage of the cancer at initial diagnosis.

The term “hematogenous metastasis” as used herein refers to the ability of cancer cells to penetrate the walls of blood vessels, after which they are able to circulate through the bloodstream (circulating tumor cells) to other sites and tissues in the body.

The term “lymphatic metastasis” as used herein refers to the ability of cancer cells to penetrate lymph vessels and drain into blood vessels.

In the context of the disclosure, the term “treating” or “treatment”, as used herein, means reversing, alleviating, inhibiting the progress of, or preventing the disorder or condition to which such term applies, or one or more symptoms of such disorder or condition. By the term “treating cancer” as used herein is meant the inhibition of the growth and/or proliferation of cancer cells.

In one embodiment, the compositions and methods described herein are used to treat metastasis in a subject having metastatic cancer.

The term “preventing cancer” or “prevention of cancer” refers to delaying, inhibiting, or preventing the onset of a cancer in a mammal in which the onset of oncogenesis or tumorigenesis is not evidenced but a predisposition for cancer is identified whether determined by genetic screening, for example, or otherwise. The term also encompasses treating a mammal having premalignant conditions to stop the progression of, or cause regression of, the premalignant conditions towards malignancy. Examples of premalignant conditions include hyperplasia, dysplasia, and metaplasia. In some embodiments, preventing cancer is used in reference to a subject who is in remission from cancer.

A variety of cancers, including malignant or benign and/or primary or secondary, may be treated or prevented with a method according to the disclosure. Examples of such cancers are known to those skilled in the art and listed in standard textbooks such as the Merck Manual of Diagnosis and Therapy (published by Merck).

The term “subject” as used herein, refers to a mammal (e.g., a human).

The term “administering” as used herein refers to a method of giving a dosage of an antibody or fragment thereof, or a composition (e.g., a pharmaceutical composition) to a subject.

The method of administration can vary depending on various factors (e.g., the binding protein or the pharmaceutical composition being administered and the severity of the condition, disease, or disorder being treated).

The term “effective amount” as used herein refers to the amount of an antibody or pharmaceutical composition provided herein which is sufficient to result in the desired outcome.

The terms “about” and “approximately” mean within 20%, within 15%, within 10%, within 9%, within 8%, within 7%, within 6%, within 5%, within 4%, within 3%, within 2%, within 1%, or less of a given value or range.

The term “identity,” or “homology” as used interchangeable herein, may be to calculations of “identity,” “homology,” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % homology=# of identical positions/total # of positions×100). For example, a position in the first sequence may be occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology between two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. A non-limiting example of such a mathematical algorithm may be described in Karlin, S. and Altschul, S., Proc. Natl. Acad. Sci. USA, 90-5873-5877 (1993). Such an algorithm may be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In another embodiment, the percent identity between two amino acid sequences can be accomplished using, for example, the GAP program in the GCG software package (Accelrys, Cambridge, UK).

The term “manufacturability,” as used herein, refers to the stability of a particular protein during recombinant expression and purification of that protein. Manufacturability is believed to be due to the intrinsic properties of the molecule under conditions of expression and purification. Examples of improved manufacturability characteristics include uniform glycosylation of a protein, increased cell titer, growth and protein expression during recombinant production of the protein, improved purification properties, less propensity of aggregation or non-aggregation, and improved stability, including, but not limited to, thermal stability and stability at low pH. In some embodiments are provided TM4SF1 binding proteins that demonstrate the manufacturability, along with retention of in vitro and in vivo activity, compared with other TM4SF1 antibodies. In some embodiments, humanization of a parent TM4SF1 binding protein, by making amino acid substitutions in the CDR or framework regions, can confer additional manufacturability benefits.

In some embodiments are provided TM4SF1 binding proteins that demonstrate improved developability characteristics, including, but not limited to improved purification yield, for example, after protein A purification or size exclusion chromatography, improved homogeneity after purification, improved thermal stability. In some cases, the improvement is with respect to an anti-TM4SF1 antibody produced by a hybridoma mouse cell line 8G4-5-13-13F (PTA-120523), as determined by HLA molecule binding.

In some examples, binding affinity is determined by Scatchard analysis, which comprises generating a Scatchard plot, which is a plot of the ratio of concentrations of bound ligand to unbound ligand versus the bound ligand concentration.

The term “vascular toxicity” refers to any effect of an anti-TM4SF1 antibody-therapeutic molecule conjugate (also referred to herein as anti-TM4SF1 ADC or TM4SF1 targeted ADC) which leads to vascular injury either directly due to the antibody or the therapeutic molecule effects on antigen-bearing cells or indirectly through activation of the immune system and resulting inflammation. Such vascular injury may include, but is not limited to, damage or inflammation affecting vascular endothelial cells or underlying smooth muscle cells or pericytes or the basement membrane of any blood vessel, including the endocardium (lining of the heart). Such vascular injury may affect arteries, including major arteries such as the aorta, elastic arteries (such as the aorta), muscular arteries of varying sizes, such as coronary artery, pulmonary artery, carotid artery, arterioles, capillaries, arteries of the brain or retina; venues, veins; or it may affect angiogenic vessels including vessels serving hair follicles, the digestive tract, and bone marrow. Such vascular injury may include microvascular dysfunction or damage in the heart, lung, kidney, retina, brain, skin, liver, digestive tract, bone marrow, endocrine glands, testes or ovaries, endometrium, and other target organs and may include renal, retinal or cerebrovascular circulation dysfunction.

The term “antibody-dependent cell-mediated cytotoxicity (ADCC)” as used herein refers to the killing of an antibody-coated target cell by a cytotoxic effector cell through a nonphagocytic process, characterized by the release of the content of cytotoxic granules or by the expression of cell death-inducing molecules. ADCC is triggered through interaction of target-bound antibodies (belonging to IgG or IgA or IgE classes) with certain Fc receptors (FcRs), glycoproteins present on the effector cell surface that bind the Fc region of immunoglobulins (Ig). Effector cells that mediate ADCC include natural killer (NK) cells, monocytes, macrophages, neutrophils, eosinophils and dendritic cells. ADCC is a rapid effector mechanism whose efficacy is dependent on a number of parameters (density and stability of the antigen on the surface of the target cell; antibody affinity and FcR-binding affinity). PBMC-based ADCC assays and natural kill cell-based ADCC assays can be used to detect ADCC. The readout in these assays is endpoint-driven (target cell lysis).

The term “complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay (See, e.g., Gazzano-Santoro et al., 1996, J. Immunol. Methods 202:163) may be performed. Polypeptide variants with altered Fc region amino acid sequences (polypeptides with a variant Fc region) and increased or decreased C1q binding capability have been described (see, e.g., U.S. Pat. No. 6,194,551; WO 1999/51642; Idusogie et al., 2000, J. Immunol. 164: 4178-84). Antibodies (or fragments) with little or no CDC activity may be selected for use.

The term “effector function” as used herein refers to a function contributed by an Fc effector domain(s) of an IgG (e.g., the Fc region of an immunoglobulin). Such function can be affected by, for example, binding of an Fc effector domain(s) to an Fc receptor on an immune cell with phagocytic or lytic activity or by binding of an Fc effector domain(s) to components of the complement system. Examples of antibody effector functions include: C1q binding and complement dependent cytotoxicity (CDC); Fc receptor binding; antibody-dependent cell-mediated cytotoxicity (ADCC); phagocytosis (ADCP); down regulation of cell surface receptors (e.g. B cell receptor); and B cell activation.

The terms “reduce” or “ablate” as used herein refers to the ability to cause an overall decrease preferably of 20% or greater, more preferably of 50% or greater, and most preferably of 75%, 85%, 90%, 95%, or greater. Reduce or ablate can refer to binding affinity of two molecules, for example the binding of immunoglobulins to C1q or to Fc receptors; or can refer to the symptoms of the disorder (e.g., cancer) being treated, such as the presence or size of metastases or the size of the primary tumor.

The term “reduced ADCC/CDC function,” as used herein refers to a reduction of a specific effector function, e.g. ADCC and/or CDC, in comparison to a control (for example an antibody with a Fc region not including the mutation(s)), by at least about 5%, at least about 10%, at least about 15%, at least about 20%, at least about 30%, at least about 40%, at least about 50%, at least about 60%, at least about 70%, at least about 80% at least, at least about 90% or more.

For all amino acid positions discussed in the present disclosure, in the context of antibodies or antigen binding fragments thereof, numbering is according to the EU index. The “EU index” or “EU index as in Kabat et al.” or “EU numbering scheme” refers to the numbering of the EU antibody (See Edelman et al., 1969; Kabat et al., 1991).

### II. Anti-TM4SF1 Antibody or Antigen Binding Fragments Thereof

In some embodiments, provided herein is a combination comprising an anti-angiogenic antibody or an antigen binding fragment thereof, wherein the anti-angiogenic antibody or an antigen binding fragment thereof comprises an anti-TM4SF1 antibody or an antigen binding fragment thereof, as described herein.

TM4SF1 is a small plasma membrane glycoprotein (NCBI Ref Seq No. NP_055035.1) with tetraspanin topology but not homology (Wright et al. Protein Sci. 9: 1594-1600, 2000). It forms TM4SF1-enriched domains (TMED) on plasma membranes, where, like genuine tetraspanins, it serves as a molecular facilitator that recruits functionally related membrane and cytosolic molecules (Shih et al. Cancer Res. 69: 3272-3277, 2009; Zukauskas et al., Angiogenesis. 14: 345-354, 2011), and plays important roles in cancer cell growth (Hellstrom et al. Cancer Res. 46: 391 7-3923, 1986), motility (Chang et al. Int J Cancer. 1 16: 243-252, 2005), and metastasis (Richman et al. Cancer Res. 5916s-5920s, 1995). The amino acid sequence of human TM4SF1 protein (NCBI Ref Seq No. NP_055035.1) is shown below as SEQ ID NO: 165.

(SEQ ID NO: 165)
MCYGKCARCI GHSLVGLALL CIAANILLYF PNGETKYASE
NHLSRFVWFF SGIVGGGLLM LLPAFVFIGL EQDDCCGCCG
HENCGKRCAM LSSVLAALIG IAGSGYCVIV
AALGLAEGPLCLDSLGQWNYTFASTEGQYLLDTSTWSECTEPKHIVEWNVSLFSILLAL
G GIEFILCLIQVINGVLGGIC GFCCSHQQQY DC

In some embodiments, the anti-TM4SF1 antibodies and antigen binding fragments thereof, of the disclosure are specific to the ECL2 domain of TM4SF1. The amino acid sequence of human TM4SF1 ECL2 domain is

(SEQ ID NO: 166)
EGPLCLDSLGQWNYTFASTEGQYLLDTSTWSECTEPKHIVEWNVSLFS.

Example antibodies that are part of the combination provided herein include TM4SF1 specific antibodies provided in Table 2. The example anti-TM4SF1 antibodies described in Table 2 are monoclonal murine antibodies AGX-A03, AGX-A04, AGX-A05, AGX-A07, AGX-A08, AGX-A09, and AGX-A11, which can bind the ECL2 region of TM4SF1. Further provided in Table 2 below are humanized version of some of these antibodies, h AGX-A07 and h AGX-A01.

In some embodiments, the anti-TM4SF1 antibodies or antigen-binding fragments thereof, comprise an IgG heavy chain constant region comprising an amino acid sequence set forth in SEQ ID NO: 87 or 88, or a sequence that is at least about 60%, at least about 65%, at least about 70%, at least about 75%, at least about, at least about 80%, at least about 85%, at least about 90%, at least about 95%, at least about 96%, at least about 97%, at least about 98%, at least about 99%, or 100% identical to SEQ ID NO: 87 or 88.

In another embodiment, the anti-TM4SF1 antibody or antigen-binding fragment thereof, comprises a light chain constant region comprising the amino acid sequence set forth in SEQ ID NO: 89, or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 89.

In another embodiment, the anti-TM4SF1 antibody or antigen-binding fragment thereof, comprises a heavy chain variable domain comprising the amino acid sequence set forth in SEQ ID NO: 3, 15, 27, 39, 51, 63, or 75, or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 3, 15, 27, 39, 51, 63, or 75.

In another embodiment, the anti-TM4SF1 antibody or antigen-binding fragment thereof is humanized and, comprises a heavy chain comprising the amino acid sequence set forth in SEQ ID NO: 90 or 92 or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 90 or 92.

In another embodiment, the anti-TM4SF1 antibody or antigen-binding fragment thereof is humanized and, comprises a heavy chain comprising the amino acid sequence set forth in SEQ ID NO: 112 or 114, or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 112 or 114.

In another embodiment, the anti-TM4SF1 antibody or antigen-binding fragment thereof, comprises a light chain variable domain comprising the amino acid sequence set forth in SEQ ID NO: 9, 21, 33, 45, 57, 69, or 81, or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 9, 21, 33, 45, 57, 69, or 81.

In another embodiment, the anti-TM4SF1 antibody or antigen-binding fragment thereof is humanized and, comprises a light chain variable domain comprising the amino acid sequence set forth in SEQ ID NO: 97, 99, 101, 103, or 105 or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 97, 99, 101, 103 or 105. In another embodiment, the antibody or antigen-binding fragment thereof is humanized and, comprises a light chain variable domain comprising the amino acid sequence set forth in SEQ ID NO: 97, 99, or 101 or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 97, 99, or 101.

In another embodiment, the anti-TM4SF1 antibody or antigen-binding fragment thereof is humanized and, comprises a light chain variable domain comprising the amino acid sequence set forth in SEQ ID NO: 122, or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 122.

In some embodiments, the anti-TM4SF1 antibody or antigen binding fragment thereof comprises a heavy chain CDR1 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 6, 18, 30, 42, 54, 66, 78, 94, 115, or 160. In some embodiments, the anti-TM4SF1 antibody or antigen binding fragment thereof comprises a heavy chain CDR2 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 7, 19, 31, 43, 55, 67, 79, 95, 116, 117, or 161. In some embodiments, the anti-TM4SF1 antibody or antigen binding fragment thereof comprises a heavy chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 8, 20, 32, 44, 56, 68, 80, 96, 118, 119, 120, 121, or 162.

In some embodiments, the anti-TM4SF1 antibody or antigen binding fragment thereof comprises a light chain CDR1 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 12, 24, 36, 48, 60, 72, 84, 107, 108, 124, 125, 126, 127, or 157. In some embodiments, the anti-TM4SF1 antibody or antigen binding fragment thereof comprises a light chain CDR2 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 13, 25, 37, 49, 61, 73, 85, 109, 128, or 158.

In some embodiments, the anti-TM4SF1 antibody or antigen binding fragment thereof comprises a light chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 14, 26, 38, 50, 62, 74, 86, 110, 129, or 159.

In some embodiments, the anti-TM4SF1 antibody or antigen binding fragment thereof is humanized and comprises a heavy chain CDR1 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 94 or SEQ ID NO: 115. In some embodiments, the anti-TM4SF1 antibody or antigen binding fragment thereof is humanized and comprises a heavy chain CDR2 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 95, SEQ ID NO: 116, or SEQ ID NO: 117. In some embodiments, the anti-TM4SF1 antibody or antigen binding fragment thereof is humanized and comprises a heavy chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 96, SEQ ID NO: 118, SEQ ID NO: 119, SEQ ID NO: 120, or SEQ ID NO: 121.

In some embodiments, the anti-TM4SF1 antibody or antigen binding fragment thereof is humanized and comprises a light chain CDR1 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 107, SEQ ID NO: 108, SEQ ID NO: 124, SEQ ID NO: 125, SEQ ID NO: 126, or SEQ ID NO: 127. In some embodiments, the anti-TM4SF1 antibody or antigen binding fragment thereof is humanized comprises a light chain CDR2 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 109 or SEQ ID NO: 128. In some embodiments, the anti-TM4SF1 antibody or antigen binding fragment thereof is humanized and comprises a light chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 110, SEQ ID NO: 111, or SEQ ID NO: 129. In some embodiments, the anti-TM4SF1 antibody or antigen binding fragment thereof is humanized and comprises a light chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 110, or SEQ ID NO: 129.

In some embodiments, the anti-TM4SF1 antibody or antigen binding fragment thereof is humanized and comprises a light chain CDR1 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 157. In some embodiments, the anti-TM4SF1 antibody or antigen binding fragment thereof is humanized comprises a light chain CDR2 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 158. In some embodiments, the anti-TM4SF1 antibody or antigen binding fragment thereof is humanized and comprises a light chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 159.

In some embodiments, the anti-TM4SF1 antibody or antigen binding fragment thereof is humanized and comprises a heavy chain CDR1 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 160. In some embodiments, the anti-TM4SF1 antibody or antigen binding fragment thereof is humanized comprises a heavy chain CDR2 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 161. In some embodiments, the anti-TM4SF1 antibody or antigen binding fragment thereof is humanized and comprises a heavy chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 162.

The amino acid sequences of murine monoclonal antibody AGX-A03 are described in Table 2. Specifically, the heavy chain CDR sequences are set forth in SEQ ID Nos: 6, 7, and 8 (CDR1, CDR2, and CDR3), and the light chain CDR amino acid sequences are set forth in SEQ ID Nos: 12, 13, and 14 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 6, 7, and 8 and/or a light chain variable region comprising CDRs as set forth in the amino acid sequences of SEQ ID Nos: 12, 13, and 14. Included in the disclosure are humanized antibodies or antigen binding fragments comprising the CDRs of AGX-A03. Further, the heavy chain variable amino acid sequences and the light chain variable amino acid sequences of AGX-A03 are described in SEQ ID NOS: 3 and 9, respectively.

The amino acid sequences of murine monoclonal antibody AGX-A04 are described in Table 1. Specifically, the heavy chain CDR sequences are set forth in SEQ ID Nos: 18, 19, and 20 (CDR1, CDR2, and CDR3), and the light chain CDR amino acid sequences are set forth in SEQ ID Nos: 24, 25, and 26 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 18, 19, and 20 and/or a light chain variable region comprising CDRs as set forth in the amino acid sequences of SEQ ID Nos: 24, 25, and 26. Included in the disclosure are humanized antibodies or antigen binding fragments comprising the CDRs of AGX-A04. Further, the heavy chain variable amino acid sequences and the light chain variable amino acid sequences of AGX-A04 are described in SEQ ID NOS: 15 and 21, respectively.

The amino acid sequences of murine monoclonal antibody AGX-A05 are described in Table 1. Specifically, the heavy chain CDR sequences are set forth in SEQ ID Nos: 30, 31, and 32 (CDR1, CDR2, and CDR3), and the light chain CDR amino acid sequences are set forth in SEQ ID Nos: 36, 37, and 38 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 30, 31, and 32 and/or a light chain variable region comprising CDRs as set forth in the amino acid sequences of SEQ ID Nos: 36, 37, and 38. Included in the disclosure are humanized antibodies or antigen binding fragments comprising the CDRs of AGX-A05. Further, the heavy chain variable amino acid sequences and the light chain variable amino acid sequences of AGX-A05 are described in SEQ ID NOS: 27 and 33, respectively. The amino acid sequences of murine monoclonal antibody AGX-A07 are described in Table 1. Specifically, the heavy chain CDR sequences are set forth in SEQ ID Nos: 42, 43, and 44 (CDR1, CDR2, and CDR3), and the light chain CDR amino acid sequences are set forth in SEQ ID Nos: 48, 49, and 50 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 42, 43, and 44 and/or a light chain variable region comprising CDRs as set forth in the amino acid sequences of SEQ ID Nos: 48, 49, and 50. Included in the disclosure are humanized antibodies or antigen binding fragments comprising the CDRs of AGX-A07. Further, the heavy chain variable amino acid sequences and the light chain variable amino acid sequences of AGX-A07 are described in SEQ ID NOs: 39 and 45, respectively.

In one embodiment, a humanized AGX-A07 (h AGX-A07) antibody or antigen binding fragments thereof is provided, comprising a heavy chain sequence as forth in the amino acid sequence of SEQ ID NO: 90. In some embodiments, the humanized AGX-A07 antibody or antigen binding fragments thereof is a humanized mutated AGX-A07 (hm AGX-A07) antibody or antigen binding fragments thereof, comprising a heavy chain sequence comprising one or more substitutions in the sequence as set forth in the amino acid sequence of SEQ ID NO: 90. As shown in Table 1, the heavy chain sequence set forth in SEQ ID NO: 90 is also referred to herein as AGX-A07 H2. In some embodiments, the humanized AGX-A07 antibody or antigen binding fragments thereof is a humanized mutated AGX-A07 antibody or antigen binding fragments thereof, comprising a heavy chain sequence comprising one or more substitutions in the sequence as set forth in the amino acid sequence of SEQ ID NO: 90, wherein the one or more substitutions are in amino acid positions 1, 44, and 80 of SEQ ID NO: 90. In some cases, the humanized mutated AGX-A07 antibody or antigen binding fragments thereof comprises an E1Q (glutamic acid to glutamine substitution at position 1 of the heavy chain, SEQ ID NO: 90). In some cases, the humanized mutated AGX-A07 antibody or antigen binding fragments thereof comprises a D44G (aspartate to glycine substitution at position 44 of the heavy chain, SEQ ID NO: 90). In some cases, the humanized mutated AGX-A07 antibody or antigen binding fragments thereof comprises a F80Y (phenyl alanine to tyrosine substitution at position 80 of the heavy chain, SEQ ID NO: 90). In some embodiments, a humanized mutated AGX-A07 antibody or antigen binding fragments is provided, comprising a heavy chain sequence as forth in the amino acid sequence of SEQ ID NO: 92. As shown in Table 1, the heavy chain sequence set forth in SEQ ID NO: 92 is also referred to herein as AGX-A07 H2v1. In some embodiments, humanized AGX-A07 antibodies or antigen binding fragments are provided, comprising a light chain sequence as forth in the amino acid sequence of SEQ ID NO: 97. As shown in Table 1, the light chain sequence set forth in SEQ ID NO: 97 is also referred to herein as AGX-A07 L5. In some embodiments, the humanized AGX-A07 antibody or antigen binding fragments thereof is a humanized mutated AGX-A07 antibody or antigen binding fragments thereof, comprising a light chain sequence comprising one or more substitutions in the sequence as set forth in the amino acid sequence of SEQ ID NO: 97. In some embodiments, the humanized AGX-A07 antibodies or antigen binding fragments thereof is a humanized mutated AGX-A07 antibody or antigen binding fragments thereof, comprising a light chain sequence comprising one or more substitutions in the sequence as set forth in the amino acid sequence of SEQ ID NO: 97, wherein the one or more substitutions are in amino acid positions 3, 26, 62, and 90 of SEQ ID NO: 97. In some cases, the humanized mutated AGX-A07 antibody or antigen binding fragments thereof comprises an I3V (isoleucine to valine substitution at position 3 of the light chain, SEQ ID NO: 97). In some cases, the humanized mutated AGX-A07 antibody or antigen binding fragments thereof comprises a N26Q (asparagine to glutamine substitution at position 26 of the light chain, SEQ ID NO: 97). In some cases, the humanized mutated AGX-A07 antibody or antigen binding fragments thereof comprises a N26S (asparagine to serine substitution at position 26 of the light chain, SEQ ID NO: 97). In some cases, the humanized mutated AGX-A07 antibody or antigen binding fragments thereof comprises a G62S (glycine to serine substitution at position 62 of the light chain, SEQ ID NO: 97). In some cases, the humanized mutated AGX-A07 antibody or antigen binding fragments thereof comprises a W90Y (tryptophan to tyrosine substitution at position 90 of the light chain, SEQ ID NO: 97). In some embodiments, humanized mutated AGX-A07 antibodies or antigen binding fragments are provided, comprising a light chain sequence as forth in an amino acid sequence selected from the group consisting of SEQ ID NO: 99, SEQ ID NO: 101, SEQ ID NO: 103, and SEQ ID NO: 105. As shown in Table 1, the light chain sequence set forth in SEQ ID NO: 99 is also referred to herein as AGX-A07 L5v1, the light chain sequence set forth in SEQ ID NO: 101 is also referred to herein as AGX-A07 L5v2, the light chain sequence set forth in SEQ ID NO: 103 is also referred to herein as AGX-A07 L5v3, and the light chain sequence set forth in SEQ ID NO: 105 is also referred to herein as AGX-A07 L5v4. Exemplary coding sequence for the heavy chain of a humanized AGX-A07 antibody or antigen binding fragment thereof is provided in SEQ ID NO: 91. Exemplary coding sequence for the heavy chain of a humanized mutated AGX-A07 antibody or antigen binding fragment thereof is provided in SEQ ID NO: 93. Exemplary coding sequence for the light chain of a humanized AGX-A07 antibody or antigen binding fragment thereof is provided in SEQ ID NO: 98 (AGX-A07 L5). Exemplary coding sequences for the light chain of a humanized mutated AGX-A07 antibody or antigen binding fragment thereof are provided in SEQ ID NO: 100 (AGX-A07 L5v1), SEQ ID NO: 102 (AGX-A07 L5v2), SEQ ID NO: 104 (AGX-A07 L5v3), and SEQ ID NO: 106 (AGX-A07 L5v4).

In one embodiment, a humanized AGX-A07 antibody or antigen binding fragments thereof is provided, comprising a heavy chain variable domain sequence as forth in the amino acid sequence of SEQ ID NO: 130 or SEQ ID NO: 132. In some embodiments, the humanized AGX-A07 antibody or antigen binding fragments thereof is a humanized mutated AGX-A07 antibody or antigen binding fragments thereof, comprising a heavy chain variable domain sequence comprising one or more substitutions in the sequence as set forth in the amino acid sequence of SEQ ID NO: 130 or SEQ ID NO: 132. In one embodiment, a humanized AGX-A07 antibody or antigen binding fragments thereof is provided, comprising a light chain variable domain sequence as forth in the amino acid sequence of SEQ ID NO: 131 or SEQ ID NO: 133. In some embodiments, the humanized AGX-A07 antibody or antigen binding fragments thereof is a humanized mutated AGX-A07 antibody or antigen binding fragments thereof, comprising a light chain variable domain sequence comprising one or more substitutions in the sequence as set forth in the amino acid sequence of SEQ ID NO: 131 or SEQ ID NO: 133.

In some embodiments, the humanized AGX-A07 antibody or antigen binding fragment thereof is a humanized mutated AGX-A07 antibody or antigen binding fragment thereof comprising a light chain variable domain sequence comprising the sequence as set forth in the amino acid sequence of SEQ ID NO: 131 and a heavy chain variable domain sequence comprising the sequence as set forth in the amino acid sequence of SEQ ID NO: 130. In some embodiments, the humanized AGX-A07 antibody or antigen binding fragment thereof is a humanized mutated AGX-A07 antibody or antigen binding fragments thereof, comprising a light chain variable domain sequence comprising one or more substitutions in the sequence as set forth in the amino acid sequence of SEQ ID NO: 131 and a heavy chain variable domain sequence comprises one or more substitutions in the sequence as set forth in the amino acid sequence of SEQ ID NO: 130. In some embodiments, the humanized AGX-A07 antibody or antigen binding fragments thereof is a humanized mutated AGX-A07 antibody or antigen binding fragments thereof comprising a light chain variable domain sequence comprising the sequence as set forth in the amino acid sequence of SEQ ID NO: 133 and a heavy chain variable domain sequence comprising the sequence as set forth in the amino acid sequence of SEQ ID NO: 132. In some embodiments, the humanized AGX-A07 antibody or antigen binding fragments thereof is a humanized mutated AGX-A07 antibody or antigen binding fragments thereof, comprising a light chain variable domain sequence comprising one or more substitutions in the sequence as set forth in the amino acid sequence of SEQ ID NO: 133 and a heavy chain variable domain sequence comprises one or more substitutions in the sequence as set forth in the amino acid sequence of SEQ ID NO: 132. In some embodiments, the humanized AGX-A07 antibody or antigen binding fragments thereof is a humanized mutated AGX-A07 antibody or antigen binding fragments thereof comprising a heavy chain sequence comprising the sequence as set forth in the amino acid sequence of SEQ ID NO: 156, or a sequence comprising one of more substitutions in the amino acid sequence of SEQ ID NO: 156.

In some cases, the humanized AGX-A07 antibodies or antigen binding fragments thereof comprise heavy chain CDR sequences as set forth in SEQ ID Nos: 94, 95, and 96 (CDR1, CDR2, and CDR3), or CDR sequences comprising one or more substitutions in the sequences as set forth in SEQ ID Nos: 94, 95, and 96 (CDR1, CDR2, and CDR3). In some cases, the humanized mutated AGX-A07 antibodies or antigen binding fragments thereof comprises heavy chain CDR sequences as set forth in SEQ ID Nos: 94, 95, and 96 (CDR1, CDR2, and CDR3), or CDR sequences comprising one or more substitutions in the sequences as set forth in SEQ ID Nos: 94, 95, and 96 (CDR1, CDR2, and CDR3).

In some cases, the humanized mutated AGX-A07 antibodies or antigen binding fragments thereof comprise heavy chain CDR1 sequence as set forth in SEQ ID NO: 94, or a heavy chain CDR1 sequence comprising one or more substitutions in the sequences as set forth in SEQ ID NO: 94. In some cases, the humanized mutated AGX-A07 antibodies or antigen binding fragments thereof comprise a heavy chain CDR2 sequence as set forth in SEQ ID NO: 95, or a heavy chain CDR2 sequence comprising one or more substitutions in the sequences as set forth in SEQ ID NO: 95. In some cases, the humanized mutated AGX-A07 antibodies or antigen binding fragments thereof comprise a heavy chain CDR3 sequence as set forth in SEQ ID NO: 96, or a heavy chain CDR3 sequence comprising one or more substitutions in the sequences as set forth in SEQ ID NO: 96.

In some cases, the humanized AGX-A07 antibodies or antigen binding fragments thereof comprise light chain CDR sequences as set forth in SEQ ID Nos: 107, 109, and 110 (CDR1, CDR2, and CDR3), or CDR sequences comprising one or more substitutions in the sequences as set forth in SEQ ID Nos: 107, 109, and 110 (CDR1, CDR2, and CDR3). In some cases, the humanized AGX-A07 antibodies or antigen binding fragments thereof comprise light chain CDR sequences as set forth in SEQ ID Nos: 107, 109, and 111 (CDR1, CDR2, and CDR3), or CDR sequences comprising one or more substitutions in the sequences as set forth in SEQ ID Nos: 107, 109, and 111 (CDR1, CDR2, and CDR3). In some cases, the humanized AGX-A07 antibodies or antigen binding fragments thereof comprise light chain CDR sequences as set forth in SEQ ID Nos: 108, 109, and 110 (CDR1, CDR2, and CDR3), or CDR sequences comprising one or more substitutions in the sequences as set forth in SEQ ID Nos: 108, 109, and 110 (CDR1, CDR2, and CDR3). In some cases, the humanized AGX-A07 antibodies or antigen binding fragments thereof comprise light chain CDR sequences as set forth in SEQ ID Nos: 108, 109, and 111 (CDR1, CDR2, and CDR3), or CDR sequences comprising one or more substitutions in the sequences as set forth in SEQ ID Nos: 108, 109, and 111 (CDR1, CDR2, and CDR3).

In some cases, the humanized mutated AGX-A07 antibodies or antigen binding fragments thereof comprise light chain CDR1 sequence as set forth in SEQ ID Nos: 107 or 108, or light chain CDR1 sequence comprising one or more substitutions in the sequences as set forth in SEQ ID Nos: 107 or 108. In some cases, the humanized mutated AGX-A07 antibodies or antigen binding fragments thereof comprise light chain CDR2 sequence as set forth in SEQ ID NO: 109, or light chain CDR2 sequence comprising one or more substitutions in the sequences as set forth in SEQ ID NO: 109. In some cases, the humanized mutated AGX-A07 antibodies or antigen binding fragments thereof comprise light chain CDR3 sequence as set forth in SEQ ID Nos: 110 or 111, or light chain CDR1 sequence comprising one or more substitutions in the sequences as set forth in SEQ ID Nos: 110 or 111. In some cases, the humanized mutated AGX-A07 antibodies or antigen binding fragments thereof comprise light chain CDR3 sequence as set forth in SEQ ID NO: 110, or light chain CDR1 sequence comprising one or more substitutions in the sequences as set forth in SEQ ID Nos: 110.

In some embodiments, the humanized mutated AGX-A07 comprises a heavy chain variable region comprising the following amino acid substitutions: Q1E, D44G, F80Y in SEQ ID NO: 132 (also referred to herein as AGX-A07 H2), and a light chain variable region comprising the following amino acid substitutions: I3V, N26Q, G62S in SEQ ID NO: 133 (also referred to herein as AGX-A07 L5). In some embodiments, the humanized mutated AGX-A07 comprises a heavy chain variable region comprising the following amino acid substitutions: Q1E, D44G, F80Y in SEQ ID NO: 132, and a light chain variable region comprising the following amino acid substitutions: I3V, N26Q, G62S in SEQ ID NO: 133, wherein the heavy chain comprises CDR1 (SEQ ID NO: 94), CDR2 (SEQ ID NO: 95), and CDR3 (SEQ ID NO: 96), and the light chain comprises CDR1 (SEQ ID NO: 108), CDR2 (SEQ ID NO: 109), and CDR3 (SEQ ID NO: 110). In some embodiments, the humanized mutated AGX-A07 is AGX-A07 H2v1L5v2 and comprises a heavy chain comprising the amino acid sequence as set forth in SEQ ID NO: 130 (also referred to herein as AGX-A07 H2v1), and a light chain comprising the amino acid sequence as set forth in SEQ ID NO: 131 (also referred to herein as AGX-A07 L5v2). In some embodiments, the humanized mutated AGX-A07 comprises a heavy chain comprising the amino acid sequence as set forth in SEQ ID NO: 92, and a light chain comprising the amino acid sequence as set forth in SEQ ID NO: 101.

The amino acid sequences of murine monoclonal antibody AGX-A08 are described in Table 1. Specifically, the heavy chain CDR sequences are set forth in SEQ ID Nos: 54, 55, and 56 (CDR1, CDR2, and CDR3), and the light chain CDR amino acid sequences are set forth in SEQ ID Nos: 60, 61, and 62 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 54, 55, and 56 and/or a light chain variable region comprising CDRs as set forth in the amino acid sequences of SEQ ID Nos: 60, 61, and 62. Included in the disclosure are humanized antibodies or antigen binding fragments comprising the CDRs of AGX-A08. Further, the heavy chain variable amino acid sequences and the light chain variable amino acid sequences of AGX-A08 are described in SEQ ID NOs: 51 and 57, respectively.

The amino acid sequences of murine monoclonal antibody AGX-A09 are described in Table 1. Specifically, the heavy chain CDR sequences are set forth in SEQ ID Nos: 66, 67, and 68 (CDR1, CDR2, and CDR3), and the light chain CDR amino acid sequences are set forth in SEQ ID Nos: 72, 73, and 74 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 66, 67, and 68 and/or a light chain variable region comprising CDRs as set forth in the amino acid sequences of SEQ ID Nos: 72, 73, and 74. Included in the disclosure are humanized antibodies or antigen binding fragments comprising the CDRs of AGX-A09. Further, the heavy chain variable amino acid sequences and the light chain variable amino acid sequences of AGX-A09 are described in SEQ ID NOs: 63 and 69, respectively.

The amino acid sequences of murine monoclonal antibody AGX-A11 are described in Table 1. Specifically, the heavy chain CDR sequences are set forth in SEQ ID Nos: 78, 79, and 80 (CDR1, CDR2, and CDR3), and the light chain CDR amino acid sequences are set forth in SEQ ID Nos: 84, 85, and 86 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 78, 79, and 80 and/or a light chain variable region comprising CDRs as set forth in the amino acid sequences of SEQ ID Nos: 84, 85, and 862. Included in the disclosure are humanized antibodies or antigen binding fragments comprising the CDRs of AGX-A11. Further, the heavy chain variable amino acid sequences and the light chain variable amino acid sequences of AGX-A11 are described in SEQ ID NOS: 75 and 81, respectively.

The amino acid sequences of a humanized antibody AGX-A01 (h AGX-A01) are described in Table 1. As shown in Table 1, the heavy chain sequence set forth is SEQ ID NO: 112 is also referred to herein as AGX-A01 H1. Specifically, the heavy chain CDR sequences are set forth in SEQ ID Nos: 115, 116, and 118 (CDR1, CDR2, and CDR3) and the light chain CDR amino acid sequences are set forth in SEQ ID Nos: 124, 128, and 129 (CDR1, CDR2, and CDR3). Further, exemplary heavy chain amino acid sequence and the light chain amino acid sequence of the humanized AGX-A01 are described in SEQ ID Nos: 112 and 122, respectively. Exemplary coding sequences for the heavy chain and the light chain of the humanized AGX-A01 are described in SEQ ID Nos: 113 and 123, respectively.

In some embodiments, the humanized AGX-A01 antibody or antigen binding fragments thereof is a humanized mutated AGX-A01 (hm AGX-A01) antibody or antigen binding fragments thereof, comprising a heavy chain sequence comprising one or more substitutions in the sequence as set forth in the amino acid sequence of SEQ ID NO: 112. In some embodiments, the humanized AGX-A01 antibody or antigen binding fragments thereof is a humanized mutated AGX-A01 antibody or antigen binding fragments thereof, comprising a heavy chain sequence comprising one or more substitutions in the sequence as set forth in the amino acid sequence of SEQ ID NO: 112, wherein the one or more substitutions are in amino acid positions 63 and 106 of SEQ ID NO: 112. In some cases, the humanized mutated AGX-A01 antibody or antigen binding fragments thereof comprises a G63S (glycine to serine substitution at position 63 of the heavy chain, SEQ ID NO: 112). In some cases, the humanized mutated AGX-A01 antibody or antigen binding fragments thereof comprises a D106E (aspartate to glutamic acid substitution at position 106 of the heavy chain, SEQ ID NO: 112). In some cases, the humanized mutated AGX-A01 antibody or antigen binding fragments thereof comprises a D106S (aspartate to serine substitution at position 106 of the heavy chain, SEQ ID NO: 112). In some embodiments, a humanized mutated AGX-A01 antibody or antigen binding fragments is provided, comprising a heavy chain sequence as forth in the amino acid sequence of SEQ ID NO: 114. As shown in Table 1, the heavy chain sequence set forth is SEQ ID NO: 114 is also referred to herein as AGX-A01 H1v1.

In some embodiments, humanized AGX-A01 antibodies or antigen binding fragments are provided, comprising a light chain sequence as forth in the amino acid sequence of SEQ ID NO: 122. As shown in Table 1, the light chain sequence set forth is SEQ ID NO: 122 is also referred to herein as AGX-A01 L10. In some embodiments, the humanized AGX-A01 antibody or antigen binding fragments thereof is a humanized mutated AGX-A01 antibody or antigen binding fragments thereof, comprising a light chain sequence comprising one or more substitutions in the sequence as set forth in the amino acid sequence of SEQ ID NO: 122. In some embodiments, the humanized mutated AGX-A01 antibody or antigen binding fragments thereof is a humanized mutated AGX-A01 antibody or antigen binding fragments thereof, comprising a light chain sequence comprising one or more substitutions in the sequence as set forth in the amino acid sequence of SEQ ID NO: 122, wherein the one or more substitutions are in one or more amino acid positions selected from amino acid positions 1, 33, 42, 51, 86, and 90 of SEQ ID NO: 122. In some embodiments, the humanized mutated AGX-A01 antibody or antigen binding fragments thereof is a humanized mutated AGX-A01 antibody or antigen binding fragments thereof, comprising a light chain sequence comprising one or more substitutions in the sequence as set forth in the amino acid sequence of SEQ ID NO: 122, wherein the one or more substitutions are in one or more amino acid positions selected from amino acid positions 1, 33, 42, 51, and 86 of SEQ ID NO: 122. In some cases, the humanized mutated AGX-A01 antibody or antigen binding fragments thereof comprises an A1E (alanine to glutamic acid substitution at position 1 of the light chain, SEQ ID NO: 122). In some cases, the humanized mutated AGX-A01 antibody or antigen binding fragments thereof comprises a N33S (asparagine to serine substitution at position 33 of the light chain, SEQ ID NO: 122). In some cases, the humanized mutated AGX-A01 antibody or antigen binding fragments thereof comprises a M42Q (methionine to glutamine substitution at position 42 of the light chain, SEQ ID NO: 122). In some cases, the humanized mutated AGX-A01 antibody or antigen binding fragments thereof comprises a V51L (valine to leucine substitution at position 51 of the light chain, SEQ ID NO: 122). In some cases, the humanized mutated AGX-A01 antibody or antigen binding fragments thereof comprises a D86E (aspartate to glutamic acid substitution at position 86 of the light chain, SEQ ID NO: 122). In some cases, the humanized mutated AGX-A01 antibody or antigen binding fragments thereof comprises an I90V (isoleucine to valine substitution at position 90 of the light chain, SEQ ID NO: 122).

In some cases, the humanized AGX-A01 antibodies or antigen binding fragments thereof comprise heavy chain CDR sequences as set forth in SEQ ID Nos: 115 (CDR1); 116 (CDR2); and 118 (CDR3), or CDR sequences comprising one or more substitutions in the sequences as set forth in SEQ ID Nos: 115 (CDR1); 116 (CDR2); and 118 (CDR3). In some cases, the humanized mutated AGX-A01 antibodies or antigen binding fragments thereof comprise heavy chain CDR sequences as set forth in SEQ ID Nos: 115 (CDR1); 116 or 117 (CDR2); and 118, 119, 120, or 121 (CDR3), or CDR sequences comprising one or more substitutions in the sequences as set forth in SEQ ID Nos: 115 (CDR1); 116 or 117 (CDR2); and 118, 119, 120, or 121 (CDR3).

In some cases, the humanized mutated AGX-A01 antibodies or antigen binding fragments thereof comprise heavy chain CDR1 sequence as set forth in SEQ ID NO: 115, or a heavy chain CDR1 sequence comprising one or more substitutions in the sequences as set forth in SEQ ID NO: 115. In some cases, the humanized mutated AGX-A01 antibodies or antigen binding fragments thereof comprise a heavy chain CDR2 sequence as set forth in SEQ ID NO: 116, or a heavy chain CDR2 sequence comprising one or more substitutions in the sequences as set forth in SEQ ID NO: 116. In some cases, the humanized mutated AGX-A01 antibodies or antigen binding fragments thereof comprise a heavy chain CDR2 sequence as set forth in SEQ ID NO: 117, or a heavy chain CDR2 sequence comprising one or more substitutions in the sequences as set forth in SEQ ID NO: 117. In some cases, the humanized mutated AGX-A01 antibodies or antigen binding fragments thereof comprise a heavy chain CDR3 sequence as set forth in a sequence selected from SEQ ID Nos: 118, 119, 120 and 121, or a heavy chain CDR3 sequence comprising one or more substitutions in a sequence selected from SEQ ID Nos: 118, 119, 120, and 121.

In some cases, the humanized AGX-A01 antibodies or antigen binding fragments thereof comprise light chain CDR sequences as set forth in SEQ ID Nos: 124 (CDR1); 128 (CDR2); and 129 (CDR3), or CDR sequences comprising one or more substitutions in the sequences as set forth in SEQ ID Nos: 124 (CDR1); 128 (CDR2); and 129 (CDR3). In some cases, the humanized mutated AGX-A01 antibodies or antigen binding fragments thereof comprise light chain CDR sequences as set forth in SEQ ID Nos: 124, 125, 126, or 127 (CDR1); 128 (CDR2); and 129 (CDR3), or CDR sequences comprising one or more substitutions in the sequences as set forth in SEQ ID Nos: 124, 125, 126, or 127 (CDR1); 128 (CDR2); and 129 (CDR3).

In some cases, the humanized mutated AGX-A01 antibodies or antigen binding fragments thereof comprise light chain CDR1 sequence as set forth in SEQ ID Nos: 125, 126, 127, or 128, or light chain CDR1 sequence comprising one or more substitutions in the sequences as set forth in SEQ ID Nos: 125, 126, 127, or 128. In some cases, the humanized mutated AGX-A01 antibodies or antigen binding fragments thereof comprise light chain CDR2 sequence as set forth in SEQ ID NO: 129, or light chain CDR2 sequence comprising one or more substitutions in the sequences as set forth in SEQ ID NO: 129. In some cases, the humanized mutated AGX-A01 antibodies or antigen binding fragments thereof comprise light chain CDR3 sequence as set forth in SEQ ID Nos: 130, or light chain CDR1 sequence comprising one or more substitutions in the sequences as set forth in SEQ ID Nos: 130.

In one embodiment, the disclosure provides an anti-TM4SF1 antibody, or antigen-binding fragment thereof, that comprises a heavy chain variable domain encoded by a nucleic acid sequence as set forth in SEQ ID NO: 3, and a light chain variable domain encoded by a nucleic acid sequence as set forth in SEQ ID NO: 9. In one embodiment, the disclosure provides an anti-TM4SF1 antibody, or antigen-binding fragment thereof, that comprises a heavy chain variable domain encoded by a nucleic acid sequence as set forth in SEQ ID NO: 15, and a light chain variable domain encoded by a nucleic acid sequence as set forth in SEQ ID NO: 21 In one embodiment, the disclosure provides an anti-TM4SF1 antibody, or antigen-binding fragment thereof, that comprises a heavy chain variable domain encoded by a nucleic acid sequence as set forth in SEQ ID NO: 27, and a light chain variable domain encoded by a nucleic acid sequence as set forth in SEQ ID NO: 33. In one embodiment, the disclosure provides an anti-TM4SF1 antibody, or antigen-binding fragment thereof, that comprises a heavy chain variable domain encoded by a nucleic acid sequence as set forth in SEQ ID NO: 39, and a light chain variable domain encoded by a nucleic acid sequence as set forth in SEQ ID NO: 45. In one embodiment, the disclosure provides an anti-TM4SF1 antibody, or antigen-binding fragment thereof, that comprises a heavy chain variable domain encoded by a nucleic acid sequence as set forth in SEQ ID NO: 51, and a light chain variable domain encoded by a nucleic acid sequence as set forth in SEQ ID NO: 57. In one embodiment, the disclosure provides an anti-TM4SF1 antibody, or antigen-binding fragment thereof, that comprises a heavy chain variable domain encoded by a nucleic acid sequence as set forth in SEQ ID NO: 63, and a light chain variable domain encoded by a nucleic acid sequence as set forth in SEQ ID NO: 69. In one embodiment, the disclosure provides an anti-TM4SF1 antibody, or antigen-binding fragment thereof, that comprises a heavy chain variable domain encoded by a nucleic acid sequence as set forth in SEQ ID NO: 75, and a light chain variable domain encoded by a nucleic acid sequence as set forth in SEQ ID NO: 81. In one embodiment, the disclosure provides an anti-TM4SF1 antibody, or antigen-binding fragment thereof, that comprises a heavy chain variable domain encoded by a nucleic acid sequence as set forth in SEQ ID NO: 90, and a light chain variable domain encoded by a nucleic acid sequence as set forth in SEQ ID NO: 97. In one embodiment, the disclosure provides an anti-TM4SF1 antibody, or antigen-binding fragment thereof, that comprises a heavy chain variable domain encoded by a nucleic acid sequence as set forth in SEQ ID NO: 90, and a light chain variable domain encoded by a nucleic acid sequence as set forth in SEQ ID NO: 99. In one embodiment, the disclosure provides an anti-TM4SF1 antibody, or antigen-binding fragment thereof, that comprises a heavy chain variable domain encoded by a nucleic acid sequence as set forth in SEQ ID NO: 90, and a light chain variable domain encoded by a nucleic acid sequence as set forth in SEQ ID NO: 101. In one embodiment, the disclosure provides an anti-TM4SF1 antibody, or antigen-binding fragment thereof, that comprises a heavy chain variable domain encoded by a nucleic acid sequence as set forth in SEQ ID NO: 90, and a light chain variable domain encoded by a nucleic acid sequence as set forth in SEQ ID NO: 103. In one embodiment, the disclosure provides an anti-TM4SF1 antibody, or antigen-binding fragment thereof, that comprises a heavy chain variable domain encoded by a nucleic acid sequence as set forth in SEQ ID NO: 90, and a light chain variable domain encoded by a nucleic acid sequence as set forth in SEQ ID NO: 105. In one embodiment, the disclosure provides an anti-TM4SF1 antibody, or antigen-binding fragment thereof, that comprises a heavy chain variable domain encoded by a nucleic acid sequence as set forth in SEQ ID NO: 92, and a light chain variable domain encoded by a nucleic acid sequence as set forth in SEQ ID NO: 97. In one embodiment, the disclosure provides an anti-TM4SF1 antibody, or antigen-binding fragment thereof, that comprises a heavy chain variable domain encoded by a nucleic acid sequence as set forth in SEQ ID NO: 92, and a light chain variable domain encoded by a nucleic acid sequence as set forth in SEQ ID NO: 99. In one embodiment, the disclosure provides an anti-TM4SF1 antibody, or antigen-binding fragment thereof, that comprises a heavy chain variable domain encoded by a nucleic acid sequence as set forth in SEQ ID NO: 92, and a light chain variable domain encoded by a nucleic acid sequence as set forth in SEQ ID NO: 101. In one embodiment, the disclosure provides an anti-TM4SF1 antibody, or antigen-binding fragment thereof, that comprises a heavy chain variable domain encoded by a nucleic acid sequence as set forth in SEQ ID NO: 92, and a light chain variable domain encoded by a nucleic acid sequence as set forth in SEQ ID NO: 103. In one embodiment, the disclosure provides an anti-TM4SF1 antibody, or antigen-binding fragment thereof, that comprises a heavy chain variable domain encoded by a nucleic acid sequence as set forth in SEQ ID NO: 92, and a light chain variable domain encoded by a nucleic acid sequence as set forth in SEQ ID NO: 105.

In one embodiment, the present disclosure provides an anti-TM4SF1 antibody, or antigen-binding fragment thereof, that has a heavy chain variable domain sequence that is at least 95% identical, at least 96% identical, at least 97% identical, at least 98% identical, at least 99% identical, or 100% identical to an amino acid sequence selected from SEQ ID NO: 3, SEQ ID NO: 15, SEQ ID NO: 27, SEQ ID NO: 39, SEQ ID NO: 51, SEQ ID NO: 63, SEQ ID NO: 75, SEQ ID NO: 90, SEQ ID NO: 92, SEQ ID NO: 112, or SEQ ID NO: 114; and that has a light chain variable domain sequence that is at least 95% identical, at least 96% identical, at least 97% identical, at least 98% identical, at least 99% identical, or 100% identical to an amino acid sequence selected from SEQ ID NO: 9, SEQ ID NO: 21, SEQ ID NO: 33, SEQ ID NO: 45, SEQ ID NO: 57, SEQ ID NO: 69, SEQ ID NO: 81, SEQ ID NO: 97, SEQ ID NO: 99, SEQ ID NO: 101, SEQ ID NO: 103, SEQ ID NO: 105, or SEQ ID NO: 122. In one embodiment, the present disclosure provides an anti-TM4SF1 antibody, or antigen-binding fragment thereof, that has a heavy chain variable domain sequence that is at least 95% identical, at least 96% identical, at least 97% identical, at least 98% identical, at least 99% identical, or 100% identical to an amino acid sequence selected from SEQ ID NO: 3, SEQ ID NO: 15, SEQ ID NO: 27, SEQ ID NO: 39, SEQ ID NO: 51, SEQ ID NO: 63, SEQ ID NO: 75, SEQ ID NO: 90, SEQ ID NO: 92, SEQ ID NO: 112, or SEQ ID NO: 114; and that has a light chain variable domain sequence that is at least 95% identical, at least 96% identical, at least 97% identical, at least 98% identical, at least 99% identical, or 100% identical to an amino acid sequence selected from SEQ ID NO: 9, SEQ ID NO: 21, SEQ ID NO: 33, SEQ ID NO: 45, SEQ ID NO: 57, SEQ ID NO: 69, SEQ ID NO: 81, SEQ ID NO: 97, SEQ ID NO: 99, SEQ ID NO: 101, or SEQ ID NO: 122.

In one embodiment, the disclosure includes an anti-TM4SF1 antibody which is an IgG and comprises four polypeptide chains including two heavy chains each comprising a heavy chain variable domain and heavy chain constant regions CH1, CH2 and CH3, and two light chains each comprising a light chain variable domain and a light chain constant region (CL). In certain embodiments, the antibody is a human IgG1, IgG2, or an IgG4. In certain embodiments, the antibody is a human IgG1. In other embodiments, the antibody is an IgG2. The heavy and light chain variable domain sequences may contain CDRs as set forth in Table 1.

Complementarity determining regions (CDRs) are known as hypervariable regions both in the light chain and the heavy chain variable domains. The more highly conserved portions of variable domains are called the framework (FR). CDRs and framework regions (FR) of a given antibody may be identified using the system described by Kabat et al. supra; Lefranc et al., supra and/or Honegger and Pluckthun, supra. Also familiar to those in the art is the numbering system described in Kabat et al. (1991, NIH Publication 91-3242, National Technical Information Service, Springfield, Va.). In this regard Kabat et al. defined a numbering system for variable domain sequences, including the identification of CDRs, that is applicable to any antibody.

One or more CDRs may be incorporated into a molecule either covalently or noncovalently to make it an antigen binding protein.

An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest. The CDR3, in particular, is known to play an important role in antigen binding of an antibody or antibody fragment.

In one embodiment, the disclosure provides an anti-TM4SF1 antibody, or an antigen-binding fragment thereof, comprising a heavy chain comprising a CDR3 domain as set forth in any one of SEQ ID NO: 8, SEQ ID NO: 20, SEQ ID NO: 32, SEQ ID NO: 44, SEQ ID NO: 56, SEQ ID NO: 68, or SEQ ID NO: 80 and comprising a variable domain comprising an amino acid sequence that has at least about 60%, at least about 65%, at least about 70%, at least about 75%, at least about 80%, at least about 85%, at least about 90%, at least about 95%, at least about 96%, at least about 97%, at least about 98%, at least about 99%, or 100% identical to a sequence as set forth in any one of SEQ ID NO: 3, SEQ ID NO: 15, SEQ ID NO: 27, SEQ ID NO: 39, SEQ ID NO: 51, SEQ ID NO: 63, or SEQ ID NO: 75. In one embodiment, the disclosure provides an anti-TM4SF1 antibody, or an antigen-binding fragment thereof, comprising a light chain comprising a CDR3 domain as set forth in any one of SEQ ID NO: 14, SEQ ID NO: 26, SEQ ID NO: 38, SEQ ID NO: 50, SEQ ID NO: 62, SEQ ID NO: 74, or SEQ ID NO: 86, and having a light chain variable domain comprising an amino acid sequence that has at least at least about 60%, at least about 65%, at least about 70%, at least about 75%, at least about 80%, at least about 85%, at least about 90%, at least about 95%, at least about 96%, at least about 97%, at least about 98%, or at least about 99%, or 100% identical to a sequence as set forth in any one of SEQ ID NO: 9, SEQ ID NO: 21, SEQ ID NO: 33, SEQ ID NO: 45, SEQ ID NO: 57, SEQ ID NO: 69, or SEQ ID NO: 81. Thus, in certain embodiments, the CDR3 domain is held constant, while variability may be introduced into the remaining CDRs and/or framework regions of the heavy and/or light chains, while the antibody, or antigen binding fragment thereof, retains the ability to bind to TM4SF1 and retains the functional characteristics, e.g., binding affinity, of the parent, or has improved functional characteristic, e.g., binding affinity, compared to the parent.

In one embodiment, the disclosure provides an anti-TM4SF1 antibody, or an antigen-binding fragment thereof, comprising a heavy chain comprising a CDR2 domain as set forth in any one of SEQ ID NO: 7, SEQ ID NO: 19, SEQ ID NO: 31, SEQ ID NO: 43, SEQ ID NO: 55, SEQ ID NO: 67, or SEQ ID NO: 79 and comprising a variable domain comprising an amino acid sequence that has at least at least about 60%, at least about 65%, at least about 70%, at least about 75%, at least about 80%, at least about 85%, at least about 90%, at least about 95%, at least about 96%, at least about 97%, at least about 98%, or at least about 99%, or 100% identical to a sequence as set forth in any one of SEQ ID NO: 3, SEQ ID NO: 15, SEQ ID NO: 27, SEQ ID NO: 39, SEQ ID NO: 51, SEQ ID NO: 63, or SEQ ID NO: 75. In one embodiment, the disclosure provides an anti-TM4SF1 antibody, or an antigen-binding fragment thereof, comprising a light chain comprising a CDR2 domain as set forth in any one of SEQ ID NO: 13, SEQ ID NO: 25, SEQ ID NO: 37, SEQ ID NO: 49, SEQ ID NO: 61, SEQ ID NO: 73, or SEQ ID NO: 85, and having a light chain variable domain comprising an amino acid sequence that has at least at least about 60%, at least about 65%, at least about 70%, at least about 75%, at least about 80%, at least about 85%, at least about 90%, at least about 95%, at least about 96%, at least about 97%, at least about 98%, or at least about 99%, or 100% identical to a sequence as set forth in any one of SEQ ID NO: 9, SEQ ID NO: 21, SEQ ID NO: 33, SEQ ID NO: 45, SEQ ID NO: 57, SEQ ID NO: 69, or SEQ ID NO: 81. Thus, in certain embodiments, the CDR2 domain is held constant, while variability may be introduced into the remaining CDRs and/or framework regions of the heavy and/or light chains, while the antibody, or antigen binding fragment thereof, retains the ability to bind to TM4SF1 and retains the functional characteristics, e.g., binding affinity, of the parent, or has improved functional characteristic, e.g., binding affinity, compared to the parent.

In one embodiment, the disclosure provides an anti-TM4SF1 antibody, or an antigen-binding fragment thereof, comprising a heavy chain comprising a CDR1 domain as set forth in any one of SEQ ID NO: 6, SEQ ID NO: 18, SEQ ID NO: 30, SEQ ID NO: 42, SEQ ID NO: 54, SEQ ID NO: 66, or SEQ ID NO: 78 and comprising a variable domain comprising an amino acid sequence that has at least at least about 60%, at least about 65%, at least about 70%, at least about 75%, at least about 80%, at least about 85%, at least about 90%, at least about 95%, at least about 96%, at least about 97%, at least about 98%, or at least about 99%, or 100% identical to a sequence as set forth in any one of SEQ ID NO: 3, SEQ ID NO: 15, SEQ ID NO: 27, SEQ ID NO: 39, SEQ ID NO: 45, SEQ ID NO: 69, or SEQ ID NO: 81. In one embodiment, the disclosure provides an anti-TM4SF1 antibody, or an antigen-binding fragment thereof, comprising a light chain comprising a CDR1 domain as set forth in any one of SEQ ID NO: 12, SEQ ID NO: 24, SEQ ID NO: 36, SEQ ID NO: 48, SEQ ID NO: 60, SEQ ID NO: 72, or SEQ ID NO: 84, and having a light chain variable domain comprising an amino acid sequence that has at least at least about 60%, at least about 65%, at least about 70%, at least about 75%, at least about 80%, at least about 85%, at least about 90%, at least about 95%, at least about 96%, at least about 97%, at least about 98%, or at least about 99%, or 100% identical to a sequence a set forth in any one of SEQ ID NO: 9, SEQ ID NO: 21, SEQ ID NO: 33, SEQ ID NO: 45, SEQ ID NO: 57, SEQ ID NO: 69, or SEQ ID NO: 81. Thus, in certain embodiments, the CDR1 domain is held constant, while variability may be introduced into the remaining CDRs and/or framework regions of the heavy and/or light chains, while the antibody, or antigen binding fragment thereof, retains the ability to bind to TM4SF1 and retains the functional characteristics, e.g., binding affinity, of the parent.

In some embodiments, an anti-TM4SF1 antibody of this disclosure comprises a heavy chain comprising an Fc region, wherein said Fc region comprises a sequence selected from the group consisting of: SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 151, SEQ ID NO: 152, and SEQ ID NO: 153; or wherein said Fc region comprises a sequence comprising one or more substitutions in a sequence selected from the group consisting of: SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 151, SEQ ID NO: 152, and SEQ ID NO: 153. For instance, in some embodiments, an anti-TM4SF1 antibody of this disclosure comprises an Fc region, wherein said Fc region comprises a sequence that is at least about 70% to about 100%, such as at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to a sequence selected from the group consisting of: SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 151, SEQ ID NO: 152, and SEQ ID NO: 153.

In some embodiments, an anti-TM4SF1 antibody of this disclosure comprises a heavy chain comprising a sequence selected from the group consisting of: SEQ ID NO: 146, SEQ ID NO: 147, SEQ ID NO: 148, SEQ ID NO: 149, SEQ ID NO: 150, SEQ ID NO: 154, SEQ ID NO: 155, and SEQ ID NO: 156; or wherein said heavy chain comprises a sequence comprising one or more substitutions in a sequence selected from the group consisting of: SEQ ID NO: 146, SEQ ID NO: 147, SEQ ID NO: 148, SEQ ID NO: 149, SEQ ID NO: 150, SEQ ID NO: 154, SEQ ID NO: 155, and SEQ ID NO: 156. For instance, in some embodiments, an anti-TM4SF1 antibody of this disclosure comprises a heavy chain comprising a sequence that is at least about 70% to about 100%, such as at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to a sequence selected from the group consisting of: SEQ ID NO: 146, SEQ ID NO: 147, SEQ ID NO: 148, SEQ ID NO: 149, SEQ ID NO: 150, SEQ ID NO: 154, SEQ ID NO: 155, and SEQ ID NO: 156.

The anti-TM4SF1 antibodies and fragments described in Table 1 may also be humanized. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (See, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, VL6 subgroup I (VL6 I) and VH subgroup III (VHIII). In another method, human germline genes are used as the source of the framework regions.

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Human framework regions that may be used for humanization include but are not limited to: framework regions selected using the “best-fit” method (see, e.g., Sims, et al., J. Immunol. 151 (1993) 2296); framework regions derived from the consensus sequence of human antibodies of a particular subgroup of light or heavy chain variable regions (see, e.g., Carter, et al., Proc. Natl. Acad. Sci. USA, 89 (1992) 4285; and Presta, et al., J. Immunol., 151 (1993) 2623); human mature (somatically mutated) framework regions or human germline framework regions (see, e.g., Almagro, and Fransson, Front. Biosci. 13 (2008) 1619-1633); and framework regions derived from screening FR libraries (see, e.g., Baca, et al., J. Biol. Chem. 272 (1997) 10678-10684 and Rosok, et al., J. Biol. Chem. 271 (1996) 22611-22618).

Humanized antibodies and methods of making them are reviewed, e.g., in Almagro, and Fransson, Front. Biosci. 13 (2008) 1619-1633, and are further described, e.g., in Riechmann, et al., Nature 332 (1988) 323-329; Queen, et al., Proc. Nat'l Acad. Sci. USA 86 (1989) 10029-10033; U.S. Pat. Nos. 5,821,337, 7,527,791, 6,982,321, and 7,087,409; Kashmiri, et al., Methods 36 (2005) 25-34 (describing SDR (a-CDR) grafting); Padlan, Mol. Immunol. 28 (1991) 489-498 (describing “resurfacing”); Dall'Acqua, et al., Methods 36 (2005) 43-60 (describing “FR shuffling”); and Osbourn, et al., Methods 36 (2005)61-68 and Klimka, et al., Br. J. Cancer, 83 (2000) 252-260 (describing the “guided selection” approach to FR shuffling).

In one embodiment, an anti-TM4SF1 antibody, or antigen-binding fragment thereof, of the disclosure binds to cynomolgus TM4SF1 with a KD about 1×10⁻⁶ M or less.

An anti-TM4SF1 antibody, or antigen-binding fragment thereof, of the disclosure, in certain embodiments, binds to an epitope on the ECL2 loop of human TM4SF1 with a KD about 5×10⁻⁸ M or less as determined in a standard flow cytometry assay using HUVEC cells.

An anti-TM4SF1 antibody, or antigen-binding fragment thereof, of the disclosure, in certain embodiments, binds to human TM4SF1 with a KD of about 1×10⁻⁸ M or less in a standard flow cytometry assay using HUVEC cells.

An anti-TM4SF1 antibody, or antigen-binding fragment thereof, of the disclosure, in certain embodiments, binds to human TM4SF1 with a KD of about 1×10⁻³ M to about 1×10⁻⁴ M, about 1×10⁻⁴ M to about 1×10⁻⁵ M, about 1×10⁻⁵ M to about 1×10⁻⁶ M, about 1×10⁻⁶ to about 1×10⁻⁷ M, about 1×10⁻⁷ to about 1×10⁻⁷ M, about 1×10⁻⁷ M to about 1×10⁻⁹ M, about 1×10⁻⁹ M to about 1×10⁻¹⁰ M, about 1×10⁻¹⁰M to about 1×10⁻¹¹ M, about 1×10⁻¹¹ M to about 1×10⁻¹² M, about 2×10⁻³ M to about 2×10⁻⁴ M, about 2×10⁻⁴ M to about 2×10⁻⁵ M, about 2×10⁻⁵ M to about 2×10⁻⁶ M, about 2×10⁻⁶ to about 2×10⁻⁷ M, about 2×10⁻⁷ to about 2×10⁻⁸ M, about 2×10⁻⁸ M to about 2×10⁻⁹ M, about 2×10⁻⁹ M to about 2×10⁻¹⁰ M, about 2×10⁻¹⁰M to about 2×10⁻¹¹ M, about 2×10⁻¹¹ M to about 2×10⁻¹² M, about 3×10⁻³ M to about 3×10⁻⁴ M, about 3×10⁻⁴ M to about 3×10⁻⁵ M, about 3×10⁻⁵ M to about 3×10⁻⁶ M, about 3×10⁻⁶ to about 3×10⁻⁷ M, about 3×10⁻⁷ to about 3×10⁻⁸ M, about 3×10⁻⁸ M to about 3×10⁻⁹ M, about 3×10⁻⁹ M to about 3×10⁻¹⁰ M, about 3×10⁻¹⁰M to about 3×10⁻¹¹ M, about 3×10⁻¹¹ M to about 3×10⁻¹² M, about 4×10⁻³ M to about 4×10⁻⁴ M, about 4×10⁻⁴ M to about 4×10⁻⁵ M, about 4×10⁻⁵ M to about 4×10⁻⁶ M, about 4×10⁻⁶ to about 4×10⁻⁷ M, about 4×10⁻⁷ to about 4×10⁻⁸ M, about 4×10⁻⁸ M to about 4×10⁻⁹ M, about 4×10⁻⁹ M to about 4×10⁻¹⁰ M, about 4×10⁻¹⁰ M to about 4×10⁻¹¹ M, about 4×10⁻¹¹ M to about 4×10⁻¹² M, about 5×10⁻³ M to about 5×10⁻⁴ M, about 5×10⁻⁴ M to about 5×10⁻⁵ M, about 5×10⁻⁵ M to about 5×10⁻⁶ M, about 5×10⁻⁶ to about 5×10⁻⁷ M, about 5×10⁻⁷ to about 5×10⁻⁸ M, about 5×10⁻⁸ M to about 5×10⁻⁹ M, about 5×10⁻⁹ M to about 5×10⁻¹⁰ M, about 5×10⁻¹⁰ M to about 5×10⁻¹¹ M, about 5×10⁻¹¹ M to about 5×10⁻¹² M, about 5×10⁻⁷ M to about 5×10⁻¹¹ M, about 5×10⁻⁷ M, about 1×10⁻⁷ M, about 5×10⁻⁸ M, about 1×10⁻⁸ M, about 5×10⁻⁹ M, about 1×10⁻⁹ M, about 5×10⁻¹⁰ M, about 1×10⁻¹⁰ M, about 5×10⁻¹¹ M or about 1×10⁻¹¹ M. In some embodiments, the KD is determined in a standard flow cytometry assay using HUVEC cells.

An anti-TM4SF1 antibody, or antigen-binding fragment thereof, of the disclosure, in certain embodiments, binds to human TM4SF1 with a KD of about 5×10⁻¹⁰ M or less in a standard flow cytometry assay using HUVEC cells.

An anti-TM4SF1 antibody, or antigen-binding fragment thereof, of the disclosure, in certain embodiments, binds to cynomolgus TM4SF1 with a KD about 1×10⁻⁶ M or less in a standard flow cytometry assay using HEK293 overexpressing cells. In one embodiment, the HEK293 cells are transfected to express cynomolgus TM4SF1. In a further embodiment, HEK293 cells express cynomolgus TM4SF1 at about 600 mRNA copies per 10⁶ copies 18S rRNA.

Methods of determining the KD of an antibody or antibody fragment are known in the art. For example, surface plasmon resonance may be used to determine the KD of the antibody to the antigen (e.g., using a BIACORE 2000 or a BIACORE 3000 (BIAcore, Inc., Piscataway, N.J.) at 25° C. with immobilized antigen or Fc receptor CM5 chips at about 10 response units (RU)). In certain embodiments FACS or flow cytometry is used to determine the KD, whereby cells, such as HEK293 cells or HUVEC cells, that express TM4SF1 are used to bind the antibody or fragment and measure the KD according to standard methods. Affinity determination of antibodies using flow cytometry is described, for example, in Geuijen et al (2005) J Immunol Methods. 302(1-2):68-77. In certain embodiments, FACS is used to determine affinity of antibodies.

In one embodiment, the disclosure features an anti-TM4SF1 antibody or antigen binding fragment thereof, having CDR amino acid sequences described herein with conservative amino acid substitutions, such that the anti-TM4SF1 antibody or antigen binding fragment thereof comprises an amino acid sequence of a CDR that is at least 95% identical (or at least 96% identical, or at least 97% identical, or at least 98% identical, or at least 99% identical) to a CDR amino acid sequence set forth in Table 1. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

The disclosure further features in one aspect an anti-TM4SF1 antibody, or antigen-binding fragment thereof, that binds to an epitope on the ECL2 loop of human TM4SF1 with a KD of about 5×10⁻⁸ M or less as determined in a standard flow cytometry assay using HUVEC cells, wherein the anti-TM4SF1 antibody, or antigen-binding fragment thereof, comprises a light chain variable region comprising a human IgG framework region and comprises a heavy chain variable region comprising a human IgG framework region. In one embodiment, the anti-TM4SF1 antibody, or antigen-binding fragment thereof, is humanized. In one embodiment, the anti-TM4SF1 antibody, or antigen-binding fragment thereof, cross reacts with cynomolgus TM4SF1.

In another aspect of the disclosure, the anti-TM4SF1 antibody, or antigen-binding fragment thereof, is a humanized anti-TM4SF1 antibody, or antigen-binding fragment thereof, that binds to an epitope on the ECL2 loop of human TM4SF1 with a KD about 5×10⁻⁸ M or less as determined in a standard flow cytometry assay using HUVEC cells. In one embodiment, the anti-TM4SF1 antibody, or antigen-binding fragment thereof, binds to cynomolgus TM4SF1 with a KD about 1×10⁻⁶ M or less in a standard flow cytometry assay using HEK293 overexpressing cells. In one embodiment, the anti-TM4SF1 antibody, or antigen-binding fragment thereof, binds to human TM4SF1 with a KD of about 1×10⁻⁸ M or less in a standard flow cytometry assay using HUVEC cells. In one embodiment, the anti-TM4SF1 antibody, or antigen-binding fragment thereof, binds to human TM4SF1 with a KD of 1×10⁻³ M to about 1×10⁻⁴ M, about 1×10⁻⁴ M to about 1×10⁻⁵ M, about 1×10⁻⁵ M to about 1×10⁻⁶ M, about 1×10⁻⁶ to about 1×10⁻⁷ M, about 1×10⁻⁷ to about 1×10⁻⁸ M, about 1×10⁻⁸ M to about 1×10⁻⁹ M, about 1×10⁻⁹ M to about 1×10⁻¹⁰ M, about 1×10⁻¹⁰M to about 1×10⁻¹¹ M, about 1×10⁻¹¹ M to about 1×10⁻¹² M, about 2×10⁻³ M to about 2×10⁻⁴ M, about 2×10⁻⁴ M to about 2×10⁻⁵ M, about 2×10⁻⁵ M to about 2×10⁻⁶ M, about 2×10⁻⁶ to about 2×10⁻⁷ M, about 2×10⁻⁷ to about 2×10⁻⁸ M, about 2×10⁻⁸ M to about 2×10⁻⁹ M, about 2×10⁻⁹ M to about 2×10⁻¹⁰ M, about 2×10⁻¹⁰M to about 2×10⁻¹¹ M, about 2×10⁻¹¹ M to about 2×10⁻¹² M, about 3×10⁻³ M to about 3×10⁻⁴ M, about 3×10⁻⁴ M to about 3×10⁻⁵ M, about 3×10⁻⁵ M to about 3×10⁻⁶ M, about 3×10⁻⁶ to about 3×10⁻⁷ M, about 3×10⁻⁷ to about 3×10⁻⁸ M, about 3×10⁻⁸ M to about 3×10⁻⁹ M, about 3×10⁻⁹ M to about 3×10⁻¹⁰ M, about 3×10⁻¹⁰M to about 3×10⁻¹¹ M, about 3×10⁻¹¹ M to about 3×10⁻¹² M, about 4×10⁻³ M to about 4×10⁻⁴ M, about 4×10⁻⁴ M to about 4×10⁻⁵ M, about 4×10⁻⁵ M to about 4×10⁻⁶ M, about 4×10⁻⁶ to about 4×10⁻⁷ M, about 4×10⁻⁷ to about 4×10⁻⁸ M, about 4×10⁻⁸ M to about 4×10⁻⁹ M, about 4×10⁻⁹ M to about 4×10⁻¹⁰ M, about 4×10⁻¹⁰ M to about 4×10⁻¹¹ M, about 4×10⁻¹¹ M to about 4×10⁻¹² M, about 5×10⁻³ M to about 5×10⁻⁴ M, about 5×10⁻⁴ M to about 5×10⁻⁵ M, about 5×10⁻⁵ M to about 5×10⁻⁶ M, about 5×10⁻⁶ to about 5×10⁻⁷ M, about 5×10⁻⁷ to about 5×10⁻⁸ M, about 5×10⁻⁸ M to about 5×10⁻⁹ M, about 5×10⁻⁹ M to about 5×10⁻¹⁰ M, about 5×10⁻¹⁰M to about 5×10⁻¹¹ M, about 5×10⁻¹¹ M to about 5×10⁻¹² M, about 5×10⁻⁷ M to about 5×10⁻¹¹ M, about 5×10⁻⁷ M, about 1×10⁻⁷ M, about 5×10⁻⁸ M, about 1×10⁻⁸ M, about 5×10⁻⁹ M, about 1×10⁻⁹ M, about 5×10⁻¹⁰ M, about 1×10⁻¹⁰ M, about 5×10⁻¹¹ M or about 1×10⁻¹¹ M. In some embodiments, the KD is determined in a standard flow cytometry assay using HUVEC cells. In one embodiment, the anti-TM4SF1 antibody, or antigen-binding fragment thereof, binds to human TM4SF1 with a KD of about 5×10⁻¹⁰ M or less in a standard flow cytometry assay using TM4SF1 expressing HUVEC cells.

In one embodiment, binding of an anti-TM4SF1 antibody, or antigen binding fragment, of the disclosure to human TM4SF1 is not dependent on glycosylation of the ECL2 loop of human TM4SF1, i.e., binding of the antibody is independent of glycosylation of TM4SF1 within the ECL2 loop (SEQ ID NO: 77).

The anti-TM4SF1 antibodies, or antigen-binding fragments thereof, of the disclosure may be any of any isotype (for example, but not limited to IgG, IgM, and IgE). In certain embodiments, antibodies, or antigen-binding fragments thereof, of the disclosure are IgG isotypes. In a specific embodiment, antibodies, or antigen-binding fragments thereof, of the disclosure are of the IgG1, IgG2 or IgG4 isotype. In certain embodiments, the anti-TM4SF1 antibody, or antigen-binding fragment thereof, are human IgG1, human IgG2, or human IgG4 isotype.

IgG2 is naturally the lowest in ADCC and/or CDC activity (An et al., MAbs. 2009 November-December; 1(6): 572-579). Accordingly, in certain embodiments it IgG2 is advantageously used. However, IgG2 has two extra cysteines (leading to 4 inter-hinge disulfide bonds) which make it prone to aggregation via formation of inter-antibody disulfide bonds. In a related embodiment, mutations to the IgG2 cysteines are made to decrease aggregation.

The present disclosure provides antibody fragments that bind to TM4SF1. In certain circumstances there are advantages of using antibody fragments, rather than whole antibodies. The smaller size of the fragments allows for rapid clearance, and may lead to improved access to cells, tissues, or organs. For a review of certain antibody fragments, see Hudson et al., 2003, Nature Med. 9:129-34.

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. Fab, Fv, and scFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., 1992, Bio/Technology 10:163-67). According to another approach, F(ab′)2 fragments can be isolated directly from recombinant host cell culture. Fab and F(ab′)2 fragment with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

In certain embodiments, the antigen binding fragment is selected from the group consisting of a Fab, a Fab′, a F(ab′)2, an Fv, and an scFv.

Anti-TM4SF1 antibodies (and fragments) that, for example, have a high affinity for human TM4SF1, can be identified using screening techniques known in the art. For example, monoclonal antibodies may be made using the hybridoma method first described by Kohler et al., 1975, Nature 256:495-97, or may be made by recombinant DNA methods (see, e.g., U.S. Pat. No. 4,816,567).

In the hybridoma method, a mouse or other appropriate host animal, such as a hamster, is immunized using, for example, the ECL2 loop of human TM4SF1 or cells expressing TM4SF1 (whereby the ECL2 loop is expressed on the cell surface), to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice 59-103 (1986)).

The hybridoma cells thus prepared are seeded and grown in a suitable culture medium which, in certain embodiments, contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which prevent the growth of HGPRT-deficient cells.

Exemplary fusion partner myeloma cells are those that fuse efficiently, support stable high-level production of antibody by the selected antibody-producing cells, and are sensitive to a selective medium that selects against the unfused parental cells. Exemplary myeloma cell lines are murine myeloma lines, such as SP-2 and derivatives, for example, X63-Ag8-653 cells available from the American Type Culture Collection (Manassas, Va.), and those derived from MOPC-21 and MPC-11 mouse tumors available from the Salk Institute Cell Distribution Center (San Diego, Calif.). Human myeloma and mouse-human heteromyeloma cell lines also have been described for the production of human monoclonal antibodies (Kozbor, 1984, Immunol. 133:3001-05; and Brodeur et al., Monoclonal Antibody Production Techniques and Applications 51-63 (1987)).

Culture medium in which hybridoma cells are growing is assayed for production of monoclonal antibodies directed against the antigen. The binding specificity of monoclonal antibodies produced by hybridoma cells is determined by immunoprecipitation or by an in vitro binding assay, such as RIA or ELISA. The binding affinity of the monoclonal antibody can, for example, be determined by the Scatchard analysis described in Munson et al., 1980, Anal. Biochem. 107:220-39.

Once hybridoma cells that produce antibodies of the desired specificity, affinity, and/or activity are identified, the clones may be subcloned by limiting dilution procedures and grown by standard methods (Goding, supra). Suitable culture media for this purpose include, for example, DMEM or RPMI-1640 medium. In addition, the hybridoma cells may be grown in vivo as ascites tumors in an animal, for example, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells can serve as a source of such DNA. Once isolated, the DNA may be placed into expression vectors, which are then transfected into host cells, such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., 1993, Curr. Opinion in Immunol. 5:256-62 and Pluckthun, 1992, Immunol. Revs. 130:151-88.

In a further embodiment, monoclonal antibodies or antibody fragments can be isolated from antibody phage libraries generated using the techniques described in, for example, Antibody Phage Display: Methods and Protocols (O'Brien and Aitken eds., 2002). In principle, synthetic antibody clones are selected by screening phage libraries containing phages that display various fragments of antibody variable region (Fv) fused to phage coat protein. Such phage libraries are screened against the desired antigen. Clones expressing Fv fragments capable of binding to the desired antigen are adsorbed to the antigen and thus separated from the non-binding clones in the library. The binding clones are then eluted from the antigen and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., 1994, Ann. Rev. Immunol. 12:433-55.

Repertoires of VH and VL genes can be separately cloned by PCR and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self-antigens without any immunization as described by Griffiths et al., 1993, EMBO J 12:725-34. Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, 1992, J. Mol. Biol. 227:381-88.

Screening of the libraries can be accomplished by various techniques known in the art. For example, TM4SF1 (e.g., a soluble form of the ECL2 loop or cells expressing said loop) can be used to coat the wells of adsorption plates, expressed on host cells affixed to adsorption plates or used in cell sorting, conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., 1990, Proteins 8:309-14 and WO 92/09690, and by use of a low coating density of antigen as described in Marks et al., 1992, Biotechnol. 10:779-83.

Anti-TM4SF1 antibodies can be obtained by designing a suitable antigen screening procedure to select for the phage clone of interest followed by construction of a full length anti-TM4SF1 antibody clone using VH and/or VL sequences (e.g., the Fv sequences), or various CDR sequences from VH and VL sequences, from the phage clone of interest and suitable constant region (e.g., Fc) sequences described in Kabat et al., supra.

Screening of anti-TM4SF1 antibodies can be performed using binding assays known in the art and described herein for determining whether the antibody has a therapeutic affinity for the ECL2 loop of TM4SF1. The ability of the antibody to inhibit or decrease metastatic cell activity can be measured using standard assays in the art, as well as those described herein. Preclinical assays require use of an animal model of metastasis, commonly of one of three types: (i) injection of metastatic mouse tumor cells such as B16F10 melanoma TCs into mice, commonly via tail vein injection to generate lung metastases, via portal vein or intrasplenic injection to generate liver metastases, or via left ventricular cardiac injection to generate bone and other metastases; (ii) orthotopic transplantation of metastatic tumor cells or intact tumor fragments into mice, which methods often require later surgical resection of the primary tumor to prevent morbidity associated with primary tumor growth; and (iii) genetically engineered mouse models of spontaneous metastasis, of which the most common is the MMTV-Pyt (mouse mammary tumor virus-polyomavirus middle T Antigen) mouse mammary carcinoma model which provides a highly realistic mouse model of human cancer metastasis; greater than 85% of hemizygous MMTV-PyMT females spontaneously develop palpable mammary tumors which metastasize to the lung at age to 8-16 weeks. Quantifying the metastatic burden in the lung, either by live animal imaging or direct counting of metastatic nodules in the lungs of sacrificed animals, as a function of the degree of TM4SF1 immunoblockade and achieving a therapeutic level, e.g., at least a 50% reduction in lung metastasis, would be indicative, for example, of a therapeutic antibody that could be used in the methods of the disclosure. Further, cross-species reactivity assays are known in the art. Examples of assays that can be used are described, for example, in Khanna and Hunter (Carcinogenesis. 2005 March; 26(3):513-23) and Saxena and Christofori (Mol Oncol. 2013 April; 7(2):283-96), incorporated by reference in their entireties herein.

In some embodiments, the anti-TM4SF1 antibodies and antigen binding fragments thereof can be used, e.g., to treat or prevent cancer. In certain embodiments, the anti-TM4SF1 antibodies and antigen binding fragments of the disclosure can be used to prevent tumor cells from metastasizing. The anti-TM4SF1 antibodies and antigen binding fragments thereof, of this disclosure, in some examples, prevent tumor cell metastasis by interfering with the interaction between tumor cells and blood vessel endothelial cells.

One embodiment of the disclosure provides combinations comprising an anti-angiogenic antibody, such as an anti-TM4SF1 antibody or an antigen binding fragment thereof and an immunotherapeutic agent, wherein the anti-TM4SF1 antibody or antigen binding fragment thereof comprises a modified Fc region, such as a modified IgG region (e.g., IgG1, IgG2, IgG3, IgG4) comprising one or more mutations. In some cases, said one or more mutations in the Fc region leads to improvements in a drug comprising such a modified Fc region, in areas of improvement such as: 1) reduction of effector functions, 2) half-life modulation, 3) stability, and 4) downstream processes. In some cases, the modified Fc region can comprise one or more mutations that will reduce or ablate interactions between the antibodies and the immune system. Key interactions may include interactions of the antibody Fc with Fcγ receptors on white blood cells and platelets, and with C1q of the complement system leading to complement dependent cytotoxicity.

The present disclosure provides, in some cases, a combination comprising an anti-TM4SF1 antibody or an antigen binding fragment thereof that includes immune ablating mutations, for example, in the Fc region which in such cases is a modified Fc region, for example, a modified IgG Fc region. In some embodiments, the modified Fc region comprises a modification at position N297. In some embodiments, the modified Fc region comprises a modified IgG Fc region (e.g., a modified IgG1, IgG2, IgG3, or IgG4 Fc region) comprising one or more mutations at positions E233, L234 or F234, L235, G237, P238, F243, T250, M252, S254, T256, E258, D259, V264, D265, K288, N297, T299, T307, V308, Q311, K322, L328, P329, A330, P331, T356, K370, A378, R409, V427, M428, H433, N434, and H435, or any combinations thereof. In some embodiments, the Fc region comprises an extension of residues at its C-terminus, such that positive charge is maintained at the C-terminus (e.g., in some cases, if the anti-TM4SF1 antibody or antigen binding fragment comprises two heavy chains then at least one heavy chain comprises an extension of residues at the C-terminus). Such extension of residues can comprise addition of one or more amino acids, such as, arginine, lysine, proline, or any combinations thereof. In some examples, the extended C-terminus of the Fc regions leads to reduced CDC function of the anti-TM4SF1 antibody or antigen binding fragment thereof, comprising the anti-TM4SF1 antibody or antigen binding fragment thereof. Such an effect is seen, in some cases, by addition of KP residues after K447 of Fc in IgG1 or IgG4, alone or in combination with other mutations (e.g., K322A, P331G-IgG1).

In some embodiments, a combination is provided comprising an anti-TM4SF1 antibody or an antigen binding fragment thereof can comprise an antibody with reduced effector function, including substitution of one or more of Fc region residues 238, 265, 269, 270, 297, 327 and 329 (See, e.g., U.S. Pat. No. 6,737,056). In some cases, such mutations in the Fc region may comprise substitutions at two or more of amino acid positions 265, 269, 270, 297 and 327, for example, substitution of residues 265 and 297 to alanine (DANA mutations, i.e., D265A and N297A) (See, e.g., U.S. Pat. No. 7,332,581). In some cases, mutations in the Fc region may comprises substitutions at one or more amino acid positions E233, L234, L235, G237, D265, N297, K322, and P331. In some cases, mutations in the Fc region may comprises at least one of E233P, L234A, L235A, G237A, D265A, N297A, K322A, and P331G, or any combinations thereof. For instance, the mutations in the Fc region can comprise L234A/L235A/G237A (IgG1), or F234A/L235E (IgG4), and an anti-TM4SF1 antibody or antigen binding fragment comprising such mutations may exhibit altered FcgRI interactions.

In some embodiments, a combination is provided comprising an anti-TM4SF1 antibody or antigen binding fragment thereof may include an Fc variant comprising the following mutations: an amino acid substitution at position M428 and N434 (M428L, N434S) (See, e.g., U.S. Pat. No. 9,803,023). In some embodiments, an anti-TM4SF1 antibody or antigen binding fragment thereof may include an Fc variant comprising the following mutations: an amino acid substitution at position T250 and M428 (T250Q, M428L) (See, e.g., U.S. Pat. No. 9,803,023).

In some embodiments, a combination is provided comprising an TM4SF1 antibody or antigen binding fragment thereof comprising mutations D265A and N297A. In some cases, the proline at position 329 (P329) of a wild-type human Fc region may be substituted with glycine or arginine or an amino acid residue large enough to destroy the proline sandwich within the Fc/Fcγ receptor interface, that is formed between the P329 of the Fc and tryptophan residues W87 and W1 of FcgRIII (See, e.g., Sondermann et al., Nature 406, 267-273 (20 Jul. 2000)). In a further embodiment, the mutations in the Fc region may comprise one or more amino acid substitutions such as S228P (IgG4), E233P, L234A, L235A, L235E, N297A, N297D, or P331S and in still in other embodiments: L234A and L235A of the human IgG1 Fc region or S228P and F234A, L235A, or L235E of the human IgG4 Fc region.

In some embodiments, a combination is provided comprising an anti-TM4SF1 antibody or antigen binding fragment thereof including a modified Fc region which is an Fc variant of a wild-type human IgG Fc region wherein P329 of the human IgG Fc region substituted with glycine and wherein the Fc variant comprises at least two further amino acid substitutions at L234A and L235A of the human IgG1 Fc region or S228P and L235E of the human IgG4 Fc region, and wherein the residues are numbered according to the EU numbering (See, e.g., U.S. Pat. No. 8,969,526). The polypeptide comprising the P329G, L234A and L235A substitutions may exhibit a reduced affinity to the human FcγRIIIA and FcγRIIA, for down-modulation of ADCC to at least 20% of the ADCC induced by the polypeptide comprising the wildtype human IgG Fc region, and/or for down-modulation of ADCP (See, e.g., U.S. Pat. No. 8,969,526).

In some embodiments, a combination is provided comprising an anti-TM4SF1 antibody or antigen binding fragment thereof that includes an Fc variant comprising triple mutations: an amino acid substitution at position P329, a L234A and a L235A mutation (P329/LALA) (See, e.g., U.S. Pat. No. 8,969,526).

Certain anti-TM4SF1 antibodies or antigen binding fragments of this disclosure, in some embodiments of the combination provided herein, can comprise mutations that exhibit improved or diminished binding to FcRs. (See, e.g., U.S. Pat. No. 6,737,056; WO 2004/056312, and Shields et al., J. Biol. Chem. 9(2): 6591-6604 (2001).)

In some instances, a combination is provided comprising an anti-TM4SF1 antibody or antigen binding fragment may include an Fc region with one or more amino acid substitutions which improve ADCC, e.g., substitutions at positions 298, 333, and/or 334 of the Fc region. Alterations may be made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. (2000) J. Immunol. 164: 4178-4184.

Antibodies with increased half-lives and improved binding to the neonatal Fc receptor (FcRn) are also provided herein in some embodiments of the combination. FcRn, named after its function for the transfer of maternal IgGs to the fetus, also serves to prevent antibodies from being degraded in lysosomes, by capturing them in endosomes and returning them to circulation. (See, e.g., Guyer et al., J. Immunol. 117:587 (1976) and Kim et al., J. Immunol. 24:249 (1994)), are described in US2005/0014934. Without being bound by any particular theory, it is contemplated that antibodies with improved binding to FcRn detach from TM4SF1 and bind to FcRn, which then recycles the antibody back to circulation, thus reducing vascular toxicity. In some embodiments herein are provided a combination comprising anti-TM4SF1 antibodies or antigen binding fragments that comprise an Fc region with one or more substitutions that enhance FcRn recycling. In some embodiments herein are provided a combination comprising anti-TM4SF1 antibodies or antigen binding fragments thereof that comprise an Fc region with one or more substitutions therein which improve binding of the Fc region to FcRn, such as, substitutions at one or more of positions: 238, 250, 252, 254, 256, 265, 272, 286, 303, 305, 307, 311, 312, 317, 340, 356, 360, 362, 376, 378, 380, 382, 413, 428, 424, 434, and 435, e.g., substitution of Fc region residue 434 (U.S. Pat. No. 7,371,826) according to EU numbering. See also Duncan &amp; Winter, Nature 322:738-40 (1988); U.S. Pat. Nos. 5,648,260; 5,624,821; US2005/0014934 and WO 94/29351 concerning other examples of Fc region variants, the entirety of which are incorporated herein by reference.

In some embodiments, provided herein are combinations comprising anti-TM4SF1 antibodies or antigen binding fragments thereof that have pH dependent FcRn binding affinities. Without being bound by any particular theory, it is contemplated that antibodies or antigen binding fragments thereof with pH dependent FcRn binding affinity detach from FcRn at pH >7, and bind to FcRn at pH 6. Accordingly, FcRn in acidic pH subcellular organelles, e.g., endosomes, binds such antibodies and carries the antibodies back to the cell membrane, and release the antibodies into plasma at pH >7, recycling the antibody.

In certain embodiments, herein are provided combinations comprising anti-TM4SF1 antibodies or antigen binding fragments thereof that comprise an Fc region with one or more substitutions therein which modulate FcRn recycling. In some embodiments herein are provided combinations comprising anti-TM4SF1 antibodies or antigen binding fragments thereof that comprise one or more substitutions that enhance FcRn binding at acidic pH, e.g., pH 6, and does not affect FcRn binding at neutral or basic pH, e.g. pH 7. In some embodiments, a combination as provided herein comprises an anti-TM4SF1 antibody or antigen binding fragment thereof comprising substitutions at one or more of positions 250, 252, 254, 256, 428, and 434 according to EU numbering. In some embodiments, a combination as provided herein comprises an anti-TM4SF1 antibody or antigen binding fragment thereof comprising an Fc variant comprising one or more of substitutions T250Q, M252Y, S254T, T256E, M428L, and N434S. In some embodiments, a combination as provided herein comprises an anti-TM4SF1 antibody or antigen binding fragment thereof comprising an IgG1 Fc variant comprising substitutions T250Q and M428L (the “QL mutant”). In some embodiments, a combination as provided herein comprises an anti-TM4SF1 antibody or antigen binding fragment thereof may include an IgG4 Fc variant comprising substitutions T250Q and M428L (the “QL mutant”). In some embodiments, a combination as provided herein comprises an anti-TM4SF1 antibody or antigen binding fragment thereof comprising an IgG1 Fc variant comprising substitutions M252Y, S254T, and T256E (the “YTE mutant”). In some embodiments, a combination provided herein comprises an anti-TM4SF1 antibody or antigen binding fragment thereof comprising an IgG1 Fc variant comprising substitutions M428L and N434S (the “LS mutant”). In some embodiments, a combination as provided herein comprises an anti-TM4SF1 antibody or antigen binding fragment thereof comprising an IgG4 Fc variant comprising substitutions M428L and N434S (the “LS mutant”). Effects of amino acid substitutions in the Fc region that modulate FcRn recycling are described in, e.g., Hamblett et al., Mol. Pharm. 13(7): 2387-96 (2016); Dall'Acqua et al., J. Biol. Chem. 281(33): 23514-24 (2006), Hinton et al., J. Biol. Chem. 279(8): 6213-6 (2003), Hinton et al., J. Immunol., 176(1): 346-56 (2006), US20080181887, U.S. Pat. No. 7,361,740, and EP2235059, the entirety of which are incorporated herein by reference.

In certain embodiments, a combination as provided herein comprises an anti-TM4SF1 antibody, or antigen-binding fragment thereof, which is an IgG1 isotype and comprises an Fc region comprising one or more substitutions selected from the group consisting of T250Q, M252Y, S254T, T256E, M428L, and N434S. In some embodiments, a combination as provided herein comprises an anti-TM4SF1 antibody, or antigen binding fragment thereof, which is an IgG4 isotype and comprises an Fc region comprising one or more substitutions selected from the group consisting of T250Q, M252Y, S254T, T256E, M428L, and N434S. In some embodiments, a combination as provided herein comprises an anti-TM4SF1 antibody or antigen binding fragment thereof which is an IgG1 isotype and comprises an Fc region comprising substitutions T250Q and M428L. In some embodiments, a combination as provided herein comprises an anti-TM4SF1 antibody or antigen binding fragment thereof which is an IgG1 isotype and comprises an Fc variant comprising substitutions M252Y, S254T, and T256E. In some embodiments, a combination as provided herein comprises an anti-TM4SF1 antibody or antigen binding fragment thereof which is an IgG4 isotype and comprises an Fc variant comprising substitutions M252Y, S254T, and T256E. In some embodiments, a combination as provided herein comprises an anti-TM4SF1 antibody or antigen binding fragment thereof which is an IgG1 isotype and comprises an Fc variant comprising substitutions M428L and N434S. In some embodiments, a combination as provided herein comprises an anti-TM4SF1 antibody or antigen binding fragment thereof which is an IgG4 isotype and comprises an Fc variant comprising substitutions M428L and N434S.

In some embodiments, the combinations disclosed herein comprise anti-TM4SF1 antibodies or antigen binding fragments thereof comprising mutated Fc regions that have increased FcRn binding affinity and increased serum half-life. In certain embodiments, combinations as provided herein comprise anti-TM4SF1 antibodies or antigen binding fragment thereof comprising mutated Fc regions that have serum half-life of at least 10 days, at least 15 days, at least 20 days, at least 25 days, at least 30 days, at least 35 days, at least 40 days, at least 50 days, at least 60 days, at least 70 days, at least 80 days, at least 90 days, at least 100 days or more.

In certain embodiments, the combinations comprise anti-TM4SF1 antibodies or antigen binding fragment thereof that exhibit reduced vascular toxicity, improved therapeutic margin, or both. In certain embodiments the combinations of this disclosure comprise anti-TM4SF1 antibodies or antigen binding fragments thereof comprising mutated Fc regions that have reduced or ablated affinity for an Fc ligand responsible for facilitating effector function compared to an antibody having the same amino acid sequence as the antibody of the disclosure but not comprising the addition, substitution, or deletion of at least one amino acid residue to the Fc region (also referred to herein as an “unmodified antibody”).

In one embodiment, a combination provided herein comprises an anti-TM4SF1 antibody, or antigen-binding fragment thereof comprising an Fc region comprising at least two mutations that reduce or ablate ADCC and/or CDC effector function of the antibody, or antigen-binding fragment thereof. In further embodiments, a combination provided herein comprises the anti-TM4SF1 antibody, or antigen-binding fragment thereof, comprises an Fc region comprising at least three, at least four, at least five, at least six, at least seven, at least eight, at least nine, at least ten or more mutations that reduce or ablate ADCC and/or CDC effector function of the antibody, or antigen-binding fragment thereof.

In certain embodiments, a combination provided herein comprises an anti-TM4SF1 antibody, or antigen-binding fragment thereof, which is an IgG1 isotype and comprises an Fc region comprising one or more mutations selected from the group consisting of E233P, L234V, L234A, L235A, G236Delta (deletion), G237A, V263L, N297A, N297D, N297G, N297Q, K322A, A327G, P329A, P329G, P329R, A330S, P331A, P331G, and P331S.

In one embodiment, a combination provided herein comprises an anti-TM4SF1 antibody, or antigen-binding fragment thereof, which is an IgG1 isotype and comprises an Fc region comprising an L234A/L235A mutation, with or without a G237A mutation. In one embodiment, the anti-TM4SF1 antibody, or antigen-binding fragment thereof, is an IgG1 isotype and comprises an Fc region comprising L234A, L235A, and G237A mutations.

In one embodiment, a combination provided herein comprises an anti-TM4SF1 antibody, or antigen-binding fragment thereof, which is an IgG1 isotype and comprises an Fc region comprising an A327G/A330S/P331S mutation.

In one embodiment, a combination provided herein comprises an anti-TM4SF1 antibody, or antigen-binding fragment thereof, which is an IgG1 isotype and comprises an Fc region comprising an E233P/L234V/L235A/delta G236 (deletion) mutation, which provides reduced binding to FcγRI (also referred to herein as FcgRI), FcγRIIA (also referred to herein as FcgRIIA), FcγRIIIA (also referred to herein as FcgRIIIAI) and reduced ADCC and CDC effector function, as described, for example, in An Z et al. Mabs 2009 November-Ec; 1(6):572-9, incorporated by reference in its entirety herein.

In one embodiment, a combination provided herein comprises an anti-TM4SF1 antibody, or antigen-binding fragment thereof, which is an IgG1 isotype and comprises an Fc region comprising an N297x mutation, where x=A, D, G, Q.

In one embodiment, a combination provided herein comprises an anti-TM4SF1 antibody, or antigen-binding fragment thereof, which is an IgG1 isotype and comprises an Fc region comprising an A327G/A330S/P331S mutation.

In one embodiment, a combination provided herein comprises an anti-TM4SF1 antibody, or antigen-binding fragment thereof, which is an IgG1 isotype and comprises an Fc region comprising a mutation in one or more of K322A, P329A, and P331A, which provides reduced binding to C1q, as described, for example, in Canfield &Morrison. J Exp Med (1991) 173(6):1483-91.10.1084, incorporated by reference in its entirety herein.

In one embodiment, a combination provided herein comprises an anti-TM4SF1 antibody, or antigen-binding fragment thereof, is an IgG1 isotype and comprises an Fc region comprising a V263L mutation, which provides enhanced binding to FcγRIIB (also referred to herein as FcgRIIB) and enhanced ADCC, as described in, for example, Hezareh et al. J Virol. 2001 December;75(24):12161-8, incorporated by reference in its entirety herein.

In other embodiments, a combination provided herein comprises an anti-TM4SF1 antibody or antigen-binding fragment thereof, which is an IgG1 isotype and comprises an Fc region comprising a L234A/L235A, G237A or L235E mutation.

In other embodiments, a combination provided herein comprises an anti-TM4SF1 antibody, or antigen-binding fragment thereof, which is an IgG1 isotype and comprises an Fc region comprising a L234F, L235E or P331S mutation.

In certain embodiments, a combination provided herein comprises an anti-TM4SF1 antibody, or antigen-binding fragment thereof, which is an IgG2 isotype and comprises an Fc region comprising one or more mutations selected from the group consisting of V234A, G237A, P238S, H268A or H268Q, V309L, A330S and P331S.

In one embodiment, a combination provided herein comprises an anti-TM4SF1 antibody, or antigen-binding fragment thereof, which is an IgG2 isotype and comprises an Fc region comprising an A330S/P331S mutation.

In one embodiment, a combination provided herein comprises an anti-TM4SF1 antibody, or antigen-binding fragment thereof, which is an IgG2 isotype and comprises an Fc region comprising an A330S/P331S, V234A/G237A/P238S/H268A/V309L/A330S/P331S or H268Q/V309L/A330S/P331S mutation.

In other embodiments, a combination provided herein comprises an anti-TM4SF1 antibody, or antigen-binding fragment thereof, which is an IgG4 isotype and comprises an Fc region comprising a one or more mutations selected from the group consisting of S228P, E233P, F234A, F234V, L235E, L235A, G236Delta (deletion), N297A, N297D, N297G, N297Q, P329G, P329R.

In certain embodiments, a combination provided herein comprises an anti-TM4SF1 antibody, or antigen-binding fragment thereof, which is an IgG4 isotype and comprises an Fc region comprising an S228P mutation, which provides reduced Fab-arm exchange and reduced aggregation, as described for example in Chappel et al. Proc Natl Acad Sci USA (1991) 88(20):9036-40, incorporated by reference in its entirety herein.

In one embodiment, a combination provided herein comprises an anti-TM4SF1 antibody, or antigen-binding fragment thereof, which is an IgG4 isotype and comprises an Fc region comprising an S228P/L235E mutation.

In one embodiment, a combination provided herein comprises an anti-TM4SF1 antibody, or antigen-binding fragment thereof, which is an IgG4 isotype and comprises an Fc region comprising an S228P/E233P/F234V/L235A/delta G236 (deletion) mutation.

In one embodiment, a combination provided herein comprises an anti-TM4SF1 antibody, or antigen-binding fragment thereof, which is an IgG4 isotype and comprises an Fc region comprising an N297x mutation, where x=A, D, G, Q.

In one embodiment, a combination provided herein comprises an anti-TM4SF1 antibody, or antigen-binding fragment thereof, which is an IgG4 isotype and comprises an Fc region comprising an S228P/F234A/L235A mutation.

In one embodiment, a combination provided herein comprises an anti-TM4SF1 antibody, or antigen-binding fragment thereof, which is an IgG4 isotype and comprises an Fc region comprising a L235E mutation, which provides reduced binding to FcγRI, FcγRIIA, FcγRIIIA and reduced ADCC and CDC effector activity, as described in, for example, Saxena et al. Front Immunol. 2016 Dec. 12; 7:580.

In other embodiments, a combination provided herein comprises an anti-TM4SF1 antibody, or antigen-binding fragment thereof, which is an IgG4 isotype and comprises an Fc region comprising a S228P/F234A/L235A or E233P/L235A/G236D mutation.

In one embodiment, a combination provided herein comprises an anti-TM4SF1 antibody, or antigen-binding fragment thereof, which is an IgG4 isotype and comprises an Fc region comprising at least a S228P mutation. See, e.g., Angal et al. (Mol Immunol. 1993 January; 30(1):105-8) describe an analysis of the hinge sequences of human IgG4 heavy chains to determine that the presence of serine at residue 241 (according to EU numbering system, and now corresponding to residue 228 in Kabat numbering,) as the cause of heterogeneity of the inter-heavy chain disulfide bridges in the hinge region in a proportion of secreted human IgG4.

See Silva et al. (J Biol Chem. 2015 Feb. 27; 290(9):5462-9) describe the S228P mutation in human IgG4 that prevents in vivo and in vitro IgG4 Fab-arm exchange.

In other embodiments, a combination provided herein comprises an anti-TM4SF1 antibody, or antigen-binding fragment thereof, which is an IgG4 isotype and comprises an Fc region comprising a L235E or S228P mutation.

In other embodiments, a combination provided herein comprises an anti-TM4SF1 antibody, or antigen-binding fragment thereof, which is an IgG4 or IgG1 isotype and comprises an Fc region comprising a N297A, N297D or N297G mutation.

In other embodiments, a combination provided herein comprises an anti-TM4SF1 antibody, or antigen-binding fragment thereof, which is an IgG4 or IgG1 isotype and comprises an Fc region comprising a P329G, P329R mutation.

In one exemplary embodiment, the mutated Fc region of any IgG isotype comprises one or more mutations at positions 234, 235, 236, 237, 297, 318, 320, 322 (as described in WO1988007089, incorporated by reference in its entirety herein). Other possible mutations in the Fc region, including substitutions, deletions and additions are also described in, for example, US20140170140, WO2009100309, US20090136494 and U.S. Pat. No. 8,969,526, incorporated by reference in their entireties herein.

In vitro and/or in vivo cytotoxicity assays can be conducted to confirm the reduction or ablation of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, RII and RIII. Non-limiting examples of in vitro assays to assess ADCC activity of a molecule of interest is described in U.S. Pat. No. 5,500,362 (see, e.g. Hellstrom, I., et al., Proc. Nat'l Acad. Sci. USA 83 (1986) 7059-7063) and Hellstrom, I., et al., Proc. Nat'l Acad. Sci. USA 82 (1985) 1499-1502; U.S. Pat. No. 5,821,337 (see Bruggemann, M., et al., J. Exp. Med. 166 (1987) 1351-1361). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes, et al., Proc. Nat'l Acad. Sci. USA 95 (1998) 652-656. C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro, et al., J. Immunol. Methods 202 (1996) 163; Cragg, M. S., et al., Blood 101 (2003) 1045-1052; and Cragg, M. S., and Glennie, M. J., Blood 103 (2004) 2738-2743). FcRn binding and in vivo clearance/half-life determinations can also be performed using methods known in the art (see, e.g., Petkova, S. B., et al., Int'l. Immunol. 18(12) (2006) 1759-1769).

In some embodiments, the mutated Fc region of any IgG isotype comprises a mutation at position L328, such as L328M, L328D, L328E, L328N, L328Q, L328F, L3281, L328V, L328T, L328H, L328A (see e.g., US20050054832)

In one embodiment, antibodies, or antigen-binding fragments thereof, of the combination provided in this disclosure exhibit reduced or ablated ADCC effector function as compared to unmodified antibodies. In another embodiment, antibodies, or antigen-binding fragments thereof, of the combination provided in this disclosure exhibit reduced ADCC effector function that is at least 2 fold, or at least 3 fold, or at least 5 fold or at least 10 fold or at least 50 fold or at least 100 fold less than that of an unmodified antibody. In still another embodiment, antibodies or antigen binding fragments thereof, of the combination provided in this disclosure exhibit ADCC effector function that is reduced by at least 10%, or at least 20%, or by at least 30%, or by at least 40%, or by at least 50%, or by at least 60%, or by at least 70%, or by at least 80%, or by at least 90%, or by at least 100%, relative to an unmodified antibody. In a further aspect of the combination provided in this disclosure, the reduction or down-modulation of ADCC effector function induced by the antibodies, or antigen-binding fragments thereof, of the present disclosure, is a reduction to 0, 2.5, 5, 10, 20, 50 or 75% of the value observed for induction of ADCC by unmodified antibodies. In certain embodiments, the reduction and/or ablation of ADCC activity may be attributed to the reduced affinity of the antibodies, or antigen-binding fragments thereof, of the disclosure for Fc ligands and/or receptors.

One embodiment of the disclosure provides a combination comprising an anti-TM4SF1 antibody or an antigen binding fragment thereof and an immunotherapeutic agent, wherein the anti-TM4SF1 antibody or antigen binding fragment thereof exhibit pH dependent binding affinity to TM4SF1. In some instances, an anti-TM4SF1 antibody or antigen binding fragment thereof binds to TM4SF1 with higher affinity at certain pH range as compared to other pH ranges. For example, an anti-TM4SF1 antibody or antigen binding fragment thereof may bind to TM4SF1 with different affinity at an acidic pH than at a neutral pH or a basic pH. In some embodiments, an anti-TM4SF1 antibody or antigen binding fragment thereof binds to TM4SF1 with higher affinity at an acidic pH than at a neutral or basic pH. In some embodiments, an anti-TM4SF1 antibody or antigen binding fragment thereof binds to TM4SF1 with lower affinity at an acidic pH than at a neutral or basic pH. In some embodiments, an anti-TM4SF1 antibody or antigen binding fragment thereof binds to TM4SF1 at acidic pH and dissociates from TM4SF1 at neutral or basic pH. In some embodiments, an anti-TM4SF1 antibody or antigen binding fragment thereof binds to TM4SF1 at pH7 or higher and detaches from TM4SF1 at pH6 or lower. In subcellular compartments such as plasma, cytosol, and nucleus, the pH is neutral or basic. In lysosomes or endosomes, the pH is acidic. Without being bound by any theory, an anti-TM4SF1 antibody or antigen binding fragment thereof bind to the antigen and subsequently internalized in the membrane of an endosome. A pH-dependent anti-TM4SF1 antibody or antigen binding fragment thereof can detach from TM4SF1 in an endosome and bind to FcRn receptors within the endosome, and can be recycled by the FcRn receptor back into circulation rather than degraded in a lysosome that the endosome progresses to. Accordingly, a pH dependent anti-TM4SF1 antibody or antigen binding fragment thereof can bind to TM4SF1 antigen multiple times. Accordingly, a pH dependent anti-TM4SF1 antibody and the associated therapeutic molecule or payload therewith can be recycled by FcRn receptors, without releasing the payload in the lysosome.

Also disclosed herein are methods of making an anti-TM4SF1 antibody or antigen binding fragment thereof, for a combination provided herein, that has increased half-life and/or pharmacodynamic effect by regulating antibody-TM4SF1 binding affinity in a pH dependent manner, comprising selecting for antibody CDR histidine residues or other residues that optimize the microenvironment affecting pKa of the antibody, such that the antibody-TM4SF1 binding has a Kd ratio and/or Koff ratio at pH6.0/pH7.4 that is at least 2, 3, 4, 8, 10, 16, or more, or ranges between 2, 3, 4, 8, 10, 16, or more. In some embodiments, the method comprises introducing amino acid substitutions into an anti-TM4SF1 antibody or antigen binding fragment thereof to achieve TM4SF1 affinity with a KD at pH 7.4 of at least 100 nM as measured at 25° C. In certain embodiments, said method comprises generating an antibody library enriched for histidines in CDR residues or other residues that optimize the microenvironment affecting pKa.

In some embodiments, the antibody library comprises anti-TM4SF1 antibodies or antigen binding fragments thereof with histidine residues introduced into a CDR position. In some embodiments, the antibody library comprises a series of anti-TM4SF1 antibodies or antigen binding fragments thereof, wherein each anti-TM4SF1 antibody in the antibody library comprises a single histidine substitution at a different CDR position. In some embodiments, the antibody library comprises a series of anti-TM4SF1 antibodies or antigen binding fragments thereof, each comprising 1, 2, 3, 4, 5, 6, 7, 8, 9, 10, 11, 12, 13, 14, 15, 16, or 17 mutations to histidine residues. In some embodiments, every CDR position is mutated to histidine in at least one of the TM4SF1 antibodies or antigen fragments of the antibody library.

In some embodiments, a combination provided herein comprises an anti-TM4SF1 antibody or antigen binding fragment thereof comprising 1, 2, 3, 4, 5, or more histidine substitutions in a CDR region. A histidine residue can be engineered into different positions of an anti-TM4SF1 antibody light chain (LC) or heavy chain (HC) for pH dependent binding affinity. Accordingly, in some embodiments, provided herein are combinations with histidine engineered anti-TM4SF1 antibody or antigen binding fragment thereof. In some embodiments, an anti-TM4SF1 antibody or antigen binding fragment thereof comprises one or more histidine residues in CDR1, CDR2, and/or CDR3 of the light chain variable region (VL). In some embodiments, an anti-TM4SF1 antibody or antigen binding fragment thereof comprises one or more histidine residues in CDR1 of the light chain variable region (VL). In some embodiments, an anti-TM4SF1 antibody or antigen binding fragment thereof comprises one or more histidine residues in CDR2 of the light chain variable region (VL). In some embodiments, an anti-TM4SF1 antibody or antigen binding fragment thereof comprises one or more histidine residues in CDR3 of the light chain variable region (VL). In some embodiments, an anti-TM4SF1 antibody or antigen binding fragment thereof comprises one or more histidine residues in CDR1, CDR2, and/or CDR3 of the heavy chain variable region (VH). In some embodiments, an anti-TM4SF1 antibody or antigen binding fragment thereof comprises one or more histidine residues in CDR1 of the heavy chain variable region (VH). In some embodiments, an anti-TM4SF1 antibody or antigen binding fragment thereof comprises one or more histidine residues in CDR2 of the heavy chain variable region (VH). In some embodiments, an anti-TM4SF1 antibody or antigen binding fragment thereof comprises one or more histidine residues in CDR3 of the heavy chain variable region (VH). Accordingly, in some embodiments, the combinations of the present disclosure comprise a histidine engineered anti-TM4SF1 antibody or antigen binding fragment thereof.

In some embodiments, an anti-TM4SF1 antibody or antigen binding fragment thereof comprises one or more histidine residues in CDR1, CDR2, and/or CDR3 of the light chain, for instance, in one or more of positions 30 (S30H), 92 (S92H), and 93 (N93H) of SEQ ID No. 101 or SEQ ID No. 131. In some embodiments, an anti-TM4SF1 antibody or antigen binding fragment thereof comprises one or more histidine residues in CDR1, CDR2, and/or CDR3 of the heavy chain, for instance in one or more of positions 28 (T28H), 31 (N31H), 32 (Y32H), 52 (N52H), 54 (Y54H), 57 (N57H), 100 (Q100H), and 101 (Y101H), of SEQ ID No. 92 or SEQ ID No. 130.

### III. Antibody Drug Conjugates

Provided herein, in one embodiment is a combination comprising an immunotherapeutic agent and an anti-angiogenic antibody or an antigen binding fragment thereof linked to a payload. In some embodiments, the antibody is an anti-TM4SF1 antibody or an antigen binding fragment thereof. In some embodiments, the payload is a small molecule, protein, peptide, antibody, or any combination thereof. In some embodiments, provided are combinations comprising ADCs comprising anti-angiogenic antibodies (such as anti-TM4SF1 antibodies or antigen binding fragments thereof), conjugated to therapeutic or diagnostic agents.

In some embodiments, the therapeutic agent is a biologically active moiety. In some embodiments, the biologically active moiety comprises a radioactive isotope, a cytotoxic agent, a chemotherapeutic agent, a protein, a peptide, an antibody, a growth inhibitory agent, a prodrug activating enzyme, and an anti-hormonal agent. In some embodiments, a therapeutic molecule can be a small molecule (e.g., both for cancer and for non-cancer angiogenic indications); a V-ATPase inhibitor; a pro-apoptotic agent; a Bcl2 inhibitor; an MCL1 inhibitor; a HSP90 inhibitor; an IAP inhibitor; an mTor inhibitor; a microtubule stabilizer; a microtubule destabilizer; an auristatin; a dolastatin; a maytansinoid; a MetAP (methionine aminopeptidase); an inhibitor of nuclear export of proteins CRM1; a DPPIV inhibitor; proteasome inhibitors; inhibitors of phosphoryl transfer reactions in mitochondria; a protein synthesis inhibitor; a kinase inhibitor (such as, a CDK2 inhibitor, a CDK9 inhibitor); a kinesin inhibitor, an HDAC inhibitor, a DNA damaging agent, a DNA alkylating agent, a DNA intercalator, a DNA minor groove binder, a DHFR inhibitor, a nucleic acid, a CRISPR enzyme; degraders (such as agents that induce protein degradation, (e.g., HSP90 inhibitor, selective estrogen receptor degraders (SERDs), selective androgen receptor degraders (SARDs); hydrophobic tags that can be used to recruit chaperones to a protein of interest, e.g., Adamantane, Arg-Boc3; E3 ligase recruiting ligands, e.g., Nutlin-3a (MDM2 ligand), Bestatin (cIAP ligand), VHL ligand, Pomalidomide (CRBN ligand); proteolysis-targeting chimeras (PROTACs) that may utilize different D3 ligases to target a protein of interest for degradation)) (see, e.g., Lai A C, Crews C M. Induced protein degradation: an emerging drug discovery paradigm. Nat Rev Drug Discov. 2016; 16(2):101-114); antisense oligonucleotides; RNAi agents (such as siRNA), CRISPR-Cas9 gene editing systems; RNA molecules; DNA e.g., plasmids; an anti-cancer agent, an anti-inflammatory agent, an anti-infective agent (e.g., anti-fungal, antibacterial, anti-parasitic, anti-viral), an anesthetic agent; RNA polymerase II inhibitor; a DNA intercalating agent, a DNA cross-linking agent; an anti-tubulin agent; a cytotoxic drug, a tumor vaccine, an antibody, a peptide, pepti-bodies, a chemotherapeutic agent, a cytotoxic agent; a cytostatic agent; an immunological modifiers, an interferon, an interleukin, an immuno stimulatory growth hormone, a cytokine, a vitamin, a mineral, an aromatase inhibitor, a Histone Deacetylase (HDAC), an HDAC inhibitor; a lipid nanoparticle to encapsulate one or more therapeutic molecule.

In some embodiments, the radioactive isotope may be one or more kinds selected from the group consisting of ²¹¹At, ¹³¹I, ¹²⁵I, ⁹⁰Y, ¹⁸⁶Re, ¹⁸⁸Re, ¹⁵³Sm, ²¹²Bi, ³²P, and radioactive isotopes of Lu, but not limited thereto. In some embodiments, the prodrug-activating enzyme is one or more kinds selected from the group consisting of: an alkaline phosphatase, an arylsulfatase, a cytosine deaminase, a protease, a D-alanylcarboxy-peptidase, a carbohydrate-cleaving enzyme, a β-lactamase and a penicillin amidase, but not limited thereto.

The cytotoxic agent, ins some embodiments, comprises one or more selected from the group consisting of: ricin, saporin, gelonin, momordin, debouganin, diphtheria toxin, pseudomonas toxin, etc., but not limited thereto. The cytotoxic agent, in some instances is one or more kinds selected from the group consisting of: cisplatin, carboplatin, oxaliplatin, paclitaxel, melphalan, doxorubicin, methotrexate, 5-fluorouracil, etoposide, mechlorethamine, cyclophosphamide, bleomycin, a calicheamicin, a maytansine, a trichothene, CC1065, diphtheria A chain, Pseudomonas aeruginosa exotoxin A chain, ricin A chain, abrin A chain, modeccin A chain, alpha-sarcin, Aleuritesfordii proteins, dianthin proteins, Phytolaca americana proteins, Momordica charantia inhibitors, curcin, crotin, Sapaonaria officinalis inhibitors, gelonin, mitogellin, restrictocin, phenomycin, enomycin, tricothecenes, ribonucleases and deoxyribonucleases, but not limited thereto. In some embodiments, the cytotoxic agent is one or more kinds selected from the group consisting of: duocarmycin, monomethyl auristatin E (MMAE), monomethyl auristatin F (MMAF), N2′-deacetyl-N2′-(3-mercapto-1-oxopropyl) maytansine (DM1), PBD (Pyrrolobenzodiazepine) dimer, duocarmycin, monomethyl auristatin E (MMAE), monomethyl auristatin F (MMAF), but not limited thereto. In some embodiments, the cytotoxic agent comprises a ribosome inactivating protein, a histone deacetylase (HDAC) inhibitor, a tubulin inhibitor, an alkylating agent, an antibiotic, an antineoplastic agent, an antiproliferative agent, an antimetabolite, a topoisomerase I or II inhibitor, a hormonal agonist or antagonist, an immunomodulator, a DNA minor groove binder, and a radioactive agent. In certain embodiments, the ribosome inactivating protein is saporin. In some embodiments, the diagnostic agent is a label. In some embodiments, the label is a fluorescent label, a chromogenic label, or a radiolabel. In some embodiments, the agent is directly conjugated to the anti-TM4SF1 antibody or antigen binding fragment thereof. In other embodiments, the agent is indirectly conjugated to the anti-TM4SF1 antibody or antigen binding fragment thereof, optionally by a linker.

In some embodiments, an ADC of this disclosure comprises an anti-TM4SF1 antibody or antigen binding fragment thereof and one or more agents (e.g., 1, 2, 3, or 4 or more agents), such as therapeutic agents, that act additively or synergistically with the anti-TM4SF1 antibody or antigen binding fragment thereof, for example, to kill or inhibit tumor cells (TCs) and/or tumor vasculature endothelial cells (ECs) in the treatment of a disorder associated with pathological angiogenesis, such as cancer. The therapeutic agent, for example, can be a biologically active moiety, such as a cytotoxic agent, a chemotherapeutic agent, a protein, a peptide, an antibody, a growth inhibitory agent, and/or an anti-hormonal agent.

Examples of tubulin inhibitors that can be conjugated, either directly or indirectly, to an anti-TM4SF1 antibody or antigen binding fragment thereof, can include, without limitation, polymerization inhibitors (e.g., vinblastine, vincristine, vinorelbine, vinflunine, cryptophycin 52, hallchondrins, dolastatins, hemiasterlins that can bind to the vinca domain of tubulin; colchine, combretastatins, 2-methoxy-estradiol, E7010 that can bind to the cholchicine domain of tubulin; depolymerization inhibitors, such as paclitaxel, docetaxel, epothilon, discodermolide that can bind to the taxane site).

Exemplary chemotherapeutic agents include, but are not limited to, methotrexate, adriamicin, vinca alkaloids (vincristine, vinblastine, etoposide), doxorubicin, melphalan, mitomycin C, chlorambucil, daunorubicin or other intercalating agents; enzymes and fragments thereof such as nucleolytic enzymes, antibiotics, and toxins such as small molecule toxins or enzymatically active toxins of bacterial, fungal, plant or animal origin, including fragments and/or variants thereof. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In addition, a variety of radionuclides can be used for conjugation of the anti-TM4SF1 antibodies or antigen binding fragments to the therapeutic agents, to generate the ADCs of this disclosure. Examples include At211, I131, I125, Y90, Rel86, Sm153, Bi212, P32, and radioactive isotopes of Lu. Alternatively, the anti-TM4SF1 antibodies or antigen binding fragments can be conjugated to one or more smaller molecule toxins, such as a calicheamicin, maytansinoids, dolastatins, aurostatins, a trichothecene, and CC1065, and the derivatives of these toxins that have toxin activity, are also contemplated herein. Other therapeutic agents that can be conjugated to TM4SF1 binding protein of the disclosure include, in various examples, BCNU, streptozoicin, vincristine and 5-fluorouracil etc.

The diagnostic agent for conjugation, in some embodiments, is a label, such as a fluorescent label, a chromogenic label, or a radiolabel. Accordingly, the label may be used for detection purposes, and may be a fluorescent compound, an enzyme, a prosthetic group, a luminescent material, a bioluminescent material, or a radioactive material. The radiolabel, for example, may comprise a radioactive atom for scintigraphic studies, for example Tc99m or I123, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

The one or more agents (e.g., therapeutic agents and/or diagnostic agents) may be directly conjugated to anti-TM4SF1 antibodies or antigen binding fragments (e.g., by way of a direct covalent or non-covalent interaction), such that the agent is immediately conjugated to the protein. An agent may be directly conjugated to a binding protein of the disclosure, for example, by a direct peptide bond. In other instances, the direct conjugation is by way of a direct non-covalent interaction, such as an interaction between the anti-TM4SF1 antibodies or antigen binding fragments and an agent that specifically binds to the anti-TM4SF1 antibodies or antigen binding fragments.

In some embodiments, the therapeutic agent comprises a proteasome inhibitor selected from group consisting of. bortezomib (Velcade, PS-341), PR-171 (carfilzomib), ixazomib (Ninlaro®), delanzomib, marizomib, oprozomib, VR23, PI-1840, (benzyloxycarbonyl)-Leu-Leu-phenylalaninal, 2,3,5a,6-tetrahydro-6-hydroxy-3-(hydroxymthyl)-2-methyl-10H-3a,10a-epidithio-pyrazinol[1,2a]indole-1,4-dione, 4-hydroxy-3-nitrophenylacetyl-Leu-Leu-Leu-vinyl sulphone, sapojargon, Ac-hFLFL-epoxide, aclacinomycin A, aclarubicin, ACM, AdaK(Bio)Ahx3L3VS, AdaLys(Bio)Ahx3L3VS, Adamantane-acetyl-(6-aminohexanoyl)-3-(leucunyl)-3-vinyl-(methyl)-sulphone, ALLM, ALLN, Calpain Inhibitor I, Calpain Inhibitor II, Carbobenzoxy-L-leucyl-L-leucyl-L-leucinal, Carbobenzoxy-L-leucyl-L-leucyl-L-norvalinal, gliotoxin, isovalery-L-tyrosyl-L-valyl-DL-tyrosinal, clasto-lactacystin-p-lactone, Z-LL-Nva-CHO, Ubiquitin Aldehyde, YU101, MP-LLL-VS, LDN-57444, Z-GPFL-CHO, Z-LLL-CHO, lovastatin, α-methyl-clasto-lactacystin-p-lactone, mevinolin, MK-803, NIP-L3VS, NP-LLL-VS, NPI-0052 (salinosporamide A), MLN519 (PS-519), NLVS (trileucine vinyl-sulfone), ritonavir, Ro106-9920, Z-LLF-CHO, Z-LL-B(OH)2, RRRPRPPYLPR, Tyropeptin A, ZL3VS, PR-11, PR-39, 0106-9920, Proteasome Inhibitor I, Proteasome Inhibitor II, Proteasome Inhibitor III, Proteasome Inhibitor IV, AdaAhx3L3VS, efrapeptin, MG-132, MG-262, MG-115, α-methylomuralide, MG-101, epoxomicin, omuralide, lactacystin, NEOSH101, or analogues thereof. In a specific embodiment, said proteasome inhibitor is carfilzomib.

In some embodiments, the therapeutic agent is an AKT kinase inhibitor, wherein the AKT kinase inhibitor can include, but is not limited to, ATP-competitive AKT kinase inhibitors, isoquinoline-5-sulfonamides (H-8, H-89, and NL-71-101), azepane derivatives (derived from (−)-balanol), aminofurazans (GSK690693), heterocyclic rings (7-azaindole derivatives, 6-phenylpurine derivatives, pyrrolo[2,3-d]pyrimidine derivatives, CCT128930, 3-aminopyrrolidine, anilinotriazole derivatives, spiroindoline derivatives, AZD5363, ipatasertib (GDC-0068, RG7440), A-674563, and A-443654), phenylpyrazole derivatives (AT7867, AT13148), thiophenecarboxamide derivatives (afuresertib (GSK2110183), uprosertib (GSK2141795), 2-pyrimidyl-5-amidothiophene derivative (DC120)), allosteric AKT kinase inhibitors, 2,3-diphenylquinoxaline analogues (2,3-diphenylquinoxaline derivatives, triazolo[3,4-f][1,6]naphthyridin-3(2H)-one derivative (MK-2206)), alkylphospholipids (Edelfosine (1-O-octadecyl-2-O-methyl-rac-glycero-3-phosphocholine, ET-18-OCH3), ilmofosine (BM 41.440), miltefosine (hexadecylphosphocholine, HePC), erucylphosphocholine (ErPC), erufosine (ErPC3), erucylphosphohomocholine), sulfonamide derivatives (PH-315, PHT-427), indole-3-carbinol analogues (indole-3-carbinol, 3-chloroacetylindole, diindolylmethane, OSU-A9, diethyl 6-methoxy-5,7-dihydroindolo[2,3-b]carbazole-2,10-dicarboxylate (SR13668)), Thiourea derivatives (N-[(1-methyl-1H-pyrazol-4-yl)carbonyl]-N′-(3-bromophenyl)-thiourea, PIT-1, PIT-2, DM-PIT-1), purine derivatives (triciribine (TCN, NSC 154020), triciribine mono-phosphate active analogue (TCN-P), 4-amino-pyrido[2,3-d]pyrimidine derivative API-1, ARQ 092, 3-phenyl-3H-imidazo[4,5-b]pyridine derivatives), BAY 1125976, 3-methyl-xanthine, quinoline-4-carboxamide, 2-[4-(cyclohexa-1,3-dien-1-yl)-1H-pyrazol-3-yl]phenol, 3-oxo-tirucallic acid, 3α- and 3β-acetoxy-tirucallic acids, acetoxy-tirucallic acid, irreversible inhibitors (lactoquinomycin, Frenolicin B, kalafungin, medermycin, Boc-Phe-vinyl ketone, 4-hydroxynonenal (4-HNE), 1,6-naphthyridinone derivatives, imidazo-1,2-pyridine derivatives), and more. Some examples of small molecule AKT inhibitors are GSK690693, Capiversertib, Ipatasertib, Afuresertib, Uprosertib, Miransertib, Miltefosine, Triciribine, Perifosine, Tehranolide, (E)-1-(2,4-Dihydroxyphenyl)-3-(4-hydroxyphenyl)prop-2-en-1-one (Isoliquiritigenin), 7-(D-D-glucopyranuronosyloxy)-5,6-dihydroxy-2-(4-hydroxyphenyl)-4H-1-benzopyran-4-one (Scutellarin), 7a,20-epoxy-1a,6b,7,14-tetrahydroxy-Kaur-16-en-15-one (oridonin), 3,5,7,8-tetrahydroxy-2-(4-hydroxyphenyl)chromen-4-one (herbacetin), (E)-1-(4-hydroxy-3-methoxyphenyl)dec-4-en-3-one ([6]-shogaol).

In some embodiments, the therapeutic agent is an antimitotic tetrapeptide. In some embodiments, the antimitotic tetrapeptide can include, but is not limited to, Tubulysins A-I, U, V, Y, z, and pretubulysin, and a derivative thereof. As used herein, the term “derivative of tubulin” generally refers to analogs of tubulysin, including but not limited to, oxazole analogs of tubulysin in which an oxazole ring replaces the thiazole ring. In some embodiments, the antimitotic tetrapeptide is Tubulysins A or D, or a derivative thereof. Without being bound by theory, it is expected that an anti-TM4SF1 antibody conjugated to a tubulysin and its derivatives would reduce drug-associated toxicity.

### IV. Polynucleotides

Also provided, in some embodiments, are polynucleotides encoding an anti-TM4SF1 antibody or an antigen binding fragment thereof. In some embodiments, the polynucleotide molecules are provided as a DNA construct. In other embodiments, the polynucleotide molecules are provided as a messenger RNA transcript.

In some examples, an anti-TM4SF1 antibody of the present disclosure comprises a heavy chain variable domain encoded by a nucleic acid sequence as set forth in any one of SEQ ID NOs: 4, 16, 28, 40, 52, 64, or 76. In some examples, an anti-TM4SF1 antibody of the present disclosure comprises a light chain variable domain encoded by a nucleic acid sequence as set forth in any one of SEQ ID NOs: 10, 22, 34, 46, 58, 70, or 82.

In some embodiments are provided nucleic acid sequences that are codon optimized for expression in a host cell, e.g., a bacterium, such as E. coli, or a eukaryotic cell, such as a CHO cell. In some examples, the nucleic acid sequences are codon optimized for expression in CHO cells. In some examples, an anti-TM4SF1 antibody of the present disclosure comprises a heavy chain variable domain encoded by a codon optimized nucleic acid sequence as set forth in any one of SEQ ID NOs: 5, 17, 29, 41, 53, 65, or 77. In some examples, an anti-TM4SF1 antibody of the present disclosure comprises a light chain variable domain encoded by a codon optimized nucleic acid sequence as set forth in any one of SEQ ID NOs: 11, 23, 35, 47, 59, 71, or 83. In certain instances, the nucleic acid sequence of any one of SEQ ID NOs: 5, 17, 29, 41, 53, 65, or 77 is a nucleic acid sequence codon optimized for expression in CHO cell. In certain instances, the nucleic acid sequence of any one of SEQ ID NOs: 11, 23, 35, 47, 59, 71, or 83 is a nucleic acid sequence codon optimized for expression in CHO cell.

The polynucleotide molecules are constructed by known methods such as by incorporating the genes encoding the binding proteins into a genetic construct linked to a suitable promoter, and optionally a suitable transcription terminator, and expressing it in bacteria or other appropriate expression system such as, for example CHO cells. Depending on the vector system and host utilized, any number of suitable transcription and translation elements, including constitutive and inducible promoters, may be used. The promoter is selected such that it drives the expression of the polynucleotide in the respective host cell.

In some embodiments, a polynucleotide as described herein is inserted into a vector, preferably an expression vector, which represents a further embodiment. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described TM4SF1 binding protein. Examples of expression vectors for expression in E. coli are pSKK (Le Gall et al., J Immunol Methods. (2004) 285(1):111-27) or pcDNAS (Invitrogen) for expression in mammalian cells.

Thus, the TM4SF1 binding proteins as described herein, in some embodiments, are produced by introducing a vector encoding the protein as described above into a host cell and culturing said host cell under conditions whereby the protein domains are expressed, may be isolated and, optionally, further purified.

### V. Immunotherapeutic Agents

In some embodiments, the combination of this disclosure comprises i) an anti-TM4SF1 antibody that are conjugated with cytotoxic payload and ii) an immunotherapeutic agent. Combination of an anti-angiogenic ADC and an immunotherapeutic can improve the treatment outcome of immunotherapeutic agents. Anti-angiogenesis ADC can facilitate immune cell infiltration to tumor microenvironment, leading to improved treatment outcomes of immunotherapeutic agents by turning cold tumors to hot. Such treatment outcomes, may include, but are not limited to increased immune cell infiltration or a decrease in T-reg cell population. The ADC could also be, in some instances, used in the context of vascular injury for wound healing.

In some embodiments, immunotherapeutic agent is an antibody, or an antigen binding fragment thereof directed against a target present in a cell of a myeloid lineage, a tumor cell, a cell of a lymphoid lineage, a molecule present in a tumor microenvironment. In some embodiments, the immunotherapeutic agent is an antibody, or an antigen binding fragment thereof directed against at least one of: PD-1, CTLA-4, CD40, CSF1/CSF1R, SIRPα, CLEC-1. In some embodiments, the immunotherapeutic agent is an antibody or an antigen binding fragment thereof directed against at least one of: CCR4, CTLA-4, AIR, A2AR, A3R, TIM-3, BTLA, VISTA, TIGIT, LAG-3, ILRa/CD25, ITGB1/CD29, Ly 24/CD44, CD48, CEACAM1/CD66a, Nt5e/CD73, CD94/NKG2A, FAS/CD95, SLAF1/CD150, NRP1/CD304, GITR/CD357, ICOS, Tnfrs4/OX40, Folr4/JUNO, P2X7, ANXA2, IDO, B7-H6, KIR, GARP (LRRC32), TNFR2. In some embodiments, the immunotherapeutic agent is an antibody, or an antigen binding fragment thereof directed against at least one of: PD-L1, PD-L2, B7-H3, B7-H4, CD47, TDO, DcR3. In some embodiments, the immunotherapeutic agent is an antibody, or an antigen binding fragment thereof directed against at least one of: an exosome, a cytokine, an interleukin, or a chemokine. In some embodiments, the immunotherapeutic agent is an antibody, or an antigen binding fragment thereof directed against at least one of: PS, STING.

The immunotherapeutic agent, in some embodiments, is a cytokine, such as IL-2 and IL-15, which can function in aiding the proliferation and differentiation of B cells, T cells, and NK cells. In some embodiments, the immunotherapeutic agent comprises a cellular therapy, cancer vaccine, hormone, epitope, cytokine, tumor antigen, CD4 cell stimulator, NKT cell agonist, or adjuvant. For example, the immunotherapeutic agent can be an interferon, interleukin, tumor necrosis factor, ovalabumin, Neuvenge, Oncophage, CimaVax-EGF, Mobilan, a-Gal glycolipid, a-Galactosylceramide (a-GalCer), β-mannosylceramide (β-ManCer), adenovirus delivered vaccines, Celldex's CDX1307 and CDX1401; GRNVAC1, viral based vaccines, MVA-BN, PROSTVAC®, Advaxis®; ADXSII-001, ADXS31-001, ADXS31-164, BiovaxID, folate binding protein (E39), Granulocyte macrophage colony stimulating factor (GM-CSF) with and without E75 (NeuVax) or OncoVEX, trastuzumab, Ae-37, IMA901, SC1B 1, Stimuvax, peptides that can elicit cytotoxic lymphocyte response, peptide vaccines including telomerase peptide vaccine (GV1001), survivin peptide, MUC1 peptide, ras peptide, TARP 29-37-9V Peptide epitope enhanced peptide, DNA Vector pPRA-PSM with synthetic peptides E-PRA and E-PSM.

In some embodiment, the immunotherapeutic agent comprises an adoptive immunotherapy, which involves the transfer of autologous antigen-specific T cells generated ex vivo, The T cells used for adoptive immunotherapy can be generated either by expansion of antigen-specific T cells or redirection of T cells through genetic engineering. Isolation and transfer of tumor specific T cells has been shown to be successful in treating melanoma. Novel specificities in T cells have been successfully generated through the genetic transfer of transgenic T cell receptors or chimeric antigen receptors (CARs). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains, CARs have successfully allowed T cells to be redirected against antigens expressed at the surface of tumor cells from various malignancies including lymphomas and solid tumors.

The immunotherapeutic agent, in some embodiments, is an inhibitor of an inhibitory molecule of an immune checkpoint molecule. The term “immune checkpoints” refers to a group of molecules on the cell surface of CD4 and CD8 T cells. These molecules can effectively serve as “brakes” to down-modulate or inhibit an anti-tumor immune response. Inhibition of an inhibitory molecule can be performed by inhibition at the DNA, RNA or protein level. In embodiments, an inhibitory nucleic acid (e.g., a dsRNA, siRNA or shRNA), can be used to inhibit expression of an inhibitory molecule. In other embodiments, the inhibitor of an inhibitory signal is, a polypeptide e.g., a soluble ligand, or an antibody or antigen-binding fragment thereof, that binds to the inhibitory molecule. An immune checkpoint inhibitor can suppress anti-tumor immunity and/or enhance visibility of tumor neoantigens.

In some embodiments, the immunotherapeutic agent can be an immune check point inhibitor. In some embodiments, the immune check point inhibitor is an antibody or antigen binding fragment directed against a target expressed on myeloid cells, lymphoid cells, tumor cells, or other small particles of the tumor microenvironment. In some embodiments the small particles of the tumor microenvironment comprise exosomes, cytokines, interleukins, or chemokines. Exemplary targets expressed on myeloid cells can be, but are not limited to, PD-1, CD40, CSF1, CSF1-R, SIRPα, and/or CLEC1. Exemplary targets expressed on lymphoid cells can be, but are limited to, CCR4, CTLA-4, AIR, A2AR, A3R, TIM-3, BTLA, VISTA, TIGIT, LAG-3, ILRa/CD25, ITGB1/CD29, Ly 24/CD44, CD48, CEACAM1/CD66a, Nt5e/CD73, CD94/NKG2A, FAS/CD95, SLAF1/CD150, NRP1/CD304, GITR/CD357, ICOS, Tnfrs4/OX40, Folr4/JUNO, P2X7, ANXA2, IDO, B7-H6, KIR, GARP (LRRC32), or TNFR2. Exemplary targets expressed on tumors cells can be, but are not limited to PD-L1, PD-L2, B7-H3, B7-H4, CD47, TDO, or DcR3. Exemplary small particles in the tumor microenvironment can be, but are not limited to, PS, or STING. In some embodiments, the immune checkpoint inhibitor is a protein that inhibits T cell activation such as CTLA-4, PD-1, PD-L1, PD-L2, GITR, and LAG-3, Galectin 9, CEACAM-1, BTLA, CD69, Galectin-1, TIGIT, CD113, GPR56, VISTA, B7-H3, B7-H4, 2B4, CD48, GARP, PD1H, LAIR1, TIM-1, TIM-3, and TIM-4. In some embodiments, the anti-LAG-3 antibody is BMS-986016. BMS-986016 (also referred to as BMS986016; Bristol-Myers Squibb) is a monoclonal antibody that binds to LAG-3. BMS-986016 and other humanized anti-LAG-3 antibodies are disclosed in US 2011/0150892, WO2010/019570, and WO2014/008218.

Other exemplary immunotherapeutic agents can be, but are not limited to, alemtuzumab, rituximab, tosituimomab, obinutuzumab, ofatumumab, ibitumomab tiuxetan, dinutuximab, blinatumomab, daratumumab, isatuximab ipilimumab, satuximab-irfc, elotuzumab, cetuximab, panitumumab, necitumumb, catumaxomab, trastuzumab, pertuzumab, olaratumab, bevacizmab, ramucinumab, imiquimod, tocilzumab, nivolumab, pembrolizumab, ipilmumub, tremelimumab, nivolumab, cemiplimab, pembrolizumab, avelumab, atezolizumab, durvalumab, tremelimumab, spartalizumab, avelumab, sintilimab, toripalimab, NKTR-214, tisageniecluecel, axicabtagene ciloleucel, aldesleukin, inerteron alfa-3a/2b, peridaratinib, sipuleucel-T, HIOI, Talimogene laherparepvec, MGA012, MGD013, MGD019, enoblituzumab, MGD009, MGC018, MEDI0680, PDR001, FAZ053, TSR022, MBG453, relatlinab (BMS986016), LAG525, IMP321, REGN2810 (cemiplimab), REGN3767, pexidartinib, LY3022855, FPA008, BMS-986226, BMS-986207, BLZ945, BMS-986315, GDC0919, epacadostat, indoximid, BMS986205, CPI-444, MED19447, PBF509, lirilumab, IMC-001, Monalizumab, and combinations thereof. The antagonist may be an antibody, an antigen binding fragment thereof, an immunoadhesin, a fusion protein, or oligopeptide.

The immunotherapeutic agent may be an antibody drug conjugate. Exemplary antibody drug conjugates may include, but are not limited to, moxetumomab, bretuximab, trastuzumab, inotuzumab, gemtuzumab, tagraxofusp-erza, polatuzumab, enfortumab vedotin-ejfv, trastuzumab deruxtecan, sacituzumab govitecan-hziy.

Exemplary immunotherapeutic agents also include, but are not limited to: YERVOY® (ipilimumab) or Tremelimumab (to CTLA-4), galiximab (to B7.1), BMS-936558 (to PD-1), MK-3475 (to PD-1), atezolizumab (TECENTRIQ®), AMP224 (to B7DC), BMS-936559 (to B7-H1), MPDL3280A (to B7-H1), MEDI-570 (to ICOS), AMG557 (to B7H2), MGA271 (to B7H3), IMP321 (to LAG-3), BMS-663513 (to CD137), PF-05082566 (to CD137), CDX-1127 (to CD27), anti-OX40 (Providence Health Services), huMAbOX40L (to OX40L), Atacicept (to TACT), CP-870893 (to CD40), Lucatumumab (to CD40), Dacetuzumab (to CD40), Muromonab-CD3 (to CD3); anti-GITR antibodies MK4166, TRX518, Medi1873, INBRX-110, LK2-145, GWN-323, GITRL-Fc, or any combination thereof.

Exemplary immunotherapeutic agents can also include, but are not limited to, ipilimumab, nivolumab, pembrolizumab, atezolizumab, durvalumab, tremelimumab, spartalizumab, avelumab, sintilimab, toripalimab, MGA012, MGD013, MGD019, enoblituzumab, MGD009, MGC018, MEDI0680, PDR001, FAZ053, TSR022, MBG453, relatlinab (BMS986016), LAG525, IMP321, REGN2810 (cemiplimab), REGN3767, pexidartinib, LY3022855, FPA008, BLZ945, GDC0919, epacadostat, indoximid, BMS986205, CPI-444, MED19447, PBF509, lirilumab, IMC-001, Monalizumab, and combinations thereof.

### VI. Methods of Treatment

Provided herein are methods of treatment comprising administering a combination comprising an anti-TM4SF1 antibody or an antigen binding fragment, or an ADC containing such an antibody, and an immunotherapeutic agent.

In some embodiments, this disclosure provides a method for inhibiting cell-cell interactions that are endothelial cell (EC) specific, for example, but not limited to EC-EC, EC-mesenchymal stem cell, EC-fibroblast, EC-smooth muscle cell, EC-tumor cell, EC-leukocyte, EC-adipose cell and EC-neuronal cell interactions.

By “combination” or “in combination with,” it is not intended to imply that the therapy or the therapeutic agents must be administered at the same time and/or formulated for delivery together (e.g., in the same composition), although these methods and compositions are within the scope described herein. The immunomodulator and the second therapeutic agent can be administered concurrently with, prior to, or subsequent to, one or more other additional therapies or therapeutic agents. The agents in the combination can be administered in any order. In general, each agent will be administered at a dose and/or on a time schedule determined for that agent. As will further be appreciated that the additional therapeutic agent utilized in this combination may be administered together in a single composition or administered separately in different compositions. In general, it is expected that additional therapeutic agents utilized in combination can be utilized at levels that do not exceed the levels at which they are utilized individually. In some embodiments, the levels utilized in combination can be lower than those utilized individually.

In some embodiments, methods disclosed herein comprising administering an anti-TM4SF1 antibody or an antigen binding fragment thereof or an ADC as described herein, in combination with one or more immunotherapeutic agents (e.g., an anti-PD-1 or PD-L1 antibody molecule, an anti-LAG-3, or anti-TIM-3 antibody molecule).

The components of the combination, an anti-TM4SF1 antibody or an antigen binding fragment thereof, or an ADC containing such an antibody, in combination with an immunotherapeutic agent, are in some cases administered concurrently. Concurrent administration means each component is administered at the same time or within 8-12 hours of each other. Administration of a second component more than 12 hours after the first component can be considered a sequential administration. The components of the combination, an anti-TM4SF1 antibody or an antigen binding fragment thereof, or an ADC containing such an antibody, in combination with an immunotherapeutic agent, are in some cases administered sequentially (e.g., one, two, three, four, five, six, seven, eight, or nine days apart; one, two, three, or four weeks apart; one, two, three, four, five, six, seven, eight, or nine weeks apart; or one, two, three, four, five, six, or more years apart; or the like), in any order and in any combination. In some embodiments, when administered sequentially, the anti-TM4SF1 antibody or antigen binding fragment thereof, or an ADC containing such an antibody is administered first and the immunotherapeutic agent is administered second. In other embodiments, the immunotherapeutic agent is administered first and the anti-TM4SF1 antibody or antigen binding fragment thereof, or an ADC containing such an antibody is administered second.

Additional Agents

In some embodiments the combination may further comprise additional agents such as tyrosine kinase inhibitors, Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-β inhibitor)), a RAF-1 inhibitor, a KIT inhibitor and a RET inhibitor. In some embodiments, the anti-cancer agent used in combination with the hedgehog inhibitor is selected from the group consisting of axitinib (AG013736), bosutinib (SKI-606). cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), yandetanib (ZACTIMA®, ZD6474), vatalanib (PTK787, PTK/ZK), trastuzumab (HERCEPTTN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TK1258, CHIR-258), BTBW 2992 (TOVOK®), SGX523, PF-04217903, PF-02341066, PF-299804, BMS-777607, ABT-869, MP470, BIBF 1120 (V ARGATEF®), AP24534, JNJ-26483327, MGCD265, DCC-2036, BMS-690154, C EP-11981, tivozanib (AV-951), OSI-930, MM-121, XL-84, XL-647, XL228, AEE788, AG-490, AST-6, BMS-599626, CUDC-101, PD153035, pelitinib (EKB-569), vandetanib (zactima), WZ3146, W74002, WZ8040. ABT-869 (linifanib), AEE788, AP24534 (ponatinib), AV-951 (tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, K18751, masitinib (ABIOIO), MGCD-265, motesanib diphosphate (AMG-706), MP-470, OSI-930, Pazopanib Hydrochloride, PD173074, Sorafenib Tosylate(Bay 43-9006), SU 5402, TSU-68(SU6668), vatalanib, XL880 (GSR1363089, EXEL-2880). Further examples of hedgehog inhibitors include, but are not limited to, vismodegib (2-chloro-N-[4-chloro-3-(2-pyridinyl)phenyl]-4-(methylsulfonyl)-benzamide, GDC-0449, described in PCT Publication No. WO 06/028958); 1-(4-Chloro-3-(trifluoromethyl)phenyl)-3-((3-(4-fluorophenyl)-3,4-dihydro-4-oxo-2-quinazolinyl)methyl)-urea (CAS 330796-24-2); N-[(2S,3R,3′R,3aS,4′aR6S,6′aR,6′bS,7aR,12′aS,12′bS)-2′,3′,3a,4,4′,4′a,5,5′,6,6′,6′a,6′b,7.7′,7a,8′,10′,12′12′a,12′b-Eicosahydro-3,6,11′,12′b-tetramethylspiro[furo[3,2-b]pyridine-2(3H),9(1′H)-naphth[2,1-a]azulen]-3′-yl]-methanesulfonamide (TP1926, CAS 1037210-93-7); and 4-Fluoro-N-methyl-N-[1-[4-(1-methyl-1H-pyrazol-5-yl)-1-phthalazinyl]-4-piperidinyl]-2-(trifluoromethyl)-benzamide (LY2940680, CAS 1258861-20-9); and Erismodegib (LDE225). Selected tyrosine kinase inhibitors are chosen from gefitinib; erlotinib hydrochloride (Tarceva®); linifanib (N-[4-(3-amino-1H-indazol-4-yl)phenyl]-N′-(2-fluoro-5-methylphenyl)urea, also known as ABT 869, available from Genentech); sunitinib malate (Sutent®); bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, described in U.S. Pat. No. 6,780,996); dasatinib (Sprycel®); pazopanib (Votrient®); sorafenib (Nexavar®); zactina (7D6474); and imatinib or imatinib mesylate (Gilvec® and Gleevec®).

In some embodiments the combination may further comprise additional agents such as Vascular Endothelial Growth Factor (VEGF) receptor inhibitors. Examples of VEGF inhibitor can include but not limited to, Bevacizumab (Avastin®), axitinib (Inlyta®); Brivanib alaninate (BMS-582664, (5)-((R)-1-(4-(4-Fluoro-2-methyl-1-indol-5-yloxy)-5-methylpyrrolo 2,1-/][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF 1120, CAS 928326-83-4); Foretinib (GSK1363089); Telatinib (BA-Y57-9352, CAS 332012-40-5); Apatinib (YN968D1, CAS 811803-05-1); Imatinib (Gleeveca); Ponatinib (AP24534, CAS 943319-70-8); Tivozanib (AV951, CAS 475108-18-0); Regorafenib (BAY73-4506, CAS 755037-03-7), Vatalanib dihydrochloride (PTK787, CAS 212141-51-0); Brivanib (BMS-540215, CAS 649735-46-6), Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No, WO 02/066470); Dovitinib dilactic acid (TK1258, CAS 852433-84-2); Linfanib (ABT869, CAS 796967-16-3); Cabozantinib (XL184, CAS 84921 7-68-1); Lestaurtinib (CAS 111358-88-4); N-[5-[[[5-(1,1-Dimethylethyl)-2-oxazolyl]methyl] thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aa,5p,6aa)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-J]pyrimidin-4-yl]amino]-N-[3 trifluoromethyl)phenyl]-benzamide (BHG712, CAS 940310-85-0); and Aflibercept (Eylea®). Exemplary anti-VEGF antibodies include, but are not limited to, a monoclonal antibody that binds to the same epitope as the monoclonal anti-VEGF antibody A4.6.1 produced by hybridoma ATCC HB 10709; a recombinant humanized anti-VEGF monoclonal antibody generated according to Presta et al. (1997) Cancer Res. 57:4593-4599. In one embodiment, the anti-VEGF antibody is Bevacizumab (BV), also known as rhuMAb VEGF or AVASTIN®. It comprises mutated human IgG1 framework regions and antigen-binding complementarity-determining regions from the murine anti-hVEGF monoclonal antibody A.4.6.1 that blocks binding of human VEGF to its receptors. Bevacizumab and other humanized anti-VEGF antibodies are further described in U.S. Pat. No. 6,884,879 issued Feb. 26, 2005. Additional antibodies include the G6 or B20 series antibodies (e.g., G6-31, B20-4.1), as described in PCT Publication No. WO2005/012359, PCT Publication No. WO2005/044853, the contents of these patent applications are expressly incorporated herein by reference. For additional antibodies see U.S. Pat. Nos. 7,060,269, 6,582,959, 6,703,020, 6,054,297, WO98/45331 WO 96/30046, WO94/10202, EP 0666868B1, U.S. Patent Application Publication Nos. 2006009360, 20050186208, 20030206899, 20030190317, 20030203409, and 20050112126; and Popkov et al, Journal of Immunological Methods 288: 149-164 (2004). Other antibodies include those that bind to a functional epitope on human VEGF comprising of residues F17, M18, D19, Y21, Y25, Q89, 191, Kl 01, El 03, and C104 or, alternatively, comprising residues F17, Y21, Q22, Y25, D63, 183 and Q89.

In some embodiments the combination may further comprise additional agents such as PI3K inhibitors. An example PI3K inhibitor is an inhibitor of delta and gamma isoforms of PI3K. Further examples of PI3K inhibitors include, but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC 0941, described in PCT Publication Nos. WO 09/036082 and WO 09/055730), 2-Methyl-2-[4-[3-methyl-2-oxo-8-(quinolin-3-yl)-2,3-dihydroimidazo[4,5-c]quinolin-1-yl]phenyl] propionitrile (also known as BEZ235 or NVP-BEZ 235, described in PCT Publication No. WO 06/122806); 4-(trifluoromethyl)-5-(2,6-dimorpholinopyrimidin-4-yl)pyridin-2-amine (also known as BKM120 or NVP-BKM120, described in PCT Publication No. WO2007/084786); Tozasertib (VX680 or MK-0457, CAS 639089-54-6); (5Z)-5-[[4-(4-Pyrdinyl)-6-quinolinyl]methylene]-2,4-thiazolidinedione (GSK1059615, CAS 958852-01-2); (1E,4S,4aR,5R,6aS,9aR)-5-(Acetyloxv)-1-[(di-2-propenylamino)methylene]-4,4a,5,6,6a,8,9,9a-octahydro-1 1-hydroxy-4-(methoxy methyl)-4a,6a-dimethlyl-cyclopenta[5,6]naphtho[1,2-c]pyran-2,7,10(1H)-trione (PX866, CAS 502632-66-8); 8-Phenyl-2-(morpholin-4-yl)-chromen-4-one (LY294002, CAS 154447-36-6); 2-Amino-8-ethyl-4-methyl-6-(1H-pyrazol-5-yl)pyrido[2,3-d]pyrimidin-7(8H)-one (SAR 245409 or XL 765); 1,3-Dihydro-8-(6-methoxy-3-pyridinyl)-3-methyl-1-[4-(1-piperazinyl)-3-(trifluoromethyl)phenyl]-2H-imidazo [4,5-c] quinolin-2-one, (2Z)-2-butenedioate (1:1) (BGT 226); 5-Fluoro-3-phenyl-2-[(S)--(9H-purin-6-ylamino)ethyl]-4(31-1)-quinazolinone (CAL101); 2-Amino-N-[3-[N-[3-[(2-chloro-5-methoxyphenyl)amino]quinoxalin-2-yl]sulfamoyl]phenyl]-2-methylpropanamitde (SAR 245408 or XL 147); and (S)-Pyrrolidine-1,2-dicarboxylic acid 2-amide 1-({4-methyl-5-[2-(2,2,2-trifluoro-1,1-dimethyl-ethyl)-pyridin-4-vi]-thiazol-2-y}-amide) (BYL719).

In some embodiments the combination may further comprise additional agents such as mTOR inhibitors, e.g., one or more mTOR inhibitors chosen from one or more of rapamycin, temsiroiimus (TORISEL®), AZD8055, BEZ235, BGT226, XL765, PF-4691502, GDC0980, SF1126, OSI-027, GSK1059615, KU-0063794, WYE-354, Palomid 529 (P529), PF-04691502, or PK1-587, ridaforolimus (formally known as deforolimus, (1R,2R,4S)-4-[(2R)-2 [(1R,9S,12S,15R,16E,18R,19R,21R. 23S,24E,26E,25Z,30S,32S,35R)-1.18-dihydroxy-19,30-dimethoxy-15,17,21,23, 29,35-hexamethyl-2,3,10,14,20-pentaoxo-11,36-dioxa-4-azatricyclo[30.3.1.04′91 hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimusk); simapimod (CAS 164301-51-3); emsirolimus, (5-{2,4-Bis[(3S)-3-methylmorpholin-4-yl]pyrido[2,3-JJpyrimidin-7-yl}-2-methoxyphenyl)methanol (AZD8055); 2-Amino-8-[iraw5,-4-(2-hydroxyethoxy)cyclohexyl]-6-(6-methoxy-3-pyridinyl)-4-methyl-pyrido[2,3-JJpyrimidin-7(8H)-one (PF04691502, CAS 1013101-36-4); and N2-[,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SF] 126, CAS 936487-67-1), (1r,4r)-4-(4-amino-5-(7-methoxy-1H-indol-2-yl)imidazo[1,5-f] 11,2,4]triazin-7-yl)cyclohexanecarboxylic acid (OSI-027); and XL765.

In some embodiments the combination may further comprise additional agents such as a BRAF inhibitor, eg., GSK2118436, RG7204, PLX4032, GDC-0879, PLX4720, and sorafenib tosylate (Bay 43-9006). In further embodiments, a BRAF inhibitor includes, but is not limited to, regorafenib (BAY73-4506, CAS 755037-03-7); tuvizanib (AV951, CAS 475108-18-0); vemurafenib (Zelboraf®, PLX-4032, CAS 918504-65-1); encorafenib (also known as LGX818); 1-Methyl-5-[[2-[5-(trifluoromethyl)-111-imidazol-2-yl]-4-pyridinyl] oxy]-N-[4-(trifluoromethyl)phenyl-1H-benzimidazol-2-amine (RAF265, CAS 927880-90-8); 5-[1-(2-Hydroxyethyl)-3-(pyridin-4-yl)-1H-pyrazol-4-yl]-2,3-dihydroinden-1-one oxime (GDC-0879, CAS 905281-76-7); 5-[2-[4-[2-(Dimethylamino)ethoxy]phenyl]-5-(4-pyridinyl)-1H-imidazol-4-yl]-2,3-dihydro-1H-Inden-1-one oxime (GSK2118436 or SB590885); (+/−)-Methyl (5-(2-(5-chloro-2-methylphenyl)-1-hydroxy-3-oxo-2,3-dihydro-1H-isoindol-1-yl)-1H-benzimidazol-2-yl)carbamate (also known as XL-281 and BMS908662) and N-(3-(5-chloro-1-pyrrolo[2,3-b]pyridine-3-carbonyl)-2,4-difluorophenyl)propane-1-sulfonamide (also known as PLX4720).

In some embodiments the combination may further comprise additional agents such as a MEK inhibitor. Any MEK inhibitor can be used in combination including, but not limited to, selumetinib (5-[(4-bromo-2-chiorophenyl)amino]-4-fluoro-N-(2-hydroxyethoxy)-1-methyl-1H-benzimidazole-6-carboxamide, also known as AZD6244 or ARRY 142886, described in PCT Publication No. WO2003077914); trametinib dimethyl sulfoxide (GSK-1120212, CAS 1204531-25-80), RDEA436; N-[3,4-Difluoro-2-[(2-fluoro-4-iodophenyl)aminol-6-methoxyphenyl]-1-[(2R)-2,3-dihydroxypropyl]-cyclopropanesulfonamide (also known as RDEA119 or BAY869766, described in PCT Publication No. WO2007014011); AS703026; BIX 02188; BIX 02189; 2-1(2-Chloro-4-iodophenyl)amino]-N-(cyclopropylmethoxy)-3,4-difluoro-benzamide (also known as CT-1040 or PD184352, described in PCT Publication No. WO2000035436); N-[(2R)-2,3-Dihydroxypropoxy]-3,4-difluoro-2-[(2-fluoro-4-iodophenyl)aminol-benzamide (also known as PD0325901 and described in PCT Publication No. WO2002006213); 2′-amino-3-methoxyflavone (also known as PD98059 available from Biiaffin GmbH & Co., KG, Germany); 2,3-bis[amino[(2-aminophenyl)thio]methylen-e]butanedinitrile (also known as U0126 and described in U.S. Pat. No. 2,779,780): XL-518 (also known as GDC-0973, Cas No. 1029872-29-4, available from ACC Corp.); G-38963; and G02443714 (also known as AS703206), or a pharmaceutically acceptable salt or solvate thereof. Additional examples of MEK inhibitors are disclosed in WO 2013/019906, WO 03/077914, WO 2005/121142, WO 2007/04415, WO 2008/024725 and WO 2009/085983, the contents of which are incorporated herein by reference. Further examples of MEK inhibitors include, but are not limited to, benimetinib (6-(4-bromo-2-fluorophenylamino)-7-fluoro-3-methyl-31-1-benzoimidazole-5-carboxylic acid (2-hydroxyethyoxy)-amide, also known as MEK162, CAS 1073666-70-2, described in PCT Publication No. WO2003077914); 2,3-Bis[amino[(2-aminophenyl)thio]methylene]-butanedinitrile (also known as U0126 and described in U.S. Pat. No. 2,779,780); (3S,4R,5Z,8S,9S,11E)-14-(Ethylamino)-8,9,16-trihydroxy-3,4-dimethyl-3,4,9, 19-tetrahydro-1H-2-benzoxacyclotetradecine-1,7(8H)-dione] (also known as E6201, described in PCT Publication No. WO2003076424); vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); pimasertib (AS-703026, CAS 1204531-26-9); 2-(2-Fluoro-4-iodophenylamino)-N-(2-hydroxyethoxy)-1,5-dimethyl-6-oxo-1,6-dihydropyridine-3-carboxamide (AZD 8330); and 3,4-Difiuoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655).

In some embodiments the combination may further comprise additional agents such as a JAK inhibitor., e.g., Exemplary JAK inhibitors include, but are not limited to, r CEP-701, INCB18424, CP-690550 (tasocitinib), uxolitinib (Jakafi); tofacitinib (CP690550); axitinib (AG013736, CAS 319460-85-0); 5-Chloro-N2-I(IS)-1-(5-fluoro-2-pyrimidinyl)ethyl]-N4-(5-methyl-1H-pyrazol-3-y)-12,4-pyrimidinediamine (AZD1480, CAS 935666-88-9); (9E)-15-[2-(1-Pyrrolidinyl)ethoxy]-7, 12,26-trioxa-19,21,24-triazatetracyclo[18.3,1,12,5,114,18]-hexacosa-1(24),2,4,9,14,16,18(25),20,22-nonaene (SB-1578, CAS 937273-04-6); momelotinib (CYT 387); baricitinib (INCB-028050 or LY-3009104); pacritinib (SB1518); (16E)-14-Methyl-20-oxa-5,7,14,27-tetraazatetracyclo[19.3.1,12,6,18,12]heptacosa-1(25),2,4,6(27)8, 10,12(26),16,21,23-decaene (SB 1317); gandotinib (LY 2784544); and N,N-cicyclopropyl-4-[(1,5-dimethyl-1H-pyrazol-3-yl)amino]-6-ethyl-1,6-dihydro-1-methyl-imidazo[4,5-dipyrrolo[2,3-b]pyridine-7-carboxamide (BMS 911543).

In some embodiments the combination may further comprise additional agents such as an antibody against a Killer-cell Immunoglobulin-like Receptors (also referred to herein as an “anti-KIR antibody”). In some embodiments the combination may further comprise administering a cellular immunotherapy (e.g., Provenge (e.g., Sipuleucel)), and optionally in combination with cyclophosphamide. In some embodiments the combination may further comprise additional agents such as a vaccine, e.g., a dendritic cell renal carcinoma (DC-RCC) vaccine. In some embodiments the combination may further comprise additional agents such as a paclitaxel or a paclitaxel agent, e.g., TAXOL®, protein-bound paclitaxel (e.g., ABRAXANE®). Exemplary paclitaxel agents include, but are not limited to, nanoparticle albumin-bound paclitaxel (ABRAXANE, marketed by Abraxis Bioscience), docosahexaenoic acid bound-paclitaxel (DIHA-paclitaxel, Taxoprexin, marketed by Protarga), polygiutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et ah, Biopolymers (2007) 87:225-230), and glucose-conjugated paclitaxel (e.g.,-paclitaxel methyl 2-glucopyranosyl succinate, see Liu et ah, Bioorganic & Medicinal Chemistry Letters (2007) 17:617-620).

In some embodiments the combination may further comprise additional agents such as a chemotherapy, and/or an immunotherapy. For example, a combination comprising an anti-TM4SF1 antibody or an antigen binding fragment thereof or an ADC comprising the same, in combination with an immunotherapeutic agent can be used to treat a myeloma, alone or in combination with one or more of: chemotherapy or other anti-cancer agents (e.g., thalidomide analogs, e.g., lenalidomide), tumor antigen-pulsed dendritic cells, fusions (e.g., electrofusions) of tumor cells and dendritic cells, or vaccination with immunoglobulin idiotype produced by malignant plasma cells.

Cancers

In some embodiments of the method, the cancer treated with the combination is selected from the group consisting of prostate cancer, liver cancer, colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung, non-small cell lung), sarcoma (soft tissue sarcoma and osteosarcoma), melanoma, non melanoma skin cancer (squamous and basal cell carcinoma), glioma, renal cancer, lymphoma (NHL or HL), Acute myeloid leukemia (AML), T cell Acute Lymphoblastic Leukemia (T-ALL), Diffuse Large B cell lymphoma, testicular germ cell tumors, mesothelioma, esophageal cancer, Merkel Cells cancer, MSI-high cancer, KRAS mutant tumors, adult T-cell leukemia/lymphoma, and Myelodysplastic syndromes (MDS). In some embodiments of the method, the cancer is selected from the group consisting of cancer triple negative breast cancer, stomach (gastric) cancer, lung cancer (small cell lung, non-small cell lung), Merkel Cells cancer, MSI-high cancer, KRAS mutant tumors, adult T-cell leukemia/lymphoma, and Myelodysplastic syndromes (MDS). In some embodiments of the method, the cancer is selected horn the group consisting of cancer triple negative breast cancer, stomach (gastric) cancer, lung cancer (small cell lung, non-small cell lung), Merkel Cells cancer, and MSI-high cancer. In certain embodiments, the cancer includes a BRAF mutation (e.g., a BRAF V600E mutation), a BRAF wildtype, a KRAS wildtype or an activating KRAS mutation. The cancer may be at an early, intermediate or late stage.

In one embodiment, the combination provided herein is further combined with a chemotherapy to treat a lung cancer, e.g., non-small cell lung cancer. In one embodiment, the combination is used with platinum doublet therapy to treat lung cancer. In yet another embodiment, the combination is used to treat a renal cancer, e.g., renal cell carcinoma (RCC) (e.g., clear cell renal cell carcinoma (CCRCC) or metastatic RCC. The combination administered in combination with an additional agent, comprising one or more of: an immune-based strategy (e.g., interleukin-2 or interferon-a), a targeted agent (e.g., a VEGF inhibitor such as a monoclonal antibody to VEGF); a. VEGF tyrosine kinase inhibitor such as sunitinib, sorafenib, axitinib and pazopanib; an RNAi inhibitor), or an inhibitor of a downstream mediator of VEGF signaling, e.g., an inhibitor of the mammalian target of rapamycin (mTOR), e.g., everolimus and temnsiroiimus.n example of suitable therapeutics for use in combination for treatment of small cell lung cancer includes, but is not limited to, a chemotherapeutic agent, e.g., etoposide, carboplatin, cisplatin, irinotecan. topotecan, gemcitabine, liposomal SN-38, bendamustine, temozolomide, belotecan, NK012, FR901228, flavopiridol); tyrosine kinase inhibitor (e.g., EGFR inhibitor (e.g., erlotinib, gefitinib, cetuximab, panitumumab); multikinase inhibitor (e.g., sorafenib, sunitinib); VEGF inhibitor (e.g., bevacizumab, vandetanib); cancer vaccine (e.g., GVAX); Bcl-2 inhibitor (e.g., oblimersen sodium, ABT-263); proteasome inhibitor (e.g., bortezomib (Velcade), NPI-0052), paclitaxel or a paclitaxel agent; docetaxel; IGF-1 receptor inhibitor (e.g., AMG 479); HGF/SF inhibitor (e.g., AMG 102, MK-0646); chloroquine; Aurora kinase inhibitor (e.g., MLN8237); radioimmunotherapy (e.g., TF2); HSP90 inhibitor (e.g., tanespimycin, STA-9090), mTOR inhibitor (e.g., everolimus); Ep-CAM-/CD3-bispecific antibody (e.g., MT110); CK-2 inhibitor (e.g., CX-4945); HDAC inhibitor (e.g., belinostat); S MO antagonist (e.g., BMS 833923); peptide cancer vaccine, and radiation therapy (e.g., intensity-modulated radiation therapy (IMRT), hypofractionated radiotherapy, hypoxia-guided radiotherapy), surgery, and combinations thereof.

An example of additional agents for use with an anti-TM4SF1 antibody or antigen binding fragment thereof or an ADC containing such an antibody, in combination with an immunotherapeutic agent, for treatment of non-small cell lung cancer includes, but is not limited to, a chemotherapeutic agent, e.g., vinorelbine, cisplatin, docetaxel, pemetrexed disodium, etoposide, gemcitabine, carboplatin, liposomal SN-38, TLK286, temozolomide, topotecan, pemetrexed disodium, azacitidine, irinotecan, tegafur-gimeracil-oteracil potassium, sapacitabine); tyrosine kinase inhibitor (eg., EGFR inhibitor (e.g., erlotinib, gefitinib, cetuximab, panitumumab, necitumumab, PF-00299804, nimotuzumab, R05083945). MET inhibitor (e.g., PF-02341066, ARQ 197), PDK kinase inhibitor (e.g., XL147, GDC-0941), Raf/MEK dual kinase inhibitor (e.g., R05126766), PI3K/mTOR dual kinase inhibitor (e.g., XL765), SRC inhibitor (e.g., dasatinib), dual inhibitor (e.g., BTBW 2992, GSKI 363089, ZD6474, ALD0530, AG-013736, lapatinib, MEHD7945A, linifanib), multikinase inhibitor (e.g., sorafemb, sunitinib, pazopanib, AMG 706, XL184, MGCD265, BMS-690514, R935788), VEGF inhibitor (e.g., endostar, endostatin, bevacizumab, cediraniib, BIBF 1120, axitinib, tivozaiib, AZD2171), cancer vaccine (e.g., BLP25 liposome vaccine, GVAX, recombinant DNA and adenovirus expressing L523S protein), Bcl-2 inhibitor (e.g. oblimersen sodium), proteasome inhibitor (e.g., bortezomib, carfilzomib, NPI-0052, MLN9708), paclitaxel or a paclitaxel agent, docetaxel, IGF-1 receptor inhibitor (e.g., cixutumumab, MK-0646, OST 906, CP-751,871, B11E022), hydroxychloroquine, HSP90 inhibitor (e.g., tanespirmycin, STA-9090, AUY922, XL888), mTOR inhibitor (e.g., everolimus, temsirolimus, ridaforolimus), Ep-CAM-/CD3-bispecific antibody (e.g., MT110), CK-2 inhibitor (e.g., CX-4945), HDAC inhibitor (e.g., MS 275, LEH589, vorinostat, valproic acid, FR901228), DHFR inhibitor (e.g., pralatrexate), retinoid (e.g., bexarotene, tretinoin), antibody-drug conjugate (e.g., SGN-15), bisphosphonate (e.g., zoledronic acid), cancer vaccine (e.g., belagenpumatucel-L), low molecular weight heparin (LMWH) (e.g., tinzaparin, enoxaparin), GSK1572932A, melatonin, talactoferrin, dimesna, topoisomerase inhibitor (e.g., amrubicin, etoposide, karenitecin), nelfinavir, cilengitide, ErbB3 inhibitor (e.g., MM-121, U3-1287), survivin inhibitor (e.g., YM155, LY2181308), eribulin mesylate, COX-2 inhibitor (e.g., celecoxib), pegfilgrastim, Polo-like kinase I inhibitor (e.g., BT 6727), TRAIL receptor 2 (TR-2) agonist (eg., CS-1008), CNGRC peptide-TNF alpha conjugate, dichloroacetate (DC A), HGF inhibitor (e.g., SCH 900105), SAR240550, PPAR-gamma agonist (e.g., CS-7017), gamma-secretase inhibitor (e.g., R04929097), epigenetic therapy (e.g., 5-azacitidine), nitroglycerin, MEK inhibitor (e.g., AZD6244), cyclin-dependent kinase inhibitor (e.g., UCN-01), cholesterol-Fusl, antitubulin agent (e.g., E7389), farnesyl-OH-transferase inhibitor (e.g., lonafarnib), immunotoxin (e.g., BB-10901, SSI (dsFv) PE38), fondaparinux, vascular-disrupting agent (e.g., AVE8062), PD-Li inhibitor (e.g., MDX-1105, MDX-1106), beta-glucan, NGR-hTNF, EMD 521873, MEK inhibitor (e.g., GSKI 120212), epothilone analog (e.g., ixabepilone), kinesin-spindle inhibitor (e.g., 4SC-205), telomere targeting agent (e.g., KML-001), P70 pathway inhibitor (e.g., LY2584702), AKT inhibitor (e.g., MIK-2206), angiogenesis inhibitor (e.g., lenalidomide), Notch signaling inhibitor (e.g., OMP-21 M18), radiation therapy, surgery, and combinations thereof. An example of suitable therapeutics for use in combination for treatment of ovarian cancer includes, but is not limited to, a chemotherapeutic agent (e.g., paclitaxel or a pachltaxel agent; docetaxel; carboplatin; gemcitabine; doxorubicin; topotecan; cisplatin; irinotecan, TLK286, ifosfaride, olaparib, oxaliplatin, melphalan, pemetrexed disodiun SJG-136, cyclophosphamide, etoposide, decitabine); ghrelin antagonist (e.g., AEZS-130), immunotherapy (e.g., APC8024, oregovomab, OPT-821), tyrosine kinase inhibitor (e.g., EGFR inhibitor (e.g., erlotinib), dual inhibitor (e.g., E7080), muitikinase inhibitor (e.g., AZD0530, JI-101, sorafenib, sunitinib, pazopanib), ON 01910.Na), VEGF inhibitor (e.g., bevacizumab, BIBF 1120, cediranib, AZD27I1), PDGFR inhibitor (e.g., IMC-3G3), paclitaxel, topoisonrerase inhibitor (e.g., kareniecin, Trinotecan), HDAC inhibitor (e.g., valproate, vorinostat), folate receptor inhibitor (e.g., farletuzumab), angiopoietin inhibitor (e.g., AMG 386), epothilone analog (e.g., ixabepilone), proteasoine inhibitor (e.g., carfilzorib), IGF-1 receptor inhibitor (e.g., OSI 906, AMG 479). PARP inhibitor (e.g., veliparib, AG014699, iniparib, MK-4827), Aurora kinase inhibitor (e.g., MLN8237, ENMD-2076), angiogenesis inhibitor (e.g., lenalidomide), DHFR inhibitor (e.g. pralatrexate), radioimmunotherapeutic agent (e.g., Hu3S193), statin (e.g., lovastatin), topoisomerase I inhibitor (e g., NKTR-102), cancer vaccine (e.g., p53 synthetic long peptides vaccine, autologous OC-DC vaccine), mTOR inhibitor (e.g., temsirolimus, everolimus). BCR/ABL inhibitor (e.g., imatinib), ET-A receptor antagonist (e.g., ZD4054), TRAIL receptor 2 (TR-2) agonist (e.g., CS-1008), HGF/SF inhibitor (e.g., AMG 102), EGEN-001, Polo-like kinase I inhibitor (e.g., BT 6727), gamma-secretase inhibitor (e.g., R04929097), Wee-1 inhibitor (e.g., MK-1775), antitubulin agent (e.g., vinorelbine, E7389), immunotoxin (e.g., denileukin diftitox), SB-485232, vascular-disrupting agent (e.g., AVE8062), integrin inhibitor (e.g., EMD 525797), kinesin-spindle inhibitor (e.g., 4SC-205), revlimid, HER2 inhibitor (e.g., MGAHT22), ErrB3 inhibitor (e.g., MM-121), radiation therapy: and combinations thereof.

An example of additional agents for use with an anti-TM4SF1 antibody or antigen binding fragment thereof or an ADC containing such an antibody, in combination with an immunotherapeutic agent, for treatment of myeloma includes, but is not limited to, a. chemotherapeutic agent, but is not limited to: chemotherapy or other anti-cancer agents (e.g., thalidomide analogs, e.g., lenalidomide), HSCT (Cook, R. (2008) J Manag Care Pharm. 14(7 Suppl): 19-25), an anti-TIM3 antibody (Hallett, W H D et al. (2011)/of American Society for Blood and Marrow Transplantation 17(8): 1133-145), tumor antigen-pulsed dendritic cells, fusions (e.g., electrofusions) of tumor cells and dendritic cells, or vaccination with immunoglobulin idiotype produced by malignant plasma cells (reviewed in Yi, Q. (2009) Cancer J. 15(6):502-10).

An example of additional agents for use with an anti-TM4SF1 antibody or antigen binding fragment thereof or an ADC containing such an antibody, in combination with an immunotherapeutic agent, for treatment of renal cell carcinoma or metastatic renal cell carcinoma includes, but is not limited to, a chemotherapeutic agent, examples of which are provided above.

The anti-PD-1 antibody molecule can be administered in combination with one or more of an immune-based strategy (e.g., interleukin-2 or interferon-a), a targeted agent (e.g., a VEGF inhibitor such as a monoclonal antibody to VEGF, e.g., bevacizumab (Rini, B. I. et al. (2010) J. Clin. Oncol. 28(13):2137-2143)); a VEGF tyrosine kinase inhibitor such as sunitinib, sorafenib, axitinib and pazopanib (reviewed in Pal S. K. et al. (2014) Clin, Advances in Hematology & Oncology 12(2):90-99)); an RNAi inhibitor), or an inhibitor of a downstream mediator of VEGF signaling, e.g., an inhibitor of the mammalian target of rapamycin (mTOR), e.g., everolimus and temsirolimus (Hudes, G. et al. (2007) N. Engl. J. Med. 356(22): 2271-2281, Motzer, R. J. et al. (2008) Lancet 372: 449-456).

An example of additional agents for use with an anti-TM4SF1 antibody or antigen binding fragment thereof or an ADC containing such an antibody, in combination with an immunotherapeutic agent, for treatment of chronic myelogenous leukemia (AML) according to the invention includes, but is not limited to, a chemotherapeutic (e.g., cytarabine, hydroxyurea, clofarabine, melphalan, thiotepa, fludarabine, busulfan, etoposide, cordycepin, pentostatin, capecitabine, azacitidine, cyclophosphamide, cladribine, topotecan), tyrosine kinase inhibitor (e.g., BCR/ABL inhibitor (e.g., imatinib, nilotinib), ON 01910.Na, dual inhibitor (e.g., dasatinib, bosutinib), multikinase inhibitor (e.g., DCC-2036, ponatinib, sorafenib, sunitinib. RGB-286638)), interferon alfa, steroids, apoptotic agent (e.g., omacetaxine mepesuccinat), immunotherapy (e.g., allogeneic CD4+ memory Th1-like T cells/microparticle-bound anti-CD3/anti-CD28, autologous cytokine induced killer cells (CIK), AHN-12), CD52 targeting agent (e.g., alemtuzumab), HSP90 inhibitor (e.g., tanespimycin, STA-9090, AUY922, XL888), mTOR inhibitor (e.g., everolimus), SMO antagonist (e.g., BMS 833923), ribonucleotide reductase inhibitor (e.g., 3-AP), JAK-2 inhibitor (e.g., INCB018424). Hydroxychloroquine, retinoid (e.g., fenretinide), cyclin-dependent kinase inhibitor (e.g., UCN-01), HDAC inhibitor (e.g., belinostat, vorinostat, JNJ-26481585), PARP inhibitor (e.g., veliparib), MDM2 antagonist (e.g., R05045337), Aurora B kinase inhibitor (e.g., T AK-901), radioimmunotherapy (e g., actinium-225-labeled anti-CD33 antibody Hui 1195), Hedgehog inhibitor (e.g., PF-04449913), STAT3 inhibitor (e.g., OPB-31121), KB004, cancer vaccine (e.g., AG858), bone marrow transplantation, stein cell transplantation, radiation therapy, and combinations thereof.

An example of additional agents for use with an anti-TM4SF1 antibody or antigen binding fragment thereof or an ADC containing such an antibody, in combination with an immunotherapeutic agent, for treatment of chronic lymphocytic leukemia (CLL) includes, but is not limited to, a chemotherapeutic agent (e.g., fludarabine, cyclophosphamide, doxorubicin, vincristine, chlorambucil, bendamustine, chlorambucil, busulfan, gencitabine, melphalan, pentostartin, mitoxantrone, 5-azacytidine, pernetrexed disodiun), tyrosine kinase inhibitor (e.g., EGFR inhibitor (e.g., erlotinib), BTK inhibitor (eg PCI-32765), multikinase inhibitor (e.g., MGCD265, RGB-286638), CD-20 targeting agent (e.g., rituximab, ofatumumab, RO5072759, LFB-R603), CD52 targeting agent (e.g., alerntuzuab), prednisolone, darbepoetin alfa, lenalidomide, Bel-2 inhibitor (e.g., ABT-263), immunotherapy (e.g., allogeneic CD4+ memory Th1-like T cells/microparticle-bound anti-CD3/anti-CD28, autologous cytokine induced killer cells (CIK)), HDAC inhibitor (e.g., vorinostat, valproic acid, LBH589, JNJ-26481585, AR-42), XAP inhibitor (e.g., AEG35156), CD-74 targeting agent (e.g., milatuzumab), nTOR inhibitor (e.g., everolimus), AT-101, immunotoxin (e.g., CAT-8015, anti-Tac(Fv)-PE38 (LMB-2)), CD37 targeting agent (e.g., T1RU-016), radioimmunotherapy (e.g., 131-tositumomab), hydroxychloroquine, perifosine, SRC inhibitor (e.g., dasatinib), thalidomide, PI3K delta inhibitor (e.g., CAL-101), retinoid (e.g., fenretinide), MDM2 antagonist (e.g., R05045337), plerixafor, Aurora kinase inhibitor (e.g., MLN8237, TAK-90l), proteasome inhibitor (e.g., bortezoinib), CD-19 targeting agent (e.g., MEDI-551, MOR208), MEK inhibitor (e.g., ABT-348), JAK-2 inhibitor (e.g., INCB018424), hypoxia-activated prodrug (e.g., TH.-302), paclitaxel or a paclitaxel agent, HSP90 inhibitor, AKT inhibitor (e.g., MK2206), HMG-CoA inhibitor (e.g., simvastatin), GNKG0186, radiation therapy, bone marrow transplantation, stem cell transplantation, and a combination thereof.

An example of additional agents for use with an anti-TM4SF1 antibody or antigen binding fragment thereof or an ADC containing such an antibody, in combination with an immunotherapeutic agent, for treatment of acute lymphocytic leukemia (ALL) includes, but is not limited to, a chemotherapeutic agent (e.g., prednisolone, dexamethasone, vincristine, asparaginase, daunorubicin, cyclophosphamide, cytarabine, etoposide, thioguanine, mercaptopurine, clofarabine, liposomal ansamycin, busulfan, etoposide, capecitabine, decitabine, azacitidine, topotecan, temozolomide), tyrosine kinase inhibitor (e.g., BCR/ABL inhibitor (e.g., imatinib, nilotinib), ON 01910. Na, multikinase inhibitor (e.g., sorafenib)), CD-20 targeting agent (e.g., rituximab), CD52 targeting agent (e.g., alemtuzumab), HSP90 inhibitor (e.g., STA-9090), mTOR inhibitor (e.g., everolimus, rapamycin), JAK-2 inhibitor (e.g., INCB 018424), HER2/neu receptor inhibitor (e.g., trastuzumab), proteasome inhibitor (e.g., bortezomib), methotrexate, asparaginase, CD-22 targeting agent (e.g., epratuzumab, inotuzumab), immunotherapy (e.g., autologous cytokine induced killer cells (CIK), AHN-12), blinatumomab, cyclin-dependent kinase inhibitor (e.g., UCN-01), CD45 targeting agent (e.g., BC8), MDM2 antagonist (e.g., R05045337), immunotoxin (e.g., CAT-8015, DT2219ARL), HDAC inhibitor (e.g. JNJ-26481585), JVRS-100, paclitaxel or a paclitaxel agent, STAT3 inhibitor (e.g., OPB-31121), PARP inhibitor (e.g., veliparib), EZN-2285, radiation therapy, steroid, bone marrow transplantation, stern cell transplantation, or a combination thereof.

An example of additional agents for use with an ant-TM4SF1 antibody or antigen binding fragment thereof or an ADC containing such an antibody, in combination with an immunotherapeutic agent, for treatment of acute myeloid leukemia (AML) includes, but is not limited to, a chemotherapeutic agent (e.g., cytarabine, daunorubicin, idarubicin, clofarabine, decitabine, vosaroxin, azacitidine, clofarabine, ribavirin, CPX-351, treosulfan, elacytarabine, azacitidine), tyrosine kinase inhibitor (e.g., BCR/ABL inhibitor (e.g., imatinib, nilotinib), ON 01910.Na., multikinase inhibitor (e.g., midostaurin, SU 11248, quizartinib, soratinib)), immunotoxin (e.g., gemtuzumab ozogamicin). DT3881L3 fusion protein, HDAC inhibitor (e.g., vorinostat, LBH589), plerixafor, mTOR inhibitor (e.g., everolimus), SRC inhibitor (e.g., dasatinib), HSP90 inhibitor (e.g., STA-9090), retinoid (e.g., bexarotene, Aurora kinase inhibitor (e.g., BI 811283), JAK-2 inhibitor (e.g., INCB018424), Polo-like kinase inhibitor (e.g., BT 6727), cenersen, CD45 targeting agent (e.g., BC8), cyclin-dependent kinase inhibitor (e.g., UCN-01), MDM2 antagonist (e.g., R05045337), mTOR inhibitor (e.g., everolimus), LY573636-sodium, ZRx-101, MLN4924, lenalidomide, immunotherapy (e.g., AHN-12), histamine dihydrochloride, radiation therapy, bone marrow transplantation, stem cell transplantation, and a combination thereof.

An example of additional agents for use with an anti-TM4SF1 antibody or antigen binding fragment thereof or an ADC containing such an antibody, in combination with an immunotherapeutic agent, for treatment of multiple myeloma (MM) includes, but is not limited to, a chemotherapeutic agent (e.g., melphalan, amifostine, cyclophosphamide, doxorubicin, clofarabine, bendamustine, fludarabine, adriamycin, SyB L-0501), thalidomide, lenalidomide, dexamethasone, prednisone, pomalidomide, proteasome inhibitor (e.g., bortezomib, carfilzomib. MLN9708), cancer vaccine (e.g., GVAX), CD-40 targeting agent (e.g., SGN-40, CHIR-12.12), perifosine, zoledronic acid, Immunotherapy (e.g., MAGE-A3, NY-ESO-1, HuMax-CD38), HDAC inhibitor (e.g., vorinostat, LBH589, AR-42), aplidin, cycline-dependent kinase inhibitor (e.g., PD-0332991, dinaciclib), arsenic trioxide, CB3304, HSP90 inhibitor (e.g., KW-2478), tyrosine kinase inhibitor (e.g., EGFR inhibitor (e.g., cetuximab), multikinase inhibitor (e.g. AT9283)), VEGF inhibitor (e.g., bevacizumab), plerixafor, MEK inhibitor (e.g., AZD6244), IPH2101, atorvastatin, immunotoxin (e.g., BB-10901), NPI-0052, radioimmunotherapeutic (e.g., yttrium Y 90 ibritumomab tiuxetan), STAT3inhibitor (e.g., OPB-31121), MLN4924, Aurora kinase inhibitor (e.g., ENMD-2076), IMGN901, ACE-041, CK-2 inhibitor (e.g., CX-4945), radiation therapy, bone marrow transplantation, stem cell transplantation, and a combination thereof.

An example of additional agents for use with an anti-TM4SF1 antibody or antigen binding fragment thereof or an ADC containing such an antibody, in combination with an immunotherapeutic agent, for treatment of prostate cancer, includes, but is not limited to, a chemotherapeutic agent (e.g., docetaxel, carboplatin, fludarabine), abiraterone, hormonal therapy (e.g., flutamide, bicalutamide, nilutamide, cyproterone acetate, ketoconazole, aminoglutethimide, abarelix, degarelix, leuprolide, goserelin, triptorelin, buserelin), tyrosine kinase inhibitor (e.g., dual kinase inhibitor (e.g., lapatanib), multikinase inhibitor (e.g. sorafenib, sunitinib)), VEGF inhibitor (e.g., bevacizumab), TAK-700, cancer vaccine (e.g., BPX-101, PEP223), lenalidomide, TOK-001, IGF-1 receptor inhibitor (e.g., cixutumumab), TRC105, Aurora A kinase inhibitor (e.g., MLN8237), proteasome inhibitor (e.g., bortezomib). OGX-011, radioimmunotherapy (e.g., HuJ591-GS), HDAC inhibitor (e.g., valproic acid, SB939, LBH589), hydroxychloroquine, mTOR inhibitor (e.g., everolimus), dovitinib lactate, diindolylmethane, efavirenz, OGX-427, genistein, IMC-3G3, bafetinib, CP-675,206, radiation therapy, surgery, or a combination thereof.

### VII. Pharmaceutical Compositions

The anti-TM4SF1 antibodies, the ADCs, the immunotherapeutic agents of this disclosure, can, in some embodiments, be included in a single or separate compositions (e.g., pharmaceutical compositions). The pharmaceutical compositions of the disclosure may further include a pharmaceutically acceptable carrier, excipient, or diluent.

The term “pharmaceutical composition” as used herein refers to a composition containing a TM4SF1 binding protein described herein formulated with a pharmaceutically acceptable carrier, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The term “pharmaceutically acceptable carrier” as used herein refers to a carrier which is physiologically acceptable to a treated mammal (e.g., a human) while retaining the therapeutic properties of the protein with which it is administered. One exemplary pharmaceutically acceptable carrier is physiological saline. Other physiologically acceptable carriers and their formulations are known to one skilled in the art and described, for example, in Remington's Pharmaceutical Sciences (18th edition, A. Gennaro, 1990, Mack Publishing Company, Easton, PA), incorporated herein by reference.

The pharmaceutical compositions are, in some embodiments, prepared as solutions, dispersions in glycerol, liquid polyethylene glycols, and any combinations thereof in oils, in solid dosage forms, as inhalable dosage forms, as intranasal dosage forms, as liposomal formulations, dosage forms comprising nanoparticles, dosage forms comprising microparticles, polymeric dosage forms, or any combinations thereof.

A pharmaceutically acceptable excipient is, in some examples, an excipient described in the Handbook of Pharmaceutical Excipients, American Pharmaceutical Association (1986). Non-limiting examples of suitable excipients include a buffering agent, a preservative, a stabilizer, a binder, a compaction agent, a lubricant, a chelator, a dispersion enhancer, a disintegration agent, a flavoring agent, a sweetener, a coloring agent.

In some embodiments an excipient is a buffering agent. Non-limiting examples of suitable buffering agents include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof is used, in some embodiments, in a pharmaceutical composition of the present disclosure.

In some embodiments an excipient comprises a preservative. Non-limiting examples of suitable preservatives include antioxidants, such as alpha-tocopherol and ascorbate, and antimicrobials, such as parabens, chlorobutanol, and phenol. In some examples, antioxidants further include but are not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances preservatives include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some embodiments a pharmaceutical composition as described herein comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof. The binders used in a pharmaceutical formulation are, in some examples, selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or any combinations thereof.

In some embodiments a pharmaceutical composition as described herein comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that are used in a pharmaceutical formulation, in some embodiments, are be selected from metallic stearates (such as magnesium stearate, calcium stearate, aluminium stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments a pharmaceutical formulation comprises a dispersion enhancer as an excipient. Non-limiting examples of suitable dispersants include, in some examples, starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments a pharmaceutical composition as described herein comprises a disintegrant as an excipient. In some embodiments a disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments a disintegrant is an effervescent disintegrant. Non-limiting examples of suitable effervescent disintegrants include sodium bicarbonate in combination with citric acid, and sodium bicarbonate in combination with tartaric acid.

In some embodiments an excipient comprises a flavoring agent. Flavoring agents incorporated into an outer layer are, in some examples, chosen from synthetic flavor oils and flavoring aromatics; natural oils; extracts from plants, leaves, flowers, and fruits; and combinations thereof. In some embodiments a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments an excipient comprises a sweetener. Non-limiting examples of suitable sweeteners include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like.

In some instances, a pharmaceutical composition as described herein comprises a coloring agent. Non-limiting examples of suitable color agents include food, drug and cosmetic colors (FD&C), drug and cosmetic colors (D&C), and external drug and cosmetic colors (Ext. D&C). Coloring agents can be used as dyes or their corresponding lakes.

In some instances, a pharmaceutical composition as described herein comprises a chelator. In some cases, a chelator is a fungicidal chelator. Examples include, but are not limited to: ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA; trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-tetraacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); 0,0′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephosphoric acid); 7,19,30-trioxa-1,4,10,13,16,22,27,33-octaazabicyclo[11,11,11] pentatriacontane hexahydrobromide; or triethylenetetramine-N,N,N′,N″,N′″,N′″-hexaacetic acid.

Also contemplated are compositions that include one or more other antimicrobial or antifungal agents, for example, polyenes such as amphotericin B, amphotericin B lipid complex (ABCD), liposomal amphotericin B (L-AMB), and liposomal nystatin, azoles and triazoles such as voriconazole, fluconazole, ketoconazole, itraconazole, pozaconazole and the like; glucan synthase inhibitors such as caspofungin, micafungin (FK463), and V-echinocandin (LY303366); griseofulvin; allylamines such as terbinafine; flucytosine or other antifungal agents, including those described herein. In addition, it is contemplated that a peptide can be combined with topical antifungal agents such as ciclopirox olamine, haloprogin, tolnaftate, undecylenate, topical nysatin, amorolfine, butenafine, naftifine, terbinafine, and other topical agents. In some instances, a pharmaceutical composition comprises an additional agent. In some cases, an additional agent is present in a therapeutically effective amount in a pharmaceutical composition.

Under ordinary conditions of storage and use, the pharmaceutical compositions as described herein comprise a preservative to prevent the growth of microorganisms. In certain examples, the pharmaceutical compositions as described herein do not comprise a preservative. The pharmaceutical forms suitable for injectable use include sterile aqueous solutions or dispersions and sterile powders for the extemporaneous preparation of sterile injectable solutions or dispersions. The pharmaceutical compositions comprise a carrier which is a solvent or a dispersion medium containing, for example, water, ethanol, polyol (e.g., glycerol, propylene glycol, and liquid polyethylene glycol, and the like), and/or vegetable oils, or any combinations thereof. Proper fluidity is maintained, for example, by the use of a coating, such as lecithin, by the maintenance of the required particle size in the case of dispersion and by the use of surfactants. The prevention of the action of microorganisms is brought about by various antibacterial and antifungal agents, for example, parabens, chlorobutanol, phenol, sorbic acid, thimerosal, and the like. In many cases, isotonic agents are included, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the liquid dosage form is suitably buffered if necessary and the liquid diluent rendered isotonic with sufficient saline or glucose. The liquid dosage forms are especially suitable for intravenous, intramuscular, subcutaneous, intratumoral, and intraperitoneal administration. In this connection, sterile aqueous media that can be employed will be known to those of skill in the art in light of the present disclosure. For example, one dosage is dissolved, in certain cases, in 1 mL to 20 mL of isotonic NaCl solution and either added to 100 mL to 1000 mL of a fluid, e.g., sodium-bicarbonate buffered saline, or injected at the proposed site of infusion.

In certain embodiments, sterile injectable solutions is prepared by incorporating a immunotherapy agent, in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. The compositions disclosed herein are, in some instances, formulated in a neutral or salt form. Pharmaceutically-acceptable salts include, for example, the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups are, in some cases, derived from inorganic bases such as, for example, sodium, potassium, ammonium, calcium, or ferric hydroxides, and such organic bases as isopropylamine, trimethylamine, histidine, procaine and the like. Upon formulation, the pharmaceutical compositions are administered, in some embodiments, in a manner compatible with the dosage formulation and in such amount as is therapeutically effective.

In certain embodiments, a pharmaceutical composition of this disclosure comprises a pharmaceutically acceptable carrier. “Pharmaceutically acceptable,” as used herein, includes any carrier which does not interfere with the effectiveness of the biological activity of the active ingredients and/or that is not toxic to the patient to whom it is administered. Non-limiting examples of suitable pharmaceutical carriers include phosphate buffered saline solutions, water, emulsions, such as oil/water emulsions, various types of wetting agents and sterile solutions. Additional non-limiting examples of pharmaceutically compatible carriers can include gels, bioadsorbable matrix materials, implantation elements containing the immunotherapeutic agents or any other suitable vehicle, delivery or dispensing means or material. Such carriers are formulated, for example, by conventional methods and administered to the subject at an effective amount.

### VIII. Kits

In some embodiments, the disclosure provides kits that include a composition (e.g., a pharmaceutical composition) of the disclosure (e.g., a composition including an anti-TM4SF1 antibody or antigen binding fragment thereof, or an ADC containing such an antibody, and an immunotherapeutic agent). The kits include instructions to allow a clinician (e.g., a physician or nurse) to administer the composition contained therein to a subject to treat a disorder associated with pathological angiogenesis (e.g., cancer).

In certain embodiments, the kits include a package of a single-dose pharmaceutical composition(s) containing an effective amount of an antibody of the disclosure. Optionally, instruments or devices necessary for administering the pharmaceutical composition(s) may be included in the kits. For instance, a kit of this disclosure may provide one or more pre-filled syringes containing an effective amount of a vaccine, vector, stabilized trimer, or optimized viral polypeptide of the disclosure. Furthermore, the kits may also include additional components such as instructions regarding administration schedules for a subject having a disorder associated with pathological angiogenesis (e.g., cancer) to use the pharmaceutical composition(s) containing a TM4SF1 binding protein or polynucleotide of the disclosure.

### EXAMPLES

### Example 1: Characterization of Anti-TM4SF1 Antibodies

### In Vitro Binding to Cell Lines

Antigen binding of an exemplary anti-TM4SF1 antibody 1 which included a human IgG1 constant region containing the YTEC mutations was assessed using a cell-based flow cytometry assay on different murine cell lines: MS1 (murine pancreatic islet endothelial derived cell line), CT26 (murine colon carcinoma derived cell line), and B16F10 (murine melanoma derived cell line).

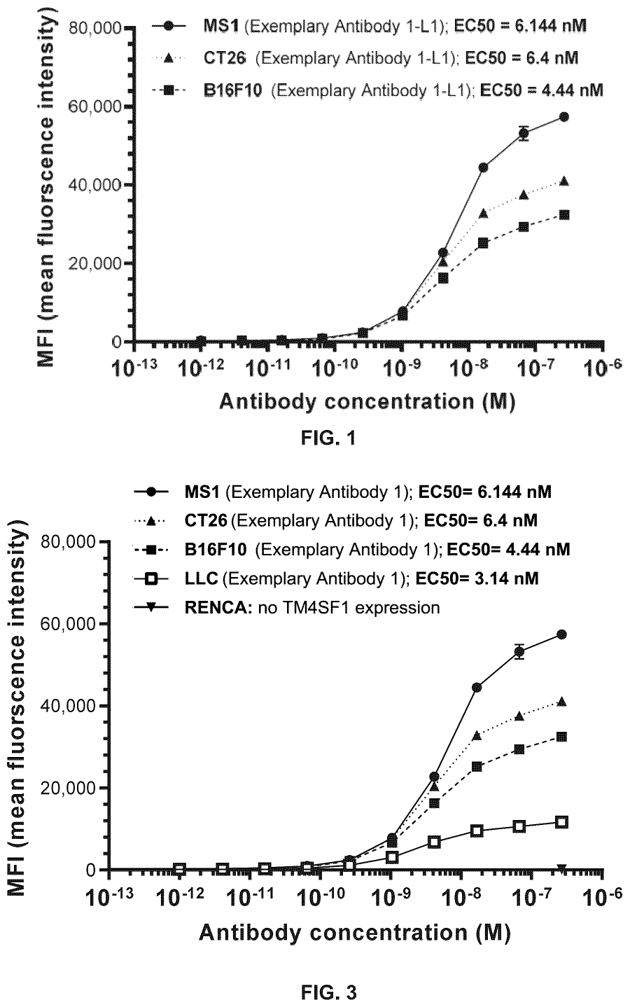

The EC₅₀ values for binding are show in the legend of FIG. 1. Maximal binding of Exemplary Antibody 1 indicated that expression of TM4SF1 is greatest in MS1, followed by CT26, followed by B16F10.

### In Vitro Cell Proliferation Inhibition Assay

The effects of antibody drug-conjugates (drug to antibody ratio (DAR) of about 2) containing a drug (maytansine) and either Exemplary Antibody 1 or Exemplary Antibody 2 were respectively assessed in cultured mouse (MS1, CT26, B16F10) and human (MiaPaCa2, A549, SKOV3, and HUVEC) cells. Both Exemplary Antibody 1 and Exemplary Antibody 2 contains human IgG1 constant region with YTEC mutation. Cells were treated incubated for 5 days with the antibody drug conjugate before assessing cell viability on day 5.

TABLE 1 shows the representative EC50 values from cell proliferation inhibition activities of Exemplary Antibody 1 conjugated to a maytansine payload via linker 1 (L1) (Exemplary Antibody 1-L1), Exemplary Antibody 2 conjugated to a maytansine payload via linker 1 (L1) (Exemplary Antibody 2-L1), or via other linkers, on different cell lines. Collectively: MS1 (0.05 nM), B16F10 (0.12 nM) and CT26 (no killing) via Exemplary Antibody 1-L1, and MiaPaCa2 (0.04 nM), A549 (0.06 nM), SKOV3 (0.81 nM), and HUVEC (0.10 nM) via Exemplary Antibody 2-L1. Although TM4SF1 is highly expressed in CT26 cells, the cell was resistant to Exemplary Antibody 1-L1 antibody mediated killing.

Table 1: Exemplary Antibody 1 target cells from mouse origin only. Conversely, Exemplary Antibody 2 only targets cells from human origin. Variants of the exemplary antibodies were generated to produce different antibodies that are either capped and naked antibody or conjugated with maytansine payload via seven different linkers. Exemplary Antibody 1-L1 ADC was subsequently used for all immune-oncology (10) related studies in mice.

TABLE 1

| | In vitro cell proliferation inhibition activity (EC₅₀; nM) | | | | | | |
|---|---|---|---|---|---|---|---|
| | Exemplary Antibody 1 | | | Exemplary Antibody 2 | | | |
| | (targets mouse cells) | | | (targets human cells) | | | |
| | | | Endothelial | | | | Endothelial |
| | Tumor cell | | cell | Tumor cells | | | cell |
| Linker | B16F10 | CT26 | MS1 | MiaPaca2 | A549 | SKOV3 | HUVEC |
| Capped | — | — | — | — | — | — | — |
| Linker 1 | 0.12 | — | 0.05 | 0.04 | 0.06 | 0.81 | 0.10 |
| (L1) | | | | | | | |
| Linker 2 | 3.91 | | 0.07 | 0.03 | 0.07 | 1.13 | 0.10 |
| Linker 3 | 6.50 | | 0.14 | 0.05 | 0.19 | 23.48 | 0.75 |
| Linker 4 | 250.70 | | 0.29 | 0.03 | 0.10 | 1.39 | 0.15 |
| Linker 5 | 1.22 | | 0.15 | 0.03 | 0.06 | 0.15 | 0.09 |
| Linker 6 | — | | 192.60 | 0.03 | 0.04 | 0.05 | 0.11 |
| Linker 7 | 146.50 | | 0.30 | 0.04 | not | not | 0.31 |
| | | | | | tested | tested | |

Symbol “—”: no killing activity

Data presented above represents EC50 from seven different linkers that are conjugated to maytansine payload.
Linker 1: PEG4-Maytansine; BA-PEG4-N-Methyl-Alanine-Maytansine
Linker 2: PEG4Ahx-maytansine; BA-PEG4-Ahx-N-Methyl-Alanine-Maytansine
Linker 3: Glc-Maytansine; BA-Glucuronide-N-Methyl-Alanine-Maytansine
Linker 4: Glu(tb)PEG4-Glc-maytansine; BA-Glu-(Ot-butyl)-Glucoronide-N-Methyl-Glycine-N-Methyl-Alanine-Maytansine
Linker 5: Glu-tb-maytansine; BA-PEG4-D-Glu-Ot-butyl-Ahx-N-Methyl-Alanine-Maytansine
Linker 6: GluAhx-maytansine; BA-PEG4-D-Glu-Ahx-N-Methyl-Alanine-Maytansine
Linker 7: PEG4Glu-maytansine; BA-PEG4-D-Glu-N-Methyl-Alanine-Maytansine

### Example 2: Tumor Regression Using an Anti-TM4SF1 ADC and Anti-Mouse PD-1 Antibody

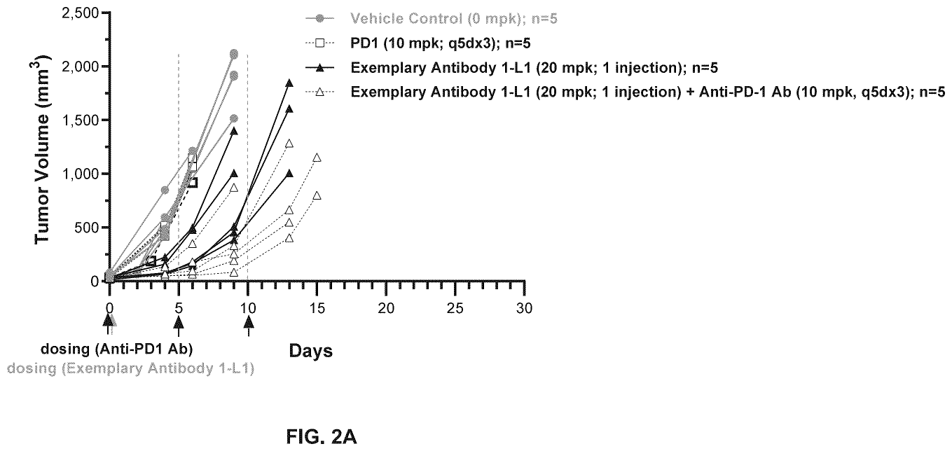

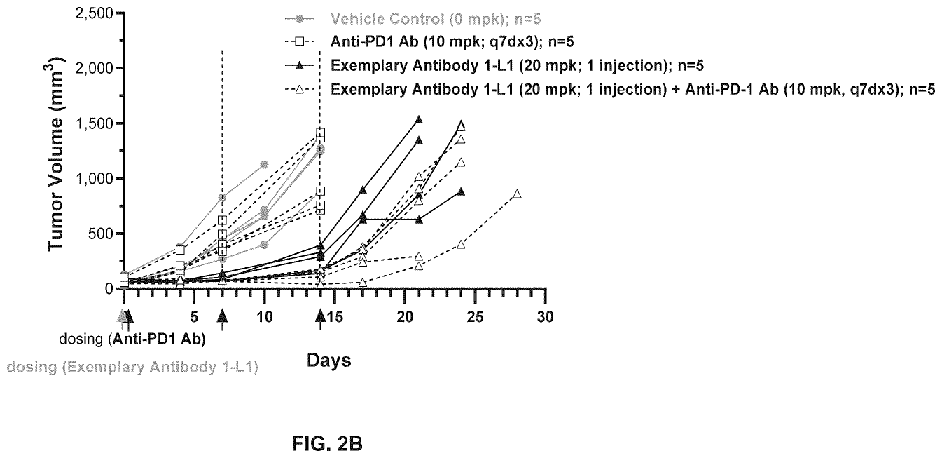

For this study, cell-derived syngeneic mouse models were used to assess the effects of a treatment comprising an anti-TM4SF1 ADC (Exemplary Antibody 1-L1) and an anti-PD-1 antibody combination. The syngeneic models assessed were B16F10 derived tumor in a C57BL/6 mouse (FIG. 2A) and a CT26 derived tumor model in a BALB/c mouse (FIG. 2B).

In FIG. 2A, a B16F10 C57BL/6 syngeneic model was separated into multiple treatment groups to assess tumor volume (mm³) in the presence of different treatments: 1) Vehicle (Control), 2) anti-mouse PD-1 antibodies (BioXcell, clone RMP1-14, 10 mg/kg), 3) Exemplary Antibody 1-L1 (20 mg/kg), and 4) Exemplary Antibody 1-L1 (20 mg/kg)+ anti-mouse PD1 (10 mg/kg). Exemplary Antibody 1-L1 was administered once at the start of the study. The anti-mouse PD1 antibody was administered three times, at days 0, 5, and 10 after treatment. Over the course of the treatment, tumor volumes were measured on days 4, 6, 9, 13, and 15. Within 10 days, treatments using the anti-mouse PD-1 antibody alone (Treatment 2), did not inhibit tumor growth compared to the Vehicle control (Treatment 1). Treatments that contained 20 mg/kg of the Exemplary Antibody 1-L1 retarded tumor growth (Treatment 3) and showed synergistic tumor regression activity in the presence of anti-mouse PD-1 antibodies (Treatment 4).

In FIG. 2B, a CT26 BALB/c syngeneic model was separated into multiple treatment groups to assess tumor volume (mm³) in the presence of different treatments: 1) Vehicle (Control), 2) anti-mouse PD-1 antibody (BioXcell, clone RMP1-14, 10 mg/kg), 3) Exemplary Antibody 1-L1 (20 mg/kg), and 4) Exemplary Antibody Exemplary Antibody 1-L1 (20 mg/kg)+ anti-mouse PD-1 antibody (10 mg/kg). Similar to FIG. 2A, the anti-TM4SF1 antibody treatments were administered once at the start of treatment, whereas the anti-mouse PD-1 antibody injections were administered 3 times at Days 0, 7, and 14. Treatment with only the anti-mouse PD-1 antibody resulted in tumors that progressed similarly to the Vehicle control, and the animals were sacrificed by day 14. Treatment of mice using 20 mg/kg of the Exemplary Antibody 1-L1 improved the tumor regression potential than the Vehicle control, however addition of the administration of the anti-mouse PD-1 antibody further increased the inhibition of tumor growth as far as 28 days after the start of the treatment.

FIGS. 2A and 2B show that the tested ADC (Exemplary Antibody 1-L1) has a synergistic tumor regression effect with anti-mouse PD-1 antibody in both B16F10 (C57BL/6 mice) and CT26 (BALB/c mice) syngeneic tumor models respectively. This synergistic tumor growth retardation outcome indicates tumor vessel targeting via Exemplary Antibody 1-L1 likely activates immune response in tumors by facilitating leukocyte infiltration to the tumor via tumor vascular wound. This combo approach of tumor vessel targeting along with immune checkpoint blockade approach is expected to provide immense opportunity to turn many different types of solid tumors from immunologically inactive to active state.

Likely due to spindle check point mutations, some tumor cell lines such as CT26 are resistant to maytansine payloads. Therefore, the tumor regression outcome with Exemplary Antibody 1-L1 antibody in CT26 syngeneic tumor (FIG. 2B) is solely via targeting tumor vessels. Such tumor regression was further improved in the presence of anti-mouse PD-1 immune blockade antibody.

### Example 4: Characterization of Anti-TM4SF1 Antibodies

### In Vitro Binding to Cell Lines

Antigen binding of an exemplary anti-TM4SF1 antibody drug conjugate (maytansine) which included a human IgG1 constant region containing the YTEC mutations was assessed using a cell-based flow cytometry assay on different murine cell lines: MS1 (murine pancreatic islet endothelial derived cell line), CT26 (murine colon carcinoma derived cell line), B16F10 (murine melanoma derived cell line), and LLC (murine lung carcinoma). The RENCA cell line was used as a negative control as no expression of TM4SF1 is present.

The EC₅₀ values for binding are shown in the legend of FIG. 3. Maximal binding of Exemplary Antibody 1 indicated that expression of TM4SF1 is greatest in MS1, followed by CT26, followed by B16F10, followed by LLC. Renca does not express TM4SF1. Typically, the binding affinities for antibodies in the Exemplary Antibody 1 group is at 3-7 nM EC50 in cell-based assays

### In Vitro Cell Proliferation Inhibition Assay

The effects of antibody drug-conjugates (drug to antibody ratio (DAR) of about 2) containing a drug (maytansine) and either Exemplary Antibody 1 or Exemplary Antibody 2 were respectively assessed in cultured mouse (MS1, CT26, B16F10) and human (MiaPaCa2, A549, SKOV3, and HUVEC) cells. Both Exemplary Antibody 1 and Exemplary Antibody 2 contains human IgG1 constant region with YTEC mutation. Cells were treated incubated for 4 days with the antibody drug conjugate before assessing cell viability on day 5.

TABLE 3 shows the representative EC50 values from cell proliferation inhibition activities of Exemplary Antibody 1 conjugated to a maytansine payload via linker 1 (L1) (Exemplary Antibody 1-L1), Exemplary Antibody 2 conjugated to a maytansine payload via linker 1 (L1) (Exemplary Antibody 2-L1), or via other linkers, on different cell lines. Collectively: MS1 (0.05 nM), B16F10 (0.12 nM), RENCA (no killing) and CT26 (no killing) via Exemplary Antibody 1-L1, and MiaPaCa2 (0.04 nM), A549 (0.06 nM), SKOV3 (0.81 nM), and HUVEC (0.10 nM) via Exemplary Antibody 2-L1. Although TM4SF1 is highly expressed in CT26 cells, the cell was resistant to Exemplary Antibody 1-L1 antibody mediated killing.

TABLE 3

| | In vitro cell proliferation inhibition activity (EC50; nM) | | | | | | | | |
|---|---|---|---|---|---|---|---|---|---|
| | Exemplary Antibody 1 | | | | | Exemplary Antibody 2 | | | |
| | (targets mouse cells) | | | | | (targets human cells) | | | |
| | | | | | Endothelial | | | | Endothelial |
| | Tumor cell | | | | cell | Tumor cells | | | cell |
| Linker | B16F10 | CT26 | LLC | RENCA | MS1 | MiaPaca2 | A549 | SKOV3 | HUVEC |
| Capped | — | — | | | — | — | — | — | — |
| Linker 1 | 0.12 | — | 98.13 | — | 0.05 | 0.04 | 0.06 | 0.81 | 0.1 |
| (L1) | | | | | | | | | |
| Linker 2 | 3.91 | | | | 0.07 | 0.03 | 0.07 | 1.13 | 0.1 |
| Linker 3 | 6.5 | | | | 0.14 | 0.05 | 0.19 | 23.48 | 0.75 |
| Linker 4 | 250.7 | | | | 0.29 | 0.03 | 0.1 | 1.39 | 0.15 |
| Linker 5 | 1.22 | | | | 0.15 | 0.03 | 0.06 | 0.15 | 0.09 |
| Linker 6 | — | | | | 192.6 | 0.03 | 0.04 | 0.05 | 0.11 |
| Linker 7 | 146.5 | | | | 0.3 | 0.04 | not | not | 0.31 |
| | | | | | | | tested | tested | |

Symbol “—”: no killing activity

Data presented above represents EC50 from seven different linkers that are conjugated to maytansine payload.
Linker 1: PEG4-Maytansine; BA-PEG4-N-Methyl-Alanine-Maytansine
Linker 2: PEG4Ahx-maytansine; BA-PEG4-Ahx-N-Methyl-Alanine-Maytansine
Linker 3: Glc-Maytansine; BA-Glucuronide-N-Methyl-Alanine-Maytansine
Linker 4: Glu(tb)PEG4-Glc-maytansine; BA-Glu-(Ot-butyl)-Glucoronide-N-Methyl-Glycine-N-Methyl-Alanine-Maytansine
Linker 5: Glu-tb-maytansine; BA-PEG4-D-Glu-Ot-butyl-Ahx-N-Methyl-Alanine-Maytansine
Linker 6: GluAhx-maytansine; BA-PEG4-D-Glu-Ahx-N-Methyl-Alanine-Maytansine
Linker 7: PEG4Glu-maytansine; BA-PEG4-D-Glu-N-Methyl-Alanine-Maytansine

### Example 5: Tumor Regression Using an Anti-TM4SF1 ADC and Anti-Mouse CTLA-4 Antibody in a CT26 Syngeneic Model

For this study, a cell-derived syngeneic CT26 mouse model was used to assess the effects of a treatment comprising an anti-TM4SF1 ADC (Exemplary Antibody 1-L1) and/or an anti-mouse CTLA-4 antibody (Clone 9H10; BioXCell) combination.

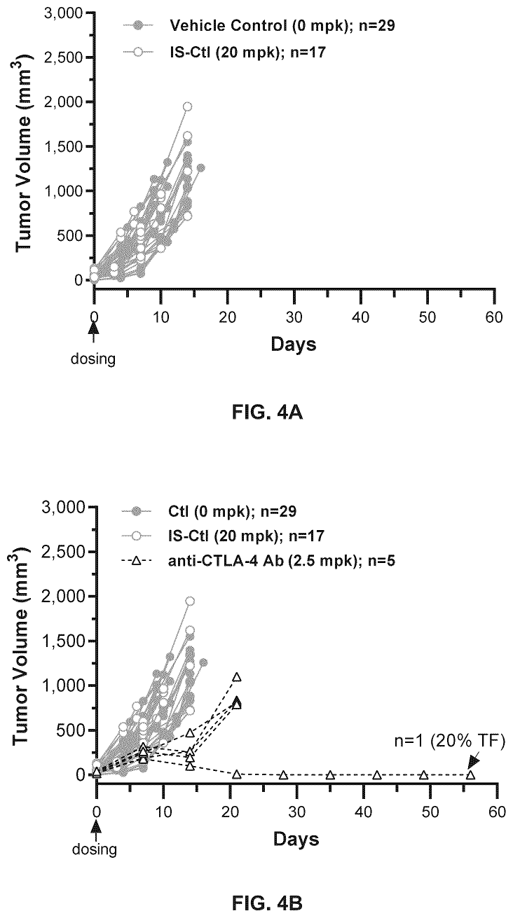

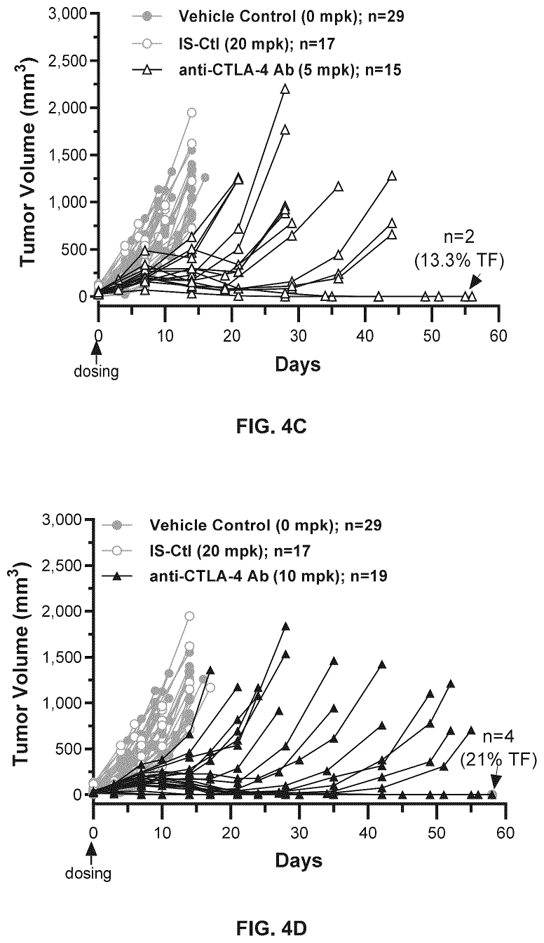

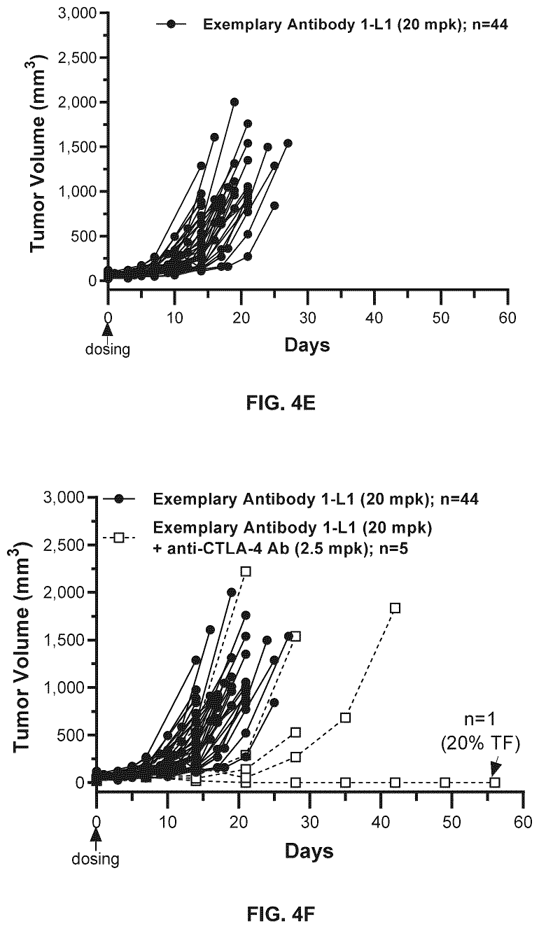

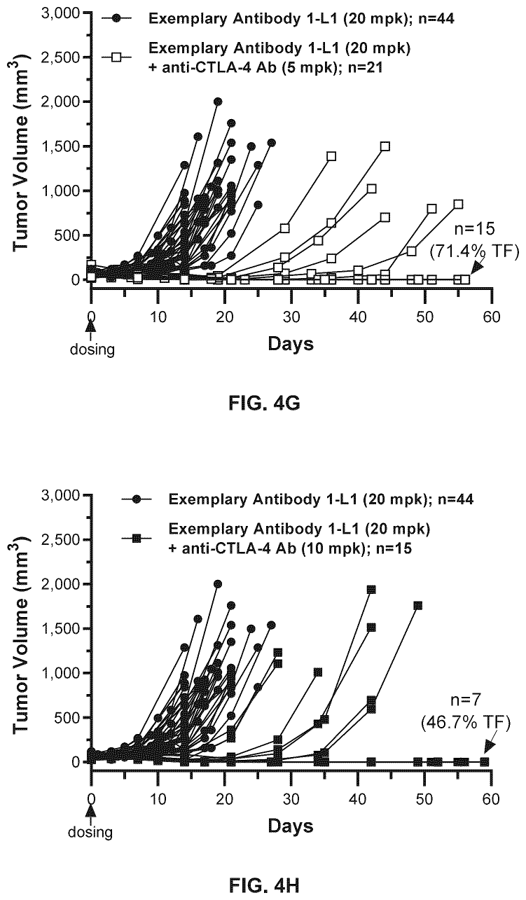

A CT26 mouse colon cancer derived tumor model in a BALB/c mouse (FIGS. 4A-4H). In FIGS. 4A-4D, 0 mpk of the anti-TM4SF1 ADC (Exemplary Antibody 1-L1) was administered, and anti-mouse CTLA-4 antibody was administered at 0, 2.5, 5, and 10 mpk, respectively. FIGS. 4E-4H show syngeneic models administered 20 mpk of the anti-TM4SF1 ADC (Exemplary Antibody 1-L1), and anti-mouse CTLA-4 antibody was administered at 0, 2.5, 5, and 10 mpk, respectively. All dosing was administered once at day 0 of the study.

In FIG. 4A, a CT26 BALB/c syngeneic model (n=40) was separated into 2 treatment groups to assess tumor volume (mm³) in the presence of different treatments: 1) Vehicle (Control) and 2) isotype-matched control (IS-Ctl), which were administered once at day 0 of the study. Over the course of the treatment, tumor volumes were measured 2-3 times a week as indicated by the dots in FIG. 4A. Neither treatment led to any tumor regression. In FIG. 4B, a third CT26 BALB/c treatment group (n=5), in which the anti-mouse CTLA-4 antibody was administered at 2.5 mpk once at day 0 of the study. Treatment with only the anti-mouse CTLA-4 antibody resulted in some level of tumor growth retardation with 1 animal experienced tumor free (TF) (20% TF) by day 14, and persisted until at least after day 50. In FIG. 4C, a CT26 BALB/c treatment group (n=15), in which the anti-mouse CTLA-4 antibody was administered at 5 mpk once at day 0 of the study. Treatment with the 5 mpk anti-mouse CTLA-4 antibody resulted in significantly better tumor growth retardation than the 2.5 mpk alone with 2 mice experienced tumor free (13.3% TF) by day 21, and persisted until at least after day 50. In FIG. 4D, a CT26 BALB/c treatment group (n=19), in which the anti-mouse CTLA-4 antibody is administered at 10 mpk once at day 0 of the study. Treatment with the 10 mpk anti-mouse CTLA-4 antibody resulted in the best tumor growth retardation in comparison to 2.5 or 5 mpk groups with 4 mice experienced tumor free. 4 animals experienced tumor free (21% TF) by day 21, and persisted until at least after day 55.

In FIG. 4E, a CT26 BALB/c treatment group (n=35), in which the Exemplary Antibody 1-L1 antibody is administered at 20 mpk at day 0 of the study. Treatment with only the anti-TM4SF1 antibody resulted in tumors that progressed about one week delay from the control group of FIG. 4A. Treatment with only the anti-TM4SF1 antibody resulted in no animals exhibited tumor free outcome and needed to be removed from the study due to tumor burden. In FIGS. 4F-4H, a second treatment group was added consisting of mice treated with the anti-TM4SF1 antibody (20 mpk) and the anti-mouse CTLA-4 antibody (2.5, 5 and 10 mpk, respectively). In FIG. 4F, one mouse from the exemplary anti-TM4SF1 ADC/anti-mouse CTLA-4 combination treatment group (2.5 mpk, n=5) exhibited tumor free (20% TF), whereas the other mice exhibited tumor growth and needed to be removed from the study due to tumor burden. In FIG. 4G, fifteen mice from the exemplary anti-TM4SF1 ADC/anti-mouse CTLA-4 combination treatment group (5 mpk, n=21) exhibited tumor free (71.4% TF), whereas the six other mice exhibited tumor growth over time and needed to be removed from the study due to tumor burden. In FIG. 4H, seven mice from the exemplary anti-TM4SF1 ADC/anti-mouse CTLA-4 combination treatment group (10 mpk, n=15) exhibited tumor free (46.7% TF), whereas the other eight mice exhibited tumor growth over time and needed to be removed from the study due to tumor burden. In sum, our studies demonstrated that treatment of mouse syngeneic tumor models with a vascular targeted cytotoxic anti-TM4SF1 ADC in combination with blockade of anti-CTLA-4 antibodies generates potent anti-tumor immune activation. In a majority of mice with CT26 tumors, a single combined treatment of anti-TM4SF1 ADC with anti-CTLA-4 antibody eliminated the tumor; contrariwise, in control mice treated with anti-CTLA-4 antibody only, most mice experienced tumor growth and needed to be sacrificed due to tumor burden. Specifically, under treatment with 2.5 mpk, 5 mpk, and 10 mpk anti-CTLA-4 antibody respectively, 20%, 13.3%, and 21% of BALB/C mice with CT26 tumors became tumor free over the course of 60-70 day evaluation time; while with the same dose of anti-CTLA-4 antibody combined with 20 mpk anti-TM4SF1 ADC, 20%, 71.4%, and 46.7% of mice in the respective groups achieved tumor free status over the course of 60-70 day evaluation period. This profound enhancement of anti-tumor immunity may be responsible for increasing the tumor-free fraction from 13.3% to 71.4% at 5 mpk anti-CTLA-4 antibody by damaging the tumor endothelium, turning an immunologically cold “tumor wound” into an immunologically warm and closely surveilled “chronic wound”.

### CT26 Rechallenge

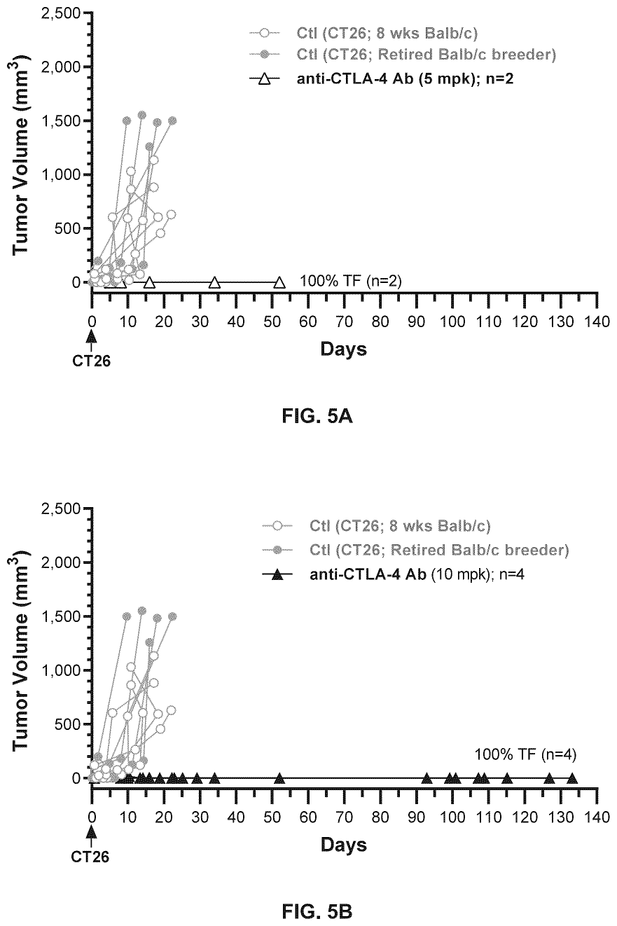

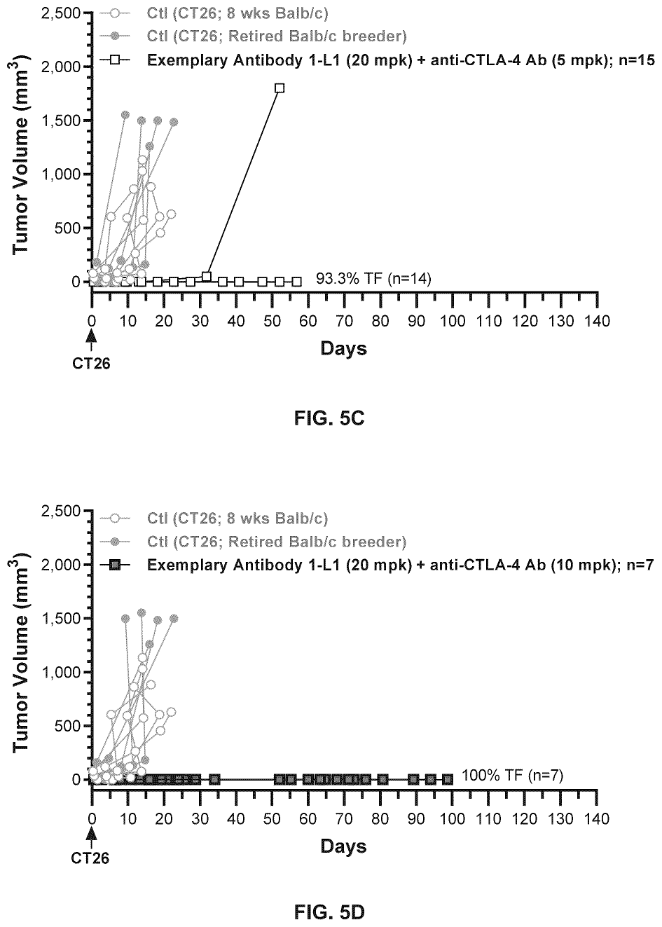

To determine whether tumor free (TF) mice that were achieved via the treatment of immune checkpoint inhibitor anti-CTLA-4 antibody is a long term effect, mice that no longer exhibited CT26 tumor near 60 to 70 days after the initial dosing in FIGS. 4A-4H were re-injected with CT26 tumor cells. For example, the CTLA-4 mice used in FIG. 5A were taken from the mice showing TF in respective treatment condition (FIG. 4B). In each rechallenge condition, 2 groups of control mice were used: 1) 8-week-old BALB/c and 2) retired BALB/c breeder (˜ 7-8 months old). At day 0, each of control and tumor free mice received a million CT26 tumor cells subcutaneously in their left side of upper flank which is reverse side from where original tumor cells were injected in FIG. 4B. Results showed that all tumor free mice, except one outlier in FIG. 5C, continuously exhibited as tumor free. In contrast, tumors grew rapidly in both control mice groups and needed to be sacrificed due to tumor burden within 20 days after the tumor cells were introduced. Specifically, mice exhibiting TF after rechallenging are FIG. 5A (100%, 2/2), FIG. 5B (100%, 4/4), FIG. 5C (93.3%, 1/15) and FIG. 5D (100%, 7/7). Thus, this currently ongoing study shows that CT26 tumor cells were unable to grow into a tumor when it was re-introduced to the same mice that had already formed T-cell memory to eliminate CT26 tumor cells.

The outcome of this currently ongoing study (FIG. 5C) shows that only one out of 15 mice in the tumor free group showed tumor re-growth. This suggests that once T-cell memory is properly formed in mice with the aid of immune checkpoint anti-mouse CTLA-4 antibody and became tumor free, the mice can eradicate the tumor cells when they are being re-challenged later. Our combination therapy of anti-TM4SF1 antibody 1-L1 with 5 mpk immune checkpoint anti-mouse CTLA-4 antibody enhanced ability for mice to form memory T-cell and became tumor free.

### Example 6: Tumor Regression Using an Anti-TM4SF1 ADC and Anti-Mouse CTLA-4 Antibody in a Renca Syngeneic Model

For this study, cell-derived syngeneic Renca mouse models were used to assess the effects of a treatment comprising an anti-TM4SF1 ADC (Exemplary Antibody 1-L1) and/or an anti-mouse CTLA-4 antibody (Clone 9H10; BioXCell) combination.

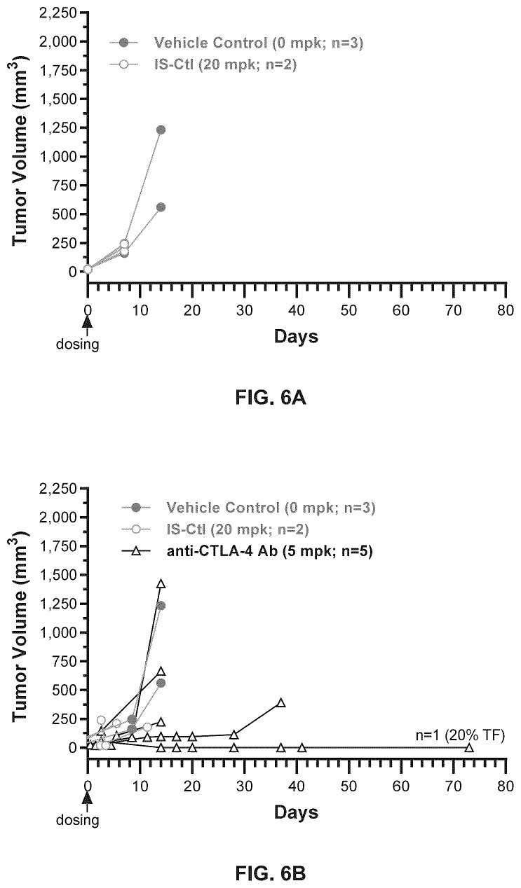

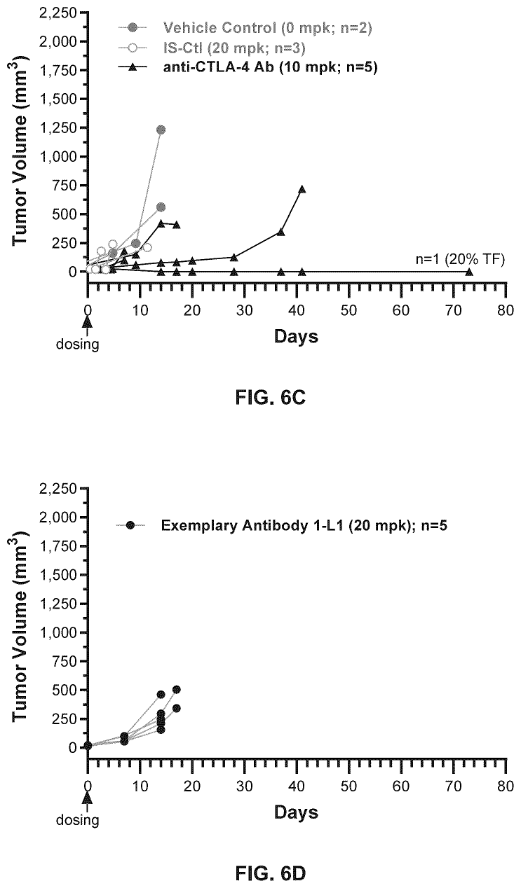

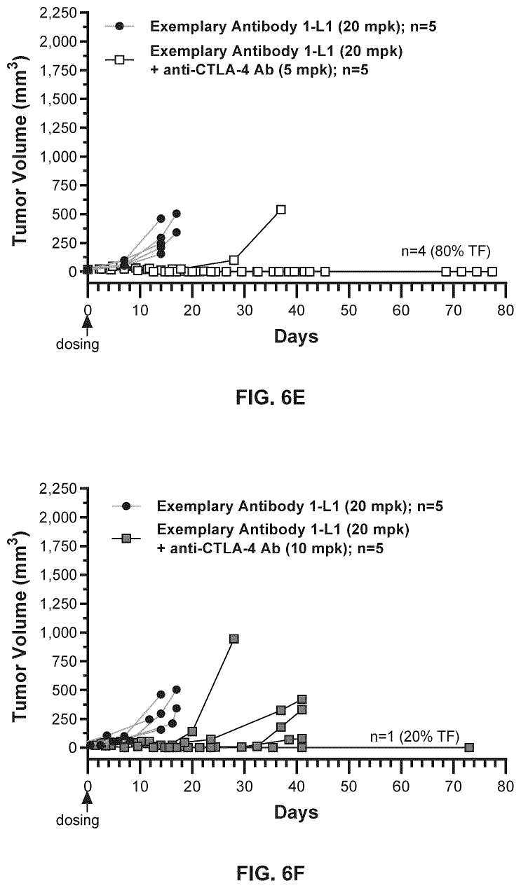

A syngeneic model assessed was a Renca derived tumor model in a BALB/c mouse (FIGS. 6A-6F). In FIGS. 6A-6C, 0 mpk of the anti-TM4SF1 ADC (Exemplary Antibody 1-L1) was administered, and anti-mouse CTLA-4 antibody was administered at 0, 5, and 10 mpk, respectively. FIGS. 6D-6F show syngeneic models administered a combination of 20 mpk of the anti-TM4SF1 ADC (Exemplary Antibody 1-L1), and anti-mouse CTLA-4 antibody was administered at 0, 2.5, 5, and 10 mpk, respectively. All dosing was administered day 0 of the study.

In FIG. 6A, a Renca BALB/c syngeneic model (n=5) was separated into 2 treatment groups to assess tumor volume (mm3) in the presence of different treatments: 1) Vehicle (Control) and 2) isotype-matched control (IS-Ctl), which were administered once at the start of the study. Over the course of the treatment, tumor volumes were measured regularly as indicated by the dots. Both treatments did not cause any tumor regression. In FIG. 6B, a third Renca BALB/c treatment group (n=5), in which the anti-mouse CTLA-4 antibody is administered at 5 mpk. The anti-mouse CTLA-4 antibody treatment was administered once at the start of treatment. Treatment with only the anti-mouse CTLA-4 antibody resulted in tumors that progressed similarly to the no treatment control with 1 animal exhibited tumor free (20% TF) by day 14, and persisted until at least after day 70. In FIG. 6C, a Renca BALB/c treatment group (n=5), in which the anti-mouse CTLA-4 antibody is administered at 10 mpk. The anti-mouse CTLA-4 antibody treatment were administered once at the start of treatment. Treatment with only the anti-mouse CTLA-4 antibody resulted in tumors that progressed more slowly as compared to the no treatment control with 1 mouse experienced tumor free (20% TF) by day 14, and persisted until at least after day 70. In FIG. 6D, a Renca BALB/c treatment group (n=5), in which the anti-TM4SF1 ADC is administered at 20 mpk at the start of the study. In FIG. 6E, a Renca BALB/c treatment group (n=5), in which the Exemplary Antibody 1-L1 antibody is administered at 20 mpk in combination with anti-mouse CTLA-4 antibody (5 mpk) at day 0 of the study. The combination treatment caused tumors to grow more slowly with 80% of mice exhibiting tumor free, with some retaining TF after at least 70 days. In FIG. 6F, a Renca BALB/c treatment group (n=5), in which the Exemplary Antibody 1-L1 antibody is administered at 20 mpk in combination with anti-mouse CTLA-4 antibody (10 mpk) at day 0 of the study. The combination treatment caused tumors to grow more slowly with 20% of mice exhibiting tumor free during the entire course of at least 70 days of observation period. In a similar outcome to studies of the CT26 tumor model, studies of the Renca tumor model demonstrated that treatment of mouse syngeneic tumor models with a vascular targeted cytotoxic anti-TM4SF1 ADC in combination with 5 mpk antibody blockade of CTLA-4 generates potent anti-tumor immune activation. In contrary, for control mice treated with anti-CTLA-4 antibody only, most mice experienced tumor growth and needed to be sacrificed due to tumor burden. Moreover, as was observed in CT26 tumor model, combination therapy of anti-TM4SF1 ADC with the 5 mpk CTLA-4 antibody showed better tumor regression outcome than the 10 mpk CTLA-4 antibody treatment. The common observation that excessive (>5 mpk) doses of anti-CTLA-4 antibody hinder anti-tumor immunity may perhaps be explained by diversion of immune cells toward normal tissue, as CTLA-4 normally functions to suppress autoimmunity. In the clinic, CTLA-4 antibodies are dose-limited by autoimmune side effects. In the case of ipilimumab, for example, the first label-listed warnings and precautions are immune-mediated hepatitis, immune-mediated endocrinopathies, immune-mediated pneumonitis, immune-mediated nephritis, and immune-mediated encephalitis. If activation of anti-tumor immunity by a vascular targeted cytotoxic anti-TM4SF1 ADC enables CTLA-4 antibodies to be efficacious at lower doses, below the dose threshold which generates these autoimmune toxicities, then the therapeutic margin of CTLA-4 antibodies may be substantially improved and the number of patients in which efficacy is reached may be greatly increased.

### Renca Rechallenge

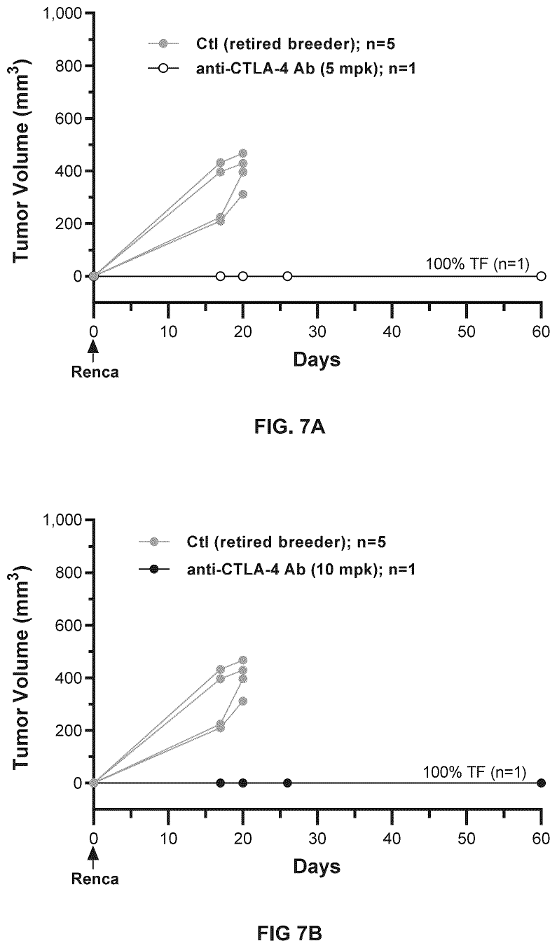

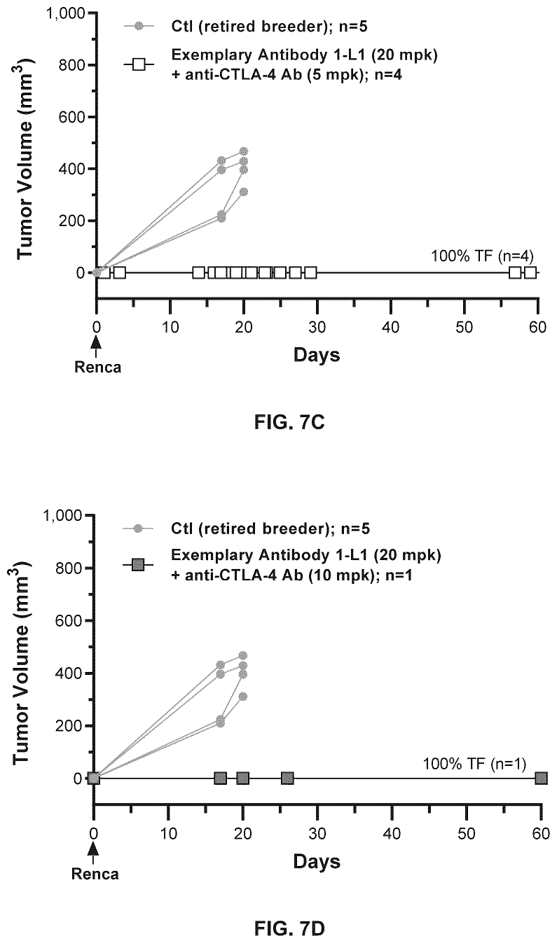

To determine whether complete tumor regression can persist during a tumor rechallenge, mice that exhibited tumor free (TF) near 60 to 70 days after the initial dosing in FIGS. 6A-6F were rechallenged with Renca. For example, the CTLA4 mice used in FIG. 7A were taken from the mice showing TF in respective treatment condition (FIG. 6B). To match mice age with the tumor free mice, 7-8 months old retired BALB/c breeder was used as control mice). At day 0, a million Renca tumor cells subcutaneously injected to upper flank of each mice and tracked tumor growth over time. As is seen in CT26 tumor rechallenge, all tumor free mice continuously exhibited as tumor free in the Renca tumor model. Conversely, tumors grew rapidly in control mice groups and needed to be sacrificed due to tumor burden within 20 days after tumor cells were introduced. Specifically, mice exhibiting TF after Renca tumor cell rechallenging are FIG. 7A (100%, 1/1), FIG. 7B (100%, 1/1), FIG. 7C (100%, 4/4) and FIG. 7D (100%, 1/1). Thus, our studies demonstrated that, following a single combination treatment of an anti-TM4SF1 ADC with an anti-CTLA-4 antibody in both CT26 and Renca tumor models in BALB/c mice, many BALB/c mice became tumor free by not only eliminating initial tumor, but also establishing a long-term T-cell memory and effectively eliminate re-introduced tumor cells without need for retreatment.

### Example 7: Tumor Regression Using an Anti-TM4SF1 ADC and Anti-Mouse CTLA-4 Antibody in a B16F10 Syngeneic Model

For this study, cell-derived syngeneic B16F10 mouse models were used to assess the effects of a treatment comprising an anti-TM4SF1 ADC (Exemplary Antibody 1-L1) and/or an anti-mouse CTLA-4 antibody (9H10) combination.

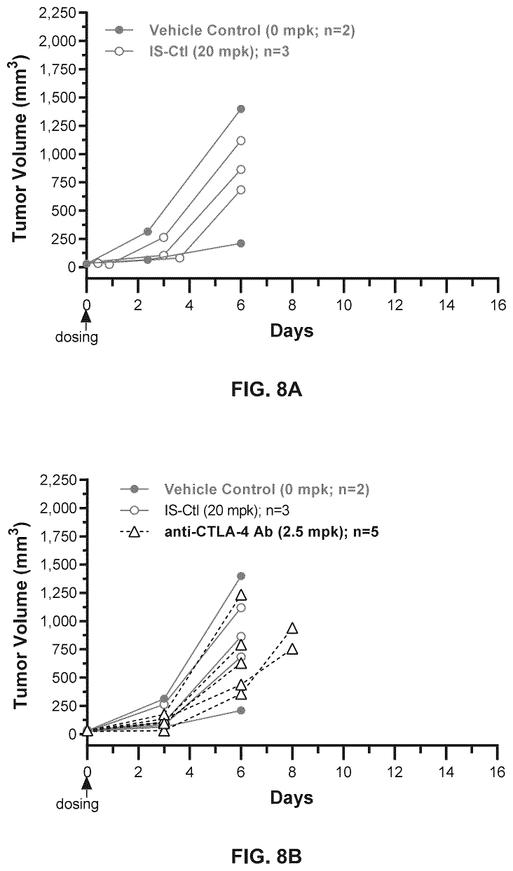

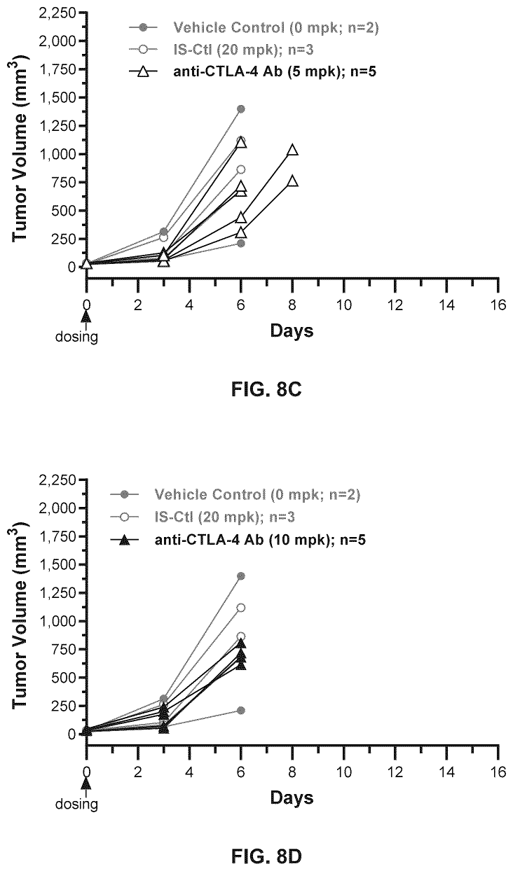

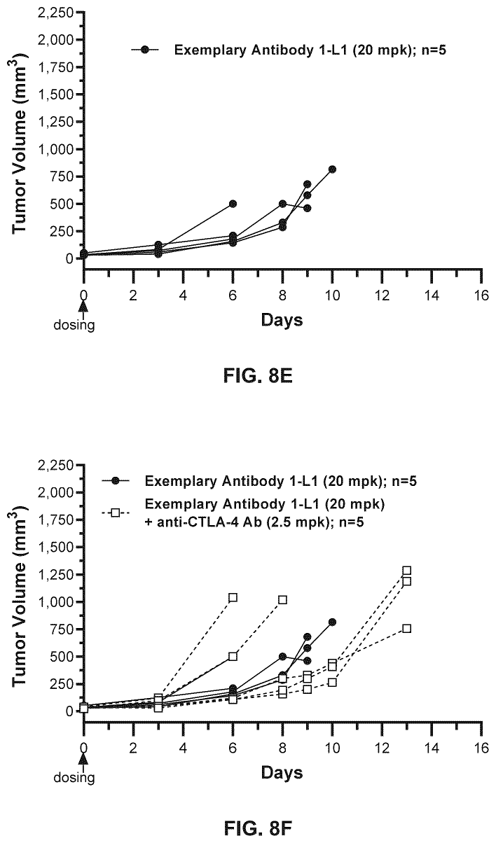

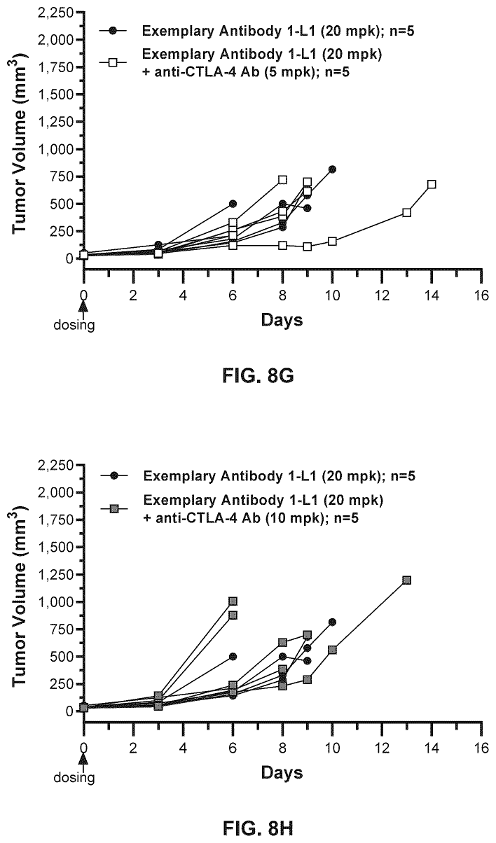

A B16F10 mouse melanoma cell derived tumor model in a C57BL/6 mouse (FIGS. 8A-8H). In FIGS. 8A-8D, 0 mpk of the anti-TM4SF1 ADC (Exemplary Antibody 1-L1) was administered, and anti-mouse CTLA-4 antibody was administered at 0, 2.5, 5, and 10 mpk, respectively. FIGS. 8E-8H show syngeneic models administered 20 mpk of the anti-TM4SF1 ADC (Exemplary Antibody 1-L1), and anti-mouse CTLA-4 antibody was administered at 0, 2.5, 5, and 10 mpk, respectively. All dosing was administered day 0 of the study.

In FIG. 8A, a B16F10 C57BL/6 syngeneic model (n=5) was separated into 2 treatment groups to assess tumor volume (mm³) in the presence of different treatments: 1) Vehicle (Control) and 2) isotype-matched control (IS-Ctl), which were administered once at the start of the study. Over the course of the treatment, tumor volumes were measured regularly as indicated by the dots in FIG. 4A. Tumor growth in both treatments was referred as control tumor growth. In FIG. 8B, a third B16F10 C57BL/6 treatment group (n=5), in which the anti-mouse CTLA-4 antibody is administered at 2.5 mpk and was administered once at the start of treatment. Treatment with only the anti-mouse CTLA-4 antibody resulted in tumors that progressed similarly to the no treatment control with no mice exhibiting tumor free. In FIG. 8C, a B16F10 C57BL/6 treatment group (n=5), in which the anti-mouse CTLA-4 antibody is administered at 5 mpk and was administered once at the start of treatment. Treatment with only the anti-mouse CTLA-4 antibody resulted in tumors that progressed similarly to the no treatment control with no mice exhibiting tumor free. In FIG. 8D, a B16F10 C57BL/6 treatment group (n=5), in which the anti-mouse CTLA-4 antibody is administered at 10 mpk and was administered once at the start of treatment. Treatment with only the anti-mouse CTLA-4 antibody resulted in tumors that progressed similarly to the no treatment control with no mice exhibiting tumor free. This outcome, unlike in BALB/c mice, anti-CTLA-4 antibody alone did not affect B16F10 tumor growth in C57BL/6 mice.

In FIG. 8E, a B16F10 C57BL/6 treatment group (n=5), in which the Exemplary Antibody 1-L1 antibody is administered at 20 mpk at day 0 of the study. Treatment with only the anti-TM4SF1 ADC showed promising delay in tumor growth in comparison to control mice. In FIGS. 8F-8H, a second treatment group was added consisting of mice treated with the anti-TM4SF1 ADC (20 mpk) and the anti-mouse CTLA-4 antibody (2.5, 5 and 10 mpk, respectively). In FIG. 8F, no mouse from the exemplary anti-TM4SF1 ADC with anti-mouse CTLA-4 antibody combination treatment group (2.5 mpk, n=5) exhibited tumor free (0% TF) with all mice exhibited tumor growth similar to the exemplary anti-TM4SF1 ADC treatment alone. In FIG. 8G, no mice from the exemplary anti-TM4SF1 ADC with anti-mouse CTLA-4 antibody combination treatment group (5 mpk, n=5) exhibited tumor free (0% TF) with all mice exhibited tumor growth similar to the exemplary anti-TM4SF1 ADC treatment alone. In FIG. 8H, no mice from the exemplary anti-TM4SF1 ADC with anti-mouse CTLA-4 antibody combination treatment group (10 mpk, n=5) exhibited tumor free (0% TF) with all mice exhibited tumor growth similar to the exemplary anti-TM4SF1 ADC treatment alone. In sum, this example demonstrated that in tumor models non-responsive to a checkpoint inhibitor, such as the B16F10 model in C57BL/6 mice which is non-responsive to CTLA-4 antibody treatment, combination therapy with an anti-TM4SF1 ADC would not make the tumor model responsive.

### Example 8: Tumor Regression Using an Anti-TM4SF1 ADC and Anti-CTLA Antibody in a LLC Syngeneic Model

For this study, cell-derived syngeneic LLC tumor model in C57BL/6 mouse strain was used to assess the effects of a treatment comprising an anti-TM4SF1 ADC (Exemplary Antibody 1-L1) and/or an anti-CTLA4 antibody (9H10) combination.

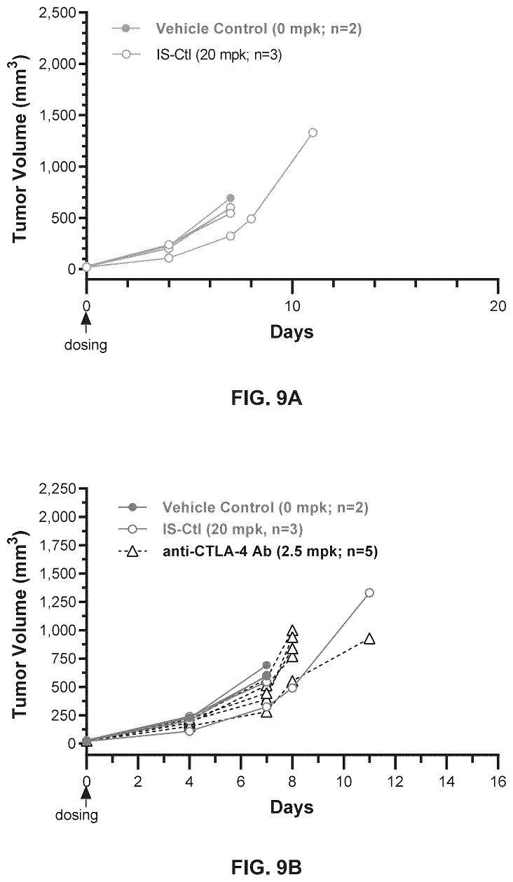

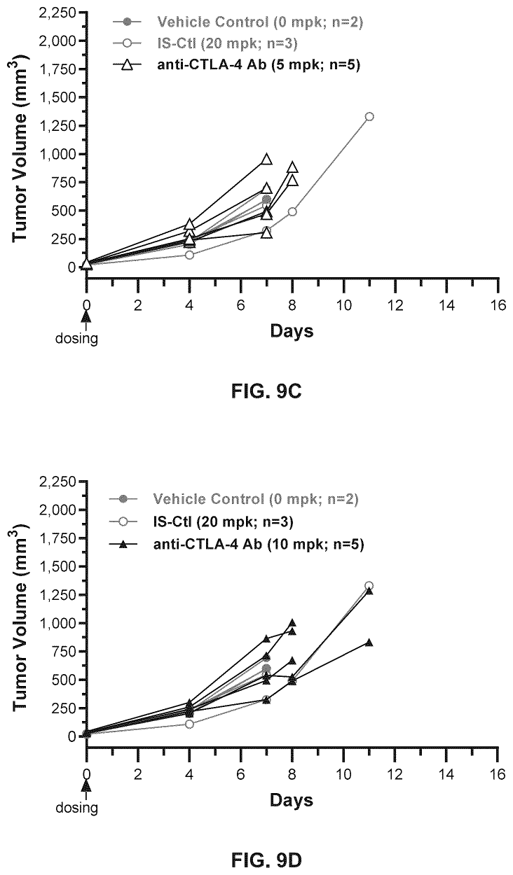

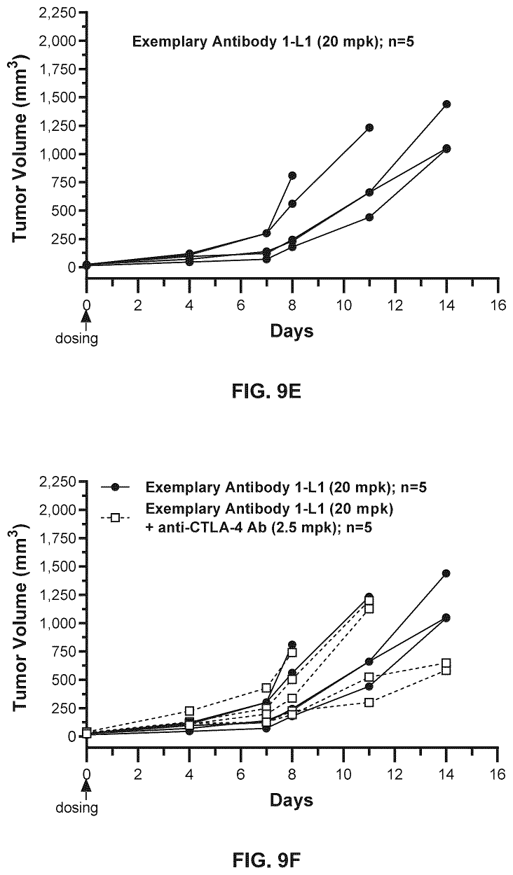

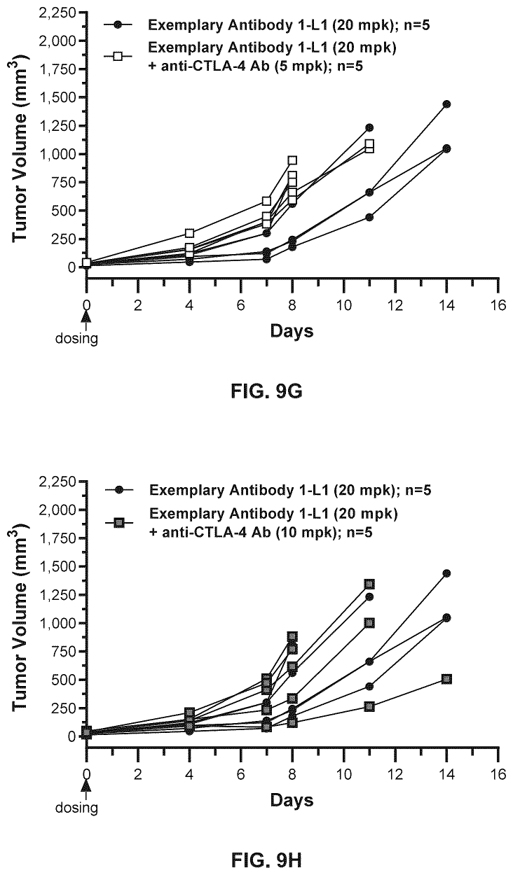

A LLC derived tumor model in a C57BL/6 mouse (FIGS. 9A-9H). In FIGS. 9A-9D, 0 mpk of the anti-TM4SF1 ADC (Exemplary Antibody 1-L1) was administered, and anti-CTLA antibody was administered at 0, 2.5, 5, and 10 mpk, respectively. FIGS. 9E-9H show syngeneic models administered 20 mpk of the anti-TM4SF1 ADC (Exemplary Antibody 1-L1), and anti-CTLA antibody was administered at 0, 2.5, 5, and 10 mpk, respectively. All dosing was administered day 0 of the study.

In FIG. 9A, a LLC C57BL/6 syngeneic model (n=5) was separated into 2 treatment groups to assess tumor volume (mm³) in the presence of different treatments: 1) Vehicle (Control) and 2) isotype-matched control (IS-Ctl), which were administered once at the start of the study. Over the course of the treatment, tumor volumes were measured regularly as indicated by the dots in FIG. 9A. Both treatments did not cause any tumor regression. In FIG. 9B, a third B16F10 C57BL/6 treatment group (n=5), in which the anti-mouse CTLA-4 antibody is administered at 2.5 mpk. The anti-mouse CTLA-4 antibody treatment were administered once at the start of treatment. Treatment with only the anti-mouse CTLA-4 antibody resulted in tumors that progressed similarly to the no treatment control with no mice exhibiting complete tumor regression. In FIG. 9C, a B16F10 C57BL/6 treatment group (n=5), in which the anti-mouse CTLA-4 antibody is administered at 5 mpk. The anti-mouse CTLA-4 antibody treatment were administered once at the start of treatment. Treatment with only the anti-mouse CTLA-4 antibody resulted in tumors that progressed similarly to the no treatment control with no mice exhibiting complete tumor regression. In FIG. 9D, a LLC C57BL/6 treatment group (n=5), in which the anti-mouse CTLA-4 antibody is administered at 10 mpk. The anti-mouse CTLA-4 antibody treatment were administered once at the start of treatment. Treatment with only the anti-mouse CTLA-4 antibody resulted in tumors that progressed similarly to the no treatment vehicle and IS-Ctl control with no mice exhibiting tumor free. Thus, LLC tumor regression outcome is closely resembling the B16F10 tumor and anti-mouse CTLA-4 antibody was unable to influence the tumor growth.

In FIG. 9E, a LLC C57BL/6 treatment group (n=5), in which the Exemplary Antibody 1-L1 antibody is administered at 20 mpk at day 0 of the study. Treatment with only the anti-TM4SF1 ADC resulted in promising delay in tumor growth in comparison to control mice. In FIGS. 9F-9H, a second treatment group was added consisting of mice treated with the anti-TM4SF1 antibody (20 mpk) and the anti-mouse CTLA-4 antibody (2.5, 5 and 10 mpk, respectively). In FIG. 9F, no mouse from the exemplary anti-TM4SF1 ADC with anti-mouse CTLA-4 antibody combination treatment group (2.5 mpk, n=5) exhibited tumor free (0% CTR) with all mice exhibited tumor growth similar to the exemplary anti-TM4SF1 ADC treatment alone. In FIG. 9G, no mice from the exemplary anti-TM4SF1 ADC anti-mouse CTLA-4 antibody combination treatment group (5 mpk, n=5) exhibited tumor free (0% CTR) with anti-mouse CTLA-4 antibody combination treatment exhibited tumor free (0% CTR) with all mice exhibited tumor growth similar to the exemplary anti-TM4SF1 ADC treatment alone. In FIG. 9H, no mice from the exemplary anti-TM4SF1 ADC with anti-mouse CTLA-4 antibody combination treatment exhibited tumor free (0% CTR) with all mice exhibited tumor growth similar to the exemplary anti-TM4SF1 ADC treatment alone. Taken together, our studies showed that in tumor models non-responsive to a checkpoint inhibitor, such as the B16F10 and LLC tumor models in C57BL/6 mice which is non-responsive to CTLA-4 antibody treatment, combination therapy with an anti-TM4SF1 ADC would not make the tumor model more responsive. However, when the tumor model had a low response rate, as in the CT26 and Renca models in BALB/c mice, combination therapy with an anti-TM4SF1 ADC substantially increased the response rate.

It will be apparent to those skilled in the art that various modifications and variations can be made in the compositions, methods, and kits of the present disclosure without departing from the spirit or scope of the disclosure. Thus, it is intended that the present disclosure cover the modifications and variations of this disclosure provided they come within the scope of the appended claims and their equivalents.

TABLE 2
SEQUENCE DESCRIPTION

| | | |
|---|---|---|
| SEQ | | |
| ID NO | Description | Sequence |

Antibody AGX-A01

| | | |
|---|---|---|
| 1 | AGX-A01 | EVILVESGGGLVKPGGSLKLSCAASGFTFSS |
| | Variable heavy (VH) chain- | FAMSWVRQTPEKRLEWVATISSGSIYIYYT |
| | amino acid | DGVKGRFTISRDNAKNTVHLQMSSLRSEDT |
| | | AMYYCARRGIYYGYDGYAMDYWGQGTSV |
| | | TVS |
| 2 | AGX-A01 | AVVMTQTPLSLPVSLGDQASISCRSSQSLVH |
| | Variable light (VL) chain- | SNGNTYLHWYMQKPGQSPKVLIYKVSNRF |
| | amino acid | SGVPDRFSGSGSGTDFTLKISRVEADDLGIY |
| | | FCSQSTHIPLAFGAGTKLELK |

Antibody AGX-A03

| | | |
|---|---|---|
| 3 | AGX-A03 | QIQLVQSGPELKKPGETVKISCKASGYSFRD |
| | Variable heavy (VH) chain- | YGMNWVKQAPGRTFKWMGWINTYTGAPV |
| | amino acid | YAADFKGRFAFSLDTSASAAFLQINNLKNE |
| | | DTATYFCARWVSYGNNRNWFFDFWGAGT |
| | | TVTVSS |
| 4 | AGX-A03 | CAGATCCAGTTGGTGCAGTCTGGACCTGA |
| | Variable heavy (VH) chain- | GCTGAAGAAGCCTGGAGAGACAGTCAAG |
| | nucleic acid | ATCTCCTGCAAGGCTTCTGGGTATTCCTTC |
| | | AGAGACTATGGAATGAACTGGGTGAAGC |
| | | AGGCTCCAGGAAGGACTTTTAAGTGGATG |
| | | GGCTGGATAAACACCTACACTGGAGCGCC |
| | | AGTATATGCTGCTGACTTCAAGGGACGGT |
| | | TTGCCTTCTCTTTGGACACCTCTGCCAGCG |
| | | CTGCCTTTTTGCAGATCAACAACCTCAAA |
| | | AATGAAGACACGGCTACATATTTCTGTGC |
| | | AAGATGGGTCTCCTACGGTAATAACCGCA |
| | | ACTGGTTCTTCGATTTTTGGGGCGCAGGG |
| | | ACCACGGTCACCGTCTCCTCA |
| 5 | AGX-A03 | CAAATTCAGTTGGTTCAATCCGGCCCTGA |
| | Variable heavy (VH) chain- | GCTCAAGAAGCCTGGAGAGACAGTGAAG |
| | codon optimized nucleic | ATAAGTTGTAAGGCTAGTGGCTATTCATT |
| | acid | TCGAGATTATGGGATGAATTGGGTCAAGC |
| | | AGGCCCCAGGGCGGACCTTCAAATGGAT |
| | | GGGGTGGATCAATACTTACACTGGCGCAC |
| | | CAGTATATGCAGCTGATTTTAAGGGTCGC |
| | | TTTGCATTTTCACTTGATACTTCAGCCAGT |
| | | GCCGCTTTTTTGCAAATCAACAATCTCAA |
| | | AAATGAAGACACTGCTACATATTTCTGCG |
| | | CCAGGTGGGTGAGCTATGGCAATAACAG |
| | | AAATTGGTTCTTTGACTTTTGGGGCGCAG |
| | | GCACCACCGTCACTGTCTCATCA |
| 6 | VH-CDR1 | GYSFRDYGMN |
| 7 | VH-CDR2 | WINTYTGAPVYAADFKG |
| 8 | VH-CDR3 | WVSYGNNRNWFFDF |
| 9 | AGX-A03 | DVLMTQTPLSLPVRLGDQASISCRSSQTLVH |
| | Variable light (VL) chain- | SNGNTYLEWYLQKPGQSPKLLIYKVSNRLS |
| | amino acid | GVPDRFSGSGSGTDFTLKISRVETEDLGVYY |
| | | CFQGSHGPWTFGGGTKLEIK |
| 10 | AGX-A03 | GATGTTTTGATGACCCAAACTCCACTCTC |
| | Variable light (VL) chain- | CCTGCCTGTCCGTCTTGGAGATCAGGCCT |
| | nucleic acid | CCATCTCTTGTAGATCTAGTCAGACCCTT |
| | | GTACATAGTAATGGAAACACCTATTTAGA |
| | | ATGGTACCTGCAGAAACCAGGCCAGTCTC |
| | | CAAAACTCTTGATCTACAAAGTTTCCAAT |
| | | CGACTTTCTGGGGTCCCAGACAGGTTCAG |
| | | TGGCAGTGGATCAGGGACAGATTTCACAC |
| | | TCAAGATCAGCAGAGTGGAGACTGAGGA |
| | | TCTGGGAGTTTATTACTGCTTTCAAGGTTC |
| | | ACATGGTCCGTGGACGTTCGGTGGAGGCA |
| | | CCAAGCTGGAAATCAAA |
| 11 | AGX-A03 | GACGTACTTATGACACAAACTCCCTTGAG |
| | Variable light (VL) chain- | CTTGCCAGTACGGCTTGGCGATCAAGCTT |
| | codon | CAATTTCATGTCGTTCTTCTCAAACACTTG |
| | optimized nucleic acid | TCCACTCAAATGGGAATACATATTTGGAA |
| | | TGGTATCTCCAAAAGCCCGGCCAATCCCC |
| | | AAAATTGTTGATTTACAAGGTGTCTAATC |
| | | GACTCTCAGGCGTCCCCGACCGATTCTCC |
| | | GGGAGCGGGTCCGGTACAGACTTCACCTT |
| | | GAAAATCTCCAGGGTAGAAACTGAAGAC |
| | | CTCGGAGTCTACTATTGTTTCCAGGGGTC |
| | | ACACGGCCCCTGGACATTTGGAGGAGGA |
| | | ACTAAGCTCGAGATCAAA |
| 12 | VL-CDR1 | RSSQTLVHSNGNTYLE |
| 13 | VL-CDR2 | KVSNRLS |
| 14 | VL-CDR3 | FQGSHGPWT |

Antibody AGX-A04

| | | |
|---|---|---|
| 15 | AGX-A04 | EVQLQQSGPELVKPGASVKISCKTSGYTFTD |
| | Variable heavy (VH) chain- | YTMHWVRQSHGKSLEWIGSFNPNNGGLTN |
| | amino acid | YNQKFKGKATLTVDKSSSTVYMDLRSLTSE |
| | | DSAVYYCTRIRATGFDSWGQGTTLTVSS |
| 16 | AGX-A04 | GAGGTCCAGCTGCAACAGTCTGGACCTGA |
| | Variable heavy (VH) chain- | GCTGGTGAAGCCTGGGGCTTCAGTGAAGA |
| | nucleic acid | TATCCTGCAAGACTTCTGGATACACATTC |
| | | ACTGATTACACCATGCACTGGGTGAGGCA |
| | | GAGCCATGGAAAGAGCCTTGAGTGGATT |
| | | GGAAGTTTTAATCCTAACAATGGTGGTCT |
| | | TACTAACTACAACCAGAAGTTCAAGGGCA |
| | | AGGCCACATTGACTGTGGACAAGTCTTCC |
| | | AGCACAGTGTACATGGACCTCCGCAGCCT |
| | | GACATCTGAGGATTCTGCAGTCTATTACT |
| | | GTACAAGAATCCGGGCTACGGGCTTTGAC |
| | | TCCTGGGGCCAGGGCACCACTCTCACAGT |
| | | CTCCTCA |
| 17 | AGX-A04 | GAGGTACAACTGCAACAGAGTGGACCTG |
| | Variable heavy (VH) chain- | AACTTGTCAAACCTGGAGCAAGTGTGAAG |
| | codon optimized nucleic acid | ATTAGCTGTAAAACCAGTGGCTACACATT |
| | | TACCGATTATACTATGCACTGGGTAAGAC |
| | | AGAGCCACGGAAAATCACTGGAGTGGAT |
| | | TGGTAGTTTCAATCCTAACAACGGAGGAT |
| | | TGACAAATTACAACCAGAAGTTCAAAGG |
| | | GAAAGCCACCTTGACAGTTGATAAGTCCT |
| | | CAAGTACCGTGTATATGGATCTGCGTTCT |
| | | CTCACAAGTGAAGATAGCGCAGTTTACTA |
| | | CTGTACCCGCATCCGAGCCACCGGGTTCG |
| | | ATTCATGGGGTCAGGGGACAACACTGACT |
| | | GTTTCTTCT |
| 18 | VH-CDR1 | GYTFTDYTMH |
| 19 | VH-CDR2 | SFNPNNGGLTNYNQKFKG |
| 20 | VH-CDR3 | IRATGFDS |
| 21 | AGX-A04 | DIVMSQSPSSLAVSAGEKVTMSCKSSQSLL |
| | Variable light (VL) chain- | NSRTRKNYLAWYQQKPGQSPKLLIYWAST |
| | amino acid | RESGVPDRFTGSGSGTDFTLTISNVQAEDLT |
| | | VYYCKQSYNPPWTFGGGTKLEIK |
| 22 | AGX-A04 | GACATTGTGATGTCACAGTCTCCATCCTC |
| | Variable light (VL) chain- | CCTGGCTGTGTCAGCAGGAGAGAAGGTC |
| | nucleic acid | ACTATGAGCTGCAAATCCAGTCAGAGTCT |
| | | GCTCAACAGTAGAACCCGAAAGAACTAC |
| | | TTGGCTTGGTACCAGCAGAAACCAGGGCA |
| | | GTCTCCTAAACTGCTGATCTACTGGGCAT |
| | | CCACTAGGGAATCTGGGGTCCCTGATCGC |
| | | TTCACAGGCAGTGGATCTGGGACAGATTT |
| | | CACTCTCACCATCAGCAATGTGCAGGCTG |
| | | AAGACCTGACAGTTTATTACTGCAAGCAA |
| | | TCTTATAATCCTCCGTGGACGTTCGGTGG |
| | | AGGCACCAAGCTGGAAATCAAA |
| 23 | AGX-A04 | GACATAGTTATGTCCCAGTCTCCATCCAG |
| | | CTTGGCTGTCAGCGCCGGAGAGAAAGTG |
| | | ACTATGAGTTGTAAATCTTCCCAGTCCCT |
| | Variable light (VL) chain- | GCTTAACTCACGTACTCGGAAGAATTATC |
| | codon optimized nucleic acid | TTGCCTGGTATCAACAAAAGCCAGGTCAA |
| | | AGTCCTAAGCTCCTTATTTACTGGGCCTC |
| | | AACACGGGAGTCAGGTGTCCCCGATCGCT |
| | | TCACAGGTAGTGGGAGTGGTACTGACTTC |
| | | ACTCTCACCATTTCAAATGTCCAAGCAGA |
| | | AGACTTGACTGTGTATTACTGTAAGCAGA |
| | | GTTACAACCCTCCTTGGACCTTTGGTGGG |
| | | GGGACCAAACTGGAGATCAAG |
| 24 | VL-CDR1 | KSSQSLLNSRTRKNYLA |
| 25 | VL-CDR2 | WASTRES |
| 26 | VL-CDR3 | KQSYNPPWT |

Antibody AGX-A05

| | | |
|---|---|---|
| 27 | AGX-A05 | EVQVQQSGPELVKPGASVKMSCKASGYTF |
| | Variable heavy (VH) chain- | TSYVMHWVKQKPGQGLEWIGYINPNNDNI |
| | amino acid | NYNEKFKGKASLTSDKSSNTVYMELSSLTS |
| | | EDSAVYYCAGYGNSGANWGQGTLVTVSA |
| 28 | AGX-A05 | GAGGTCCAGGTACAGCAGTCTGGACCTGA |
| | Variable heavy (VH) chain- | ACTGGTAAAGCCTGGGGCTTCAGTGAAGA |
| | nucleic acid | TGTCCTGTAAGGCTTCTGGATACACATTC |
| | | ACTAGCTATGTCATGCACTGGGTGAAGCA |
| | | GAAGCCTGGGCAGGGCCTTGAGTGGATTG |
| | | GATATATTAATCCTAACAATGATAATATT |
| | | AACTACAATGAGAAGTTCAAAGGCAAGG |
| | | CCTCACTGACTTCAGACAAATCCTCCAAC |
| | | ACAGTCTACATGGAGCTCAGCAGCCTGAC |
| | | CTCTGAGGACTCTGCGGTCTATTACTGTG |
| | | CAGGCTATGGTAACTCCGGAGCTAACTGG |
| | | GGCCAAGGGACTCTGGTCACTGTCTCTGC |
| | | A |
| 29 | AGX-A05 | GAAGTTCAAGTTCAGCAAAGCGGGCCTG |
| | Variable heavy (VH) chain- | AGCTTGTCAAGCCAGGCGCATCAGTCAAA |
| | codon optimized nucleic acid | ATGAGCTGTAAGGCTTCCGGGTACACCTT |
| | | CACCAGTTATGTCATGCATTGGGTAAAAC |
| | | AAAAGCCAGGACAGGGACTCGAGTGGAT |
| | | AGGATACATTAACCCAAATAACGACAAC |
| | | ATTAACTACAACGAGAAATTCAAGGGCA |
| | | AAGCATCATTGACTTCCGATAAATCCTCT |
| | | AACACCGTGTACATGGAGCTGAGTTCATT |
| | | GACCAGCGAGGATTCTGCCGTGTACTACT |
| | | GTGCAGGTTATGGCAACTCTGGTGCTAAC |
| | | TGGGGGCAGGGGACTCTGGTCACAGTCA |
| | | GCGCA |
| 30 | VH-CDR1 | GYTFTSYVMH |
| 31 | VH-CDR2 | YINPNNDNINYNEKFKG |
| 32 | VH-CDR3 | YGNSGAN |
| 33 | AGX-A05 | DIQMTQSPASLSASVGETVTITCRTSKNIFNF |
| | Variable light (VL)chain- | LAWYHQKQGRSPRLLVSHTKTLAAGVPSR |
| | amino acid | FSGSGSGTQFSLKINSLQPEDFGIYYCQHHY |
| | | GTPWTFGGGTKLEIK |
| 34 | AGX-A05 | GACATCCAGATGACTCAGTCTCCAGCCTC |
| | Variable light (VL) chain- | CCTATCTGCATCTGTGGGAGAAACTGTCA |
| | nucleic acid | CCATCACATGTCGAACAAGTAAAAATATT |
| | | TTCAATTTTTTAGCATGGTATCACCAGAA |
| | | ACAGGGAAGATCTCCTCGACTCCTGGTCT |
| | | CTCATACAAAAACCTTAGCAGCAGGTGTG |
| | | CCATCAAGGTTCAGTGGCAGTGGCTCAGG |
| | | CACACAGTTTTCTCTGAAGATCAACAGCC |
| | | TGCAGCCTGAAGATTTTGGGATTTATTAC |
| | | TGTCAACATCATTATGGTACTCCGTGGAC |
| | | GTTCGGTGGAGGCACCAAACTGGAAATC |
| | | AAA |
| 35 | AGX-A05 | GACATTCAGATGACCCAGTCACCAGCATC |
| | Variable light (VL) chain- | TTTGAGCGCATCCGTTGGGGAGACTGTGA |
| | codon optimized nucleic acid | CAATCACATGCCGAACCAGTAAGAACATC |
| | | TTCAACTTCCTCGCATGGTACCATCAAAA |
| | | GCAGGGCAGGTCTCCCAGACTGCTTGTCT |
| | | CTCACACCAAGACACTGGCAGCAGGCGTC |
| | | CCCAGCCGGTTTAGTGGTAGTGGATCTGG |
| | | CACACAGTTTAGTTTGAAAATCAATTCCC |
| | | TGCAACCCGAAGACTTCGGCATATACTAT |
| | | TGCCAGCACCACTATGGGACACCTTGGAC |
| | | TTTCGGAGGTGGTACTAAACTTGAGATTA |
| | | AA |
| 36 | VL-CDR1 | RTSKNIFNFLA |
| 37 | VL-CDR2 | HTKTLAA |
| 38 | VL-CDR3 | QHHYGTPWT |

Antibody AGX-A07

| | | |
|---|---|---|
| 39 | AGX-A07 | QIQLVQSGPELKKPGETVKISCKASGYTFTN |
| | Variable heavy (VH) chain- | YGVKWVKQAPGKDLKWMGWINTYTGNPI |
| | amino acid | YAADFKGRFAFSLETSASTAFLQINNLKNED |
| | | TATYFCVRFQYGDYRYFDVWGAGTTVTVS |
| | | S |
| 40 | AGX-A07 | CAGATCCAGTTGGTGCAGTCTGGACCTGA |
| | Variable heavy (VH) chain- | GCTGAAGAAGCCTGGAGAGACAGTCAAG |
| | nucleic acid | ATCTCCTGCAAGGCTTCTGGGTATACCTT |
| | | CACAAACTATGGAGTGAAGTGGGTGAAG |
| | | CAGGCTCCAGGAAAGGATTTAAAGTGGA |
| | | TGGGCTGGATAAACACCTACACTGGAAAT |
| | | CCAATTTATGCTGCTGACTTCAAGGGACG |
| | | GTTTGCCTTCTCTTTGGAGACCTCTGCCAG |
| | | CACTGCCTTTTTGCAGATCAACAACCTCA |
| | | AAAATGAGGACACGGCTACATATTTCTGT |
| | | GTAAGATTCCAATATGGCGATTACCGGTA |
| | | CTTCGATGTCTGGGGCGCAGGGACCACGG |
| | | TCACCGTCTCCTCA |
| 41 | AGX-A07 | CAAATCCAACTTGTCCAGAGCGGTCCCGA |
| | Variable heavy (VH) chain- | GTTGAAGAAGCCTGGCGAAACCGTGAAA |
| | codon optimized nucleic acid | ATCTCATGCAAGGCCAGTGGATATACATT |
| | | TACAAACTATGGCGTCAAGTGGGTGAAAC |
| | | AAGCCCCAGGTAAAGACTTGAAATGGAT |
| | | GGGATGGATCAACACATACACAGGGAAT |
| | | CCTATCTATGCAGCCGACTTTAAAGGCAG |
| | | ATTTGCCTTCAGTTTGGAGACATCTGCCTC |
| | | CACCGCTTTCCTGCAAATAAATAACCTGA |
| | | AAAATGAAGATACCGCTACATACTTCTGT |
| | | GTACGGTTCCAGTACGGAGATTACCGCTA |
| | | TTTCGATGTGTGGGGCGCAGGTACCACAG |
| | | TAACCGTCTCCTCA |
| 42 | VH-CDR1 | GYTFTNYGVK |
| 43 | VH-CDR2 | WINTYTGNPIYAADFKG |
| 44 | VH-CDR3 | FQYGDYRYFDV |
| 45 | AGX-A07 | QIILSQSPAILSASPGEKVTMTCRANSGISFIN |
| | Variable light (VL) chain- | WYQQKPGSSPKPWIYGTANLASGVPARFG |
| | amino acid | GSGSGTSYSLTISRVEAEDAATYYCQQWSS |
| | | NPLTFGAGTKLELR |
| 46 | AGX-A07 | CAAATTATTCTCTCCCAGTCTCCAGCAAT |
| | Variable light (VL) chain- | CCTGTCTGCATCTCCAGGGGAGAAGGTCA |
| | nucleic acid | CGATGACTTGCAGGGCCAACTCAGGTATT |
| | | AGTTTCATCAACTGGTACCAGCAGAAGCC |
| | | AGGATCCTCCCCCAAACCCTGGATTTATG |
| | | GCACAGCCAACCTGGCTTCTGGAGTCCCT |
| | | GCTCGCTTCGGTGGCAGTGGGTCTGGGAC |
| | | TTCTTACTCTCTCACAATCAGCAGAGTGG |
| | | AGGCTGAAGACGCTGCCACTTATTACTGC |
| | | CAGCAGTGGAGTAGTAACCCGCTCACGTT |
| | | CGGTGCTGGGACCAAGCTGGAGTTGAGA |
| 47 | AGX-A07 | CAAATAATTCTGTCACAGTCCCCCGCTAT |
| | Variable light (VL) chain- | ACTTAGTGCTTCACCAGGAGAAAAAGTGA |
| | codon optimized nucleic acid | CCATGACTTGTAGAGCTAATTCTGGCATA |
| | | TCATTCATCAACTGGTATCAACAAAAGCC |
| | | AGGTTCCTCCCCCAAGCCATGGATTTACG |
| | | GGACCGCCAACCTTGCTTCTGGGGTACCC |
| | | GCTCGTTTCGGCGGATCAGGTTCAGGAAC |
| | | TTCCTATAGCCTCACTATCAGTCGGGTTG |
| | | AAGCTGAGGATGCCGCTACATATTACTGC |
| | | CAGCAATGGTCTAGTAATCCACTTACCTT |
| | | TGGAGCTGGCACCAAATTGGAACTTCGT |
| 48 | VL-CDR1 | RANSGISFIN |
| 49 | VL-CDR2 | GTANLAS |
| 50 | VL-CDR3 | QQWSSNPLT |

Antibody AGX-A08

| | | |
|---|---|---|
| 51 | AGX-A08 | EVQLQQSGPELVKPGASVKLSCKASGYTVT |
| | Variable heavy (VH) chain- | SYVMHWVKQKPGQGLEWIGYINPYSDVTN |
| | amino acid | CNEKFKGKATLTSDKTSSTAYMELSSLTSE |
| | | DSAVYYCSSYGGGFAYWGQGTLVTVSA |
| 52 | AGX-A08 | GAGGTCCAGCTGCAGCAGTCTGGACCTGA |
| | Variable heavy (VH) chain- | GCTGGTAAAGCCTGGGGCTTCAGTGAAGC |
| | nucleic acid | TGTCCTGCAAGGCTTCTGGATACACAGTC |
| | | ACTAGCTATGTTATGCACTGGGTGAAGCA |
| | | GAAGCCTGGGCAGGGCCTTGAGTGGATTG |
| | | GATATATTAATCCTTACAGTGATGTTACT |
| | | AACTGCAATGAGAAGTTCAAAGGCAAGG |
| | | CCACACTGACTTCAGACAAAACCTCCAGC |
| | | ACAGCCTACATGGAGCTCAGCAGCCTGAC |
| | | CTCTGAGGACTCTGCGGTCTATTACTGTTC |
| | | CTCCTACGGTGGGGGGTTTGCTTACTGGG |
| | | GCCAAGGGACTCTGGTCACTGTCTCTGCA |
| 53 | AGX-A08 | GAAGTCCAGCTTCAGCAATCCGGCCCAGA |
| | Variable heavy (VH) chain- | ACTGGTAAAACCAGGCGCAAGTGTTAAGT |
| | codon optimized nucleic acid | TGAGTTGCAAAGCCAGTGGTTATACCGTT |
| | | ACTTCATACGTCATGCATTGGGTAAAACA |
| | | AAAGCCCGGCCAAGGGCTTGAATGGATC |
| | | GGCTACATCAACCCTTACTCTGACGTCAC |
| | | CAACTGCAACGAGAAATTCAAAGGGAAA |
| | | GCCACATTGACCTCTGACAAGACAAGCAG |
| | | TACCGCCTACATGGAGCTTTCTAGTTTGA |
| | | CTTCTGAAGACTCTGCTGTCTACTACTGTA |
| | | GCAGCTACGGCGGCGGCTTTGCTTACTGG |
| | | GGCCAGGGTACATTGGTGACTGTGAGTGC |
| | | A |
| 54 | VH-CDR1 | GYTVTSYVMH |
| 55 | VH-CDR2 | YINPYSDVTNCNEKFKG |
| 56 | VH-CDR3 | YGGGFAY |
| 57 | AGX-A08 | DIQMTQSPASLSASVGEPVTITCRASKNIYT |
| | Variable light chain(VL)- | YLAWYHQKQGKSPQFLVYNARTLAGGVPS |
| | amino acid | RLSGSGSVTQFSLNINTLHREDLGTYFCQHH |
| | | YDTPYTFGGGTNLEIK |
| 58 | AGX-A08 | GACATCCAGATGACTCAGTCTCCAGCCTC |
| | Variable light (VL) chain- | CCTATCTGCATCTGTGGGAGAACCTGTCA |
| | nucleic acid | CCATCACATGTCGAGCAAGTAAGAATATT |
| | | TACACATATTTAGCATGGTATCACCAGAA |
| | | ACAGGGAAAATCTCCTCAGTTCCTGGTCT |
| | | ATAATGCAAGAACCTTAGCAGGAGGTGT |
| | | GCCATCAAGGCTCAGTGGCAGTGGATCAG |
| | | TCACGCAGTTTTCTCTAAACATCAACACC |
| | | TTGCATCGAGAAGATTTAGGGACTTACTT |
| | | CTGTCAACATCATTATGATACTCCGTACA |
| | | CGTTCGGAGGGGGGACCAACCTGGAAAT |
| | | AAAA |
| 59 | AGX-A08 | GACATCCAGATGACACAGTCACCAGCATC |
| | Variable light (VL) chain- | CCTGTCCGCCTCAGTTGGGGAGCCTGTTA |
| | codon optimized nucleic acid | CCATAACTTGTCGGGCAAGCAAAAACATA |
| | | TACACCTATTTGGCTTGGTATCACCAAAA |
| | | GCAAGGTAAGTCACCTCAGTTTCTTGTAT |
| | | ATAATGCCCGCACACTTGCTGGCGGAGTA |
| | | CCCTCTCGATTGTCTGGATCTGGCAGCGT |
| | | TACCCAATTCAGCCTGAACATCAACACCC |
| | | TCCATCGGGAAGATTTGGGTACCTATTTC |
| | | TGTCAACATCACTACGACACCCCATACAC |
| | | CTTCGGAGGCGGCACAAATTTGGAAATTA |
| | | AA |
| 60 | VL-CDR1 | RASKNIYTYLA |
| 61 | VL-CDR2 | NARTLAG |
| 62 | VL-CDR3 | QHHYDTPYT |

Antibody AGX-A09

| | | |
|---|---|---|
| 63 | AGX-A09 | EVQLQQSGPELVKPGASVKMSCKASGYTFS |
| | Variable heavy (VH) chain- | SYVMHWVKQKPGQGLEWIGYINPYSDVTN |
| | amino acid | YNEKFKGKATLTSDRSSNTAYMELSSLTSE |
| | | DSAVYYCARNYFDWGRGTLVTVSA |
| 64 | AGX-A09 | GAGGTCCAGCTGCAGCAGTCTGGACCTGA |
| | Variable heavy (VH) chain- | GCTGGTAAAGCCTGGGGCTTCAGTGAAGA |
| | nucleic acid | TGTCCTGCAAGGCTTCTGGATACACATTC |
| | | TCTAGCTATGTTATGCACTGGGTGAAGCA |
| | | GAAGCCTGGGCAGGGCCTTGAGTGGATTG |
| | | GATATATTAATCCTTACAGTGATGTCACT |
| | | AACTACAATGAGAAGTTCAAAGGCAAGG |
| | | CCACACTGACTTCAGACAGATCCTCCAAC |
| | | ACAGCCTACATGGAACTCAGCAGCCTGAC |
| | | CTCTGAGGACTCTGCGGTCTATTACTGTG |
| | | CAAGAAATTACTTCGACTGGGGCCGAGG |
| | | GACTCTGGTCACAGTCTCTGCA |
| 65 | AGX-A09 | GAGGTACAGCTTCAGCAGAGTGGTCCAG |
| | Variable heavy (VH) chain- | AACTCGTCAAGCCTGGGGCAAGCGTTAAG |
| | codon optimized nucleic acid | ATGAGTTGTAAAGCATCCGGTTACACATT |
| | | CAGTAGCTATGTTATGCACTGGGTCAAAC |
| | | AGAAGCCTGGGCAGGGGTTGGAGTGGAT |
| | | CGGATATATAAATCCCTATTCAGACGTAA |
| | | CTAATTATAATGAAAAGTTCAAGGGGAA |
| | | AGCAACCTTGACAAGTGACCGGTCATCTA |
| | | ATACCGCATACATGGAGCTGAGCTCATTG |
| | | ACAAGTGAGGACTCTGCTGTGTATTACTG |
| | | TGCCCGGAACTACTTCGACTGGGGTAGGG |
| | | GCACACTGGTAACTGTTAGTGCA |
| 66 | VH-CDR1 | GYTFSSYVMH |
| 67 | VH-CDR2 | YINPYSDVTNYNEKFKG |
| 68 | VH-CDR3 | NYFD |
| 69 | AGX-A09 | DIQMTQSPASLSASVGETVTITCRASKNVYS |
| | Variable light (VL) chain- | YLAWFQQKQGKSPQLLVYNAKTLAEGVPS |
| | amino acid | RFSGGGSGTQFSLKINSLQPADFGSYYCQH |
| | | HYNIPFTFGSGTKLEIK |
| 70 | AGX-A09 | GACATCCAGATGACTCAGTCTCCAGCCTC |
| | Variable light (VL) chain- | CCTATCTGCATCTGTGGGAGAAACTGTCA |
| | nucleic acid | CCATCACATGTCGAGCAAGTAAAAATGTT |
| | | TACAGTTATTTAGCATGGTTTCAACAGAA |
| | | ACAGGGGAAATCTCCTCAGCTCCTGGTCT |
| | | ATAATGCTAAAACCTTAGCAGAAGGTGTG |
| | | CCATCAAGGTTCAGTGGCGGGGGATCAG |
| | | GCACACAGTTTTCTCTGAAGATCAACAGC |
| | | CTGCAGCCTGCAGATTTTGGGAGTTATTA |
| | | CTGTCAACATCATTATAATATTCCATTCAC |
| | | GTTCGGCTCGGGGACAAAGTTGGAAATA |
| | | AAA |
| 71 | AGX-A09 | GACATACAAATGACACAAAGTCCCGCTA |
| | Variable light (VL) chain- | GTCTTTCAGCCAGTGTTGGTGAGACTGTG |
| | codon optimized nucleic acid | ACAATAACCTGTAGAGCTAGCAAAAATGT |
| | | CTACTCCTATCTGGCTTGGTTCCAGCAGA |
| | | AACAAGGAAAGAGTCCTCAGTTGCTCGTA |
| | | TATAATGCTAAAACTTTGGCAGAAGGCGT |
| | | CCCTTCTCGTTTCAGTGGCGGAGGAAGTG |
| | | GGACTCAATTCTCACTGAAGATCAATAGC |
| | | CTCCAGCCCGCCGACTTTGGGAGCTACTA |
| | | TTGCCAACATCATTACAACATACCATTCA |
| | | CCTTTGGCTCAGGTACTAAACTCGAAATT |
| | | AAA |
| 72 | VL-CDR1 | RASKNVYSYLA |
| 73 | VL-CDR2 | NAKTLAE |
| 74 | VL-CDR3 | QHHYNIPFT |

Antibody AGX-A11

| | | |
|---|---|---|
| 75 | AGX-A11 | QIQLVQSGPELKKPGETVKISCKASGFTFTN |
| | Variable heavy (VH) chain- | YPMHWVKQAPGKGLKWMGWINTYSGVPT |
| | amino acid | YADDFKGRFAFSLETSASTAYLQINNLKNE |
| | | DMATYFCARGGYDGSREFAYWGQGTLVT |
| | | VS |
| 76 | AGX-A11 | CAGATCCAGTTGGTGCAGTCTGGACCTGA |
| | Variable heavy (VH) chain- | GCTGAAGAAGCCTGGAGAGACAGTCAAG |
| | nucleic acid | ATCTCCTGCAAGGCTTCTGGGTTTACCTTC |
| | | ACAAACTATCCAATGCACTGGGTGAAGCA |
| | | GGCTCCAGGAAAGGGTTTAAAGTGGATG |
| | | GGCTGGATAAACACCTACTCTGGAGTGCC |
| | | AACATATGCAGATGACTTCAAGGGACGGT |
| | | TTGCCTTCTCTTTGGAAACCTCTGCCAGCA |
| | | CTGCATATTTGCAGATCAACAACCTCAAA |
| | | AATGAGGACATGGCTACATATTTCTGTGC |
| | | AAGAGGGGGCTACGATGGTAGCAGGGAG |
| | | TTTGCTTACTGGGGCCAAGGGACTCTGGT |
| | | CACTGTCTCT |
| 77 | AGX-A11 | CAGATACAACTCGTCCAGTCAGGTCCAGA |
| | Variable heavy (VH) chain- | GTTGAAGAAACCCGGAGAAACTGTGAAG |
| | codon optimized nucleic acid | ATATCCTGTAAAGCCAGCGGCTTTACTTT |
| | | CACAAACTACCCCATGCATTGGGTGAAGC |
| | | AGGCCCCCGGAAAAGGACTCAAATGGAT |
| | | GGGATGGATCAACACATACAGTGGGGTG |
| | | CCTACTTACGCAGACGATTTCAAAGGAAG |
| | | GTTCGCATTTAGCTTGGAAACTAGCGCAT |
| | | CTACAGCATATCTCCAGATTAACAATCTT |
| | | AAAAATGAGGATATGGCAACATACTTCTG |
| | | CGCTAGGGGAGGTTACGATGGGAGCAGG |
| | | GAGTTCGCTTATTGGGGGCAAGGGACTCT |
| | | TGTGACTGTAAGT |
| 78 | VH-CDR1 | GFTFTNYPMH |
| 79 | VH-CDR2 | WINTYSGVPTYADDFKG |
| 80 | VH-CDR3 | GGYDGSREFAY |
| 81 | AGX-A11 | DIVLTQSPASLAASLGQRATTSYRASKSVST |
| | Variable light (VL) chain- | SGYSYMHWNQQKPGQPPRLLIYLVSNLESG |
| | amino acid | VPARFSGSGSGTDFTLNIHPVEEEDAATYYC |
| | | QHIRELTTFGGGTKLEIK |
| 82 | AGX-A11 | GACATTGTGCTGACACAGTCTCCTGCTTC |
| | Variable light (VL) chain- | CTTAGCTGCATCTCTGGGGCAGAGGGCCA |
| | nucleic acid | CCACCTCATACAGGGCCAGCAAAAGTGTC |
| | | AGTACATCTGGCTATAGTTATATGCACTG |
| | | GAACCAACAGAAACCAGGACAGCCACCC |
| | | AGACTCCTCATCTATCTTGTATCCAACCTA |
| | | GAATCTGGGGTCCCTGCCAGGTTCAGTGG |
| | | CAGTGGGTCTGGGACAGACTTCACCCTCA |
| | | ACATCCATCCTGTGGAGGAGGAGGATGCT |
| | | GCAACCTATTACTGTCAGCACATTAGGGA |
| | | GCTTACCACGTTCGGAGGGGGGACCAAG |
| | | CTGGAAATAAAA |
| 83 | AGX-A11 | GACATAGTGCTCACTCAGAGCCCTGCATC |
| | Variable light (VL) chain- | CCTTGCCGCCTCCCTCGGACAACGAGCTA |
| | codon optimized nucleic acid | CTACAAGCTACCGGGCATCAAAGTCCGTT |
| | | AGCACATCAGGATACAGCTATATGCACTG |
| | | GAATCAGCAAAAGCCAGGCCAACCACCC |
| | | CGTCTTCTCATCTACCTCGTAAGTAATCTG |
| | | GAATCAGGCGTGCCAGCCCGATTCAGTGG |
| | | GTCAGGGTCTGGGACAGATTTCACCCTCA |
| | | ACATCCATCCAGTAGAGGAAGAGGACGC |
| | | AGCAACATATTACTGCCAACACATTAGAG |
| | | AACTTACCACTTTCGGAGGAGGAACTAAA |
| | | TTGGAGATCAAA |
| 84 | VL-CDR1 | RASKSVSTSGYSYMH |
| 85 | VL-CDR2 | LVSNLES |
| 86 | VL-CDR3 | QHIRELT |

Constant Region Sequences

| | | |
|---|---|---|
| 87 | IgG1 G1m17* (heavy chain | ASTKGPSVFPLAPSSKSTSGGTAALGCLVKD |
| | constant region) | YFPEPVTVSWNSGALTSGVHTFPAVLQSSG |
| | *with L234A/L235A/G237A | LYSLSSVVTVPSSSLGTQTYICNVNHKPSNT |
| | mutations | KVDKKVEPKSCDKTHTCPPCPAPEAAGAPS |
| | SEQ ID NO: 88 is sequence | VFLFPPKPKDTLMISRTPEVTCVVVDVSHED |
| | without the terminal lysine | PEVKFNWYVDGVEVHNAKTKPREEQYNST |
| | | YRVVSVLTVLHQDWLNGKEYKCKVSNKA |
| | | LPAPIEKTISKAKGQPREPQVYTLPPSREEM |
| | | TKNQVSLTCLVKGFYPSDIAVEWESNGQPE |
| | | NNYKTTPPVLDSDGSFFLYSKLTVDKSRWQ |
| | | QGNVFSCSVMHEALHNHYTQKSLSLSPGK |
| 88 | IgG1 Glm17* (heavy chain | ASTKGPSVFPLAPSSKSTSGGTAALGCLVKD |
| | constant region) | YFPEPVTVSWNSGALTSGVHTFPAVLQSSG |
| | *with L234A/L235A/G237A | LYSLSSVVTVPSSSLGTQTYICNVNHKPSNT |
| | mutations | KVDKKVEPKSCDKTHTCPPCPAPEAAGAPS |
| | | VFLFPPKPKDTLMISRTPEVTCVVVDVSHED |
| | | PEVKFNWYVDGVEVHNAKTKPREEQYNST |
| | | YRVVSVLTVLHQDWLNGKEYKCKVSNKA |
| | | LPAPIEKTISKAKGQPREPQVYTLPPSREEM |
| | | TKNQVSLTCLVKGFYPSDIAVEWESNGQPE |
| | | NNYKTTPPVLDSDGSFFLYSKLTVDKSRWQ |
| | | QGNVFSCSVMHEALHNHYTQKSLSLSPG |
| 89 | IgG1 Km3 (light chain constant | RTVAAPSVFIFPPSDEQLKSGTASVVCLLNN |
| | region) | FYPREAKVQWKVDNALQSGNSQESVTEQD |
| | | SKDSTYSLSSTLTLSKADYEKHKVYACEVT |
| | | HQGLSSPVTKSFNRGEC |

Humanized AGX-A07 sequences

| | | |
|---|---|---|
| 90 | AGX-A07 (humanized) H2 | QVQLVQSGAEVKKPGASVKVSCKASGYTF |
| | Heavy chain amino acid | TNYGVKWVRQAPGQDLEWMGWINTYTGN |
| | | PIYAADFKGRVTMTTDTSTSTAFMELRSLRS |
| | | DDTAVYYCVRFQYGDYRYFDVWGQGTLV |
| | | TVSSASTKGPSVFPLAPSSKSTSGGTAALGC |
| | | LVKDYFPEPVTVSWNSGALTSGVHTFPAVL |
| | | QSSGLYSLSSVVTVPSSSLGTQTYICNVNHK |
| | | PSNTKVDKKVEPKSCDKTHTCPPCPAPEAA |
| | | GAPSVFLFPPKPKDTLMISRTPEVTCVVVDV |
| | | SHEDPEVKFNWYVDGVEVHNAKTKPREEQ |
| | | YNSTYRVVSVLTVLHQDWLNGKEYKCKVS |
| | | NKALPAPIEKTISKAKGQPREPQVYTLPPSR |
| | | EEMTKNQVSLTCLVKGFYPSDIAVEWESNG |
| | | QPENNYKTTPPVLDSDGSFFLYSKLTVDKS |
| | | RWQQGNVFSCSVMHEALHNHYTQKSLSLS |
| | | PGK |
| 91 | AGX-A07 (humanized) H2 | TCTACCGGACAGGTGCAGTTGGTTCAGTC |
| | Heavy chain nucleic acid | TGGCGCCGAAGTGAAGAAACCTGGCGCTT |
| | | CTGTGAAGGTGTCCTGCAAGGCCTCTGGC |
| | | TACACCTTTACCAACTACGGCGTGAAATG |
| | | GGTCCGACAGGCTCCTGGACAGGATCTGG |
| | | AATGGATGGGCTGGATCAACACCTACACC |
| | | GGCAATCCTATCTACGCCGCCGACTTCAA |
| | | GGGCAGAGTGACCATGACCACCGACACC |
| | | TCTACCTCCACCGCCTTCATGGAACTGCG |
| | | GTCCCTGAGATCTGACGACACCGCCGTGT |
| | | ACTACTGCGTGCGGTTTCAGTACGGCGAC |
| | | TACCGGTACTTTGATGTGTGGGGCCAGGG |
| | | CACACTGGTCACCGTTTCTTCCGCTTCTAC |
| | | CAAGGGACCCAGCGTGTTCCCTCTGGCTC |
| | | CTTCCTCTAAATCCACCTCTGGCGGAACC |
| | | GCTGCTCTGGGCTGTCTGGTCAAGGATTA |
| | | CTTCCCTGAGCCTGTGACCGTGTCCTGGA |
| | | ACTCTGGTGCTCTGACATCCGGCGTGCAC |
| | | ACCTTTCCAGCTGTGCTGCAGTCCTCTGG |
| | | CCTGTACTCTCTGTCCTCTGTCGTGACCGT |
| | | GCCTTCTAGCTCTCTGGGCACCCAGACCT |
| | | ACATCTGCAACGTGAACCACAAGCCTTCC |
| | | AACACCAAGGTGGACAAGAAGGTGGAAC |
| | | CCAAGTCCTGCGACAAGACCCACACCTGT |
| | | CCTCCATGTCCTGCTCCAGAAGCTGCTGG |
| | | CGCTCCCTCTGTGTTCCTGTTTCCTCCAAA |
| | | GCCTAAGGACACCCTGATGATCTCTCGGA |
| | | CCCCTGAAGTGACCTGCGTGGTGGTGGAT |
| | | GTGTCTCACGAGGACCCAGAAGTGAAGTT |
| | | CAATTGGTACGTGGACGGCGTGGAAGTGC |
| | | ACAACGCCAAGACCAAGCCTAGAGAGGA |
| | | ACAGTACAACTCCACCTACAGAGTGGTGT |
| | | CCGTGCTGACCGTGCTGCACCAGGATTGG |
| | | CTGAACGGCAAAGAGTACAAGTGCAAGG |
| | | TGTCCAACAAGGCACTGCCCGCTCCTATC |
| | | GAAAAGACCATCTCCAAGGCTAAGGGCC |
| | | AGCCTCGGGAACCTCAGGTTTACACCCTG |
| | | CCTCCATCTCGGGAAGAGATGACCAAGA |
| | | ACCAGGTGTCCCTGACCTGCCTCGTGAAG |
| | | GGCTTCTACCCTTCCGATATCGCCGTGGA |
| | | ATGGGAGTCCAATGGCCAGCCTGAGAAC |
| | | AACTACAAGACAACCCCTCCTGTGCTGGA |
| | | CTCCGACGGCTCATTCTTCCTGTACTCCAA |
| | | GCTGACAGTGGACAAGTCTCGGTGGCAGC |
| | | AGGGCAACGTGTTCTCCTGTTCTGTGATG |
| | | CACGAGGCCCTGCACAACCACTACACACA |
| | | GAAGTCCCTGTCTCTGTCCCCTGGCAAGT |
| | | GA |
| 92 | AGX-A07 H2v1 | EVQLVQSGAEVKKPGASVKVSCKASGYTF |
| | Heavy chain amino acid | TNYGVKWVRQAPGQGLEWMGWINTYTGN |
| | | PIYAADFKGRVTMTTDTSTSTAYMELRSLR |
| | | SDDTAVYYCVRFQYGDYRYFDVWGQGTL |
| | | VTVSSASTKGPSVFPLAPSSKSTSGGTAALG |
| | | CLVKDYFPEPVTVSWNSGALTSGVHTFPAV |
| | | LQSSGLYSLSSVVTVPSSSLGTQTYICNVNH |
| | | KPSNTKVDKKVEPKSCDKTHTCPPCPAPEA |
| | | AGAPSVFLFPPKPKDTLMISRTPEVTCVVVD |
| | | VSHEDPEVKFNWYVDGVEVHNAKTKPREE |
| | | QYNSTYRVVSVLTVLHQDWLNGKEYKCK |
| | | VSNKALPAPIEKTISKAKGQPREPQVYTLPP |
| | | SREEMTKNQVSLTCLVKGFYPSDIAVEWES |
| | | NGQPENNYKTTPPVLDSDGSFFLYSKLTVD |
| | | KSRWQQGNVFSCSVMHEALHNHYTQKSLS |
| | | LSPGK |
| 93 | AGX-A07 H2v1 | GAAGTGCAGTTGGTGCAGTCTGGCGCCGA |
| | Heavy chain nucleic acid | AGTGAAGAAACCTGGCGCTTCTGTGAAGG |
| | | TGTCCTGCAAGGCCTCTGGCTACACCTTT |
| | | ACCAACTACGGCGTGAAATGGGTCCGAC |
| | | AGGCTCCTGGACAAGGCCTGGAATGGAT |
| | | GGGCTGGATCAACACCTACACCGGCAATC |
| | | CTATCTACGCCGCCGACTTCAAGGGCAGA |
| | | GTGACCATGACCACCGACACCTCTACCTC |
| | | CACCGCCTACATGGAACTGCGGTCCCTGA |
| | | GATCTGACGACACCGCCGTGTACTACTGC |
| | | GTGCGGTTTCAGTACGGCGACTACCGGTA |
| | | CTTTGATGTGTGGGGCCAGGGCACACTGG |
| | | TCACCGTTTCTTCCGCTTCTACCAAGGGA |
| | | CCCAGCGTGTTCCCTCTGGCTCCTTCCTCT |
| | | AAATCCACCTCTGGCGGAACCGCTGCTCT |
| | | GGGCTGTCTGGTCAAGGATTACTTCCCTG |
| | | AGCCTGTGACCGTGTCCTGGAATTCTGGT |
| | | GCTCTGACATCCGGCGTGCACACCTTTCC |
| | | AGCTGTGCTGCAGTCCTCTGGCCTGTACT |
| | | CTCTGTCCTCTGTCGTGACCGTGCCTTCTA |
| | | GCTCTCTGGGCACCCAGACCTACATCTGC |
| | | AACGTGAACCACAAGCCTTCCAACACCAA |
| | | GGTGGACAAGAAGGTGGAACCCAAGTCC |
| | | TGCGACAAGACCCACACCTGTCCTCCATG |
| | | TCCTGCTCCAGAAGCTGCTGGCGCTCCCT |
| | | CTGTGTTCCTGTTTCCTCCAAAGCCTAAG |
| | | GACACCCTGATGATCTCTCGGACCCCTGA |
| | | AGTGACCTGCGTGGTGGTGGATGTGTCTC |
| | | ACGAGGACCCAGAAGTGAAGTTCAATTG |
| | | GTACGTGGACGGCGTGGAAGTGCACAAC |
| | | GCCAAGACCAAGCCTAGAGAGGAACAGT |
| | | ACAACTCCACCTACAGAGTGGTGTCCGTG |
| | | CTGACCGTGCTGCACCAGGATTGGCTGAA |
| | | CGGCAAAGAGTACAAGTGCAAGGTGTCC |
| | | AACAAGGCACTGCCCGCTCCTATCGAAAA |
| | | GACCATCTCCAAGGCTAAGGGCCAGCCTC |
| | | GGGAACCTCAGGTTTACACCCTGCCTCCA |
| | | TCTCGGGAAGAGATGACCAAGAACCAGG |
| | | TGTCCCTGACCTGCCTCGTGAAGGGCTTC |
| | | TACCCTTCCGATATCGCCGTGGAATGGGA |
| | | GTCCAATGGCCAGCCTGAGAACAACTACA |
| | | AGACAACCCCTCCTGTGCTGGACTCCGAC |
| | | GGCTCATTCTTCCTGTACTCCAAGCTGAC |
| | | AGTGGACAAGTCTCGGTGGCAGCAGGGC |
| | | AACGTGTTCTCCTGTTCTGTGATGCACGA |
| | | GGCCCTGCACAACCACTACACACAGAAGT |
| | | CCCTGTCTCTGTCCCCTGGCAAGTGA |
| 94 | VH-CDR1 | GYTFTNYGVK |
| 95 | VH-CDR2 | WINTYTGNPIYAADFK |
| 96 | VH-CDR3 | FQYGDYRYFDV |
| 97 | AGX-A07 L5 | EIILTQSPATLSLSPGERATLSCRANSGISFIN |
| | Light chain amino acid | WYQQKPGQAPRLLIYGTANLASGIPARFGG |
| | | SGSGRDFTLTISSLEPEDFAVYYCQQWSSNP |
| | | LTFGGGTKVEIKRTVAAPSVFIFPPSDEQLKS |
| | | GTASVVCLLNNFYPREAKVQWKVDNALQS |
| | | GNSQESVTEQDSKDSTYSLSSTLTLSKADYE |
| | | KHKVYACEVTHQGLSSPVTKSFNRGEC |
| 98 | AGX-A07 L5 | AAGCTTGCCACCATGGAAACCGACACACT |
| | Light chain nucleic acid | GCTGCTGTGGGTGCTGTTGTTGTGGGTGC |
| | | CAGGATCTACCGGAGAGATCATCCTGACA |
| | | CAGAGCCCCGCCACATTGTCTCTGAGTCC |
| | | TGGCGAGAGAGCTACCCTGTCCTGTAGAG |
| | | CCAACTCCGGCATCTCCTTCATCAACTGG |
| | | TATCAGCAGAAGCCCGGCCAGGCTCCTAG |
| | | ACTGCTGATCTATGGCACCGCTAACCTGG |
| | | CCTCTGGCATCCCTGCTAGATTTGGCGGC |
| | | TCTGGCTCTGGCAGAGACTTCACCCTGAC |
| | | CATCTCTAGCCTGGAACCTGAGGACTTCG |
| | | CCGTGTACTACTGCCAGCAGTGGTCTAGC |
| | | AACCCTCTGACCTTTGGCGGAGGCACCAA |
| | | GGTGGAAATCAAGAGAACCGTGGCCGCT |
| | | CCTTCCGTGTTCATCTTCCCACCATCTGAC |
| | | GAGCAGCTGAAGTCTGGCACAGCCTCTGT |
| | | CGTGTGCCTGCTGAACAACTTCTACCCTC |
| | | GGGAAGCCAAGGTGCAGTGGAAGGTGGA |
| | | CAATGCCCTGCAGTCCGGCAACTCCCAAG |
| | | AGTCTGTGACCGAGCAGGACTCCAAGGA |
| | | CTCTACCTACAGCCTGTCCTCCACACTGA |
| | | CCCTGTCTAAGGCCGACTACGAGAAGCAC |
| | | AAGGTGTACGCCTGTGAAGTGACCCACCA |
| | | GGGACTGTCTAGCCCCGTGACCAAGTCTT |
| | | TCAACCGGGGCGAGTGCTGA |
| 99 | AGX-A07 L5v1 | EIVLTQSPATLSLSPGERATLSCRANSGISFIN |
| | Light chain amino acid | WYQQKPGQAPRLLIYGTANLASGIPARFSG |
| | | SGSGRDFTLTISSLEPEDFAVYYCQQWSSNP |
| | | LTFGGGTKVEIKRTVAAPSVFIFPPSDEQLKS |
| | | GTASVVCLLNNFYPREAKVQWKVDNALQS |
| | | GNSQESVTEQDSKDSTYSLSSTLTLSKADYE |
| | | KHKVYACEVTHQGLSSPVTKSFNRGEC |
| 100 | AGX-A07 L5v1 | TCTACAGGCGAGATCGTGCTGACCCAGTC |
| | Light chain nucleic acid | TCCTGCCACATTGTCTCTGAGTCCTGGCG |
| | | AGAGAGCTACCCTGTCCTGTAGAGCCAAC |
| | | TCCGGCATCTCCTTCATCAACTGGTATCA |
| | | GCAGAAGCCCGGCCAGGCTCCTAGACTGC |
| | | TGATCTATGGCACCGCTAACCTGGCCTCT |
| | | GGCATCCCTGCTAGATTTTCCGGCTCTGG |
| | | CTCTGGCAGAGACTTCACCCTGACCATCT |
| | | CTAGCCTGGAACCTGAGGACTTCGCCGTG |
| | | TACTACTGCCAGCAGTGGTCTAGCAACCC |
| | | TCTGACCTTTGGCGGAGGCACCAAGGTGG |
| | | AAATCAAGAGAACCGTGGCCGCTCCTTCC |
| | | GTGTTCATCTTCCCACCATCTGACGAGCA |
| | | GCTGAAGTCTGGCACAGCCTCTGTCGTGT |
| | | GCCTGCTGAACAACTTCTACCCTCGGGAA |
| | | GCCAAGGTGCAGTGGAAGGTGGACAATG |
| | | CCCTGCAGTCCGGCAACTCCCAAGAGTCT |
| | | GTGACCGAGCAGGACTCCAAGGACTCTAC |
| | | CTACAGCCTGTCCTCCACACTGACCCTGT |
| | | CTAAGGCCGACTACGAGAAGCACAAGGT |
| | | GTACGCCTGTGAAGTGACCCACCAGGGAC |
| | | TGTCTAGCCCCGTGACCAAGTCTTTCAAC |
| | | CGGGGCGAGTGCTGA |
| 101 | AGX-A07 L5v2 | EIVLTQSPATLSLSPGERATLSCRAQSGISFIN |
| | Light chain amino acid | WYQQKPGQAPRLLIYGTANLASGIPARFSG |
| | | SGSGRDFTLTISSLEPEDFAVYYCQQWSSNP |
| | | LTFGGGTKVEIKRTVAAPSVFIFPPSDEQLKS |
| | | GTASVVCLLNNFYPREAKVQWKVDNALQS |
| | | GNSQESVTEQDSKDSTYSLSSTLTLSKADYE |
| | | KHKVYACEVTHQGLSSPVTKSFNRGEC |
| 102 | AGX-A07 L5v2 | TCTACAGGCGAGATCGTGCTGACCCAGTC |
| | Light chain nucleic acid | TCCTGCCACATTGTCTCTGAGTCCTGGCG |
| | | AGAGAGCTACCCTGTCTTGTAGAGCCCAG |
| | | TCCGGCATCTCCTTCATCAACTGGTATCA |
| | | GCAGAAGCCCGGCCAGGCTCCTAGACTGC |
| | | TGATCTATGGCACCGCTAACCTGGCCTCT |
| | | GGCATCCCTGCTAGATTTTCCGGCTCTGG |
| | | CTCTGGCAGAGACTTCACCCTGACCATCT |
| | | CTAGCCTGGAACCTGAGGACTTCGCCGTG |
| | | TACTACTGCCAGCAGTGGTCTAGCAACCC |
| | | TCTGACCTTTGGCGGAGGCACCAAGGTGG |
| | | AAATCAAGAGAACCGTGGCCGCTCCTTCC |
| | | GTGTTCATCTTCCCACCATCTGACGAGCA |
| | | GCTGAAGTCTGGCACAGCCTCTGTCGTGT |
| | | GCCTGCTGAACAACTTCTACCCTCGGGAA |
| | | GCCAAGGTGCAGTGGAAGGTGGACAATG |
| | | CCCTGCAGTCTGGCAACTCCCAAGAGTCT |
| | | GTGACCGAGCAGGACTCCAAGGACTCTAC |
| | | CTACAGCCTGTCCTCCACACTGACCCTGT |
| | | CTAAGGCCGACTACGAGAAGCACAAGGT |
| | | GTACGCCTGTGAAGTGACCCACCAGGGAC |
| | | TGTCTAGCCCCGTGACCAAGTCTTTCAAC |
| | | CGGGGCGAGTGCTGA |
| 103 | AGX-A07 L5v3 | EIVLTQSPATLSLSPGERATLSCRANSGISFIN |
| | Light chain amino acid | WYQQKPGQAPRLLIYGTANLASGIPARFSG |
| | | SGSGRDFTLTISSLEPEDFAVYYCQQYSSNP |
| | | LTFGGGTKVEIKRTVAAPSVFIFPPSDEQLKS |
| | | GTASVVCLLNNFYPREAKVQWKVDNALQS |
| | | GNSQESVTEQDSKDSTYSLSSTLTLSKADYE |
| | | KHKVYACEVTHQGLSSPVTKSFNRGEC |
| 104 | AGX-A07 L5v3 | TCTACAGGCGAGATCGTGCTGACCCAGTC |
| | Light chain nucleic acid | TCCTGCCACATTGTCTCTGAGTCCTGGCG |
| | | AGAGAGCTACCCTGTCCTGTAGAGCCAAC |
| | | TCCGGCATCTCCTTCATCAACTGGTATCA |
| | | GCAGAAGCCCGGCCAGGCTCCTAGACTGC |
| | | TGATCTATGGCACCGCTAACCTGGCCTCT |
| | | GGCATCCCTGCTAGATTTTCCGGCTCTGG |
| | | CTCTGGCAGAGACTTCACCCTGACCATCT |
| | | CTAGCCTGGAACCTGAGGACTTCGCCGTG |
| | | TACTACTGCCAGCAGTACAGCAGCAACCC |
| | | TCTGACCTTTGGCGGAGGCACCAAGGTGG |
| | | AAATCAAGAGAACCGTGGCCGCTCCTTCC |
| | | GTGTTCATCTTCCCACCATCTGACGAGCA |
| | | GCTGAAGTCTGGCACAGCCTCTGTCGTGT |
| | | GCCTGCTGAACAACTTCTACCCTCGGGAA |
| | | GCCAAGGTGCAGTGGAAGGTGGACAATG |
| | | CCCTGCAGTCCGGCAACTCCCAAGAGTCT |
| | | GTGACCGAGCAGGACTCCAAGGACTCTAC |
| | | CTACAGCCTGTCCTCCACACTGACCCTGT |
| | | CTAAGGCCGACTACGAGAAGCACAAGGT |
| | | GTACGCCTGTGAAGTGACCCACCAGGGAC |
| | | TGTCTAGCCCCGTGACCAAGTCTTTCAAC |
| | | CGGGGCGAGTGCTGA |
| 105 | AGX-A07 L5v4 | EIVLTQSPATLSLSPGERATLSCRAQSGISFIN |
| | Light chain amino acid | WYQQKPGQAPRLLIYGTANLASGIPARFSG |
| | | SGSGRDFTLTISSLEPEDFAVYYCQQYSSNP |
| | | LTFGGGTKVEIKRTVAAPSVFIFPPSDEQLKS |
| | | GTASVVCLLNNFYPREAKVQWKVDNALQS |
| | | GNSQESVTEQDSKDSTYSLSSTLTLSKADYE |
| | | KHKVYACEVTHQGLSSPVTKSFNRGEC |
| 106 | AGX-A07 L5v4 | TCTACAGGCGAGATCGTGCTGACCCAGTC |
| | Light chain nucleic acid | TCCTGCCACATTGTCTCTGAGTCCTGGCG |
| | | AGAGAGCTACCCTGTCTTGTAGAGCCCAG |
| | | TCCGGCATCTCCTTCATCAACTGGTATCA |
| | | GCAGAAGCCCGGCCAGGCTCCTAGACTGC |
| | | TGATCTATGGCACCGCTAACCTGGCCTCT |
| | | GGCATCCCTGCTAGATTTTCCGGCTCTGG |
| | | CTCTGGCAGAGACTTCACCCTGACCATCT |
| | | CTAGCCTGGAACCTGAGGACTTCGCCGTG |
| | | TACTACTGCCAGCAGTACAGCAGCAACCC |
| | | TCTGACCTTTGGCGGAGGCACCAAGGTGG |
| | | AAATCAAGAGAACCGTGGCCGCTCCTTCC |
| | | GTGTTCATCTTCCCACCATCTGACGAGCA |
| | | GCTGAAGTCTGGCACAGCCTCTGTCGTGT |
| | | GCCTGCTGAACAACTTCTACCCTCGGGAA |
| | | GCCAAGGTGCAGTGGAAGGTGGACAATG |
| | | CCCTGCAGTCTGGCAACTCCCAAGAGTCT |
| | | GTGACCGAGCAGGACTCCAAGGACTCTAC |
| | | CTACAGCCTGTCCTCCACACTGACCCTGT |
| | | CTAAGGCCGACTACGAGAAGCACAAGGT |
| | | GTACGCCTGTGAAGTGACCCACCAGGGAC |
| | | TGTCTAGCCCCGTGACCAAGTCTTTCAAC |
| | | CGGGGCGAGTGCTGA |
| 107 | VL-CDR1 (variant 1) | RANSGISFIN |
| 108 | VL-CDR1 (variant 2) | RAQSGISFIN |
| 109 | VL-CDR2 | GTANLAS |
| 110 | VL-CDR3 (variant 1) | QQWSSNPLT |
| 111 | VL-CDR3 (variant 2) | QQYSSNPLT |

Humanized AGX-A01 sequences

| | | |
|---|---|---|
| 112 | AGX-A01 H1 | EVQLVESGGGLVKPGGSLRLSCAASGFTFSS |
| | Heavy chain amino acid | FAMSWVRQAPGKGLEWVSTISSGSIYIYYT |
| | | DGVKGRFTISRDNAKNSLYLQMNSLRAEDT |
| | | AVYYCARRGIYYGYDGYAMDYWGQGTLV |
| | | TVSSASTKGPSVFPLAPSSKSTSGGTAALGC |
| | | LVKDYFPEPVTVSWNSGALTSGVHTFPAVL |
| | | QSSGLYSLSSVVTVPSSSLGTQTYICNVNHK |
| | | PSNTKVDKKVEPKSCDKTHTCPPCPAPEAA |
| | | GAPSVFLFPPKPKDTLMISRTPEVTCVVVDV |
| | | SHEDPEVKFNWYVDGVEVHNAKTKPREEQ |
| | | YNSTYRVVSVLTVLHQDWLNGKEYKCKVS |
| | | NKALPAPIEKTISKAKGQPREPQVYTLPPSR |
| | | EEMTKNQVSLTCLVKGFYPSDIAVEWESNG |
| | | QPENNYKTTPPVLDSDGSFFLYSKLTVDKS |
| | | RWQQGNVFSCSVMHEALHNHYTQKSLSLS |
| | | PGK |
| 113 | AGX-A01 H1 | GAGGTGCAGCTGGTTGAATCTGGCGGAG |
| | Heavy chain nucleic acid | GACTTGTGAAGCCTGGCGGCTCTCTGAGA |
| | | CTGTCTTGTGCCGCCTCTGGCTTCACCTTC |
| | | TCCAGCTTTGCCATGTCCTGGGTCCGACA |
| | | GGCTCCTGGCAAAGGACTGGAATGGGTGT |
| | | CCACCATCTCCTCCGGCTCCATCTACATCT |
| | | ACTACACCGACGGCGTGAAGGGCAGATT |
| | | CACCATCAGCAGAGACAACGCCAAGAAC |
| | | TCCCTGTACCTGCAGATGAACAGCCTGAG |
| | | AGCCGAGGACACCGCCGTGTACTATTGTG |
| | | CCAGACGGGGCATCTACTATGGCTACGAC |
| | | GGCTACGCTATGGACTATTGGGGACAGGG |
| | | CACACTGGTCACCGTGTCCTCTGCTTCTAC |
| | | CAAGGGACCCAGCGTGTTCCCTCTGGCTC |
| | | CTTCCTCTAAATCCACCTCTGGCGGAACC |
| | | GCTGCTCTGGGCTGTCTGGTCAAGGATTA |
| | | CTTCCCTGAGCCTGTGACCGTGTCCTGGA |
| | | ACTCTGGTGCTCTGACATCCGGCGTGCAC |
| | | ACCTTTCCAGCTGTGCTGCAGTCCTCTGG |
| | | CCTGTACTCTCTGTCCTCTGTCGTGACCGT |
| | | GCCTTCTAGCTCTCTGGGCACCCAGACCT |
| | | ACATCTGCAACGTGAACCACAAGCCTTCC |
| | | AACACCAAGGTGGACAAGAAGGTGGAAC |
| | | CCAAGTCCTGCGACAAGACCCACACCTGT |
| | | CCTCCATGTCCTGCTCCAGAAGCTGCTGG |
| | | CGCTCCCTCTGTGTTCCTGTTTCCTCCAAA |
| | | GCCTAAGGACACCCTGATGATCTCTCGGA |
| | | CCCCTGAAGTGACCTGCGTGGTGGTGGAT |
| | | GTGTCTCACGAGGACCCAGAAGTGAAGTT |
| | | CAATTGGTACGTGGACGGCGTGGAAGTGC |
| | | ACAACGCCAAGACCAAGCCTAGAGAGGA |
| | | ACAGTACAACTCCACCTACAGAGTGGTGT |
| | | CCGTGCTGACCGTGCTGCACCAGGATTGG |
| | | CTGAACGGCAAAGAGTACAAGTGCAAGG |
| | | TGTCCAACAAGGCACTGCCCGCTCCTATC |
| | | GAAAAGACCATCTCCAAGGCTAAGGGCC |
| | | AGCCTCGGGAACCTCAGGTTTACACCCTG |
| | | CCTCCATCTCGGGAAGAGATGACCAAGA |
| | | ACCAGGTGTCCCTGACCTGCCTCGTGAAG |
| | | GGCTTCTACCCTTCCGATATCGCCGTGGA |
| | | ATGGGAGTCCAATGGCCAGCCTGAGAAC |
| | | AACTACAAGACAACCCCTCCTGTGCTGGA |
| | | CTCCGACGGCTCATTCTTCCTGTACTCCAA |
| | | GCTGACAGTGGACAAGTCTCGGTGGCAGC |
| | | AGGGCAACGTGTTCTCCTGTTCTGTGATG |
| | | CACGAGGCCCTGCACAACCACTACACACA |
| | | GAAGTCCCTGTCTCTGTCCCCTGGCAAGT |
| | | GA |
| 114 | AGX-A01 H1v1 | EVQLVESGGGLVKPGGSLRLSCAASGFTFSS |
| | Heavy chain amino acid | FAMSWVRQAPGKGLEWVSTISSGSIYIYYT |
| | | DSVKGRFTISRDNAKNSLYLQMNSLRAEDT |
| | | AVYYCARRGIYYGYEGYAMDYWGQGTLV |
| | | TVSSASTKGPSVFPLAPSSKSTSGGTAALGC |
| | | LVKDYFPEPVTVSWNSGALTSGVHTFPAVL |
| | | QSSGLYSLSSVVTVPSSSLGTQTYICNVNHK |
| | | PSNTKVDKKVEPKSCDKTHTCPPCPAPEAA |
| | | GAPSVFLFPPKPKDTLMISRTPEVTCVVVDV |
| | | SHEDPEVKFNWYVDGVEVHNAKTKPREEQ |
| | | YNSTYRVVSVLTVLHQDWLNGKEYKCKVS |
| | | NKALPAPIEKTISKAKGQPREPQVYTLPPSR |
| | | EEMTKNQVSLTCLVKGFYPSDIAVEWESNG |
| | | QPENNYKTTPPVLDSDGSFFLYSKLTVDKS |
| | | RWQQGNVFSCSVMHEALHNHYTQKSLSLS |
| | | PGK |
| 115 | VH-CDR1 | GFTFSSFAMS |
| 116 | VH-CDR2 (variant 1) | TISSGSIYIYYTDGVKG |
| 117 | VH-CDR2 (variant 2) | TISSGSIYIYYTDSVKG |
| 118 | VH-CDR3 (variant 1) | RGIYYGYDGYAMDY |
| 119 | VH-CDR3 (variant 2) | RGIYYGYEGYAMDY |
| 120 | VH-CDR3 (variant 3) | RGIYYGYSGYAMDY |
| 121 | VH-CDR3 (variant 4) | RGIYYGYAGYAMDY |
| 122 | AGX-A01 L10 | AIVLTQSPGTLSLSPGERATLSCRSSQSLVHS |
| | Light chain amino acid | NGNTYLHWYMQKPGQAPRVLIYKVSNRFS |
| | | GIPDRFSGSGSGTDFTLTISRLEPDDFAIYYC |
| | | SQSTHIPLAFGQGTKLEIKRTVAAPSVFIFPP |
| | | SDEQLKSGTASVVCLLNNFYPREAKVQWK |
| | | VDNALQSGNSQESVTEQDSKDSTYSLSSTL |
| | | TLSKADYEKHKVYACEVTHQGLSSPVTKSF |
| | | NRGEC |
| 123 | AGX-A01 L10 | GCCATCGTGTTGACCCAGTCTCCAGGCAC |
| | Light chain nucleic acid | ATTGTCTCTGAGCCCTGGCGAGAGAGCTA |
| | | CCCTGTCCTGCAGATCTTCTCAGTCCCTGG |
| | | TGCACTCCAACGGCAACACCTACCTGCAC |
| | | TGGTACATGCAGAAGCCCGGACAGGCTCC |
| | | CAGAGTGCTGATCTACAAGGTGTCCAACC |
| | | GGTTCTCTGGCATCCCCGACAGATTTTCC |
| | | GGCTCTGGCTCTGGCACCGACTTCACCCT |
| | | GACCATCTCTAGACTGGAACCCGACGACT |
| | | TCGCCATCTACTACTGCTCCCAGTCCACA |
| | | CACATCCCTCTGGCTTTTGGCCAGGGCAC |
| | | CAAGCTGGAAATCAAGAGAACCGTGGCC |
| | | GCTCCTTCCGTGTTCATCTTCCCACCATCT |
| | | GACGAGCAGCTGAAGTCCGGCACAGCTTC |
| | | TGTCGTGTGCCTGCTGAACAACTTCTACC |
| | | CTCGGGAAGCCAAGGTGCAGTGGAAGGT |
| | | GGACAATGCCCTGCAGTCCGGCAACTCCC |
| | | AAGAGTCTGTGACCGAGCAGGACTCCAA |
| | | GGACTCTACCTACAGCCTGTCCTCCACAC |
| | | TGACCCTGTCTAAGGCCGACTACGAGAAG |
| | | CACAAGGTGTACGCCTGTGAAGTGACCCA |
| | | CCAGGGCCTGTCTAGCCCTGTGACCAAGT |
| | | CTTTCAACCGGGGCGAGTGTTGA |
| 124 | VL-CDR1 (variant 1) | RSSQSLVHSNGNTYLH |
| 125 | VL-CDR1 (variant 2) | RSSQSLVHSSGNTYLH |
| 126 | VL-CDR1 (variant 3) | RSSQSLVHSTGNTYLH |
| 127 | VL-CDR1 (variant 4) | RSSQSLVHSQGNTYLH |
| 128 | VL-CDR2 | KVSNRFS |
| 129 | VL-CDR3 | SQSTHIPLA |

Humanized AGX-A07 H2v1L5v2

| | | |
|---|---|---|
| 130 | AGX-A07 H2v1 | EVQLVQSGAEVKKPGASVKVSCKASGYTF |
| | Heavy chain variable region | TNYGVKWVRQAPGQGLEWMGWINTYTGN |
| | amino acid | PIYAADFKGRVTMTTDTSTSTAYMELRSLR |
| | | SDDTAVYYCVRFQYGDYRYFDVWGQGTL |
| | | VTVSS |
| 131 | AGX-A07 H2v1L5v2 | EIVLTQSPATLSLSPGERATLSCRAQSGISFIN |
| | Light chain variable region | WYQQKPGQAPRLLIYGTANLASGIPARFSG |
| | amino acid | SGSGRDFTLTISSLEPEDFAVYYCQQWSSNP |
| | | LTFGGGTKVEIK |

Humanized AGX-A07 H2L5

| | | |
|---|---|---|
| 132 | AGX-A07 H2 | QVQLVQSGAEVKKPGASVKVSCKASGYTF |
| | Heavy chain variable region | TNYGVKWVRQAPGQDLEWMGWINTYTGN |
| | amino acid | PIYAADFKGRVTMTTDTSTSTAFMELRSLRS |
| | | DDTAVYYCVRFQYGDYRYFDVWGQGTLV |
| | | TVSS |
| 133 | AGX-A07 L5 | EIILTQSPATLSLSPGERATLSCRANSGISFIN |
| | Light chain variable region | WYQQKPGQAPRLLIYGTANLASGIPARFGG |
| | amino acid | SGSGRDFTLTISSLEPEDFAVYYCQQWSSNP |
| | | LTFGGGTKVEIK |

Fc Region Sequences

| | | |
|---|---|---|
| 135 | IgG1 L234A/L235A/G237A | ASTKGPSVFPLAPSSKSTSGGTAALGCLVKD |
| | | YFPEPVTVSWNSGALTSGVHTFPAVLQSSG |
| | | LYSLSSVVTVPSSSLGTQTYICNVNHKPSNT |
| | | KVDKKVEPKSCDKTHTCPPCPAPEAAGAPS |
| | | VFLFPPKPKDTLMISRTPEVTCVVVDVSHED |
| | | PEVKFNWYVDGVEVHNAKTKPREEQYNST |
| | | YRVVSVLTVLHQDWLNGKEYKCKVSNKA |
| | | LPAPIEKTISKAKGQPREPQVYTLPPSREEM |
| | | TKNQVSLTCLVKGFYPSDIAVEWESNGQPE |
| | | NNYKTTPPVLDSDGSFFLYSKLTVDKSRWQ |
| | | QGNVFSCSVMHEALHNHYTQKSLSLSPGK |
| 136 | IgG1 | ASTKGPSVFPLAPSSKSTSGGTAALGCLVKD |
| | L234A/L235A/G237A + N297C | YFPEPVTVSWNSGALTSGVHTFPAVLQSSG |
| | | LYSLSSVVTVPSSSLGTQTYICNVNHKPSNT |
| | | KVDKKVEPKSCDKTHTCPPCPAPEAAGAPS |
| | | VFLFPPKPKDTLMISRTPEVTCVVVDVSHED |
| | | PEVKFNWYVDGVEVHNAKTKPREEQYCST |
| | | YRVVSVLTVLHQDWLNGKEYKCKVSNKA |
| | | LPAPIEKTISKAKGQPREPQVYTLPPSREEM |
| | | TKNQVSLTCLVKGFYPSDIAVEWESNGQPE |
| | | NNYKTTPPVLDSDGSFFLYSKLTVDKSRWQ |
| | | QGNVFSCSVMHEALHNHYTQKSLSLSPGK |
| 137 | IgG1 | ASTKGPSVFPLAPSSKSTSGGTAALGCLVKD |
| | L234A/L235A/G237A + P331G | YFPEPVTVSWNSGALTSGVHTFPAVLQSSG |
| | | LYSLSSVVTVPSSSLGTQTYICNVNHKPSNT |
| | | KVDKKVEPKSCDKTHTCPPCPAPEAAGAPS |
| | | VFLFPPKPKDTLMISRTPEVTCVVVDVSHED |
| | | PEVKFNWYVDGVEVHNAKTKPREEQYNST |
| | | YRVVSVLTVLHQDWLNGKEYKCKVSNKA |
| | | LPAGIEKTISKAKGQPREPQVYTLPPSREEM |
| | | TKNQVSLTCLVKGFYPSDIAVEWESNGQPE |
| | | NNYKTTPPVLDSDGSFFLYSKLTVDKSRWQ |
| | | QGNVFSCSVMHEALHNHYTQKSLSLSPGK |
| 138 | IgG1 | ASTKGPSVFPLAPSSKSTSGGTAALGCLVKD |
| | L234A/L235A/G237A + | YFPEPVTVSWNSGALTSGVHTFPAVLQSSG |
| | N297C/P331G | LYSLSSVVTVPSSSLGTQTYICNVNHKPSNT |
| | | KVDKKVEPKSCDKTHTCPPCPAPEAAGAPS |
| | | VFLFPPKPKDTLMISRTPEVTCVVVDVSHED |
| | | PEVKFNWYVDGVEVHNAKTKPREEQYCST |
| | | YRVVSVLTVLHQDWLNGKEYKCKVSNKA |
| | | LPAGIEKTISKAKGQPREPQVYTLPPSREEM |
| | | TKNQVSLTCLVKGFYPSDIAVEWESNGQPE |
| | | NNYKTTPPVLDSDGSFFLYSKLTVDKSRWQ |
| | | QGNVFSCSVMHEALHNHYTQKSLSLSPGK |
| 139 | IgG1 | ASTKGPSVFPLAPSSKSTSGGTAALGCLVKD |
| | L234A/L235A/G237A + | YFPEPVTVSWNSGALTSGVHTFPAVLQSSG |
| | K322A/P331G | LYSLSSVVTVPSSSLGTQTYICNVNHKPSNT |
| | | KVDKKVEPKSCDKTHTCPPCPAPEAAGAPS |
| | | VFLFPPKPKDTLMISRTPEVTCVVVDVSHED |
| | | PEVKFNWYVDGVEVHNAKTKPREEQYNST |
| | | YRVVSVLTVLHQDWLNGKEYKCAVSNKAL |
| | | PAGIEKTISKAKGQPREPQVYTLPPSREEMT |
| | | KNQVSLTCLVKGFYPSDIAVEWESNGQPEN |
| | | NYKTTPPVLDSDGSFFLYSKLTVDKSRWQQ |
| | | GNVFSCSVMHEALHNHYTQKSLSLSPGK |
| 140 | IgG1 | ASTKGPSVFPLAPSSKSTSGGTAALGCLVKD |
| | L234A/L235A/G237A + | YFPEPVTVSWNSGALTSGVHTFPAVLQSSG |
| | E233P/P331G | LYSLSSVVTVPSSSLGTQTYICNVNHKPSNT |
| | | KVDKKVEPKSCDKTHTCPPCPAPPAAGAPS |
| | | VFLFPPKPKDTLMISRTPEVTCVVVDVSHED |
| | | PEVKFNWYVDGVEVHNAKTKPREEQYNST |
| | | YRVVSVLTVLHQDWLNGKEYKCKVSNKA |
| | | LPAGIEKTISKAKGQPREPQVYTLPPSREEM |
| | | TKNQVSLTCLVKGFYPSDIAVEWESNGQPE |
| | | NNYKTTPPVLDSDGSFFLYSKLTVDKSRWQ |
| | | QGNVFSCSVMHEALHNHYTQKSLSLSPGK |
| 141 | IgG1 | ASTKGPSVFPLAPSSKSTSGGTAALGCLVKD |
| | L234A/L235A/G237A + | YFPEPVTVSWNSGALTSGVHTFPAVLQSSG |
| | E233P/N297C | LYSLSSVVTVPSSSLGTQTYICNVNHKPSNT |
| | | KVDKKVEPKSCDKTHTCPPCPAPEAAGAPS |
| | | VFLFPPKPKDTLMISRTPEVTCVVVDVSHED |
| | | PEVKFNWYVDGVEVHNAKTKPREEQYCST |
| | | YRVVSVLTVLHQDWLNGKEYKCKVSNKA |
| | | LPAPIEKTISKAKGQPREPQVYTLPPSREEM |
| | | TKNQVSLTCLVKGFYPSDIAVEWESNGQPE |
| | | NNYKTTPPVLDSDGSFFLYSKLTVDKSRWQ |
| | | QGNVFSCSVMHEALHNHYTQKSLSLSPGK |
| 142 | IgG1 | ASTKGPSVFPLAPSSKSTSGGTAALGCLVKD |
| | L234A/L235A/G237A + | YFPEPVTVSWNSGALTSGVHTFPAVLQSSG |
| | N297C/K322A/P331G | LYSLSSVVTVPSSSLGTQTYICNVNHKPSNT |
| | | KVDKKVEPKSCDKTHTCPPCPAPEAAGAPS |
| | | VFLFPPKPKDTLMISRTPEVTCVVVDVSHED |
| | | PEVKFNWYVDGVEVHNAKTKPREEQYCST |
| | | YRVVSVLTVLHQDWLNGKEYKCAVSNKAL |
| | | PAGIEKTISKAKGQPREPQVYTLPPSREEMT |
| | | KNQVSLTCLVKGFYPSDIAVEWESNGQPEN |
| | | NYKTTPPVLDSDGSFFLYSKLTVDKSRWQQ |
| | | GNVFSCSVMHEALHNHYTQKSLSLSPGK |
| 143 | IgG1 | ASTKGPSVFPLAPSSKSTSGGTAALGCLVKD |
| | L234A/L235A/G237A + | YFPEPVTVSWNSGALTSGVHTFPAVLQSSG |
| | E233P/N297C/P331G | LYSLSSVVTVPSSSLGTQTYICNVNHKPSNT |
| | | KVDKKVEPKSCDKTHTCPPCPAPEAAGAPS |
| | | VFLFPPKPKDTLMISRTPEVTCVVVDVSHED |
| | | PEVKFNWYVDGVEVHNAKTKPREEQYCST |
| | | YRVVSVLTVLHQDWLNGKEYKCKVSNKA |
| | | LPAGIEKTISKAKGQPREPQVYTLPPSREEM |
| | | TKNQVSLTCLVKGFYPSDIAVEWESNGQPE |
| | | NNYKTTPPVLDSDGSFFLYSKLTVDKSRWQ |
| | | QGNVFSCSVMHEALHNHYTQKSLSLSPGK |
| 144 | IgG1 | ASTKGPSVFPLAPSSKSTSGGTAALGCLVKD |
| | L234A/L235A/G237A + | YFPEPVTVSWNSGALTSGVHTFPAVLQSSG |
| | E233P/D265A/N297C/K322A/ | LYSLSSVVTVPSSSLGTQTYICNVNHKPSNT |
| | P331G | KVDKKVEPKSCDKTHTCPPCPAPEAAGAPS |
| | | VFLFPPKPKDTLMISRTPEVTCVVVAVSHED |
| | | PEVKFNWYVDGVEVHNAKTKPREEQYCST |
| | | YRVVSVLTVLHQDWLNGKEYKCAVSNKAL |
| | | PAGIEKTISKAKGQPREPQVYTLPPSREEMT |
| | | KNQVSLTCLVKGFYPSDIAVEWESNGQPEN |
| | | NYKTTPPVLDSDGSFFLYSKLTVDKSRWQQ |
| | | GNVFSCSVMHEALHNHYTQKSLSLSPGK |
| 145 | IgG1 | ASTKGPSVFPLAPSSKSTSGGTAALGCLVKD |
| | L234A/L235A/G237A + | YFPEPVTVSWNSGALTSGVHTFPAVLQSSG |
| | E233P/D265A/N297C/K322A/ | LYSLSSVVTVPSSSLGTQTYICNVNHKPSNT |
| | P331G-PGKKP | KVDKKVEPKSCDKTHTCPPCPAPEAAGAPS |
| | | VFLFPPKPKDTLMISRTPEVTCVVVDVSHED |
| | | PEVKFNWYVDGVEVHNAKTKPREEQYCST |
| | | YRVVSVLTVLHQDWLNGKEYKCAVSNKAL |
| | | PAGIEKTISKAKGQPREPQVYTLPPSREEMT |
| | | KNQVSLTCLVKGFYPSDIAVEWESNGQPEN |
| | | NYKTTPPVLDSDGSFFLYSKLTVDKSRWQQ |
| | | GNVFSCSVMHEALHNHYTQKSLSLSPGKKP |
| 146 | IgG4 | EVQLVQSGAEVKKPGASVKVSCKASGYTF |
| | S228P (sequence includes AGX- | TNYGVKWVRQAPGQGLEWMGWINTYTGN |
| | A07 H2v1 heavy chain variable | PIYAADFKGRVTMTTDTSTSTAYMELRSLR |
| | region amino acid) | SDDTAVYYCVRFQYGDYRYFDVWGQGTL |
| | | VTVSSASTKGPSVFPLAPCSRSTSESTAALG |
| | | CLVKDYFPEPVTVSWNSGALTSGVHTFPAV |
| | | LQSSGLYSLSSVVTVPSSSLGTKTYTCNVDH |
| | | KPSNTKVDKRVESKYGPPCPPCPAPEFLGG |
| | | PSVFLFPPKPKDTLMISRTPEVTCVVVDVSQ |
| | | EDPEVQFNWYVDGVEVHNAKTKPREEQFN |
| | | STYRVVSVLTVLHQDWLNGKEYKCKVSNK |
| | | GLPSSIEKTISKAKGQPREPQVYTLPPSQEE |
| | | MTKNQVSLTCLVKGFYPSDIAVEWESNGQP |
| | | ENNYKTTPPVLDSDGSFFLYSRLTVDKSRW |
| | | QEGNVFSCSVMHEALHNHYTQKSLSLSLGK |
| 147 | IgG4 | EVQLVQSGAEVKKPGASVKVSCKASGYTF |
| | S228P/L235E | TNYGVKWVRQAPGQGLEWMGWINTYTGN |
| | (sequence includes AGX-A07 | PIYAADFKGRVTMTTDTSTSTAYMELRSLR |
| | H2v1 heavy chain variable | SDDTAVYYCVRFQYGDYRYFDVWGQGTL |
| | region amino acid) | VTVSSASTKGPSVFPLAPCSRSTSESTAALG |
| | | CLVKDYFPEPVTVSWNSGALTSGVHTFPAV |
| | | LQSSGLYSLSSVVTVPSSSLGTKTYTCNVDH |
| | | KPSNTKVDKRVESKYGPPCPPCPAPEFEGG |
| | | PSVFLFPPKPKDTLMISRTPEVTCVVVDVSQ |
| | | EDPEVQFNWYVDGVEVHNAKTKPREEQFN |
| | | STYRVVSVLTVLHQDWLNGKEYKCKVSNK |
| | | GLPSSIEKTISKAKGQPREPQVYTLPPSQEE |
| | | MTKNQVSLTCLVKGFYPSDIAVEWESNGQP |
| | | ENNYKTTPPVLDSDGSFFLYSRLTVDKSRW |
| | | QEGNVFSCSVMHEALHNHYTQKSLSLSLGK |
| 148 | IgG4 | EVQLVQSGAEVKKPGASVKVSCKASGYTF |
| | S228P/L235E/N297C | TNYGVKWVRQAPGQGLEWMGWINTYTGN |
| | (sequence includes AGX-A07 | PIYAADFKGRVTMTTDTSTSTAYMELRSLR |
| | H2v1 heavy chain variable | SDDTAVYYCVRFQYGDYRYFDVWGQGTL |
| | region amino acid) | VTVSSASTKGPSVFPLAPCSRSTSESTAALG |
| | | CLVKDYFPEPVTVSWNSGALTSGVHTFPAV |
| | | LQSSGLYSLSSVVTVPSSSLGTKTYTCNVDH |
| | | KPSNTKVDKRVESKYGPPCPPCPAPEFEGG |
| | | PSVFLFPPKPKDTLMISRTPEVTCVVVDVSQ |
| | | EDPEVQFNWYVDGVEVHNAKTKPREEQFC |
| | | STYRVVSVLTVLHQDWLNGKEYKCKVSNK |
| | | GLPSSIEKTISKAKGQPREPQVYTLPPSQEE |
| | | MTKNQVSLTCLVKGFYPSDIAVEWESNGQP |
| | | ENNYKTTPPVLDSDGSFFLYSRLTVDKSRW |
| | | QEGNVFSCSVMHEALHNHYTQKSLSLSLGK |
| 149 | IgG4 | EVQLVQSGAEVKKPGASVKVSCKASGYTF |
| | S228P/F234A/L235E/N297C | TNYGVKWVRQAPGQGLEWMGWINTYTGN |
| | (sequence includes AGX-A07 | PIYAADFKGRVTMTTDTSTSTAYMELRSLR |
| | H2v1 heavy chain variable | SDDTAVYYCVRFQYGDYRYFDVWGQGTL |
| | region amino acid) | VTVSSASTKGPSVFPLAPCSRSTSESTAALG |
| | | CLVKDYFPEPVTVSWNSGALTSGVHTFPAV |
| | | LQSSGLYSLSSVVTVPSSSLGTKTYTCNVDH |
| | | KPSNTKVDKRVESKYGPPCPPCPAPEAEGG |
| | | PSVFLFPPKPKDTLMISRTPEVTCVVVDVSQ |
| | | EDPEVQFNWYVDGVEVHNAKTKPREEQFC |
| | | STYRVVSVLTVLHQDWLNGKEYKCKVSNK |
| | | GLPSSIEKTISKAKGQPREPQVYTLPPSQEE |
| | | MTKNQVSLTCLVKGFYPSDIAVEWESNGQP |
| | | ENNYKTTPPVLDSDGSFFLYSRLTVDKSRW |
| | | QEGNVFSCSVMHEALHNHYTQKSLSLSLGK |
| 150 | IgG4 | EVQLVQSGAEVKKPGASVKVSCKASGYTF |
| | S228P/L235E/N297C-LGKKP | TNYGVKWVRQAPGQGLEWMGWINTYTGN |
| | (sequence includes AGX-A07 | PIYAADFKGRVTMTTDTSTSTAYMELRSLR |
| | H2v1 heavy chain variable | SDDTAVYYCVRFQYGDYRYFDVWGQGTL |
| | region amino acid) | VTVSSASTKGPSVFPLAPCSRSTSESTAALG |
| | | CLVKDYFPEPVTVSWNSGALTSGVHTFPAV |
| | | LQSSGLYSLSSVVTVPSSSLGTKTYTCNVDH |
| | | KPSNTKVDKRVESKYGPPCPPCPAPEFEGG |
| | | PSVFLFPPKPKDTLMISRTPEVTCVVVDVSQ |
| | | EDPEVQFNWYVDGVEVHNAKTKPREEQFC |
| | | STYRVVSVLTVLHQDWLNGKEYKCKVSNK |
| | | GLPSSIEKTISKAKGQPREPQVYTLPPSQEE |
| | | MTKNQVSLTCLVKGFYPSDIAVEWESNGQP |
| | | ENNYKTTPPVLDSDGSFFLYSRLTVDKSRW |
| | | QEGNVFSCSVMHEALHNHYTQKSLSLSLGK |
| | | KP |
| 151 | IgG1 | ASTKGPSVFPLAPSSKSTSGGTAALGCLVKD |
| | M252Y/S254T/T256E | YFPEPVTVSWNSGALTSGVHTFPAVLQSSG |
| | | LYSLSSVVTVPSSSLGTQTYICNVNHKPSNT |
| | | KVDKKVEPKSCDKTHTCPPCPAPELLGGPS |
| | | VFLFPPKPKDTLYITREPEVTCVVVDVSHED |
| | | PEVKFNWYVDGVEVHNAKTKPREEQYNST |
| | | YRVVSVLTVLHQDWLNGKEYKCKVSNKA |
| | | LPAPIEKTISKAKGQPREPQVYTLPPSREEM |
| | | TKNQVSLTCLVKGFYPSDIAVEWESNGQPE |
| | | NNYKTTPPVLDSDGSFFLYSKLTVDKSRWQ |
| | | QGNVFSCSVMHEALHNHYTQKSLSLSPGK |
| 152 | IgG1 | ASTKGPSVFPLAPSSKSTSGGTAALGCLVKD |
| | T252Q/M428L | YFPEPVTVSWNSGALTSGVHTFPAVLQSSG |
| | | LYSLSSVVTVPSSSLGTQTYICNVNHKPSNT |
| | | KVDKKVEPKSCDKTHTCPPCPAPELLGGPS |
| | | VFLFPPKPKDQLMISRTPEVTCVVVDVSHE |
| | | DPEVKFNWYVDGVEVHNAKTKPREEQYNS |
| | | TYRVVSVLTVLHQDWLNGKEYKCKVSNK |
| | | ALPAPIEKTISKAKGQPREPQVYTLPPSREE |
| | | MTKNQVSLTCLVKGFYPSDIAVEWESNGQP |
| | | ENNYKTTPPVLDSDGSFFLYSKLTVDKSRW |
| | | QQGNVFSCSVLHEALHNHYTQKSLSLSPGK |
| 153 | IgG1 | ASTKGPSVFPLAPSSKSTSGGTAALGCLVKD |
| | M428L/N434S | YFPEPVTVSWNSGALTSGVHTFPAVLQSSG |
| | | LYSLSSVVTVPSSSLGTQTYICNVNHKPSNT |
| | | KVDKKVEPKSCDKTHTCPPCPAPELLGGPS |
| | | VFLFPPKPKDTLMISRTPEVTCVVVDVSHED |
| | | PEVKFNWYVDGVEVHNAKTKPREEQYNST |
| | | YRVVSVLTVLHQDWLNGKEYKCKVSNKA |
| | | LPAPIEKTISKAKGQPREPQVYTLPPSREEM |
| | | TKNQVSLTCLVKGFYPSDIAVEWESNGQPE |
| | | NNYKTTPPVLDSDGSFFLYSKLTVDKSRWQ |
| | | QGNVFSCSVLHEALHSHYTQKSLSLSPGK |
| 154 | IgG4 | EVQLVQSGAEVKKPGASVKVSCKASGYTF |
| | T250Q/M428L | TNYGVKWVRQAPGQGLEWMGWINTYTGN |
| | (sequence includes AGX-A07 | PIYAADFKGRVTMTTDTSTSTAYMELRSLR |
| | H2v1 heavy chain variable | SDDTAVYYCVRFQYGDYRYFDVWGQGTL |
| | region amino acid) | VTVSSASTKGPSVFPLAPCSRSTSESTAALG |
| | | CLVKDYFPEPVTVSWNSGALTSGVHTFPAV |
| | | LQSSGLYSLSSVVTVPSSSLGTKTYTCNVDH |
| | | KPSNTKVDKRVESKYGPPCPSCPAPEFLGGP |
| | | SVFLFPPKPKDQLMISRTPEVTCVVVDVSQE |
| | | DPEVQFNWYVDGVEVHNAKTKPREEQFNS |
| | | TYRVVSVLTVLHQDWLNGKEYKCKVSNK |
| | | GLPSSIEKTISKAKGQPREPQVYTLPPSQEE |
| | | MTKNQVSLTCLVKGFYPSDIAVEWESNGQP |
| | | ENNYKTTPPVLDSDGSFFLYSRLTVDKSRW |
| | | QEGNVFSCSVLHEALHNHYTQKSLSLSLGK |
| 155 | IgG4 | EVQLVQSGAEVKKPGASVKVSCKASGYTF |
| | M428L/N434S | TNYGVKWVRQAPGQGLEWMGWINTYTGN |
| | (sequence includes AGX-A07 | PIYAADFKGRVTMTTDTSTSTAYMELRSLR |
| | H2v1 heavy chain region amino | SDDTAVYYCVRFQYGDYRYFDVWGQGTL |
| | acid) | VTVSSASTKGPSVFPLAPCSRSTSESTAALG |
| | | CLVKDYFPEPVTVSWNSGALTSGVHTFPAV |
| | | LQSSGLYSLSSVVTVPSSSLGTKTYTCNVDH |
| | | KPSNTKVDKRVESKYGPPCPSCPAPEFLGGP |
| | | SVFLFPPKPKDTLMISRTPEVTCVVVDVSQE |
| | | DPEVQFNWYVDGVEVHNAKTKPREEQFNS |
| | | TYRVVSVLTVLHQDWLNGKEYKCKVSNK |
| | | GLPSSIEKTISKAKGQPREPQVYTLPPSQEE |
| | | MTKNQVSLTCLVKGFYPSDIAVEWESNGQP |
| | | ENNYKTTPPVLDSDGSFFLYSRLTVDKSRW |
| | | QEGNVFSCSVLHEALHSHYTQKSLSLSLGK |
| 156 | IgG1 | EVQLVQSGAEVKKPGASVKVSCKASGYTF |
| | M252Y/S254T/T256E | TNYGVKWVRQAPGQGLEWMGWINTYTGN |
| | (sequence includes AGXA07 | PIYAADFKGRVTMTTDTSTSTAYMELRSLR |
| | H2v1 heavy chain variable | SDDTAVYYCVRFQYGDYRYFDVWGQGTL |
| | region amino acid) | VTVSSASTKGPSVFPLAPSSKSTSGGTAALG |
| | | CLVKDYFPEPVTVSWNSGALTSGVHTFPAV |
| | | LQSSGLYSLSSVVTVPSSSLGTQTYICNVNH |
| | | KPSNTKVDKKVEPKSCDKTHTCPPCPAPEL |
| | | LGGPSVFLFPPKPKDTLYITREPEVTCVVVD |
| | | VSHEDPEVKFNWYVDGVEVHNAKTKPREE |
| | | QYNSTYRVVSVLTVLHQDWLNGKEYKCK |
| | | VSNKALPAPIEKTISKAKGQPREPQVYTLPP |
| | | SREEMTKNQVSLTCLVKGFYPSDIAVEWES |
| | | NGQPENNYKTTPPVLDSDGSFFLYSKLTVD |
| | | KSRWQQGNVFSCSVMHEALHNHYTQKSLS |
| | | LSPGK |
| 157 | L6 CDR-L1 | SSVSF |
| 158 | L6 CDR-L2 | ATS |
| 159 | L6 CDR-L3 | WNSNPL |
| 160 | L6 CDR-H1 | GYTFTNY |
| 161 | L6 CDR-H2 | TYTG |
| 162 | L6 CDR-H3 | FSYGNSRYAD |
| 163 | L6 VL | QIVLSQSPAILSASPGEKVTLTCRASSVSFM |
| | | NWYQQKPGSSPKPWIYATSNLASGVPGRFS |
| | | GSGSGTSYSLAISRVEAEDAATYYCQQWNS |
| | | NPLTFGAGTKLELKR |
| 164 | L6 VH | QIQLVQSGPELKKPGETVKISCKASGYTFTN |
| | | YGMNWVKQAPGKGLKWMGWINTYTGQP |
| | | TYADDFKGRFAFSLETSAYTAYLQINNLKN |
| | | EDMATYFCARFSYGNSRYADYWGQGTTLT |
| | | VSS |
| 165 | human TM4SF1 protein (NCBI | MCYGKCARCIGHSLVGLALLCIAANILLYF |
| | Ref Seq No. NP_055035.1) | PNGETKYASENHLSRFVWFFSGIVGGGLLM |
| | | LLPAFVFIGLEQDDCCGCCGHENCGKRCAM |
| | | LSSVLAALIGIAGSGYCVIVAALGLAEGPLC |
| | | LDSLGQWNYTFASTEGQYLLDTSTWSECTE |
| | | PKHIVEWNVSLFSILLALGGIEFILCLIQVING |
| | | VLGGIC GFCCSHQQQY DC |
| 166 | Human TM4SF1 ECL2 | EGPLCLDSLGQWNYTFASTEGQYLLDTSTW |
| | | SECTEPKHIVEWNVSLFS |

1.-56. (canceled)

57. A combination comprising:
(a) an anti-TM4SF1 binding protein; and
(b) an immunotherapeutic agent;
wherein the immunotherapeutic agent is
(a) an antibody or an antigen binding fragment thereof directed against at least one of PD-1, CD40, CTLA-4, CSF1/CSF1R, SIRPα, CLEC-1, CCR4, CTLA-4, A1R, A2AR, A3R, TIM-3, BTLA, VISTA, TIGIT, LAG-3, ILRa/CD25, ITGB1/CD29, Ly 24/CD44, CD48, CEACAM1/CD66a, Nt5e/CD73, CD94/NKG2A, FAS/CD95, SLAF1/CD150, NRP1/CD304, GITR/CD357, ICOS, Tnfrs4/OX40, Folr4/JUNO, P2X7, ANXA2, IDO, B7-H6, KIR, GARP (LRRC32), TNFR2, PD-L1, PD-L2, B7-H3, B7-H4, CD47, TDO, or DcR3; or
(b) an immunotherapeutic agent selected from the group consisting of: ipilimumab, nivolumab, pembrolizumab, atezolizumab, durvalumab, tremelimumab, spartalizumab, avelumab, sintilimab, toripalimab, MGA012, MGD013, MGD019, enoblituzumab, MGD009, MGC018, MEDI0680, PDR001, FAZ053, TSR022, MBG453, relatlinab (BMS986016), LAG525, IMP321, REGN2810 (cemiplimab), REGN3767, pexidartinib, LY3022855, FPA008, BLZ945, GDC0919, epacadostat, indoximid, BMS986205, CPI-444, MEDI9447, PBF509, lirilumab, IMC-001, Monalizumab, and combinations thereof.

58. The combination of claim 57, wherein the anti-TM4SF1 binding protein comprises:
(a) a heavy chain variable domain comprising a CDR3 domain comprising an amino acid sequence of SEQ ID NO: 8, 20, 32, 44, 56, 68, 80, 96, 118, 119, 120, 121, or 162; a CDR2 domain comprising an amino acid sequence of SEQ ID NO: 7, 19, 31, 43, 55, 67, 79, 95, 116, 117, or 161; and a CDR1 domain comprising an amino acid sequence of SEQ ID NO: 6, 18, 30, 42, 54, 66, 78, 94, 115, or 160; and
(b) a light chain variable domain comprising a CDR3 domain comprising an amino acid sequence of SEQ ID NO: 14, 26, 38, 50, 62, 74, 86, 110, 129, or 159; a CDR2 domain comprising an amino acid sequence of SEQ ID NO: 13, 25, 37, 49, 61, 73, 85, or 109, 128, or 158; and a CDR1 comprising an amino acid sequence of SEQ ID NO: 12, 24, 36, 48, 60, 72, 84, 107, 108, 124, 125, 126, 127, or 157.

59. The combination of claim 57, wherein the anti-TM4SF1 binding protein comprises:
(a) a heavy chain variable domain comprising a CDR3 domain comprising the amino acid sequence of SEQ ID NO: 96; a CDR2 domain comprising the amino acid sequence of SEQ ID NO: 95; and a CDR1 domain comprising the amino acid sequence of SEQ ID NO: 94; and
(b) a light chain variable domain comprising a CDR3 domain comprising the amino acid sequence of SEQ ID NO: 110 or 111; a CDR2 domain comprising the amino acid sequence of SEQ ID NO: 109; and a CDR1 comprising the amino acid sequence of SEQ ID NO: 107 or 108.

60. The combination of claim 57, wherein the anti-TM4SF1 binding protein comprises:
(a) a heavy chain comprising an amino acid sequence selected from the group consisting of: SEQ ID NOs: 90, 92, 130 and 132; and
(b) a light chain comprising an amino acid sequence selected from the group consisting of: SEQ ID NOs: 97, 99, 101, 103, 105, 131, and 133.

61. The combination of claim 57, wherein the anti-TM4SF1 binding protein comprises an IgG Fc region, and wherein the IgG Fc region comprises at least one of the following mutations: E233P, L234A, L235A, G237A, M252Y, S254T, T250Q, T256E, D265A, N297C, K322A, P331G, M428L, N434A, and N434S; as numbered by the EU index as set forth in Rabat.

62. The combination of claim 61, wherein the IgG1 Fc region comprise the following mutations: N297C, L234A, L235A, and G237A.

63. The combination of claim 61, wherein the IgG1 Fc region comprise the following mutations: N297C, M252Y, S254T, and T256E.

64. The combination of claim 61, wherein the anti-TM4SFl binding protein is conjugated to a therapeutic molecule, forming an antibody-drug conjugate, wherein the therapeutic molecule is selected from the group consisting of: a proteasome inhibitor, a calicheamicin, a pyrrolobenzodiazepine, an auristatin, a duocarmycin, a maytansinoid, and any combination thereof.

65. The combination of claim 64, wherein the anti-TM4SFl binding protein conjugates with the therapeutic molecule via site specific conjugations at N297C or at both N297C and Q295.

66. The combination of claim 64, wherein antibody-drug conjugate has a drug-to-antibody ratio of 1 or 2.

67. A pharmaceutical composition comprising the combination of claim 57, and at least one of: a pharmaceutically acceptable carrier, an excipient, a diluent, or any combination thereof.

68. A method of treating a subject, the method comprising administering to the subject a pharmaceutical composition according to claim 67.

69. The method of claim 68, wherein the immunotherapeutic agent is PD-1 antibody, CTLA-4 antibody, or PD-L1/L2 antibody.

70. The method of claim 68, wherein the administering improves T cell function.

71. The method of claim 68, wherein the administering increases T cell infiltration in the tumor microenvironment.

72. The method of claim 68, wherein the administering increases expression of ICAM-1 and VCAM-1 in tumor vessels.

73. The method of claim 68, wherein the subject has a cancer, and wherein the cancer is prostate cancer, liver cancer, colorectal cancer, ovarian cancer, endometrial cancer, breast cancer, triple negative breast cancer, pancreatic cancer, stomach (gastric) cancer, cervical cancer, head and neck cancer, thyroid cancer, testis cancer, urothelial cancer, lung cancer (small cell lung, non-small cell lung), melanoma, non-melanoma skin cancer (squamous and basal cell carcinoma), glioma, renal cancer, lymphoma (NIL or HL), Acute myeloid leukemia (AML), T cell Acute Lymphoblastic Leukemia (T-ALL), Diffuse Large B cell lymphoma, testicular germ cell tumors, mesothelioma, esophageal cancer, Merkel Cells cancer, MSI-high cancer, KRAS mutant tumors, adult T-cell leukemia/lymphoma, and Myelodysplastic syndromes (MDS)

74. The method of claim 73, wherein the cancer comprises a cancer cell does not express TM4SF1.

75. The method of claim 73, wherein the cancer comprises a cancer cell expresses TM4SF1.

76. The method of claim 68, wherein the administering reduces tumor regression.


SEQUENCE LISTING (168 sequences)

SEQ ID NO: 1 - protein, 122 aa, synthetic construct
  REGION 1..122: note = Description of Artificial Sequence: Synthetic polypeptide
EVILVESGGGLVKPGGSLKLSCAASGFTFSSFAMSWVRQTPEKRLEWVATISSGSIYIYYTDGVKGRFTISRDNAKNTVHLQMSSLRSEDTAMYYCARRGIYYGYDGYAMDYWGQGTSVTVS

SEQ ID NO: 2 - protein, 112 aa, synthetic construct
  REGION 1..112: note = Description of Artificial Sequence: Synthetic polypeptide
AVVMTQTPLSLPVSLGDQASISCRSSQSLVHSNGNTYLHWYMQKPGQSPKVLIYKVSNRFSGVPDRFSGSGSGTDFTLKISRVEADDLGIYFCSQSTHIPLAFGAGTKLELK

SEQ ID NO: 3 - protein, 123 aa, synthetic construct
  REGION 1..123: note = Description of Artificial Sequence: Synthetic polypeptide
QIQLVQSGPELKKPGETVKISCKASGYSFRDYGMNWVKQAPGRTFKWMGWINTYTGAPVYAADFKGRFAFSLDTSASAAFLQINNLKNEDTATYFCARWVSYGNNRNWFFDFWGAGTTVTVSS

SEQ ID NO: 4 - DNA, 369 bp, synthetic construct
  misc_feature 1..369: note = Description of Artificial Sequence: Synthetic polynucleotide
  source 1..369: mol_type = other DNA
cagatccagttggtgcagtctggacctgagctgaagaagcctggagagacagtcaagatctcctgcaaggcttctgggtattccttcagagactatggaatgaactgggtgaagcaggctccaggaaggacttttaagtggatgggctggataaacacctacactggagcgccagtatatgctgctgacttcaagggacggtttgccttctctttggacacctctgccagcgctgcctttttgcagatcaacaacctcaaaaatgaagacacggctacatatttctgtgcaagatgggtctcctacggtaataaccgcaactggttcttcgatttttggggcgcagggaccacggtcaccgtctcctca

SEQ ID NO: 5 - DNA, 369 bp, synthetic construct
  misc_feature 1..369: note = Description of Artificial Sequence: Synthetic polynucleotide
  source 1..369: mol_type = other DNA
caaattcagttggttcaatccggccctgagctcaagaagcctggagagacagtgaagataagttgtaaggctagtggctattcatttcgagattatgggatgaattgggtcaagcaggccccagggcggaccttcaaatggatggggtggatcaatacttacactggcgcaccagtatatgcagctgattttaagggtcgctttgcattttcacttgatacttcagccagtgccgcttttttgcaaatcaacaatctcaaaaatgaagacactgctacatatttctgcgccaggtgggtgagctatggcaataacagaaattggttctttgacttttggggcgcaggcaccaccgtcactgtctcatca

SEQ ID NO: 6 - protein, 10 aa, synthetic construct
  REGION 1..10: note = Description of Artificial Sequence: Synthetic peptide
GYSFRDYGMN

SEQ ID NO: 7 - protein, 17 aa, synthetic construct
  REGION 1..17: note = Description of Artificial Sequence: Synthetic peptide
WINTYTGAPVYAADFKG

SEQ ID NO: 8 - protein, 14 aa, synthetic construct
  REGION 1..14: note = Description of Artificial Sequence: Synthetic peptide
WVSYGNNRNWFFDF

SEQ ID NO: 9 - protein, 112 aa, synthetic construct
  REGION 1..112: note = Description of Artificial Sequence: Synthetic polypeptide
DVLMTQTPLSLPVRLGDQASISCRSSQTLVHSNGNTYLEWYLQKPGQSPKLLIYKVSNRLSGVPDRFSGSGSGTDFTLKISRVETEDLGVYYCFQGSHGPWTFGGGTKLEIK

SEQ ID NO: 10 - DNA, 336 bp, synthetic construct
  misc_feature 1..336: note = Description of Artificial Sequence: Synthetic polynucleotide
  source 1..336: mol_type = other DNA
gatgttttgatgacccaaactccactctccctgcctgtccgtcttggagatcaggcctccatctcttgtagatctagtcagacccttgtacatagtaatggaaacacctatttagaatggtacctgcagaaaccaggccagtctccaaaactcttgatctacaaagtttccaatcgactttctggggtcccagacaggttcagtggcagtggatcagggacagatttcacactcaagatcagcagagtggagactgaggatctgggagtttattactgctttcaaggttcacatggtccgtggacgttcggtggaggcaccaagctggaaatcaaa

SEQ ID NO: 11 - DNA, 336 bp, synthetic construct
  misc_feature 1..336: note = Description of Artificial Sequence: Synthetic polynucleotide
  source 1..336: mol_type = other DNA
gacgtacttatgacacaaactcccttgagcttgccagtacggcttggcgatcaagcttcaatttcatgtcgttcttctcaaacacttgtccactcaaatgggaatacatatttggaatggtatctccaaaagcccggccaatccccaaaattgttgatttacaaggtgtctaatcgactctcaggcgtccccgaccgattctccgggagcgggtccggtacagacttcaccttgaaaatctccagggtagaaactgaagacctcggagtctactattgtttccaggggtcacacggcccctggacatttggaggaggaactaagctcgagatcaaa

SEQ ID NO: 12 - protein, 16 aa, synthetic construct
  REGION 1..16: note = Description of Artificial Sequence: Synthetic peptide
RSSQTLVHSNGNTYLE

SEQ ID NO: 13 - protein, 7 aa, synthetic construct
  REGION 1..7: note = Description of Artificial Sequence: Synthetic peptide
KVSNRLS

SEQ ID NO: 14 - protein, 9 aa, synthetic construct
  REGION 1..9: note = Description of Artificial Sequence: Synthetic peptide
FQGSHGPWT

SEQ ID NO: 15 - protein, 118 aa, synthetic construct
  REGION 1..118: note = Description of Artificial Sequence: Synthetic polypeptide
EVQLQQSGPELVKPGASVKISCKTSGYTFTDYTMHWVRQSHGKSLEWIGSFNPNNGGLTNYNQKFKGKATLTVDKSSSTVYMDLRSLTSEDSAVYYCTRIRATGFDSWGQGTTLTVSS

SEQ ID NO: 16 - DNA, 354 bp, synthetic construct
  misc_feature 1..354: note = Description of Artificial Sequence: Synthetic polynucleotide
  source 1..354: mol_type = other DNA
gaggtccagctgcaacagtctggacctgagctggtgaagcctggggcttcagtgaagatatcctgcaagacttctggatacacattcactgattacaccatgcactgggtgaggcagagccatggaaagagccttgagtggattggaagttttaatcctaacaatggtggtcttactaactacaaccagaagttcaagggcaaggccacattgactgtggacaagtcttccagcacagtgtacatggacctccgcagcctgacatctgaggattctgcagtctattactgtacaagaatccgggctacgggctttgactcctggggccagggcaccactctcacagtctcctca

SEQ ID NO: 17 - DNA, 354 bp, synthetic construct
  misc_feature 1..354: note = Description of Artificial Sequence: Synthetic polynucleotide
  source 1..354: mol_type = other DNA
gaggtacaactgcaacagagtggacctgaacttgtcaaacctggagcaagtgtgaagattagctgtaaaaccagtggctacacatttaccgattatactatgcactgggtaagacagagccacggaaaatcactggagtggattggtagtttcaatcctaacaacggaggattgacaaattacaaccagaagttcaaagggaaagccaccttgacagttgataagtcctcaagtaccgtgtatatggatctgcgttctctcacaagtgaagatagcgcagtttactactgtacccgcatccgagccaccgggttcgattcatggggtcaggggacaacactgactgtttcttct

SEQ ID NO: 18 - protein, 10 aa, synthetic construct
  REGION 1..10: note = Description of Artificial Sequence: Synthetic peptide
GYTFTDYTMH

SEQ ID NO: 19 - protein, 18 aa, synthetic construct
  REGION 1..18: note = Description of Artificial Sequence: Synthetic peptide
SFNPNNGGLTNYNQKFKG

SEQ ID NO: 20 - protein, 8 aa, synthetic construct
  REGION 1..8: note = Description of Artificial Sequence: Synthetic peptide
IRATGFDS

SEQ ID NO: 21 - protein, 113 aa, synthetic construct
  REGION 1..113: note = Description of Artificial Sequence: Synthetic polypeptide
DIVMSQSPSSLAVSAGEKVTMSCKSSQSLLNSRTRKNYLAWYQQKPGQSPKLLIYWASTRESGVPDRFTGSGSGTDFTLTISNVQAEDLTVYYCKQSYNPPWTFGGGTKLEIK

SEQ ID NO: 22 - DNA, 339 bp, synthetic construct
  misc_feature 1..339: note = Description of Artificial Sequence: Synthetic polynucleotide
  source 1..339: mol_type = other DNA
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcaatgtgcaggctgaagacctgacagtttattactgcaagcaatcttataatcctccgtggacgttcggtggaggcaccaagctggaaatcaaa

SEQ ID NO: 23 - DNA, 339 bp, synthetic construct
  misc_feature 1..339: note = Description of Artificial Sequence: Synthetic polynucleotide
  source 1..339: mol_type = other DNA
gacatagttatgtcccagtctccatccagcttggctgtcagcgccggagagaaagtgactatgagttgtaaatcttcccagtccctgcttaactcacgtactcggaagaattatcttgcctggtatcaacaaaagccaggtcaaagtcctaagctccttatttactgggcctcaacacgggagtcaggtgtccccgatcgcttcacaggtagtgggagtggtactgacttcactctcaccatttcaaatgtccaagcagaagacttgactgtgtattactgtaagcagagttacaaccctccttggacctttggtggggggaccaaactggagatcaag

SEQ ID NO: 24 - protein, 17 aa, synthetic construct
  REGION 1..17: note = Description of Artificial Sequence: Synthetic peptide
KSSQSLLNSRTRKNYLA

SEQ ID NO: 25 - protein, 7 aa, synthetic construct
  REGION 1..7: note = Description of Artificial Sequence: Synthetic peptide
WASTRES

SEQ ID NO: 26 - protein, 9 aa, synthetic construct
  REGION 1..9: note = Description of Artificial Sequence: Synthetic peptide
KQSYNPPWT

SEQ ID NO: 27 - protein, 116 aa, synthetic construct
  REGION 1..116: note = Description of Artificial Sequence: Synthetic polypeptide
EVQVQQSGPELVKPGASVKMSCKASGYTFTSYVMHWVKQKPGQGLEWIGYINPNNDNINYNEKFKGKASLTSDKSSNTVYMELSSLTSEDSAVYYCAGYGNSGANWGQGTLVTVSA

SEQ ID NO: 28 - DNA, 348 bp, synthetic construct
  misc_feature 1..348: note = Description of Artificial Sequence: Synthetic polynucleotide
  source 1..348: mol_type = other DNA
gaggtccaggtacagcagtctggacctgaactggtaaagcctggggcttcagtgaagatgtcctgtaaggcttctggatacacattcactagctatgtcatgcactgggtgaagcagaagcctgggcagggccttgagtggattggatatattaatcctaacaatgataatattaactacaatgagaagttcaaaggcaaggcctcactgacttcagacaaatcctccaacacagtctacatggagctcagcagcctgacctctgaggactctgcggtctattactgtgcaggctatggtaactccggagctaactggggccaagggactctggtcactgtctctgca

SEQ ID NO: 29 - DNA, 348 bp, synthetic construct
  misc_feature 1..348: note = Description of Artificial Sequence: Synthetic polynucleotide
  source 1..348: mol_type = other DNA
gaagttcaagttcagcaaagcgggcctgagcttgtcaagccaggcgcatcagtcaaaatgagctgtaaggcttccgggtacaccttcaccagttatgtcatgcattgggtaaaacaaaagccaggacagggactcgagtggataggatacattaacccaaataacgacaacattaactacaacgagaaattcaagggcaaagcatcattgacttccgataaatcctctaacaccgtgtacatggagctgagttcattgaccagcgaggattctgccgtgtactactgtgcaggttatggcaactctggtgctaactgggggcaggggactctggtcacagtcagcgca

SEQ ID NO: 30 - protein, 10 aa, synthetic construct
  REGION 1..10: note = Description of Artificial Sequence: Synthetic peptide
GYTFTSYVMH

SEQ ID NO: 31 - protein, 17 aa, synthetic construct
  REGION 1..17: note = Description of Artificial Sequence: Synthetic peptide
YINPNNDNINYNEKFKG

SEQ ID NO: 32 - protein, 7 aa, synthetic construct
  REGION 1..7: note = Description of Artificial Sequence: Synthetic peptide
YGNSGAN

SEQ ID NO: 33 - protein, 107 aa, synthetic construct
  REGION 1..107: note = Description of Artificial Sequence: Synthetic polypeptide
DIQMTQSPASLSASVGETVTITCRTSKNIFNFLAWYHQKQGRSPRLLVSHTKTLAAGVPSRFSGSGSGTQFSLKINSLQPEDFGIYYCQHHYGTPWTFGGGTKLEIK

SEQ ID NO: 34 - DNA, 321 bp, synthetic construct
  misc_feature 1..321: note = Description of Artificial Sequence: Synthetic polynucleotide
  source 1..321: mol_type = other DNA
gacatccagatgactcagtctccagcctccctatctgcatctgtgggagaaactgtcaccatcacatgtcgaacaagtaaaaatattttcaattttttagcatggtatcaccagaaacagggaagatctcctcgactcctggtctctcatacaaaaaccttagcagcaggtgtgccatcaaggttcagtggcagtggctcaggcacacagttttctctgaagatcaacagcctgcagcctgaagattttgggatttattactgtcaacatcattatggtactccgtggacgttcggtggaggcaccaaactggaaatcaaa

SEQ ID NO: 35 - DNA, 321 bp, synthetic construct
  misc_feature 1..321: note = Description of Artificial Sequence: Synthetic polynucleotide
  source 1..321: mol_type = other DNA
gacattcagatgacccagtcaccagcatctttgagcgcatccgttggggagactgtgacaatcacatgccgaaccagtaagaacatcttcaacttcctcgcatggtaccatcaaaagcagggcaggtctcccagactgcttgtctctcacaccaagacactggcagcaggcgtccccagccggtttagtggtagtggatctggcacacagtttagtttgaaaatcaattccctgcaacccgaagacttcggcatatactattgccagcaccactatgggacaccttggactttcggaggtggtactaaacttgagattaaa

SEQ ID NO: 36 - protein, 11 aa, synthetic construct
  REGION 1..11: note = Description of Artificial Sequence: Synthetic peptide
RTSKNIFNFLA

SEQ ID NO: 37 - protein, 7 aa, synthetic construct
  REGION 1..7: note = Description of Artificial Sequence: Synthetic peptide
HTKTLAA

SEQ ID NO: 38 - protein, 9 aa, synthetic construct
  REGION 1..9: note = Description of Artificial Sequence: Synthetic peptide
QHHYGTPWT

SEQ ID NO: 39 - protein, 120 aa, synthetic construct
  REGION 1..120: note = Description of Artificial Sequence: Synthetic polypeptide
QIQLVQSGPELKKPGETVKISCKASGYTFTNYGVKWVKQAPGKDLKWMGWINTYTGNPIYAADFKGRFAFSLETSASTAFLQINNLKNEDTATYFCVRFQYGDYRYFDVWGAGTTVTVSS

SEQ ID NO: 40 - DNA, 360 bp, synthetic construct
  misc_feature 1..360: note = Description of Artificial Sequence: Synthetic polynucleotide
  source 1..360: mol_type = other DNA
cagatccagttggtgcagtctggacctgagctgaagaagcctggagagacagtcaagatctcctgcaaggcttctgggtataccttcacaaactatggagtgaagtgggtgaagcaggctccaggaaaggatttaaagtggatgggctggataaacacctacactggaaatccaatttatgctgctgacttcaagggacggtttgccttctctttggagacctctgccagcactgcctttttgcagatcaacaacctcaaaaatgaggacacggctacatatttctgtgtaagattccaatatggcgattaccggtacttcgatgtctggggcgcagggaccacggtcaccgtctcctca

SEQ ID NO: 41 - DNA, 360 bp, synthetic construct
  misc_feature 1..360: note = Description of Artificial Sequence: Synthetic polynucleotide
  source 1..360: mol_type = other DNA
caaatccaacttgtccagagcggtcccgagttgaagaagcctggcgaaaccgtgaaaatctcatgcaaggccagtggatatacatttacaaactatggcgtcaagtgggtgaaacaagccccaggtaaagacttgaaatggatgggatggatcaacacatacacagggaatcctatctatgcagccgactttaaaggcagatttgccttcagtttggagacatctgcctccaccgctttcctgcaaataaataacctgaaaaatgaagataccgctacatacttctgtgtacggttccagtacggagattaccgctatttcgatgtgtggggcgcaggtaccacagtaaccgtctcctca

SEQ ID NO: 42 - protein, 10 aa, synthetic construct
  REGION 1..10: note = Description of Artificial Sequence: Synthetic peptide
GYTFTNYGVK

SEQ ID NO: 43 - protein, 17 aa, synthetic construct
  REGION 1..17: note = Description of Artificial Sequence: Synthetic peptide
WINTYTGNPIYAADFKG

SEQ ID NO: 44 - protein, 11 aa, synthetic construct
  REGION 1..11: note = Description of Artificial Sequence: Synthetic peptide
FQYGDYRYFDV

SEQ ID NO: 45 - protein, 106 aa, synthetic construct
  REGION 1..106: note = Description of Artificial Sequence: Synthetic polypeptide
QIILSQSPAILSASPGEKVTMTCRANSGISFINWYQQKPGSSPKPWIYGTANLASGVPARFGGSGSGTSYSLTISRVEAEDAATYYCQQWSSNPLTFGAGTKLELR

SEQ ID NO: 46 - DNA, 318 bp, synthetic construct
  misc_feature 1..318: note = Description of Artificial Sequence: Synthetic polynucleotide
  source 1..318: mol_type = other DNA
caaattattctctcccagtctccagcaatcctgtctgcatctccaggggagaaggtcacgatgacttgcagggccaactcaggtattagtttcatcaactggtaccagcagaagccaggatcctcccccaaaccctggatttatggcacagccaacctggcttctggagtccctgctcgcttcggtggcagtgggtctgggacttcttactctctcacaatcagcagagtggaggctgaagacgctgccacttattactgccagcagtggagtagtaacccgctcacgttcggtgctgggaccaagctggagttgaga

SEQ ID NO: 47 - DNA, 318 bp, synthetic construct
  misc_feature 1..318: note = Description of Artificial Sequence: Synthetic polynucleotide
  source 1..318: mol_type = other DNA
caaataattctgtcacagtcccccgctatacttagtgcttcaccaggagaaaaagtgaccatgacttgtagagctaattctggcatatcattcatcaactggtatcaacaaaagccaggttcctcccccaagccatggatttacgggaccgccaaccttgcttctggggtacccgctcgtttcggcggatcaggttcaggaacttcctatagcctcactatcagtcgggttgaagctgaggatgccgctacatattactgccagcaatggtctagtaatccacttacctttggagctggcaccaaattggaacttcgt

SEQ ID NO: 48 - protein, 10 aa, synthetic construct
  REGION 1..10: note = Description of Artificial Sequence: Synthetic peptide
RANSGISFIN

SEQ ID NO: 49 - protein, 7 aa, synthetic construct
  REGION 1..7: note = Description of Artificial Sequence: Synthetic peptide
GTANLAS

SEQ ID NO: 50 - protein, 9 aa, synthetic construct
  REGION 1..9: note = Description of Artificial Sequence: Synthetic peptide
QQWSSNPLT

SEQ ID NO: 51 - protein, 116 aa, synthetic construct
  REGION 1..116: note = Description of Artificial Sequence: Synthetic polypeptide
EVQLQQSGPELVKPGASVKLSCKASGYTVTSYVMHWVKQKPGQGLEWIGYINPYSDVTNCNEKFKGKATLTSDKTSSTAYMELSSLTSEDSAVYYCSSYGGGFAYWGQGTLVTVSA

SEQ ID NO: 52 - DNA, 348 bp, synthetic construct
  misc_feature 1..348: note = Description of Artificial Sequence: Synthetic polynucleotide
  source 1..348: mol_type = other DNA
gaggtccagctgcagcagtctggacctgagctggtaaagcctggggcttcagtgaagctgtcctgcaaggcttctggatacacagtcactagctatgttatgcactgggtgaagcagaagcctgggcagggccttgagtggattggatatattaatccttacagtgatgttactaactgcaatgagaagttcaaaggcaaggccacactgacttcagacaaaacctccagcacagcctacatggagctcagcagcctgacctctgaggactctgcggtctattactgttcctcctacggtggggggtttgcttactggggccaagggactctggtcactgtctctgca

SEQ ID NO: 53 - DNA, 348 bp, synthetic construct
  misc_feature 1..348: note = Description of Artificial Sequence: Synthetic polynucleotide
  source 1..348: mol_type = other DNA
gaagtccagcttcagcaatccggcccagaactggtaaaaccaggcgcaagtgttaagttgagttgcaaagccagtggttataccgttacttcatacgtcatgcattgggtaaaacaaaagcccggccaagggcttgaatggatcggctacatcaacccttactctgacgtcaccaactgcaacgagaaattcaaagggaaagccacattgacctctgacaagacaagcagtaccgcctacatggagctttctagtttgacttctgaagactctgctgtctactactgtagcagctacggcggcggctttgcttactggggccagggtacattggtgactgtgagtgca

SEQ ID NO: 54 - protein, 10 aa, synthetic construct
  REGION 1..10: note = Description of Artificial Sequence: Synthetic peptide
GYTVTSYVMH

SEQ ID NO: 55 - protein, 17 aa, synthetic construct
  REGION 1..17: note = Description of Artificial Sequence: Synthetic peptide
YINPYSDVTNCNEKFKG

SEQ ID NO: 56 - protein, 7 aa, synthetic construct
  REGION 1..7: note = Description of Artificial Sequence: Synthetic peptide
YGGGFAY

SEQ ID NO: 57 - protein, 107 aa, synthetic construct
  REGION 1..107: note = Description of Artificial Sequence: Synthetic polypeptide
DIQMTQSPASLSASVGEPVTITCRASKNIYTYLAWYHQKQGKSPQFLVYNARTLAGGVPSRLSGSGSVTQFSLNINTLHREDLGTYFCQHHYDTPYTFGGGTNLEIK

SEQ ID NO: 58 - DNA, 321 bp, synthetic construct
  misc_feature 1..321: note = Description of Artificial Sequence: Synthetic polynucleotide
  source 1..321: mol_type = other DNA
gacatccagatgactcagtctccagcctccctatctgcatctgtgggagaacctgtcaccatcacatgtcgagcaagtaagaatatttacacatatttagcatggtatcaccagaaacagggaaaatctcctcagttcctggtctataatgcaagaaccttagcaggaggtgtgccatcaaggctcagtggcagtggatcagtcacgcagttttctctaaacatcaacaccttgcatcgagaagatttagggacttacttctgtcaacatcattatgatactccgtacacgttcggaggggggaccaacctggaaataaaa

SEQ ID NO: 59 - DNA, 321 bp, synthetic construct
  misc_feature 1..321: note = Description of Artificial Sequence: Synthetic polynucleotide
  source 1..321: mol_type = other DNA
gacatccagatgacacagtcaccagcatccctgtccgcctcagttggggagcctgttaccataacttgtcgggcaagcaaaaacatatacacctatttggcttggtatcaccaaaagcaaggtaagtcacctcagtttcttgtatataatgcccgcacacttgctggcggagtaccctctcgattgtctggatctggcagcgttacccaattcagcctgaacatcaacaccctccatcgggaagatttgggtacctatttctgtcaacatcactacgacaccccatacaccttcggaggcggcacaaatttggaaattaaa

SEQ ID NO: 60 - protein, 11 aa, synthetic construct
  REGION 1..11: note = Description of Artificial Sequence: Synthetic peptide
RASKNIYTYLA

SEQ ID NO: 61 - protein, 7 aa, synthetic construct
  REGION 1..7: note = Description of Artificial Sequence: Synthetic peptide
NARTLAG

SEQ ID NO: 62 - protein, 9 aa, synthetic construct
  REGION 1..9: note = Description of Artificial Sequence: Synthetic peptide
QHHYDTPYT

SEQ ID NO: 63 - protein, 113 aa, synthetic construct
  REGION 1..113: note = Description of Artificial Sequence: Synthetic polypeptide
EVQLQQSGPELVKPGASVKMSCKASGYTFSSYVMHWVKQKPGQGLEWIGYINPYSDVTNYNEKFKGKATLTSDRSSNTAYMELSSLTSEDSAVYYCARNYFDWGRGTLVTVSA

SEQ ID NO: 64 - DNA, 339 bp, synthetic construct
  misc_feature 1..339: note = Description of Artificial Sequence: Synthetic polynucleotide
  source 1..339: mol_type = other DNA
gaggtccagctgcagcagtctggacctgagctggtaaagcctggggcttcagtgaagatgtcctgcaaggcttctggatacacattctctagctatgttatgcactgggtgaagcagaagcctgggcagggccttgagtggattggatatattaatccttacagtgatgtcactaactacaatgagaagttcaaaggcaaggccacactgacttcagacagatcctccaacacagcctacatggaactcagcagcctgacctctgaggactctgcggtctattactgtgcaagaaattacttcgactggggccgagggactctggtcacagtctctgca

SEQ ID NO: 65 - DNA, 339 bp, synthetic construct
  misc_feature 1..339: note = Description of Artificial Sequence: Synthetic polynucleotide
  source 1..339: mol_type = other DNA
gaggtacagcttcagcagagtggtccagaactcgtcaagcctggggcaagcgttaagatgagttgtaaagcatccggttacacattcagtagctatgttatgcactgggtcaaacagaagcctgggcaggggttggagtggatcggatatataaatccctattcagacgtaactaattataatgaaaagttcaaggggaaagcaaccttgacaagtgaccggtcatctaataccgcatacatggagctgagctcattgacaagtgaggactctgctgtgtattactgtgcccggaactacttcgactggggtaggggcacactggtaactgttagtgca

SEQ ID NO: 66 - protein, 10 aa, synthetic construct
  REGION 1..10: note = Description of Artificial Sequence: Synthetic peptide
GYTFSSYVMH

SEQ ID NO: 67 - protein, 17 aa, synthetic construct
  REGION 1..17: note = Description of Artificial Sequence: Synthetic peptide
YINPYSDVTNYNEKFKG

SEQ ID NO: 68 - protein, 4 aa, synthetic construct
  REGION 1..4: note = Description of Artificial Sequence: Synthetic peptide
NYFD

SEQ ID NO: 69 - protein, 107 aa, synthetic construct
  REGION 1..107: note = Description of Artificial Sequence: Synthetic polypeptide
DIQMTQSPASLSASVGETVTITCRASKNVYSYLAWFQQKQGKSPQLLVYNAKTLAEGVPSRFSGGGSGTQFSLKINSLQPADFGSYYCQHHYNIPFTFGSGTKLEIK

SEQ ID NO: 70 - DNA, 321 bp, synthetic construct
  misc_feature 1..321: note = Description of Artificial Sequence: Synthetic polynucleotide
  source 1..321: mol_type = other DNA
gacatccagatgactcagtctccagcctccctatctgcatctgtgggagaaactgtcaccatcacatgtcgagcaagtaaaaatgtttacagttatttagcatggtttcaacagaaacaggggaaatctcctcagctcctggtctataatgctaaaaccttagcagaaggtgtgccatcaaggttcagtggcgggggatcaggcacacagttttctctgaagatcaacagcctgcagcctgcagattttgggagttattactgtcaacatcattataatattccattcacgttcggctcggggacaaagttggaaataaaa

SEQ ID NO: 71 - DNA, 321 bp, synthetic construct
  misc_feature 1..321: note = Description of Artificial Sequence: Synthetic polynucleotide
  source 1..321: mol_type = other DNA
gacatacaaatgacacaaagtcccgctagtctttcagccagtgttggtgagactgtgacaataacctgtagagctagcaaaaatgtctactcctatctggcttggttccagcagaaacaaggaaagagtcctcagttgctcgtatataatgctaaaactttggcagaaggcgtcccttctcgtttcagtggcggaggaagtgggactcaattctcactgaagatcaatagcctccagcccgccgactttgggagctactattgccaacatcattacaacataccattcacctttggctcaggtactaaactcgaaattaaa

SEQ ID NO: 72 - protein, 11 aa, synthetic construct
  REGION 1..11: note = Description of Artificial Sequence: Synthetic peptide
RASKNVYSYLA

SEQ ID NO: 73 - protein, 7 aa, synthetic construct
  REGION 1..7: note = Description of Artificial Sequence: Synthetic peptide
NAKTLAE

SEQ ID NO: 74 - protein, 9 aa, synthetic construct
  REGION 1..9: note = Description of Artificial Sequence: Synthetic peptide
QHHYNIPFT

SEQ ID NO: 75 - protein, 119 aa, synthetic construct
  REGION 1..119: note = Description of Artificial Sequence: Synthetic polypeptide
QIQLVQSGPELKKPGETVKISCKASGFTFTNYPMHWVKQAPGKGLKWMGWINTYSGVPTYADDFKGRFAFSLETSASTAYLQINNLKNEDMATYFCARGGYDGSREFAYWGQGTLVTVS

SEQ ID NO: 76 - DNA, 357 bp, synthetic construct
  misc_feature 1..357: note = Description of Artificial Sequence: Synthetic polynucleotide
  source 1..357: mol_type = other DNA
cagatccagttggtgcagtctggacctgagctgaagaagcctggagagacagtcaagatctcctgcaaggcttctgggtttaccttcacaaactatccaatgcactgggtgaagcaggctccaggaaagggtttaaagtggatgggctggataaacacctactctggagtgccaacatatgcagatgacttcaagggacggtttgccttctctttggaaacctctgccagcactgcatatttgcagatcaacaacctcaaaaatgaggacatggctacatatttctgtgcaagagggggctacgatggtagcagggagtttgcttactggggccaagggactctggtcactgtctct

SEQ ID NO: 77 - DNA, 357 bp, synthetic construct
  misc_feature 1..357: note = Description of Artificial Sequence: Synthetic polynucleotide
  source 1..357: mol_type = other DNA
cagatacaactcgtccagtcaggtccagagttgaagaaacccggagaaactgtgaagatatcctgtaaagccagcggctttactttcacaaactaccccatgcattgggtgaagcaggcccccggaaaaggactcaaatggatgggatggatcaacacatacagtggggtgcctacttacgcagacgatttcaaaggaaggttcgcatttagcttggaaactagcgcatctacagcatatctccagattaacaatcttaaaaatgaggatatggcaacatacttctgcgctaggggaggttacgatgggagcagggagttcgcttattgggggcaagggactcttgtgactgtaagt

SEQ ID NO: 78 - protein, 10 aa, synthetic construct
  REGION 1..10: note = Description of Artificial Sequence: Synthetic peptide
GFTFTNYPMH

SEQ ID NO: 79 - protein, 17 aa, synthetic construct
  REGION 1..17: note = Description of Artificial Sequence: Synthetic peptide
WINTYSGVPTYADDFKG

SEQ ID NO: 80 - protein, 11 aa, synthetic construct
  REGION 1..11: note = Description of Artificial Sequence: Synthetic peptide
GGYDGSREFAY

SEQ ID NO: 81 - protein, 110 aa, synthetic construct
  REGION 1..110: note = Description of Artificial Sequence: Synthetic polypeptide
DIVLTQSPASLAASLGQRATTSYRASKSVSTSGYSYMHWNQQKPGQPPRLLIYLVSNLESGVPARFSGSGSGTDFTLNIHPVEEEDAATYYCQHIRELTTFGGGTKLEIK

SEQ ID NO: 82 - DNA, 330 bp, synthetic construct
  misc_feature 1..330: note = Description of Artificial Sequence: Synthetic polynucleotide
  source 1..330: mol_type = other DNA
gacattgtgctgacacagtctcctgcttccttagctgcatctctggggcagagggccaccacctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttaccacgttcggaggggggaccaagctggaaataaaa

SEQ ID NO: 83 - DNA, 330 bp, synthetic construct
  misc_feature 1..330: note = Description of Artificial Sequence: Synthetic polynucleotide
  source 1..330: mol_type = other DNA
gacatagtgctcactcagagccctgcatcccttgccgcctccctcggacaacgagctactacaagctaccgggcatcaaagtccgttagcacatcaggatacagctatatgcactggaatcagcaaaagccaggccaaccaccccgtcttctcatctacctcgtaagtaatctggaatcaggcgtgccagcccgattcagtgggtcagggtctgggacagatttcaccctcaacatccatccagtagaggaagaggacgcagcaacatattactgccaacacattagagaacttaccactttcggaggaggaactaaattggagatcaaa

SEQ ID NO: 84 - protein, 15 aa, synthetic construct
  REGION 1..15: note = Description of Artificial Sequence: Synthetic peptide
RASKSVSTSGYSYMH

SEQ ID NO: 85 - protein, 7 aa, synthetic construct
  REGION 1..7: note = Description of Artificial Sequence: Synthetic peptide
LVSNLES

SEQ ID NO: 86 - protein, 7 aa, synthetic construct
  REGION 1..7: note = Description of Artificial Sequence: Synthetic peptide
QHIRELT

SEQ ID NO: 87 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 329 aa, synthetic construct
  REGION 1..329: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 107 aa, synthetic construct [sequence omitted]

SEQ ID NO: 90 - protein, 450 aa, synthetic construct
  REGION 1..450: note = Description of Artificial Sequence: Synthetic polypeptide
QVQLVQSGAEVKKPGASVKVSCKASGYTFTNYGVKWVRQAPGQDLEWMGWINTYTGNPIYAADFKGRVTMTTDTSTSTAFMELRSLRSDDTAVYYCVRFQYGDYRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 1362 bp, synthetic construct
  misc_feature 1..1362: note = Description of Artificial Sequence: Synthetic polynucleotide
  source 1..1362: mol_type = other DNA
tctaccggacaggtgcagttggttcagtctggcgccgaagtgaagaaacctggcgcttctgtgaaggtgtcctgcaaggcctctggctacacctttaccaactacggcgtgaaatgggtccgacaggctcctggacaggatctggaatggatgggctggatcaacacctacaccggcaatcctatctacgccgccgacttcaagggcagagtgaccatgaccaccgacacctctacctccaccgccttcatggaactgcggtccctgagatctgacgacaccgccgtgtactactgcgtgcggtttcagtacggcgactaccggtactttgatgtgtggggccagggcacactggtcaccgtttcttccgcttctaccaagggacccagcgtgttccctctggctccttcctctaaatccacctctggcggaaccgctgctctgggctgtctggtcaaggattacttccctgagcctgtgaccgtgtcctggaactctggtgctctgacatccggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaagctgctggcgctccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggctaagggccagcctcgggaacctcaggtttacaccctgcctccatctcgggaagagatgaccaagaaccaggtgtccctgacctgcctcgtgaagggcttctacccttccgatatcgccgtggaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtccctgtctctgtcccctggcaagtga

SEQ ID NO: 92 - protein, 450 aa, synthetic construct
  REGION 1..450: note = Description of Artificial Sequence: Synthetic polypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - DNA, 1353 bp, synthetic construct
  misc_feature 1..1353: note = Description of Artificial Sequence: Synthetic polynucleotide
  source 1..1353: mol_type = other DNA
gaagtgcagttggtgcagtctggcgccgaagtgaagaaacctggcgcttctgtgaaggtgtcctgcaaggcctctggctacacctttaccaactacggcgtgaaatgggtccgacaggctcctggacaaggcctggaatggatgggctggatcaacacctacaccggcaatcctatctacgccgccgacttcaagggcagagtgaccatgaccaccgacacctctacctccaccgcctacatggaactgcggtccctgagatctgacgacaccgccgtgtactactgcgtgcggtttcagtacggcgactaccggtactttgatgtgtggggccagggcacactggtcaccgtttcttccgcttctaccaagggacccagcgtgttccctctggctccttcctctaaatccacctctggcggaaccgctgctctgggctgtctggtcaaggattacttccctgagcctgtgaccgtgtcctggaattctggtgctctgacatccggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaagctgctggcgctccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggctaagggccagcctcgggaacctcaggtttacaccctgcctccatctcgggaagagatgaccaagaaccaggtgtccctgacctgcctcgtgaagggcttctacccttccgatatcgccgtggaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtccctgtctctgtcccctggcaagtga

SEQ ID NO: 94 - protein, 10 aa, synthetic construct
  REGION 1..10: note = Description of Artificial Sequence: Synthetic peptide
GYTFTNYGVK

SEQ ID NO: 95 - protein, 16 aa, synthetic construct
  REGION 1..16: note = Description of Artificial Sequence: Synthetic peptide
WINTYTGNPIYAADFK

SEQ ID NO: 96 - protein, 11 aa, synthetic construct
  REGION 1..11: note = Description of Artificial Sequence: Synthetic peptide
FQYGDYRYFDV

SEQ ID NO: 97 - protein, 213 aa, synthetic construct
  REGION 1..213: note = Description of Artificial Sequence: Synthetic polypeptide
EIILTQSPATLSLSPGERATLSCRANSGISFINWYQQKPGQAPRLLIYGTANLASGIPARFGGSGSGRDFTLTISSLEPEDFAVYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - DNA, 714 bp, synthetic construct
  misc_feature 1..714: note = Description of Artificial Sequence: Synthetic polynucleotide
  source 1..714: mol_type = other DNA
aagcttgccaccatggaaaccgacacactgctgctgtgggtgctgttgttgtgggtgccaggatctaccggagagatcatcctgacacagagccccgccacattgtctctgagtcctggcgagagagctaccctgtcctgtagagccaactccggcatctccttcatcaactggtatcagcagaagcccggccaggctcctagactgctgatctatggcaccgctaacctggcctctggcatccctgctagatttggcggctctggctctggcagagacttcaccctgaccatctctagcctggaacctgaggacttcgccgtgtactactgccagcagtggtctagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 99 - protein, 213 aa, synthetic construct
  REGION 1..213: note = Description of Artificial Sequence: Synthetic polypeptide
EIVLTQSPATLSLSPGERATLSCRANSGISFINWYQQKPGQAPRLLIYGTANLASGIPARFSGSGSGRDFTLTISSLEPEDFAVYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - DNA, 651 bp, synthetic construct
  misc_feature 1..651: note = Description of Artificial Sequence: Synthetic polynucleotide
  source 1..651: mol_type = other DNA
tctacaggcgagatcgtgctgacccagtctcctgccacattgtctctgagtcctggcgagagagctaccctgtcctgtagagccaactccggcatctccttcatcaactggtatcagcagaagcccggccaggctcctagactgctgatctatggcaccgctaacctggcctctggcatccctgctagattttccggctctggctctggcagagacttcaccctgaccatctctagcctggaacctgaggacttcgccgtgtactactgccagcagtggtctagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 101 - protein, 213 aa, synthetic construct
  REGION 1..213: note = Description of Artificial Sequence: Synthetic polypeptide
EIVLTQSPATLSLSPGERATLSCRAQSGISFINWYQQKPGQAPRLLIYGTANLASGIPARFSGSGSGRDFTLTISSLEPEDFAVYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - DNA, 651 bp, synthetic construct
  misc_feature 1..651: note = Description of Artificial Sequence: Synthetic polynucleotide
  source 1..651: mol_type = other DNA
tctacaggcgagatcgtgctgacccagtctcctgccacattgtctctgagtcctggcgagagagctaccctgtcttgtagagcccagtccggcatctccttcatcaactggtatcagcagaagcccggccaggctcctagactgctgatctatggcaccgctaacctggcctctggcatccctgctagattttccggctctggctctggcagagacttcaccctgaccatctctagcctggaacctgaggacttcgccgtgtactactgccagcagtggtctagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtctggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 103 - protein, 213 aa, synthetic construct
  REGION 1..213: note = Description of Artificial Sequence: Synthetic polypeptide
EIVLTQSPATLSLSPGERATLSCRANSGISFINWYQQKPGQAPRLLIYGTANLASGIPARFSGSGSGRDFTLTISSLEPEDFAVYYCQQYSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - DNA, 651 bp, synthetic construct
  misc_feature 1..651: note = Description of Artificial Sequence: Synthetic polynucleotide
  source 1..651: mol_type = other DNA
tctacaggcgagatcgtgctgacccagtctcctgccacattgtctctgagtcctggcgagagagctaccctgtcctgtagagccaactccggcatctccttcatcaactggtatcagcagaagcccggccaggctcctagactgctgatctatggcaccgctaacctggcctctggcatccctgctagattttccggctctggctctggcagagacttcaccctgaccatctctagcctggaacctgaggacttcgccgtgtactactgccagcagtacagcagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 105 - protein, 213 aa, synthetic construct
  REGION 1..213: note = Description of Artificial Sequence: Synthetic polypeptide
EIVLTQSPATLSLSPGERATLSCRAQSGISFINWYQQKPGQAPRLLIYGTANLASGIPARFSGSGSGRDFTLTISSLEPEDFAVYYCQQYSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - DNA, 651 bp, synthetic construct
  misc_feature 1..651: note = Description of Artificial Sequence: Synthetic polynucleotide
  source 1..651: mol_type = other DNA
tctacaggcgagatcgtgctgacccagtctcctgccacattgtctctgagtcctggcgagagagctaccctgtcttgtagagcccagtccggcatctccttcatcaactggtatcagcagaagcccggccaggctcctagactgctgatctatggcaccgctaacctggcctctggcatccctgctagattttccggctctggctctggcagagacttcaccctgaccatctctagcctggaacctgaggacttcgccgtgtactactgccagcagtacagcagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtctggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 107 - protein, 10 aa, synthetic construct
  REGION 1..10: note = Description of Artificial Sequence: Synthetic peptide
RANSGISFIN

SEQ ID NO: 108 - protein, 10 aa, synthetic construct
  REGION 1..10: note = Description of Artificial Sequence: Synthetic peptide
RAQSGISFIN

SEQ ID NO: 109 - protein, 7 aa, synthetic construct
  REGION 1..7: note = Description of Artificial Sequence: Synthetic peptide
GTANLAS

SEQ ID NO: 110 - protein, 9 aa, synthetic construct
  REGION 1..9: note = Description of Artificial Sequence: Synthetic peptide
QQWSSNPLT

SEQ ID NO: 111 - protein, 9 aa, synthetic construct
  REGION 1..9: note = Description of Artificial Sequence: Synthetic peptide
QQYSSNPLT

SEQ ID NO: 112 - protein, 453 aa, synthetic construct
  REGION 1..453: note = Description of Artificial Sequence: Synthetic polypeptide
EVQLVESGGGLVKPGGSLRLSCAASGFTFSSFAMSWVRQAPGKGLEWVSTISSGSIYIYYTDGVKGRFTISRDNAKNSLYLQMNSLRAEDTAVYYCARRGIYYGYDGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - DNA, 1362 bp, synthetic construct
  misc_feature 1..1362: note = Description of Artificial Sequence: Synthetic polynucleotide
  source 1..1362: mol_type = other DNA
gaggtgcagctggttgaatctggcggaggacttgtgaagcctggcggctctctgagactgtcttgtgccgcctctggcttcaccttctccagctttgccatgtcctgggtccgacaggctcctggcaaaggactggaatgggtgtccaccatctcctccggctccatctacatctactacaccgacggcgtgaagggcagattcaccatcagcagagacaacgccaagaactccctgtacctgcagatgaacagcctgagagccgaggacaccgccgtgtactattgtgccagacggggcatctactatggctacgacggctacgctatggactattggggacagggcacactggtcaccgtgtcctctgcttctaccaagggacccagcgtgttccctctggctccttcctctaaatccacctctggcggaaccgctgctctgggctgtctggtcaaggattacttccctgagcctgtgaccgtgtcctggaactctggtgctctgacatccggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaagctgctggcgctccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggctaagggccagcctcgggaacctcaggtttacaccctgcctccatctcgggaagagatgaccaagaaccaggtgtccctgacctgcctcgtgaagggcttctacccttccgatatcgccgtggaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtccctgtctctgtcccctggcaagtga

SEQ ID NO: 114 - protein, 453 aa, synthetic construct
  REGION 1..453: note = Description of Artificial Sequence: Synthetic polypeptide
EVQLVESGGGLVKPGGSLRLSCAASGFTFSSFAMSWVRQAPGKGLEWVSTISSGSIYIYYTDSVKGRFTISRDNAKNSLYLQMNSLRAEDTAVYYCARRGIYYGYEGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 10 aa, synthetic construct
  REGION 1..10: note = Description of Artificial Sequence: Synthetic peptide
GFTFSSFAMS

SEQ ID NO: 116 - protein, 17 aa, synthetic construct
  REGION 1..17: note = Description of Artificial Sequence: Synthetic peptide
TISSGSIYIYYTDGVKG

SEQ ID NO: 117 - protein, 17 aa, synthetic construct
  REGION 1..17: note = Description of Artificial Sequence: Synthetic peptide
TISSGSIYIYYTDSVKG

SEQ ID NO: 118 - protein, 14 aa, synthetic construct
  REGION 1..14: note = Description of Artificial Sequence: Synthetic peptide
RGIYYGYDGYAMDY

SEQ ID NO: 119 - protein, 14 aa, synthetic construct
  REGION 1..14: note = Description of Artificial Sequence: Synthetic peptide
RGIYYGYEGYAMDY

SEQ ID NO: 120 - protein, 14 aa, synthetic construct
  REGION 1..14: note = Description of Artificial Sequence: Synthetic peptide
RGIYYGYSGYAMDY

SEQ ID NO: 121 - protein, 14 aa, synthetic construct
  REGION 1..14: note = Description of Artificial Sequence: Synthetic peptide
RGIYYGYAGYAMDY

SEQ ID NO: 122 - protein, 219 aa, synthetic construct
  REGION 1..219: note = Description of Artificial Sequence: Synthetic polypeptide
AIVLTQSPGTLSLSPGERATLSCRSSQSLVHSNGNTYLHWYMQKPGQAPRVLIYKVSNRFSGIPDRFSGSGSGTDFTLTISRLEPDDFAIYYCSQSTHIPLAFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - DNA, 660 bp, synthetic construct
  misc_feature 1..660: note = Description of Artificial Sequence: Synthetic polynucleotide
  source 1..660: mol_type = other DNA
gccatcgtgttgacccagtctccaggcacattgtctctgagccctggcgagagagctaccctgtcctgcagatcttctcagtccctggtgcactccaacggcaacacctacctgcactggtacatgcagaagcccggacaggctcccagagtgctgatctacaaggtgtccaaccggttctctggcatccccgacagattttccggctctggctctggcaccgacttcaccctgaccatctctagactggaacccgacgacttcgccatctactactgctcccagtccacacacatccctctggcttttggccagggcaccaagctggaaatcaagagaaccgtggccgctccttccgtgttcatcttcccaccatctgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggcctgtctagccctgtgaccaagtctttcaaccggggcgagtgttga

SEQ ID NO: 124 - protein, 16 aa, synthetic construct
  REGION 1..16: note = Description of Artificial Sequence: Synthetic peptide
RSSQSLVHSNGNTYLH

SEQ ID NO: 125 - protein, 16 aa, synthetic construct
  REGION 1..16: note = Description of Artificial Sequence: Synthetic peptide
RSSQSLVHSSGNTYLH

SEQ ID NO: 126 - protein, 16 aa, synthetic construct
  REGION 1..16: note = Description of Artificial Sequence: Synthetic peptide
RSSQSLVHSTGNTYLH

SEQ ID NO: 127 - protein, 16 aa, synthetic construct
  REGION 1..16: note = Description of Artificial Sequence: Synthetic peptide
RSSQSLVHSQGNTYLH

SEQ ID NO: 128 - protein, 7 aa, synthetic construct
  REGION 1..7: note = Description of Artificial Sequence: Synthetic peptide
KVSNRFS

SEQ ID NO: 129 - protein, 9 aa, synthetic construct
  REGION 1..9: note = Description of Artificial Sequence: Synthetic peptide
SQSTHIPLA

SEQ ID NO: 130 - protein, 120 aa, synthetic construct
  REGION 1..120: note = Description of Artificial Sequence: Synthetic polypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSS

SEQ ID NO: 131 - protein, 106 aa, synthetic construct
  REGION 1..106: note = Description of Artificial Sequence: Synthetic polypeptide
EIVLTQSPATLSLSPGERATLSCRAQSGISFINWYQQKPGQAPRLLIYGTANLASGIPARFSGSGSGRDFTLTISSLEPEDFAVYYCQQWSSNPLTFGGGTKVEIK

SEQ ID NO: 132 - protein, 120 aa, synthetic construct
  REGION 1..120: note = Description of Artificial Sequence: Synthetic polypeptide
QVQLVQSGAEVKKPGASVKVSCKASGYTFTNYGVKWVRQAPGQDLEWMGWINTYTGNPIYAADFKGRVTMTTDTSTSTAFMELRSLRSDDTAVYYCVRFQYGDYRYFDVWGQGTLVTVSS

SEQ ID NO: 133 - protein, 106 aa, synthetic construct
  REGION 1..106: note = Description of Artificial Sequence: Synthetic polypeptide
EIILTQSPATLSLSPGERATLSCRANSGISFINWYQQKPGQAPRLLIYGTANLASGIPARFGGSGSGRDFTLTISSLEPEDFAVYYCQQWSSNPLTFGGGTKVEIK

SEQ ID NO: 134 - skipped

SEQ ID NO: 135 - protein, 330 aa, synthetic construct; identical to SEQ ID NO: 87
  REGION 1..330: note = Description of Artificial Sequence: Synthetic polypeptide

SEQ ID NO: 136 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYCSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYCSTYRVVSVLTVLHQDWLNGKEYKCKVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 330 aa, synthetic construct; identical to SEQ ID NO: 136
  REGION 1..330: note = Description of Artificial Sequence: Synthetic polypeptide

SEQ ID NO: 142 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYCSTYRVVSVLTVLHQDWLNGKEYKCAVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 330 aa, synthetic construct; identical to SEQ ID NO: 138
  REGION 1..330: note = Description of Artificial Sequence: Synthetic polypeptide

SEQ ID NO: 144 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVAVSHEDPEVKFNWYVDGVEVHNAKTKPREEQYCSTYRVVSVLTVLHQDWLNGKEYKCAVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 332 aa, synthetic construct
  REGION 1..332: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYCSTYRVVSVLTVLHQDWLNGKEYKCAVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKKP

SEQ ID NO: 146 - protein, 447 aa, synthetic construct
  REGION 1..447: note = Description of Artificial Sequence: Synthetic polypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 147 - protein, 447 aa, synthetic construct
  REGION 1..447: note = Description of Artificial Sequence: Synthetic polypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 148 - protein, 447 aa, synthetic construct
  REGION 1..447: note = Description of Artificial Sequence: Synthetic polypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFCSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 149 - protein, 447 aa, synthetic construct
  REGION 1..447: note = Description of Artificial Sequence: Synthetic polypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAEGGPSVFLFPPKPKDTLMISRTPEVTCVVVDVSQEDPEVQFNWYVDGVEVHNAKTKPREEQFCSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 150 - protein, 449 aa, synthetic construct
  REGION 1..449: note = Description of Artificial Sequence: Synthetic polypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFCSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKKP

SEQ ID NO: 151 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 153 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 154 - protein, 447 aa, synthetic construct
  REGION 1..447: note = Description of Artificial Sequence: Synthetic polypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 155 - protein, 447 aa, synthetic construct
  REGION 1..447: note = Description of Artificial Sequence: Synthetic polypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 156 - protein, 450 aa, synthetic construct
  REGION 1..450: note = Description of Artificial Sequence: Synthetic polypeptide
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 5 aa, synthetic construct
  REGION 1..5: note = Description of Artificial Sequence: Synthetic peptide
SSVSF

SEQ ID NO: 158 - skipped

SEQ ID NO: 159 - protein, 6 aa, synthetic construct
  REGION 1..6: note = Description of Artificial Sequence: Synthetic peptide
WNSNPL

SEQ ID NO: 160 - protein, 7 aa, synthetic construct
  REGION 1..7: note = Description of Artificial Sequence: Synthetic peptide
GYTFTNY

SEQ ID NO: 161 - protein, 4 aa, synthetic construct
  REGION 1..4: note = Description of Artificial Sequence: Synthetic peptide
TYTG

SEQ ID NO: 162 - protein, 10 aa, synthetic construct
  REGION 1..10: note = Description of Artificial Sequence: Synthetic peptide
FSYGNSRYAD

SEQ ID NO: 163 - protein, 106 aa, synthetic construct
  REGION 1..106: note = Description of Artificial Sequence: Synthetic polypeptide
QIVLSQSPAILSASPGEKVTLTCRASSVSFMNWYQQKPGSSPKPWIYATSNLASGVPGRFSGSGSGTSYSLAISRVEAEDAATYYCQQWNSNPLTFGAGTKLELKR

SEQ ID NO: 164 - protein, 120 aa, synthetic construct
  REGION 1..120: note = Description of Artificial Sequence: Synthetic polypeptide
QIQLVQSGPELKKPGETVKISCKASGYTFTNYGMNWVKQAPGKGLKWMGWINTYTGQPTYADDFKGRFAFSLETSAYTAYLQINNLKNEDMATYFCARFSYGNSRYADYWGQGTTLTVSS

SEQ ID NO: 165 - protein, 202 aa, Homo sapiens
MCYGKCARCIGHSLVGLALLCIAANILLYFPNGETKYASENHLSRFVWFFSGIVGGGLLMLLPAFVFIGLEQDDCCGCCGHENCGKRCAMLSSVLAALIGIAGSGYCVIVAALGLAEGPLCLDSLGQWNYTFASTEGQYLLDTSTWSECTEPKHIVEWNVSLFSILLALGGIEFILCLIQVINGVLGGICGFCCSHQQQYDC

SEQ ID NO: 166 - protein, 48 aa, Homo sapiens
EGPLCLDSLGQWNYTFASTEGQYLLDTSTWSECTEPKHIVEWNVSLFS

SEQ ID NO: 167 - protein, 11 aa, synthetic construct
  REGION 1..11: note = Description of Artificial Sequence: Synthetic peptide
RRRPRPPYLPR

SEQ ID NO: 168 - protein, 4 aa, synthetic construct
  REGION 1..4: note = Description of Artificial Sequence: Synthetic peptide
RGDS